(19) United States
(12) Patent Application Publication
(10) Pub. No.: US 2022/0127621 A1
(43) Pub. Date: Apr. 28, 2022

(54) FUSION PROTEINS AND FUSION RIBONUCLEIC ACIDS FOR TRACKING AND MANIPULATING CELLULAR RNA

(71) Applicant: The Regents of the University of California, Oakland, CA (US)

(72) Inventors: Eugene Yeo, La Jolla, CA (US); Frederick Tan, Encinitas, CA (US)

(21) Appl. No.: 17/049,198

(22) PCT Filed: Apr. 22, 2019
(86) PCT No.: PCT/US19/28580

§ 371 Date: Oct. 20, 2020

Related U.S. Application Data
(60) Provisional application No. 62/660,849, filed on Apr. 20, 2018.
(60) Provisional application No. 62/665,860, filed on May 2, 2018.

(51) Int. Cl. C12N 15/62; C12N 9/22; C12N 15/11; C07K 14/47

(52) U.S. Cl. CPC C12N 15/62; C12N 9/22; C12N 15/11; C07K 14/4702; C12N 2310/3519; C12N 2800/80; C07K 2319/00; C12N 2840/203; C12N 2310/20

(57) ABSTRACT

Described herein are compositions, systems, methods, and kits utilizing RNA binding protein fusions, such as CRISPR-Cas protein fusions comprising a guide nucleotide sequence-programmable RNA binding protein, and a translation modifier protein. Also, described herein are compositions, systems, methods, and kits utilizing CRISPR-Cas associated RNA fusions comprising a guide nucleotide sequence-programmable RNA and an internal ribosome entry site (IRES). The compositions, systems, methods, and kits described herein are useful to upregulate or downregulate mRNA translation.

### CROSS-REFERENCE TO RELATED APPLICATIONS

This application claims priority to U.S. Provisional Application Ser. No. 62/660,849, filed on Apr. 20, 2018, and U.S. Provisional Application Ser. No. 62/665,860, filed on May 2, 2018, both of which are herein incorporated by reference in their entireties.

### STATEMENT OF GOVERNMENT SUPPORT

This invention was made with government support under Grant No. NS103172, awarded by the National Institutes of Health. The U.S. Government has certain rights to the invention.

### SEQUENCE LISTING

The instant application contains a Sequence Listing which has been submitted electronically in ASCII format and is hereby incorporated by reference in its entirety. Said ASCII copy, created on May 6, 2021, is named 15670-0310US1_SL.txt.

### BACKGROUND

There are currently no consolidated systems that can both upregulate and downregulate the translation of specific messenger RNA (mRNA) targets. Known methods to achieve targeted downregulation include anti-sense oligonucleotides (ASO) and short interfering RNAs (siRNA). However, both of these technologies function to destabilize a messenger RNA target and downregulate translation, rather than upregulate translation. There are few known methods to increase mRNA translation and these methods are not well characterized. As such, there is a need to provide compositions and methods for recruiting translational pre-initation complexes in trans and thereby control translation in cells and in gene therapy techniques.

### SUMMARY

This disclosure relates to compositions, systems, methods, and kits to control mRNA translation in cells using CRISPR-Cas protein fusions. These compositions, methods, systems, and kits utilize the RNA targeting abilities of CRISPR-Cas systems, which use a guide RNA to provide a simple and rapidly programmable system for recognizing RNA molecules in cells. These compositions, methods, systems, and kits further utilize the ability of CRISPR-Cas systems to bind target messenger RNA to initiate translation in trans by fusing a ribonucleic acid sequence, that recruits translational pre-initiation complexes, to the single stranded guide RNA and thereby to the bound messenger RNA. CRISPR-Cas systems also have neutral effects on messenger RNA stability, which makes any measured change to protein expression a function of the fused protein effector. The compositions, systems, methods, and kits described herein provide high utility and versatility when compared to other compositions, methods, systems, and kits for controlling mRNA expression.

In one aspect a composition comprising one or more polynucleotides encoding: (i) a guide nucleotide sequence-programmable RNA binding protein; and (ii) a translation modifier protein.

In some embodiments, the guide nucleotide sequence-programmable RNA binding protein comprises at least one of Cas9, modified Cas9, Cas13a, Cas13b, CasRX/Cas13d, CasM, and a biological equivalent of each thereof. In some embodiments, the guide nucleotide sequence-programmable RNA binding protein comprises at least one of Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9). In some embodiments, the guide nucleotide sequence-programmable RNA binding protein is nuclease inactive.

In some embodiments, the translation modifier protein is at least one of eukaryotic translation initiation factor 4E (EIF4E) (SEQ ID NO: 52-59), eukaryotic translation initiation factor 4E-binding protein (EIF4E-BP1) (SEQ ID NO: 61-62), ubiquitin-associated protein 2-like (UBAP2L) (SEQ ID NO: 64-71), and a biological equivalent of each thereof. In some embodiments, the translation modifier protein is encoded by a polynucleotide having a sequence comprising all or part of at least one of SEQ ID NO: 52-55, SEQ ID NO: 61, SEQ ID NO: 64-67, SEQ ID NO: 94-193, SEQ ID NO: 285, and a biological equivalent of each thereof. In some embodiments, wherein the translation modifier protein has an amino acid sequence comprising all or part of at least one of SEQ ID NO: 56-59, SEQ ID NO: 62, SEQ ID NO: 68-71 and a biological equivalent of each thereof.

In some embodiments, the composition further comprises a linker. In some embodiments, the linker is a peptide linker. In some embodiments, the peptide linker comprises one or more repeats of the tri-peptide GGS. In some embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the guide nucleotide sequence-programmable RNA binding protein is bound to a guide RNA (gRNA), a crisprRNA (crRNA), or a trans-activating crRNA (tracrRNA). In some embodiments, one or more kinase phosphorylation domains of the translation modifier protein is mutated.

In some embodiments, the composition further comprises a vector. In some embodiments, the vector is an adenoviral vector, an adeno-associated viral vector, or a lentiviral vector. In some embodiments, the vector further comprises an expression control element. In some embodiments the vector further comprises a selectable marker. In some embodiments, the vector further comprises a polynucleotide encoding either (i) a gRNA, or (ii) a crRNA and a tracrRNA. In some embodiments, the gRNA or the crRNA comprises a nucleotide sequence complementary to a target RNA.

In one aspect, a fusion protein comprising: (i) a guide nucleotide sequence-programmable RNA binding protein; and (i) a translation modifier protein.

In some embodiments, a system for post-transcriptional gene regulation, the system comprising: (i) a fusion protein; and (ii) a gRNA; or (iii) a crRNA and a tracrRNA; wherein the gRNA or the crRNA comprises a sequence complementary to a target mRNA.

In some embodiments, a method for post-transcriptionally regulating gene expression, the method comprising contacting a target mRNA with a fusion protein, wherein the guide nucleotide sequence-programmable RNA binding protein binds a gRNA or a crRNA that hybridizes to a region of the target RNA.

In one aspect, a fusion RNA comprising: (i) a guide nucleotide sequence-programmable RNA; and (ii) one or more internal ribosome entry sites (IRES). In some embodiments, the guide nucleotide sequence-programmable RNA is a guide RNA (gRNA) or a crisprRNA (crRNA). In some embodiments, the guide nucleotide sequence-programmable RNA is derived from a guide RNA scaffold from Steptococcus pyogenes, Staphylococcus aureus, Francisella novicida, Neisseria meningitidis, Streptococcus thermophilus, or Brevibacillus laterosporus. In some embodiments, the IRES is at least one of a Poliovirus IRES, Rhinovirus IRES, Encephalomyocarditis virus IRES (EMCV-IRES), Picornavirus IRES, Foot-and-mouth disease virus IRES (FMDV-IRES), Aphthovirus IRES, Kaposi's sarcoma-associated herpesvirus IRES (KSHV-IRES), Hepatitis A IRES, Hepatitis C IRES, Classical swine fever virus IRES, Pestivirus IRES, Bovine viral diarrhea virus IRES, Friend murine leukemia IRES, Moloney murine leukemia IRES (MMLV-IRES), Rous sarcoma virus IRES, Human immunodeficiency virus IRES (HIV-IRES), Plautia stali intestine virus IRES, Cripavirus IRES, Cricket paralysis virus IRES, Triatoma virus IRES, Rhopalosiphum padi virus IRES, Marek's disease virus IRES, Fibroblast growth factor (FGF-1 IRES and FGF-2 IRES), Platelet-derived growth factor B (PDGF/c-sis IRES), Vascular endothelial growth factor (VEGF IRES), and an Insulin-like growth factor 2 (IGF-II IRES).

In some embodiments, a method for post-transcriptionally regulating gene expression, the method comprising contacting a target mRNA with a fusion RNA and a guide nucleotide sequence-programmable RNA binding protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularly in the appended claims.

A better understanding of the features and advantages can be obtained by reference to the following detailed description that sets forth illustrative embodiments and accompanying drawings (“Figure” and “FIG.” herein), of which:

FIG. 1 depicts a nuclease dead Cas9 (dCas9) fused to a modified EIF4E protein. The schematic shows dCas9-EIF4E targeting the 3′UTR of a representative target transcript mRNA. Modified EIF4E facilitates transcript circularization and the recruitment of EIF4G and ribosomal pre-initiation complexes.

FIG. 2 depicts dCas9 fused to a modified EIF4E-BP1. The schematic shows dCas9-EIF4E-BP1 targeting the 3′UTR of a representative target transcript. Modified EIF4E-BP1 facilitates transcript mRNA circularization, and prevents the disengagement of EIF4E-BP1 from EIF4E. Constitutive binding prevents the recruitment of EIF4G and ribosomal pre-initiation complexes.

FIGS. 3A-3C depict schematics of DNA constructs for (FIG. 3A) Effector and (FIG. 3B) Reporter constructs used for characterization studies. Cas9-EIF4E expression level is correlated to a co-expressed CFP fluorophore on the Effector. YFP and RFP are co-expressed from different promoters on the Reporter. However, only YFP messenger RNA carries a target site (LUC target site) that is complementary to the spacer of the single guide RNA (sgRNA). (FIG. 3C) Results: (i) Heatmap showing how the fold change in YFP/RFP ratio relate to Reporter (x-axis) and Effector (y-axis) DNA construct levels. Datapoints used for the heatmap represent the average fluorescence of single cells that fall within defined bins. (ii) Same data as presented in (i), but with YFP/RFP ratio plotted as third variable (z-axis). (iii) Residuals for datapoints used to generate heatmap.

FIGS. 4A-4C depict schematics of DNA constructs for (FIG. 4A) Effector and (FIG. 4B) Reporter constructs used for characterization studies. Cas9-EIF4E-BP1 expression level is correlated to a co-expressed CFP fluorophore on the Effector. YFP and RFP are coexpressed from different promoters on the Reporter. However, only YFP messenger RNA carries a target site (LUC target site) that is complementary to the spacer of the single guide RNA (sgRNA). (FIG. 4C) Results: (i) Heatmap showing how the fold change in YFP/RFP ratio relate to Reporter (x-axis) and Effector (y-axis) DNA construct levels. Datapoints used for the heatmap represent the average fluorescence of single cells that fall within defined bins. (ii) Same data as presented in (i), but with YFP/RFP ratio plotted as third variable (z-axis). (iii) Residuals for datapoints used to generate heatmap.

FIG. 5 depicts a schematic of an exemplary system for modulating target mRNA translation. IRES can be used to nucleate translation initiation factors on a target messenger RNA. CRISPR/Cas proteins co-localize IRES elements to target messenger RNAs when they are fused 3′ to the targeting guide. Type I and Type II IRES elements employ a scanning mechanism to find appropriate start codons (AUG=green rectangles). Structural features of IRES stabilize pre-initiation complex on start codons (AUG), thus initiating translation in trans.

FIGS. 6A-6C show design of exemplary effector and reporter systems to test IRES activity in trans for dCas9 and dCas13b. Schematic of DNA constructs used to characterize regulation by (FIG. 6A) dCas9 and (FIG. 6B) dCas13b. Shown are exemplary (i) Effector and (ii) Reporter constructs for each CRISPR/Cas system. dCas expression level is correlated to a co-expressed CFP fluorophore on the Effector. YFP and RFP are co-expressed from different promoters on the Reporter. However, only YFP messenger RNA is targeted for post-transcriptional regulation. As a result, post-transcriptional regulation can be measured as changes in YFP expression relative to RFP expression. (FIG. 6C) Translation may prefer specific start codons (green boxes) which are found on any of three potential reading frames (+0, +1, +2). Expression from +0 reading frame: FLAG peptide expression can be profiled using ELISA or mass spectrometry. Expression from +1 reading frame: C-terminal HA tag labels all translated protein isoforms, and can be profiled using Western blot. Expression from +2 reading frame: No specific method to monitor expression of this frame. Below are the locations targeted by CRISPR guides (20nt width for dCas9, 30nt width for dCas13b).

FIGS. 7A-7B show Cas9-mediated translational initiation in trans using EMCV IRES to enhance protein production. (FIG. 7A) Location of spacers targeted by dCas9, which are used to profile changes in the expression of a 30.5 kDa protein product. (FIG. 7B) Using densitometry calculations via Western blot, changes in HA-tag signal vs. Cherry signal after dCas9 targeting by each of the spacers are plotted relative to observations using a non-targeting (NT) sgRNA-IRES.

FIG. 8 depicts transgene expression reporter constructs. RCas9 is expressed from a tetracycline responsive element (TRE) reporter. A constitutive promoter drives a polycistronic transcript containing puromycin N-acetyl transferase (Puro) and the reverse tetracycline (tet)-controlled transactivator (rtTA) separated by a P2A self-cleaving peptide, as well as CFP fused to a nuclear localization signal (NLS) preceded by an internal ribosome entry site (IRES). A second construct drives rCas9 fused to UBAP2L in the same plasmid background. rCas9 and rCas9-UBAP2L constructs were integrated into the genome at random copy number to establish stably-expressing lines. A third reporter construct harbors a U6 promoter driven single guide (sg)RNA targeting the indicated sites in the YFP reporter, which contains a YFP fused to histone H2B driven by a tet-inducible promoter, and NLS-fused RFP driving by the EF1α promoter.

FIG. 9 depicts quantitative fluorescence-activated cell sorting (FACS)-based reporter assay of the reporters transiently transfected into rCas9-UBAP2L expressing cells, normalized to rCas9 expressing cells, on each targeting site. Error bars denote standard deviation (SD) from n=2,000 rCas9-UBAP2L and n=2,000 rCas9 expressing cells per site.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or variant serotypes, e.g. AAV-DJ. The AAV structural particle is composed of 60 protein molecules made up of VP1, VP2, and VP3. Each particle contains approximately 5 VP1 proteins, 5 VP2 proteins and 50 VP3 proteins ordered into an icosahedral structure.

As used herein, the “administration” of an agent (e.g., a fusion RNA, viral particle, vector, polynucleotide, cell, population of cells, composition, or pharmaceutical composition) to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another.

Also as used herein, “and/or” refers to and encompasses any and all possible combinations of one or more of the associated listed items, as well as the lack of combinations when interpreted in the alternative (“or”).

The term “guide nucleotide sequence-programmable RNA” refers to a CRISPR-associated RNA comprising a sequence that is complementary and/or homologous to a target nucleic acid. Non-limiting examples of guide nucleotide sequence-programmable RNAs include single guide RNA (sgRNA) and crRNA, and biological equivalents thereof. In some embodiments, the guide nucleotide sequence-programmable RNA is synthetic. In some embodiments, a “scaffold” RNA refers to a guide nucleotide sequence-programmable RNA wherein the sequence that is complementary and/or homologous to a target nucleic acid in the fusion RNA can be modified.

Guide RNAs (gRNAs) of the disclosure may comprise a spacer sequence and a scaffolding sequence. In some embodiments, a guide RNA is a single guide RNA (sgRNA) comprising a contiguous spacer sequence and scaffolding sequence. The terms guide RNA (gRNA) and single guide RNA (sgRNA) are used interchangeably throughout the disclosure. In some embodiments, the spacer sequence and the scaffolding sequence are not contiguous. In some embodiments, a scaffold sequence comprises a “direct repeat” (DR) sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding Cas protein if the sequence of the associated CRISPR locus is known. In some embodiments, a sequence encoding a guide RNA or single guide RNA of the disclosure comprises or consists of a spacer sequence and a scaffolding sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50, or any number of nucleotides in between. In some embodiments, the linker sequence may comprise at least 1, 2, 3, 4, 5, 6, 7, 8, 9, 10, 15, 20, 25, 30, 35, 40, 45, 50, or any number of nucleotides in between.

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. Spacer sequences of the disclosure may comprise a CRISPR RNA (crRNA). Spacer sequences of the disclosure comprise or consist of a sequence having sufficient complementarity to a target sequence of an RNA molecule to bind selectively to the target sequence. Upon binding to a target sequence of an RNA molecule, the spacer sequence may guide one or more of a scaffolding sequence and a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence. Scaffolding sequences of the disclosure bind the RNA-binding protein of the disclosure.

Scaffolding sequences of the disclosure may comprise a trans acting RNA (tracrRNA). Scaffolding sequences of the disclosure comprise or consist of a sequence having sufficient complementarity to a target sequence of an RNA molecule to bind selectively to the target sequence. Upon binding to a target sequence of an RNA molecule, the scaffolding sequence may guide a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence. Alternatively or in addition, in some embodiments, scaffolding sequences of the disclosure comprise or consist of a sequence that binds to a first RNA binding protein or a second RNA binding protein of a fusion protein of the disclosure. In some embodiments, scaffolding sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, scaffolding sequences of the disclosure comprise at least one secondary structure or at least one tertiary structure. In some embodiments, scaffolding sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a spacer sequence of the disclosure comprises or consists of 15, 16, 17, 18, 19, 20, 21, 22, 23, 24, 25, 26, 27, 28, 29, or 30 nucleotides. In some embodiments, the spacer sequence of the disclosure comprises or consists of 20 nucleotides. In some embodiments, the spacer sequence of the disclosure comprises or consists of 21 nucleotides. In some embodiments, a scaffold sequence of the disclosure comprises or consists of between 10 and 100 nucleotides, inclusive of the endpoints. In some embodiments, a scaffold sequence of the disclosure comprises or consists of 30, 35, 40, 45, 50, 55, 60, 65, 70, 76, 80, 87, 90, 95, 100, or any number of nucleotides in between. In some embodiments, the scaffold sequence of the disclosure comprises or consists of between 85 and 95 nucleotides, inclusive of the endpoints. In some embodiments, the scaffold sequence of the disclosure comprises or consists of 85 nucleotides. In some embodiments, the scaffold sequence of the disclosure comprises or consists of 90 nucleotides. In some embodiments, the scaffold sequence of the disclosure comprises or consists of 93 nucleotides.

In some embodiments of the compositions of the disclosure, a guide RNA or a portion thereof does not comprise a nuclear localization sequence (NLS).

In some embodiments of the compositions of the disclosure, a guide RNA, or a portion thereof does not comprise a sequence complementary to a protospacer adjacent motif (PAM).

In some embodiments, therapeutic or pharmaceutical compositions of the disclosure do not comprise a PAMmer oligonucleotide. In other embodiments, optionally, non-therapeutic or non-pharmaceutical compositions may comprise a PAMmer oligonucleotide.

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the RNA binding protein may comprise a sequence isolated or derived from a Cas protein, such as, without limitation, a Cas9, Cas13b, or Cas13d protein. In some embodiments, including those wherein a guide RNA or a portion thereof comprises a sequence complementary to a PFS, the RNA binding protein may comprise a sequence encoding a Cas protein, such as, without limitation, a Cas9, Cas13b, or Cas13d protein, or an RNA-binding portion thereof. In some embodiments, the guide RNA or a portion thereof does not comprise a sequence complementary to a PFS.

In some embodiments, a sequence encoding a guide RNA of the disclosure further comprises a sequence encoding a promoter to drive expression of the guide RNA. In some embodiments, a vector comprising a sequence encoding a guide RNA of the disclosure further comprises a sequence encoding a promoter to drive expression of the guide RNA. In some embodiments, a sequence encoding a promoter to drive expression of the guide RNA comprises a sequence encoding a constitutive promoter. In some embodiments, a sequence encoding a promoter to drive expression of the guide RNA comprises a sequence encoding an inducible promoter. In some embodiments, a sequence encoding a promoter to drive expression of the guide RNA comprises a sequence encoding a hybrid or a recombinant promoter. In some embodiments, a sequence encoding a promoter to drive expression of the guide RNA comprises a sequence encoding a promoter capable of expressing the guide RNA in a mammalian cell. In some embodiments, a sequence encoding a promoter to drive expression of the guide RNA comprises a sequence encoding a promoter capable of expressing the guide RNA in a human cell. In some embodiments, a sequence encoding a promoter to drive expression of the guide RNA comprises a sequence encoding a promoter capable of expressing the guide RNA and restricting the guide RNA to the nucleus of the cell. In some embodiments, a sequence encoding a promoter to drive expression of the guide RNA comprises a sequence encoding a human RNA polymerase promoter or a sequence isolated or derived from a sequence encoding a human RNA polymerase promoter. In some embodiments, a sequence encoding a promoter to drive expression of the guide RNA comprises a sequence encoding a U6 promoter or a sequence isolated or derived from a sequence encoding a U6 promoter. In some embodiments, a sequence encoding a promoter to drive expression of the guide RNA comprises a sequence encoding a human tRNA promoter or a sequence isolated or derived from a sequence encoding a human tRNA promoter. In some embodiments, a sequence encoding a promoter to drive expression of the guide RNA comprises a sequence encoding a human valine tRNA promoter or a sequence isolated or derived from a sequence encoding a human valine tRNA promoter.

In some embodiments of the compositions of the disclosure, a sequence encoding a promoter to drive expression of the guide RNA further comprises a regulatory element. In some embodiments, a vector comprising a sequence encoding a promoter to drive expression of the guide RNA further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a sequence encoding a guide RNA, a sequence encoding a promoter to drive expression of the guide RNA and a sequence encoding a regulatory element. In some embodiments of the compositions of the disclosure, the vector further comprises a sequence encoding a fusion protein of the disclosure.

The term “guide nucleotide sequence-programmable RNA binding protein” refers to a CRISPR-associated, RNA-guided endonuclease such as, without limitation, Type II CRISPR Cas proteins such as, e.g., streptococcus pyogenes Cas9 (spCas9) and orthologs and biological equivalents thereof. Exemplary Cas9 proteins of the disclosure may be isolated or derived from any species, including, but not limited to, a bacteria or an archaea. Exemplary Cas9 proteins of the disclosure may be isolated or derived from any species, including, but not limited to, Streptococcus pyogenes, Haloferax mediteranii, Mycobacterium tuberculosis, Francisella tularensis subsp. novicida, Pasteurella multocida, Neisseria meningitidis, Campylobacter jejune, Streptococcus thermophilus, Campylobacter lari CF89-12, Mycoplasma gallisepticum str. F, Nitratifractor salsuginis str. DSM 16511, Parvibaculum lavamentivorans, Roseburia intestinalis, Neisseria cinerea, a Gluconacetobacter diazotrophicus, an Azospirillum B510, a Sphaerochaeta globus str. Buddy, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila str. Paris, Sutterella wadsworthensis, Corynebacter diphtherias, Streptococcus aureus, and Francisella novicida.

Biological equivalents of Cas9 include but are not limited to Type V systems such as a Cpfl protein, and Type VI CRISPR systems, such as Cas13a, C2c2, Cas13b, CasRx, Cas13d, and CasM which target RNA rather than DNA. A guide nucleotide sequence-programmable RNA binding protein may refer to an endonuclease that causes breaks or nicks in RNA as well as other variations such as nuclease-inactive Cas proteins such as, e.g., dead Cas9 or dCas9, which lack endonuclease activity. A guide nucleotide sequence-programmable RNA binding protein may also refer to a “split” protein in which the protein is split into two halves (e.g., C-Cas9 and N-Cas9) and fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89.

In particular embodiments, the guide nucleotide sequence-programmable RNA binding protein is modified to eliminate endonuclease activity (“nuclease dead”). For example, both RuvC and HNH nuclease domains can be rendered inactive by point mutations (e.g., D10A and H840A in SpCas9), resulting in a nuclease dead Cas9 (dCas9) molecule that cannot cleave target DNA. The dCas9 molecule retains the ability to bind to target RNA based on the gRNA targeting sequence.

Further non-limiting examples of orthologs and biological equivalents Cas9 are provided in Table 1.

TABLE 1

| Name | Protein Sequence |
|---|---|
| S. pyogenes | MDKKYSIGLDIGTNSVGWAV |
| Cas9 | ITDEYKVPSKKFKVLGNTDR |
| | HSIKKNLIGALLFDSGETAE |
| | ATRLKRTARRRYTRRKNRIC |
| | YLQEIFSNEMAKVDDSFFHR |
| | LEESFLVEEDKKHERHPIFG |
| | NIVDEVAYHEKYPTIYHLRK |
| | KLVDSTDKADLRLIYLALAH |
| | MIKFRGHFLIEGDLNPDNSD |
| | VDKLFIQLVQTYNQLFEENP |
| | INASGVDAKAILSARLSKSR |
| | RLENLIAQLPGEKKNGLFGN |
| | LIALSLGLTPNFKSNFDLAE |
| | DAKLQLSKDTYDDDLDNLLA |
| | QIGDQYADLFLAAKNLSDAI |
| | LLSDILRVNTEITKAPLSAS |
| | MIKRYDEHHQDLTLLKALVR |
| | QQLPEKYKEIFFDQSKNGYA |
| | GYIDGGASQEEFYKFIKPIL |
| | EKMDGTEELLVKLNREDLLR |
| | KQRTFDNGSIPHQIHLGELH |
| | AILRRQEDFYPFLKDNREKI |
| | EKILTFRIPYYVGPLARGNS |
| | RFAWMTRKSEETITPWNFEE |
| | VVDKGASAQSFIERMTNFDK |
| | NLPNEKVLPKHSLLYEYFTV |
| | YNELTKVKYVTEGMRKPAFL |
| | SGEQKKAIVDLLFKTNRKVT |
| | VKQLKEDYFKKIECFDSVEI |
| | SGVEDRFNASLGTYHDLLKI |
| | IKDKDFLDNEENEDILEDIV |
| | LTLTLFEDREMIEERLKTYA |
| | HLFDDKVMKQLKRRRYTGWG |
| | RLSRKLINGIRDKQSGKTIL |
| | DFLKSDGFANRNFMQLIHDD |
| | SLTFKEDIQKAQVSGQGDSL |
| | HEHIANLAGSPAIKKGILQT |
| | VKVVDELVKVMGRHKPENIV |
| | IEMARENQTTQKGQKNSRER |
| | MKRIEEGIKELGSQILKEHP |
| | VENTQLQNEKLYLYYLQNGR |
| | DMYVDQELDINRLSDYDVDH |
| | IVPQSFLKDDSIDNKVLTRS |
| | DKNRGKSDNVPSEEVVKKMK |
| | NYWRQLLNAKLITQRKFDNL |
| | TKAERGGLSELDKAGFIKRQ |
| | LVETRQITKHVAQILDSRMN |
| | TKYDENDKLIREVKVITLKS |
| | KLVSDFRKDFQFYKVREINN |
| | YHHAHDAYLNAVVGTALIKK |
| | YPKLESEFVYGDYKVYDVRK |
| | MIAKSEQEIGKATAKYFFYS |
| | NIMNFFKTEITLANGEIRKR |
| | PLIETNGETGEIVWDKGRDF |
| | ATVRKVLSMPQVNIVKKIEV |
| | QTGGFSKESILPKRNSDKLI |
| | ARKKDWDPKKYGGFDSPTVA |
| | YSVLVVAKVEKGKSKKLKSV |
| | KELLGITIMERSSFEKNPID |
| | FLEAKGYKEVKKDLIIKLPK |
| | YSLFELENGRKRMLASAGEL |
| | QKGNELALPSKYVNFLYLAS |
| | HYEKLKGSPEDNEQKQLFVE |
| | QHKHYLDEIIEQISEFSKRV |
| | ILADANLDKVLSAYNKHRDK |
| | PIREQAENIIHLFTLTNLGA |
| | PAAFKYFDTTIDRKRYTSTK |
| | EVLDATLIHQSITGLYETRI |
| | DLSQLGGD |
| | (SEQ ID NO: 1) |
| Staphylococcus | MKRNYILGLDIGITSVGYGI |
| aureus Cas9 | IDYETRDVIDAGVRLFKEAN |
| | VENNEGRRSKRGARRLKRRR |
| | RHRIQRVKKLLFDYNLLTDH |
| | SELSGINPYEARVKGLSQKL |
| | SEEEFSAALLHLAKRRGVHN |
| | VNEVEEDTGNELSTKEQISR |
| | NSKALEEKYVAELQLERLKK |
| | DGEVRGSINRFKTSDYVKEA |
| | KQLLKVQKAYHQLDQSFIDT |
| | YIDLLETRRTYYEGPGEGSP |
| | FGWKDIKEWYEMLMGHCTYF |
| | PEELRSVKYAYNADLYNALN |
| | DLNNLVITRDENEKLEYYEK |
| | FQIIENVFKQKKKPTLKQIA |
| | KEILVNEEDIKGYRVTSTGK |
| | PEFTNLKVYHDIKDITARKE |
| | IIENAELLDQIAKILTIYQS |
| | SEDIQEELTNLNSELTQEEI |
| | EQISNLKGYTGTHNLSLKAI |
| | NLILDELWHTNDNQIAIFNR |
| | LKLVPKKVDLSQQKEIPTTL |
| | VDDFILSPVVKRSFIQSIKV |
| | INAIIKKYGLPNDIIIELAR |
| | EKNSKDAQKMINEMQKRNRQ |
| | TNERIEEIIRTTGKENAKYL |
| | IEKIKLHDMQEGKCLYSLEA |
| | IPLEDLLNNPFNYEVDHIIP |
| | RSVSFDNSFNNKVLVKQEEN |
| | SKKGNRTPFQYLSSSDSKIS |
| | YETFKKHILNLAKGKGRISK |
| | TKKEYLLEERDINRFSVQKD |
| | FINRNLVDTRYATRGLMNLL |
| | RSYFRVNNLDVKVKSINGGF |
| | TSFLRRKWKFKKERNKGYKH |
| | HAEDALIIANADFIFKEWKK |
| | LDKAKKVMENQMIEEKQAES |
| | MPEIEIEQEYKEIFITPHQI |
| | KHIKDFKDYKYSHRVDKKPN |
| | RELINDTLYSTRKDDKGNTL |
| | IVNNLNGLYDKDNDKLKKLI |
| | NKSPEKLLMYHHDPQTYQKL |
| | KLIMEQYGDEKNPLYKYYEE |
| | TGNYLTKYSKKDNGPVIKKI |
| | KYYGNKLNAHLDITDDYPNS |
| | RNKVVKLSLKPYRFDVYLDN |
| | GVYKFVTVKNLDVIKKENYY |
| | EVNSKCYEEAKKLKKISNQA |
| | EFIASFYNNDLIKINGELYR |
| | VIGVNNDLLNRIEVNMIDIT |
| | YREYLENMNDKRPPRIIKTI |
| | ASKTQSIKKYSTDILGNLYE |
| | VKSKKHPQIIKKG |
| | (SEQ ID NO: 2) |
| S. thermophilus | MSDLVLGLDIGIGSVGVGIL |
| CRISPR 1 | NKVTGEIIHKNSRIFPAAQA |
| Cas9 | ENNLVRRTNRQGRRLARRKK |
| | HRRVRLNRLFEESGLITDFT |
| | KISINLNPYQLRVKGLTDEL |
| | SNEELFIALKNMVKHRGISY |
| | LDDASDDGNSSVGDYAQIVK |
| | ENSKQLETKTPGQIQLERYQ |
| | TYGQLRGDFTVEKDGKKHRL |
| | INVFPTSAYRSEALRILQTQ |
| | QEFNPQITDEFINRYLEILT |
| | GKRKYYHGPGNEKSRTDYGR |
| | YRTSGETLDNIFGILIGKCT |
| | FYPDEFRAAKASYTAQEFNL |
| | LNDLNNLTVPTETKKLSKEQ |
| | KNQIINYVKNEKAMGPAKLF |
| | KYIAKLLSCDVADIKGYRID |
| | KSGKAEIHTFEAYRKMKTLE |
| | TLDIEQMDRETLDKLAYVLT |
| | LNIEREGIQEALEHEFADGS |
| | FSQKQVDELVQFRKANSSIF |
| | GKGWHNFSVKLMMELIPELY |
| | ETSEEQMTILTRLGKQKTTS |
| | SSNKTKYIDEKLLIEEIYNP |
| | VVAKSVRQAIKIVNAAIKEY |
| | GDFDNIVIEMARETNEDDEK |
| | KAIQKIQKANKDEKDAAMLK |
| | AANQYNGKAELPHSVFHGHK |
| | QLATKIRLWHQQGERCLYTG |
| | KTISIHDLINNSNQFEVDHI |
| | LPLSITFDDSLANKVLVYAT |
| | ANQEKGQRTPYQALDSMDDA |
| | WSFRELKAFVRESKTLSNKK |
| | KEYLLIEEDISKFDVRKKFI |
| | ERNLVDTRYASRVVLNALQE |
| | HFRAHKIDTKVSVVRGQFTS |
| | QLRRHWGIEKTRDTYHHHAV |
| | DALIIAASSQLNLWKKQKNT |
| | LVSYSEDQLLDIETGELISD |
| | DEYKESVFKAPYQHFVDTLK |
| | SKEFEDSILFSYQVDSKFNR |
| | KISDATIYATRQAKVGKDKA |
| | DETYVLGKIKDIYTQDGYDA |
| | FMKIYKKDKSKFLMYRHDPQ |
| | TFEKVIEPILENYPNKQIND |
| | KGKEVPCNPFLKYKEEHGYI |
| | RKYSKKGNGPEIKSLKYYDS |
| | KLGNHIDITPKDSNNKVVLQ |
| | SVSPWRADVYFNKTTGKYEI |
| | LGLKYADLQFDKGTGTYKIS |
| | QEKYNDIKKKEGVDSDSEFK |
| | FTLYKNDLLLVKDTETKEQQ |
| | LFRFLSRTMPKQKHYVELKP |
| | YDKQKFEGGEALIKVLGNVA |
| | NSGQCKKGLGKSNISIYKVR |
| | TDVLGNQHIIKNEGDKPKLD |
| | F |
| | (SEQ ID NO: 3) |
| N. meningitidis | MAAFKPNPINYILGLDIGIA |
| Cas9 | SVGWAMVEIDEDENPICLID |
| | LGVRVFERAEVPKTGDSLAM |
| | ARRLARSVRRLTRRRAHRLL |
| | RARRLLKREGVLQAADFDEN |
| | GLIKSLPNTPWQLRAAALDR |
| | KLTPLEWSAVLLHLIKHRGY |
| | LSQRKNEGETADKELGALLK |
| | GVADNAHALQTGDFRTPAEL |
| | ALNKFEKESGHIRNQRGDYS |
| | HTFSRKDLQAELILLFEKQK |
| | EFGNPHVSGGLKEGIETLLM |
| | TQRPALSGDAVQKMLGHCTF |
| | EPAEPKAAKNTYTAERFIWL |
| | TKLNNLRILEQGSERPLTDI |
| | ERATLMDEPYRKSKLTYAQA |
| | RKLLGLEDTAFFKGLRYGKD |
| | NAEASTLMEMKAYHAISRAL |
| | EKEGLKDKKSPLNLSPELQD |
| | EIGTAFSLFKTDEDITGRLK |
| | DRIQPEILEALLKHISFDKF |
| | VQISLKALRRIVPLMEQGKR |
| | YDEACAEIYGDHYGKKNIEE |
| | KIYLPPIPADEIRNPVVLRA |
| | LSQARKVINGVVRRYGSPAR |
| | IHIETAREVGKSFKDRKEIE |
| | KRQEENRKDREKAAAKFREY |
| | FPNFVGEPKSKDILKLRLYE |
| | QQHGKCLYSGKEINLGRLNE |
| | KGYVEIDHALPFSRTWDDSF |
| | NNKVLVLGSENQNKGNQTPY |
| | EYFNGKDNSREWQEFKARVE |
| | TSRFPRSKKQRILLQKFDED |
| | GFKERNLNDTRYVNRFLCQF |
| | VADRMRLTGKGKKRVFASNG |
| | QITNLLRGFWGLRKVRAEND |
| | RHHALDAVVVACSTVAMQQK |
| | ITRFVRYKEMNAFDGKTIDK |
| | ETGEVLHQKTHFPQPWEFFA |
| | QEVMIRVFGKPDGKPEFEEA |
| | DTPEKLRTLLAEKLSSRPEA |
| | VHEYVTPLFVSRAPNRKMSG |
| | QGHMETVKSAKRLDEGVSVL |
| | RVPLTQLKLKDLEKMVNRER |
| | EPKLYEALKARLEAHKDDPA |
| | KAFAEPFYKYDKAGNRTQQV |
| | KAVRVEQVQKTGVWVRNHNG |
| | IADNATMVRVDVFEKGDKYY |
| | LVPIYSWQVAKGILPDRAVV |
| | QGKDEEDWQLIDDSFNFKFS |
| | LHPNDLVEVITKKARMFGYF |
| | ASCHRGTGNINIRIHDLDHK |
| | IGKNGILEGIGVKTALSFQK |
| | YQIDELGKEIRPCRLKKRPP |
| | VR (SEQ ID NO: 4) |
| Parvibaculum | MERIFGFDIGTTSIGFSVID |
| lavamentivorans | YSSTQSAGNIQRLGVRIFPE |
| Cas9 | ARDPDGTPLNQQRRQKRMMR |
| | RQLRRRRIRRKALNETLHEA |
| | GFLPAYGSADWPVVMADEPY |
| | ELRRRGLEEGLSAYEFGRAI |
| | YHLAQHRHFKGRELEESDTP |
| | DPDVDDEKEAANERAATLKA |
| | LKNEQTTLGAWLARRPPSDR |
| | KRGIHAHRNVVAEEFERLWE |
| | VQSKFHPALKSEEMRARISD |
| | TIFAQRPVFWRKNTLGECRF |
| | MPGEPLCPKGSWLSQQRRML |
| | EKLNNLAIAGGNARPLDAEE |
| | RDAILSKLQQQASMSWPGVR |
| | SALKALYKQRGEPGAEKSLK |
| | FNLELGGESKLLGNALEAKL |
| | ADMFGPDWPAHPRKQEIRHA |
| | VHERLWAADYGETPDKKRVI |
| | ILSEKDRKAHREAAANSFVA |
| | DFGITGEQAAQLQALKLPTG |
| | WEPYSIPALNLFLAELEKGE |
| | RFGALVNGPDWEGWRWINFP |
| | HRNQPTGEILDKLPSPASKE |
| | ERERISQLRNPTVVRTQNEL |
| | RKVVNNLIGLYGKPDRIRIE |
| | VGRDVGKSKREREEIQSGIR |
| | RNEKQRKKAIEDLIKNGIAN |
| | PSRDDVEKWILWKEGQERCP |
| | YTGDQIGFNALFREGRYEVE |
| | HIWPRSRSFDNSPRNKTLCR |
| | KDVNIEKGNRMPFEAFGHDE |
| | DRWSAIQIRLQGMVSAKGGT |
| | GMSPGKVKRFLAKTMPEDFA |
| | ARQLNDTRYAAKQILAQLKR |
| | LWPDMGPEAPVKVEAVTGQV |
| | TAQLRKLWTLNNILADDGEK |
| | TRADHRHHAIDALTVACTHP |
| | GMTNKLSRYWQLRDDPRAEK |
| | PALTPPWDTIRADAEKAVSE |
| | IVVSHRVRKKVSGPLHKETT |
| | YGDTGTDIKTKSGTYRQFVT |
| | RKKIESLSKGELDEIRDPRI |
| | KEIVAAHVAGRGGDPKKAFP |
| | PYPCVSPGGPEIRKVRLTSK |
| | QQLNLMAQTGNGYADLGSNH |
| | HIAIYRLPDGKADFEIVSLF |
| | DASRRLAQRNPIVQRTRADG |
| | ASFVMSLAAGEAIMIPEGSK |
| | KGIWIVQGVWASGQVVLERD |
| | TDADHSTTTRPMPNPILKDD |
| | AKKVSIDPIGRVRPSND |
| | (SEQ ID NO: 5) |
| Corynebacter | MKYHVGIDVGTFSVGLAAIE |
| diphtheria | VDDAGMPIKTLSLVSHIHDS |
| Cas9 | GLDPDEIKSAVTRLASSGIA |
| | RRTRRLYRRKRRRLQQLDKF |
| | IQRQGWPVIELEDYSDPLYP |
| | WKVRAELAASYIADEKERGE |
| | KLSVALRHIARHRGWRNPYA |
| | KVSSLYLPDGPSDAFKAIRE |
| | EIKRASGQPVPETATVGQMV |
| | TLCELGTLKLRGEGGVLSAR |
| | LQQSDYAREIQEICRMQEIG |
| | QELYRKIIDVVFAAESPKGS |
| | ASSRVGKDPLQPGKNRALKA |
| | SDAFQRYRIAALIGNLRVRV |
| | DGEKRILSVEEKNLVFDHLV |
| | NLTPKKEPEWVTIAEILGID |
| | RGQLIGTATMTDDGERAGAR |
| | PPTHDTNRSIVNSRIAPLVD |
| | WWKTASALEQHAMVKALSNA |
| | EVDDFDSPEGAKVQAFFADL |
| | DDDVHAKLDSLHLPVGRAAY |
| | SEDTLVRLTRRMLSDGVDLY |
| | TARLQEFGIEPSWTPPTPRI |
| | GEPVGNPAVDRVLKTVSRWL |
| | ESATKTWGAPERVIIEHVRE |
| | GFVTEKRAREMDGDMRRRAA |
| | RNAKLFQEMQEKLNVQGKPS |
| | RADLWRYQSVQRQNCQCAYC |
| | GSPITFSNSEMDHIVPRAGQ |
| | GSTNTRENLVAVCHRCNQSK |
| | GNTPFAIWAKNTSIEGVSVK |
| | EAVERTRHWVTDTGMRSTDF |
| | KKFTKAVVERFQRATMDEEI |
| | DARSMESVAWMANELRSRVA |
| | QHFASHGTTVRVYRGSLTAE |
| | ARRASGISGKLKFFDGVGKS |
| | RLDRRHHAIDAAVIAFTSDY |
| | VAETLAVRSNLKQSQAHRQE |
| | APQWREFTGKDAEHRAAWRV |
| | WCQKMEKLSALLIEDLRDDR |
| | VVVMSNVRLRLGNGSAHKET |
| | IGKLSKVKLSSQLSVSDIDK |
| | ASSEALWCALTREPGFDPKE |
| | GLPANPERHIRVNGTHVYAG |
| | DNIGLFPVSAGSIALRGGYA |
| | ELGSSFHHARVYKITSGKKP |
| | AFAMLRVYTIDLLPYRNQDL |
| | FSVELKPQTMSMRQAEKKLR |
| | DALATGNAEYLGWLVVDDEL |
| | VVDTSKIATDQVKAVEAELG |
| | TIRRWRVDGFFSPSKLRLRP |
| | LQMSKEGIKKESAPELSKIM |
| | RPGWLPAVNKLFSDGNVTVV |
| | RRDSLGRVRLESTAHLPVTW |
| | KVQ (SEQ ID NO: 6) |
| Streptococcus | MTNGKILGLDIGIASVGVGI |
| pasteurianus | IEAKTGKVVHANSRLFSAAN |
| Cas9 | AENNAERRGFRGSRRLNRRK |
| | KHRVKRVRDLFEKYGIVTDF |
| | RNLNLNPYELRVKGLTEQLK |
| | NEELFAALRTISKRRGISYL |
| | DDAEDDSTGSTDYAKSIDEN |
| | RRLLKNKTPGQIQLERLEKY |
| | GQLRGNFTVYDENGEAHRLI |
| | NVFSTSDYEKEARKILETQA |
| | DYNKKITAEFIDDYVEILTQ |
| | KRKYYHGPGNEKSRTDYGRF |
| | RTDGTTLENIFGILIGKCNF |
| | YPDEYRASKASYTAQEYNFL |
| | NDLNNLKVSTETGKLSTEQ |
| | KESLVEFAKNTATLGPAK |
| | LLKEIAKILDCKVDEIKGYR |
| | EDDKGKPDLHTFEPYRKLKF |
| | NLESINIDDLSREVIDKLAD |
| | ILTLNTEREGIEDAIKRNLP |
| | NQFTEEQISEIIKVRKSQST |
| | AFNKGWHSFSAKLMNELIPE |
| | LYATSDEQMTILTRLEKFKV |
| | NKKSSKNTKTIDEKEVTDEI |
| | YNPVVAKSVRQTIKIINAAV |
| | KKYGDFDKIVIEMPRDKNAD |
| | DEKKFIDKRNKENKKEKDDA |
| | LKRAAYLYNSSDKLPDEVFH |
| | GNKQLETKIRLWYQQGERCL |
| | YSGKPISIQELVHNSNNFEI |
| | DHILPLSLSFDDSLANKVLV |
| | YAWTNQEKGQKTPYQVIDSM |
| | DAAWSFREMKDYVLKQKGLG |
| | KKKRDYLLTTENIDKIEVKK |
| | KFIERNLVDTRYASRVVLNS |
| | LQSALRELGKDTKVSVVRGQ |
| | FTSQLRRKWKIDKSRETYHH |
| | HAVDALIIAASSQLKLWEKQ |
| | DNPMIVDYGKNQVVDKQTGE |
| | ILSVSDDEYKELVFQPPYQG |
| | FVNTISSKGFEDEILFSYQV |
| | DSKYNRKVSDATIYSTRKAK |
| | IGKDKKEETYVLGKIKDIYS |
| | QNGFDTFIKKYNKDKTQFLM |
| | YQKDSLTWENVIEVILRDYP |
| | TTKKSEDGKNDVKCNPFEEY |
| | RRENGLICKYSKKGKGTPIK |
| | SLKYYDKKLGNCIDITPEES |
| | RNKVILQSINPWRADVYFNP |
| | ETLKYELMGLKYSDLSFEKG |
| | TGNYHISQEKYDAIKEKEGI |
| | GKKSEFKFTLYRNDLILIKD |
| | IASGEQEIYRFLSRTMPNVN |
| | HYVELKPYDKEKFDNVQELV |
| | EALGEADKVGRCIKGLNKPN |
| | ISIYKVRTDVLGNKYFVKKK |
| | GDKPKLDFKNNKK |
| | (SEQ ID NO: 7) |
| Neisseria | MAAFKPNPMNYILGLDIGIA |
| cinerea | SVGWAIVEIDEEENPIRLID |
| Cas9 | LGVRVFERAEVPKTGDSLAA |
| | ARRLARSVRRLTRRRAHRLL |
| | RARRLLKREGVLQAADFDEN |
| | GLIKSLPNTPWQLRAAALDR |
| | KLTPLEWSAVLLHLIKHRGY |
| | LSQRKNEGETADKELGALLK |
| | GVADNTHALQTGDFRTPAEL |
| | ALNKFEKESGHIRNQRGDYS |
| | HTFNRKDLQAELNLLFEKQK |
| | EFGNPHVSDGLKEGIETLLM |
| | TQRPALSGDAVQKMLGHCTF |
| | EPTEPKAAKNTYTAERFVWL |
| | TKLNNLRILEQGSERPLTDT |
| | ERATLMDEPYRKSKLTYAQA |
| | RKLLDLDDTAFFKGLRYGKD |
| | NAEASTLMEMKAYHAISRAL |
| | EKEGLKDKKSPLNLSPELQD |
| | EIGTAFSLFKTDEDITGRLK |
| | DRVQPEILEALLKHISFDKF |
| | VQISLKALRRIVPLMEQGNR |
| | YDEACTEIYGDHYGKKNTEE |
| | KIYLPPIPADEIRNPVVLRA |
| | LSQARKVINGVVRRYGSPAR |
| | IHIETAREVGKSFKDRKEIE |
| | KRQEENRKDREKSAAKFREY |
| | FPNFVGEPKSKDILKLRLYE |
| | QQHGKCLYSGKEINLGRLNE |
| | KGYVEIDHALPFSRTWDDSF |
| | NNKVLALGSENQNKGNQTPY |
| | EYFNGKDNSREWQEFKARVE |
| | TSRFPRSKKQRILLQKFDED |
| | GFKERNLNDTRYINRFLCQF |
| | VADHMLLTGKGKRRVFASNG |
| | QITNLLRGFWGLRKVRAEND |
| | RHHALDAVVVACSTIAMQQK |
| | ITRFVRYKEMNAFDGKTIDK |
| | ETGEVLHQKAHFPQPWEFFA |
| | QEVMIRVFGKPDGKPEFEEA |
| | DTPEKLRTLLAEKLSSRPEA |
| | VHKYVTPLFISRAPNRKMSG |
| | QGHMETVKSAKRLDEGISVL |
| | RVPLTQLKLKDLEKMVNRER |
| | EPKLYEALKARLEAHKDDPA |
| | KAFAEPFYKYDKAGNRTQQV |
| | KAVRVEQVQKTGVWVHNHNG |
| | IADNATIVRVDVFEKGGKYY |
| | LVPIYSWQVAKGILPDRAVV |
| | QGKDEEDWTVMDDSFEFKFV |
| | LYANDLIKLTAKKNEFLGYF |
| | VSLNRATGAIDIRTHDTDST |
| | KGKNGIFQSVGVKTALSFQK |
| | YQIDELGKEIRPCRLKKRPP |
| | VR (SEQ ID NO: 8) |
| Campylobacter | MRILGFDIGINSIGWAFVEN |
| lari | DELKDCGVRIFTKAENPKNK |
| Cas9 | ESLALPRRNARSSRRRLKRR |
| | KARLIAIKRILAKELKLNYK |
| | DYVAADGELPKAYEGSLASV |
| | YELRYKALTQNLETKDLARV |
| | ILHIAKHRGYMNKNEKKSND |
| | AKKGKILSALKNNALKLENY |
| | QSVGEYFYKEFFQKYKKNTK |
| | NFIKIRNTKDNYNNCVLSSD |
| | LEKELKLILEKQKEFGYNYS |
| | EDFINEILKVAFFQRPLKDF |
| | SHLVGACTFFEEEKRACKNS |
| | YSAWEFVALTKIINEIKSLE |
| | KISGEIVPTQTINEVLNLIL |
| | DKGSITYKKFRSCINLHESI |
| | SFKSLKYDKENAENAKLIDF |
| | RKLVEFKKALGVHSLSRQEL |
| | DQISTHITLIKDNVKLKTVL |
| | EKYNLSNEQINNLLEIEFND |
| | YINLSFKALGMILPLMREGK |
| | RYDEACEIANLKPKTVDEKK |
| | DFLPAFCDSIFAHELSNPVV |
| | NRAISEYRKVLNALLKKYGK |
| | VHKIHLELARDVGLSKKARE |
| | KIEKEQKENQAVNAWALKEC |
| | ENIGLKASAKNILKLKLWKE |
| | QKEICIYSGNKISIEHLKDE |
| | KALEVDHIYPYSRSFDDSFI |
| | NKVLVFTKENQEKLNKTPFE |
| | AFGKNIEKWSKIQTLAQNLP |
| | YKKKNKILDENFKDKQQEDF |
| | ISRNLNDTRYIATLIAKYTK |
| | EYLNFLLLSENENANLKSGE |
| | KGSKIHVQTISGMLTSVLRH |
| | TWGFDKKDRNNHLHHALDAI |
| | IVAYSTNSIIKAFSDFRKNQ |
| | ELLKARFYAKELTSDNYKHQ |
| | VKFFEPFKSFREKILSKIDE |
| | IFVSKPPRKRARRALHKDTF |
| | HSENKIIDKCSYNSKEGLQI |
| | ALSCGRVRKIGTKYVENDTI |
| | VRVDIFKKQNKFYAIPIYAM |
| | DFALGILPNKIVITGKDKNN |
| | NPKQWQTIDESYEFCFSLYK |
| | NDLILLQKKNMQEPEFAYYN |
| | DFSISTSSICVEKHDNKFEN |
| | LTSNQKLLFSNAKEGSVKVE |
| | SLGIQNLKVFEKYIITPLGD |
| | KIKADFQPRENISLKTSKKY |
| | GLR (SEQ ID NO: 9) |
| T. denticola | MKKEIKDYFLGLDVGTGSVG |
| Cas9 | WAVTDTDYKLLKANRKDLWG |
| | MRCFETAETAEVRRLHRGAR |
| | RRIERRKKRIKLLQELFSQE |
| | IAKTDEGFFQRMKESPFYAE |
| | DKTILQENTLFNDKDFADKT |
| | YHKAYPTINHLIKAWIENKV |
| | KPDPRLLYLACHNIIKKRGH |
| | FLFEGDFDSENQFDTSIQAL |
| | FEYLREDMEVDIDADSQKVK |
| | EILKDSSLKNSEKQSRLNKI |
| | LGLKPSDKQKKAITNLISGN |
| | KINFADLYDNPDLKDAEKNS |
| | ISFSKDDFDALSDDLASILG |
| | DSFELLLKAKAVYNCSVLSK |
| | VIGDEQYLSFAKVKIYEKHK |
| | TDLTKLKNVIKKHFPKDYKK |
| | VFGYNKNEKNNNNYSGYVGV |
| | CKTKSKKLIINNSVNQEDFY |
| | KFLKTILSAKSEIKEVNDIL |
| | TEIETGTFLPKQISKSNAEI |
| | PYQLRKMELEKILSNAEKHF |
| | SFLKQKDEKGLSHSEKIIML |
| | LTFKIPYYIGPINDNHKKFF |
| | PDRCWVVKKEKSPSGKTTPW |
| | NFFDHIDKEKTAEAFITSWI |
| | NFCTYLVGESVLPKSSLLYS |
| | EYTVLNEINNLQIIIDGKNI |
| | CDIKLKQKIYEDLFKKYKKI |
| | TQKQISTFIKHEGICNKTDE |
| | VIILGIDKECTSSLKSYIEL |
| | KNIFGKQVDEISTKNMLEEI |
| | IRWATIYDEGEGKTILKTKI |
| | KAEYGKYCSDEQIKKILNLK |
| | FSGWGRLSRKFLETVTSEMP |
| | GFSEPVNIITAMRETQNNLM |
| | ELLSSEFTFTENIKKINSG |
| | FEDAEKQFSYD |
| | GLVKPLFLSPSVKKMLWQTL |
| | KLVKEISHITQAPPKKIFIE |
| | MAKGAELEPARTKTRLKILQ |
| | DLYNNCKNDADAFSSEIKDL |
| | SGKIENEDNLRLRSDKLYLY |
| | YTQLGKCMYCGKPIEIGHVF |
| | DTSNYDIDHIYPQSKIKDDS |
| | ISNRVLVCSSCNKNKEDKYP |
| | LKSEIQSKQRGFWNFLQRNN |
| | FISLEKLNRLTRATPISDDE |
| | TAKFIARQLVETRQATKVAA |
| | KVLEKMFPETKIVYSKAETV |
| | SMFRNKFDIVKCREINDFHH |
| | AHDAYLNIVVGNVYNTKFTN |
| | NPWNFIKEKRDNPKIADTYN |
| | YYKVFDYDVKRNNITAWEKG |
| | KTIITVKDMLKRNTPIYTRQ |
| | AACKKGELFNQTIMKKGLGQ |
| | HPLKKEGPFSNISKYGGYNK |
| | VSAAYYTLIEYEEKGNKIRS |
| | LETIPLYLVKDIQKDQDVLK |
| | SYLTDLLGKKEFKILVPKIK |
| | INSLLKINGFPCHITGKTND |
| | SFLLRPAVQFCCSNNEVLYF |
| | KKIIRFSEIRSQREKIGKTI |
| | SPYEDLSFRSYIKENLWKKT |
| | KNDEIGEKEFYDLLQKKNLE |
| | IYDMLLTKHKDTIYKKRPNS |
| | ATIDILVKGKEKFKSLIIEN |
| | QFEVILEILKLFSATRNVSD |
| | LQHIGGSKYSGVAKIGNKIS |
| | SLDNCILIYQSITGIFEKRI |
| | DLLKV |
| | (SEQ ID NO: 10) |
| S. mutans Cas9 | MKKPYSIGLDIGTNSVGWAV |
| | VTDDYKVPAKKMKVLGNTDK |
| | SHIEKNLLGALLFDSGNTAE |
| | DRRLKRTARRRYTRRRNRIL |
| | YLQEIFSEEMGKVDDSFFHR |
| | LEDSFLVTEDKRGERHPIFG |
| | NLEEEVKYHENFPTIYHLRQ |
| | YLADNPEKVDLRLVYLALAH |
| | IIKFRGHFLIEGKFDTRNND |
| | VQRLFQEFLAVYDNTFENSS |
| | LQEQNVQVEEILTDKISKSA |
| | KKDRVLKLFPNEKSNGRFAE |
| | FLKLIVGNQADFKKHFELEE |
| | KAPLQFSKDTYEEELEVLLA |
| | QIGDNYAELFLSAKKLYDSI |
| | LLSGILTVTDVGTKAPLSAS |
| | MIQRYNEHQMDLAQLKQFIR |
| | QKLSDKYNEVFSDVSKDGYA |
| | GYIDGKTNQEAFYKYLKGLL |
| | NKIEGSGYFLDKIEREDFLR |
| | KQRTFDNGSIPHQIHLQEMR |
| | AIIRRQAEFYPFLADNQDRI |
| | EKLLTFRIPYYVGPLARGKS |
| | DFAWLSRKSADKITPWNFDE |
| | IVDKESSAEAFINRMTNYDL |
| | YLPNQKVLPKHSLLYEKFTV |
| | YNELTKVKYKTEQGKTAFFD |
| | ANMKQEIFDGVFKVYRKVTK |
| | DKLMDFLEKEFDEFRIVDLT |
| | GLDKENKVFNASYGTYHDLC |
| | KILDKDFLDNSKNEKILEDI |
| | VLTLTLFEDREMIRKRLENY |
| | SDLLTKEQVKKLERRHYTGW |
| | GRLSAELIHGIRNKESRKTI |
| | LDYLIDDGNSNRNFMQLIND |
| | DALSFKEEIAKAQVIGETDN |
| | LNQVVSDIAGSPAIKKGILQ |
| | SLKIVDELVKIMGHQPENIV |
| | VEMARENQFTNQGRRNSQQR |
| | LKGLTDSIKEFGSQILKEHP |
| | VENSQLQNDRLFLYYLQNGR |
| | DMYTGEELDIDYLSQYDIDH |
| | IIPQAFIKDNSIDNRVLTSS |
| | KENRGKSDDVPSKDVVRKMK |
| | SYWSKLLSAKLITQRKFDNL |
| | TKAERGGLTDDDKAGFIKRQ |
| | LVETRQITKHVARILDERFN |
| | IETDENNKKIRQVKIVTLKS |
| | NLVSNFRKEFELYKVREIND |
| | YHHAHDAYLNAVIGKALLGV |
| | YPQLEPEFVYGDYPHFHGHK |
| | ENKATAKKFFYSNIMNFFKK |
| | DDVRTDKNGEIIWKKDEHIS |
| | NIKKVLSYPQVNIVKKVEEQ |
| | TGGFSKESILPKGNSDKLIP |
| | RKTKKFYWDTKKYGGFDSPI |
| | VAYSILVIADIEKGKSKKLK |
| | TVKALVGVTIMEKMTFERDP |
| | VAFLERKGYRNVQEENIIKL |
| | PKYSLFKLENGRKRLLASAR |
| | ELQKGNEIVLPNHLGTLLYH |
| | AKNIHKVDEPKHLDYVDKHK |
| | DEFKELLDVVSNFSKKYTLA |
| | EGNLEKIKELYAQNNGEDLK |
| | ELASSFINLLTFTAIGAPAT |
| | FKFFDKNIDRKRYTSTTEIL |
| | NATLIHQSITGLYETRIDLN |
| | KLGGD (SEQ ID NO: 11) |
| S. thermophilus | MTKPYSIGLDIGTNSVGWAV |
| CRISPR3 | TTDNYKVPSKKMKVLGNTSK |
| Cas9 | KYIKKNLLGVLLFDSGITAE |
| | GRRLKRTARRRYTRRRNRIL |
| | YLQEIFSTEMATLDDAFFQ |
| | RLDDSFLVP |
| | DDKRDSKYPIFGNLVEEKAY |
| | HDEFPTIYHLRKYLADSTKK |
| | ADLRLVYLALAHMIKYRGHF |
| | LIEGEFNSKNNDIQKNFQDF |
| | LDTYNAIFESDLSLENSKQL |
| | EEIVKDKISKLEKKDRILKL |
| | FPGEKNSGIFSEFLKLIVGN |
| | QADFRKCFNLDEKASLHFSK |
| | ESYDEDLETLLGYIGDDYSD |
| | VFLKAKKLYDAILLSGFLTV |
| | TDNETEAPLSSAMIKRYNEH |
| | KEDLALLKEYIRNISLKTYN |
| | EVFKDDTKNGYAGYIDGKTN |
| | QEDFYVYLKKLLAEFEGADY |
| | FLEKIDREDFLRKQRTFDNG |
| | SIPYQIHLQEMRAILDKQAK |
| | FYPFLAKNKERIEKILTFRI |
| | PYYVGPLARGNSDFAWSIRK |
| | RNEKITPWNFEDVIDKESSA |
| | EAFINRMTSFDLYLPEEKVL |
| | PKHSLLYETFNVYNELTKVR |
| | FIAESMRDYQFLDSKQKKDI |
| | VRLYFKDKRKVTDKDIIEYL |
| | HAIYGYDGIELKGIEKQFNS |
| | SLSTYHDLLNIINDKEFLDD |
| | SSNEAIIEEIIHTLTIFEDR |
| | EMIKQRLSKFENIFDKSVLK |
| | KLSRRHYTGWGKLSAKLING |
| | IRDEKSGNTILDYLIDDGIS |
| | NRNFMQLIHDDALSFKKKIQ |
| | KAQIIGDEDKGNIKEVVKSL |
| | PGSPAIKKGILQSIKIVDEL |
| | VKVMGGRKPESIVVEMAREN |
| | QYTNQGKSNSQQRLKRLEKS |
| | LKELGSKILKENIPAKLSKI |
| | DNNALQNDRLYLYYLQNGKD |
| | MYTGDDLDIDRLSNYDIDHI |
| | IPQAFLKDNSIDNKVLVSSA |
| | SNRGKSDDVPSLEVVKKRKT |
| | FWYQLLKSKLISQRKFDNLT |
| | KAERGGLSPEDKAGFIQRQL |
| | VETRQITKHVARLLDEKFNN |
| | KKDENNRAVRTVKIITLKST |
| | LVSQFRKDFELYKVREINDF |
| | HHAHDAYLNAVVASALLKKY |
| | PKLEPEFVYGDYPKYNSFRE |
| | RKSATEKVYFYSNIMNIFKK |
| | SISLADGRVIERPLIEVNEE |
| | TGESVWNKESDLATVRRVLS |
| | YPQVNVVKKVEEQNHGLDRG |
| | KPKGLFNANLSSKPKPNSNE |
| | NLVGAKEYLDPKKYGGYAGI |
| | SNSFTVLVKGTIEKGAKKKI |
| | TNVLEFQGISILDRINYRKD |
| | KLNFLLEKGYKDIELIIELP |
| | KYSLFELSDGSRRMLASILS |
| | TNNKRGEIHKGNQIFLSQKF |
| | VKLLYHAKRISNTINENHRK |
| | YVENHKKEFEELFYYILEFN |
| | ENYVGAKKNGKLLNSAFQSW |
| | QNHSIDELCSSFIGPTGSER |
| | KGLFELTSRGSAADFEFLGV |
| | KIPRYRDYTPSSLLKDATLI |
| | HQSVTGLYETRIDLAKLGEG |
| | (SEQ ID NO: 12) |
| C. jejuni Cas9 | MARILAFDIGISSIGWAFSE |
| | NDELKDCGVRIFTKVENPKT |
| | GESLALPRRLARSARKRLAR |
| | RKARLNHLKHLIANEFKLNY |
| | EDYQSFDESLAKAYKGSLIS |
| | PYELRFRALNELLSKQDFAR |
| | VILHIAKRRGYDDIKNSDDK |
| | EKGAILKAIKQNEEKLANYQ |
| | SVGEYLYKEYFQKFKENSKE |
| | FTNVRNKKESYERCIAQSFL |
| | KDELKLIFKKQREFGFSFSK |
| | KFEEEVLSVAFYKRALKDFS |
| | HLVGNCSFFTDEKRAPKNSP |
| | LAFMFVALTRIINLLNNLKN |
| | IEGILYTKDDLNALLNEVLK |
| | NGTLTYKQTKKLLGLSDDYE |
| | FKGEKGTYFIEFKKYKEFIK |
| | ALGEHNLSQDDLNEIAKDIT |
| | LIKDEIKLKKALAKYDLNQN |
| | QIDSLSKLEFKDHLNISFKA |
| | LKLVTPLMLEGKKYDEACNE |
| | LNLKVAINEDKKDFLPAFNE |
| | TYYKDEVTNPVVLRAIKEYR |
| | KVLNALLKKYGKVHKINIEL |
| | AREVGKNHSQRAKIEKEQNE |
| | NYKAKKDAELECEKLGLKIN |
| | SKNILKLRLFKEQKEFCAYS |
| | GEKIKISDLQDEKMLEIDHI |
| | YPYSRSFDDSYMNKVLVFTK |
| | QNQEKLNQTPFEAFGNDSAK |
| | WQKIEVLAKNLPTKKQKRIL |
| | DKNYKDKEQKNFKDRNLNDT |
| | RYIARLVLNYTKDYLDFLPL |
| | SDDENTKLNDTQKGSKVHVE |
| | AKSGMLTSALRHTWGFSAKD |
| | RNNHLHHAIDAVIIAYANNS |
| | IVKAFSDFKKEQESNSAELY |
| | AKKISELDYKNKRKFFEPFS |
| | GFRQKVLDKIDEIFVSKPER |
| | KKPSGALHEETFRKEEEFYQ |
| | SYGGKEGVLKALELGKIRKV |
| | NGKIVKNGDMIRVDIFKHKK |
| | TNKFYAVPIYTMDFALKVLP |
| | NKAVARSKKGEIKDWILMDE |
| | NYEFCFSLYKDSLILIQTKD |
| | MQEPEFVYYNAFTSSTVSLI |
| | VSKHDNKFETLSKNQKILFK |
| | NANEKEVIAKSIGIQNLKVF |
| | EKYIVSALGEVTKAEFRQRE |
| | DFKK (SEQ ID NO: 13) |
| P. multocida | MQTTNLSYILGLDLGIASVG |
| Cas9 | WAVVEINENEDPIGLIDVGV |
| | RIFERAEVPKTGESLALSRR |
| | LARSTRRLIRRRAHRLLLAK |
| | RFLKREGILSTIDLEKGLPN |
| | QAWELRVAGLERRLSAIEWG |
| | AVLLHLIKHRGYLSKRKNES |
| | QTNNKELGALLSGVAQNHQL |
| | LQSDDYRTPAELALKKFAKE |
| | EGHIRNQRGAYTHTFNRLDL |
| | LAELNLLFAQQHQFGNPHCK |
| | EHIQQYMTELLMWQKPAL |
| | SGEAILKMLG |
| | KCTHEKNEFKAAKHTYSAER |
| | FVWLTKLNNLRILEDGAERA |
| | LNEEERQLLINHPYEKSKLT |
| | YAQVRKLLGLSEQAIFKHLR |
| | YSKENAESATFMELKAWHAI |
| | RKALENQGLKDTWQDLAKKP |
| | DLLDEIGTAFSLYKTDEDIQ |
| | QYLTNKVPNSVINALLVSLN |
| | FDKFIELSLKSLRKILPLME |
| | QGKRYDQACREIYGHHYGEA |
| | NQKTSQLLPAIPAQEIRNPV |
| | VLRTLSQARKVINAIIRQYG |
| | SPARVHIETGRELGKSFKER |
| | REIQKQQEDNRTKRESAVQK |
| | FKELFSDFSSEPKSKDILKF |
| | RLYEQQHGKCLYSGKEINIH |
| | RLNEKGYVEIDHALPFSRTW |
| | DDSFNNKVLVLASENQNKGN |
| | QTPYEWLQGKINSERWKNFV |
| | ALVLGSQCSAAKKQRLLTQV |
| | IDDNKFIDRNLNDTRYIARF |
| | LSNYIQENLLLVGKNKKNVF |
| | TPNGQITALLRSRWGLIKAR |
| | ENNNRHHALDAIVVACATPS |
| | MQQKITRFIRFKEVHPYKIE |
| | NRYEMVDQESGEIISPHFPE |
| | PWAYFRQEVNIRVFDNHPDT |
| | VLKEMLPDRPQANHQFVQPL |
| | FVSRAPTRKMSGQGHMETIK |
| | SAKRLAEGISVLRIPLTQLK |
| | PNLLENMVNKEREPALYAGL |
| | KARLAEFNQDPAKAFATPFY |
| | KQGGQQVKAIRVEQVQKSGV |
| | LVRENNGVADNASIVRTDVF |
| | IKNNKFFLVPIYTWQVAKGI |
| | LPNKAIVAHKNEDEWEEMDE |
| | GAKFKFSLFPNDLVELKTKK |
| | EYFFGYYIGLDRATGNISLK |
| | EHDGEISKGKDGVYRVGVKL |
| | ALSFEKYQVDELGKNRQICR |
| | PQQRQPVR |
| | (SEQ ID NO: 14) |
| F. novicida | MNFKILPIAIDLGVKNTGVF |
| Cas9 | SAFYQKGTSLERLDNKNGKV |
| | YELSKDSYTLLMNNRTARRH |
| | QRRGIDRKQLVKRLFKLIWT |
| | EQLNLEWDKDTQQAISFLFN |
| | RRGFSFITDGYSPEYLNIVP |
| | EQVKAILMDIFDDYNGEDDL |
| | DSYLKLATEQESKISE |
| | IYNKLMQKILEF |
| | KLMKLCTDIKDDKVSTKTLK |
| | EITSYEFELLADYLANYSES |
| | LKTQKFSYTDKQGNLKELSY |
| | YHHDKYNIQEFLKRHATIND |
| | RILDTLLTDDLDIWNFNFEK |
| | FDFDKNEEKLQNQEDKDHIQ |
| | AHLHHFVFAVNKIKSEMASG |
| | GRHRSQYFQEITNVLDENNH |
| | QEGYLKNFCENLHNKKYSNL |
| | SVKNLVNLIGNLSNLELKPL |
| | RKYFNDKIHAKADHWDEQKF |
| | TETYCHWILGEWRVGVKDQDK |
| | KDGAKYSYKDLCNELKQKVT |
| | KAGLVDFLLELDPCRTIPPY |
| | LDNNNRKPPKCQSLILNPKF |
| | LDNQYPNWQQYLQELKKLQS |
| | IQNYLDSFETDLKVLKSSKD |
| | QPYFVEYKSSNQQIASGQRD |
| | YKDLDARILQFIFDRVKASD |
| | ELLLNEIYFQAKKLKQKASS |
| | ELEKLESSKKLDEVIANSQL |
| | SQILKSQHTNGIFEQGTFLH |
| | LVCKYYKQRQRARDSRLYIM |
| | PEYRYDKKLHKYNNTGRFDD |
| | DNQLLTYCNHKPRQKRYQLL |
| | NDLAGVLQVSPNFLKDKIGS |
| | DDDLFISKWLVEHIRGFKKA |
| | CEDSLKIQKDNRGLLNHKIN |
| | IARNTKGKCEKEIFNLICKI |
| | EGSEDKKGNYKHGLAYELGV |
| | LLFGEPNEASKPEFDRKIKK |
| | FNSIYSFAQIQQIAFAERKG |
| | NANTCAVCSADNAHRMQQIK |
| | IIEPVEDNKDKIILSAKAQR |
| | LPAIPTRIVDGAVKKMATIL |
| | AKNIVDDNWQNIKQVLSAKH |
| | QLHIPIITESNAFEFEPALA |
| | DVKGKSLKDRRKKALERISP |
| | ENIFKDKNNRIKEFAKGISA |
| | YSGANLTDGDFDGAKEELDH |
| | IIPRSHKKYGTLNDEANLIC |
| | VTRGDNKNKGNRIFCLRDLA |
| | DNYKLKQFETTDDLEIEKKI |
| | ADTIWDANKKDFKFGNYRSF |
| | INLTPQEQKAFRHALFLADE |
| | NPIKQAVIRAINNRNRTFVN |
| | GTQRYFAEVLANNIYLRAKK |
| | ENLNTDKISFDYFGIPTIGN |
| | GRGIAEIRQLYEKVDSDIQA |
| | YAKGDKPQASYSHLIDAMLA |
| | FCIAADEHRNDGSIGLEIDK |
| | NYSLYPLDKNTGEVFTKDIF |
| | SQIKITDNEFSDKKLVRKKA |
| | IEGFNTHRQMTRDGIYAENY |
| | LPILIHKELNEVRKGYTWKN |
| | SEEIKIFKGKKYDIQQLNNL |
| | VYCLKFVDKPISIDIQISTL |
| | EELRNILTTNNIAATAEYYY |
| | INLKTQKLHEYYIENYNTAL |
| | GYKKYSKEMEFLRSLAYRSE |
| | RVKIKSIDDVKQVLDKDSNF |
| | IIGKITLPFKKEWQRLYREW |
| | QNTTIKDDYEFLKSFFNVKS |
| | ITKLHKKVRKDFSLPISTNE |
| | GKFLVKRKTWDNNFIYQILN |
| | DSDSRADGTKPFIPAFDISK |
| | NEIVEAIIDSFTSKNIFWLP |
| | KNIELQKVDNKNIFAIDTSK |
| | WFEVETPSDLRDIGIATIQY |
| | KIDNNSRPKVRVKLDYVIDD |
| | DSKINYFMNHSLLKSRYPDK |
| | VLEILKQSTIIEFESSGFNK |
| | TIKEMLGMKLAGIYNETSNN |
| | (SEQ ID NO: 15) |
| Lactobacillus | MKVNNYHIGLDIGTSSIGWV |
| buchneri Cas9 | AIGKDGKPLRVKGKTAIGAR |
| | LFQEGNPAADRRMFRTTRRR |
| | LSRRKWRLKLLEEIFDPYIT |
| | PVDSTFFARLKQSNLSPKDS |
| | RKEFKGSMLFPDLTDMQYHK |
| | NYPTIYHLRHALMTQDKKFD |
| | IRMVYLAIHHIVKYRGNFLN |
| | STPVDSFKASKVDFVDQFKK |
| | LNELYAAINPEESFKINLAN |
| | SEDIGHQFLDPSIRKFDKKK |
| | QIPKIVPVMMNDKVTDRLNG |
| | KIASEIIHAILGYKAKLDVV |
| | LQCTPVDSKPWALKFDDEDI |
| | DAKLEKILPEMDENQQSIVA |
| | ILQNLYSQVTLNQIVPNGMS |
| | LSESMIEKYNDHHDHLKLYK |
| | KLIDQLADPKKKAVLKKAYS |
| | QYVGDDGKVIEQAEFWSSVK |
| | KNLDDSELSKQIMDLIDAEK |
| | FMPKQRTSQNGVIPHQLHQR |
| | ELDEIIEHQSKYYPWLVEIN |
| | PNKHDLHLAKYKIEQLVAFR |
| | VPYYVGPMITPKDQAESAET |
| | VFSWMERKGIETGQITPWNF |
| | DEKVDRKASANRFIKRMTTK |
| | DTYLIGEDVLPDESLLYEKF |
| | KVLNELNMVRVNGKLLKVAD |
| | KQAIFQDLFENYKHVSVKKL |
| | QNYIKAKTGLPSDPEISGLS |
| | DPEHFNNSLGTYNDFKKLFG |
| | SKVDEPDLQDDFEKIVEWST |
| | VFEDKKILREKLNEITWLSD |
| | QQKDVLESSRYQGWGRLSKK |
| | LLTGIVNDQGERIIDKLWNT |
| | NKNFMQIQSDDDFAKRIHEA |
| | NADQMQAVDVEDVLADAYTS |
| | PQNKKAIRQVVKVVDDIQKA |
| | MGGVAPKYISIEFTRSEDRN |
| | PRRTISRQRQLENTLKDTAK |
| | SLAKSINPELLSELDNAAKS |
| | KKGLTDRLYLYFTQLGKDIY |
| | TGEPINIDELNKYDIDHILP |
| | QAFIKDNSLDNRVLVLTAVN |
| | NGKSDNVPLRMFGAKMGHFW |
| | KQLAEAGLISKRKLKNLQTD |
| | PDTISKYAMHGFIRRQLVET |
| | SQVIKLVANILGDKYRNDDT |
| | KIIEITARMNHQMRDEFGFI |
| | KNREINDYHHAFDAYLTAFL |
| | GRYLYHRYIKLRPYFVYGDF |
| | KKFREDKVTMRNFNFLHDLT |
| | DDTQEKIADAETGEVIWDRE |
| | NSIQQLKDVYHYKFMLISHE |
| | VYTLRGAMFNQTVYPASDAG |
| | KRKLIPVKADRPVNVYGGYS |
| | GSADAYMAIVRIHNKKGDKY |
| | RVVGVPMRALDRLDAAKNVS |
| | DADFDRALKDVLAPQLTKTK |
| | KSRKTGEITQVIEDFEIVLG |
| | KVMYRQLMIDGDKKFMLGSS |
| | TYQYNAKQLVLSDQSVKTLA |
| | SKGRLDPLQESMDYNNVYTE |
| | ILDKVNQYFSLYDMNKFRHK |
| | LNLGFSKFISFPNHNVLDGN |
| | TKVSSGKREILQEILNGLHA |
| | NPTFGNLKDVGITTPFGQLQ |
| | QPNGILLSDETKIRYQSPTG |
| | LFERTVSLKDL |
| | (SEQ ID NO: 16) |
| Listeria | MKKPYTIGLDIGTNSVGWAV |
| innocua | LTDQYDLVKRKMKIAGDSEK |
| Cas9 | KQIKKNFWGVRLFDEGQTAA |
| | DRRMARTARRRIERRRNRIS |
| | YLQGIFAEEMSKTDANFFCR |
| | LSDSFYVDNEKRNSRHPFFA |
| | TIEEEVEYHKNYPTIYHLRE |
| | ELVNSSEKADLRLVYLALAH |
| | IIKYRGNFLIEGALDTQNTS |
| | VDGIYKQFIQTYNQVFASGI |
| | EDGSLKKLEDNKDVAKILVE |
| | KVTRKEKLERILKLYPGEKS |
| | AGMFAQFISLIVGSKGNFQK |
| | PFDLIEKSDIECAKDSYEED |
| | LESLLALIGDEYAELFVAAK |
| | NAYSAVVLSSIITVAEIETN |
| | AKLSASMIERFDTHEEDLGE |
| | LKAFIKLHLPKHYEEIFSNI |
| | EKHGYAGYIDGKTKQADFYK |
| | YMKMTLENIEGADYFIAKIE |
| | KENFLRKQRTFDNGAIPHQL |
| | HLEELEAILHQQAKYYPFLK |
| | ENYDKIKSLVTFRIPYFVGP |
| | LANGQSEFAWLTRKADGEIR |
| | PWNIEEKVDFGKSAVDFIEK |
| | MTNKDTYLPKENVLPKHSLC |
| | YQKYLVYNELTKVRYINDQG |
| | KTSYFSGQEKEQIFNDLFKQ |
| | KRKVKKKDLELFLRNMSHVE |
| | SPTIEGLEDSFNSSYSTYHD |
| | LLKVGIKQEILDNPVNIEML |
| | ENIVKILTVFEDKRMIKEQL |
| | QQFSDVLDGVVLKKLERRHY |
| | TGWGRLSAKLLMGIRDKQSH |
| | LTILDYLMNDDGLNRNLMQL |
| | INDSNLSFKSIIEKEQVTTA |
| | DKDIQSIVADLAGSPAIKKG |
| | ILQSLKIVDELVSVMGYPPQ |
| | TIVVEMARENQTTGKGKNNS |
| | RPRYKSLEKAIKEFGSQILK |
| | EHPTDNQELRNNRLYLYYLQ |
| | NGKDMYTGQDLDIHNLSNYD |
| | IDHIVPQSFITDNSIDNLVL |
| | TSSAGNREKGDDVPPLEIVR |
| | KRKVFWEKLYQGNLMSKRKF |
| | DYLTKAERGGLTEADKARFI |
| | HRQLVETRQITKNVANILHQ |
| | RFNYEKDDHGNTMKQVRIVT |
| | LKSALVSQFRKQFQLYKVRD |
| | VNDYHHAHDAYLNGVVANTL |
| | LKVYPQLEPEFVYGDYHQFD |
| | WFKANKATAKKQFYTNIMLF |
| | FAQKDRIIDENGEILWDKKY |
| | LDTVKKVMSYRQMNIVKKIE |
| | IQKGEFSKATIKPKGNSSKL |
| | IPRKTNWDPMKYGGLDSPNM |
| | AYAVVIEYAKGKNKLVFEKK |
| | IIRVTIMERKAFEKDEKAFL |
| | EEQGYRQPKVLAKLPKYTLY |
| | ECEEGRRRMLASANEAQKGN |
| | QQVLPNHLVTLLHHAANCEV |
| | SDGKSLDYIESNREMFAELL |
| | AHVSEFAKRYTLAEANLNKI |
| | NQLFEQNKEGDIKAIAQSFV |
| | DLMAFNAMGAPASFKFFETT |
| | IERKRYNNLKELLNSTIIYQ |
| | SITGLYESRKRLDD |
| | (SEQ ID NO: 17) |
| L. pneumophilia | MESSQILSPIGIDLGGKFTG |
| Cas9 | VCLSHLEAFAELPNHANTKY |
| | SVILIDHNNFQLSQAQRRAT |
| | RHRVRNKKRNQFVKRVALQL |
| | FQHILSRDLNAKEETALCHY |
| | LNNRGYTYVDTDLDEYIKDE |
| | TTINLLKELLPSESEHNFID |
| | WFLQKMQSSEFRKILVSKVE |
| | EKKDDKELKNAVKNIKNFIT |
| | GFEKNSVEGHRHRKVYFENI |
| | KSDITKDNQLDSIKKKIPSV |
| | CLSNLLGHLSNLQWKNLHRY |
| | LAKNPKQFDEQTFGNEFLRM |
| | LKNFRHLKGSQESLAVRNLI |
| | QQLEQSQDYISILEKTPPEI |
| | TIPPYEARTNTGMEKDQSLL |
| | LNPEKLNNLYPNWRNLIPGI |
| | IDAHPFLEKDLEHTKLRDRK |
| | RIISPSKQDEKRDSYILQRY |
| | LDLNKKIDKFKIKKQLSFLG |
| | QGKQLPANLIETQKEMETHF |
| | NSSLVSVLIQIASAYNKERE |
| | DAAQGIWFDNAFSLCELSNI |
| | NPPRKQKILPLLVGAILSED |
| | FINNKDKWAKFKIFWNTHKI |
| | GRTSLKSKCKEIEEARKNSG |
| | NAFKIDYEEALNHPEHSNNK |
| | ALIKIIQTIPDIIQAIQSHL |
| | GHNDSQALIYHNPFSLSQLY |
| | TILETKRDGFHKNCVAVTCE |
| | NYWRSQKTEIDPEISYASRL |
| | PADSVRPFDGVLARMMQRLA |
| | YEIAMAKWEQIKHIPDNSSL |
| | LIPIYLEQNRFEFEESFKKI |
| | KGSSSDKTLEQAIEKQNIQW |
| | EEKFQRIINASMNICPYKGA |
| | SIGGQGEIDHIYPRSLSKKH |
| | FGVIFNSEVNLIYCSSQGNR |
| | EKKEEHYLLEHLSPLYLKHQ |
| | FGTDNVSDIKNFISQNVANI |
| | KKYISFHLLTPEQQKAARHA |
| | LFLDYDDEAFKTITKFLMSQ |
| | QKARVNGTQKFLGKQIMEFL |
| | STLADSKQLQLEFSIKQITA |
| | EEVHDHRELLSKQEPKLVKS |
| | RQQSFPSHAIDATLTMSIGL |
| | KEFPQFSQELDNSWFINHLM |
| | PDEVHLNPVRSKEKYNKPNI |
| | SSTPLFKDSLYAERFIPVWV |
| | KGETFAIGFSEKDLFEIKPS |
| | NKEKLFTLLKTYSTKNPGES |
| | LQELQAKSKAKWLYFPINKT |
| | LALEFLHHYFHKEIVTPDDT |
| | TVCHFINSLRYYTKKESITV |
| | KILKEPMPVLSVKFESSKKN |
| | VLGSFKHTIALPATKDWERL |
| | FNHPNFLALKANPAPNPKEF |
| | NEFIRKYFLSDNNPNSDIPN |
| | NGHNIKPQKHKAVRKVFSLP |
| | VIPGNAGTMMRIRRKDNKGQ |
| | PLYQLQTIDDTPSMGIQINE |
| | DRLVKQEVLMDAYKTRNLST |
| | IDGINNSEGQAYATFDNWLT |
| | LPVSTFKPEIIKLEMKPHSK |
| | TRRYIRITQSLADFIKTIDE |
| | ALMIKPSDSIDDPLNMPNEI |
| | VCKNKLFGNELKPRDGKMKI |
| | VSTGKIVTYEFESDSTPQWI |
| | QTLYVTQLKKQP |
| | (SEQ ID NO: 18) |
| N. lactamica | MAAFKPNPMNYILGLDIGIA |
| Cas9 | SVGWAMVEVDEEENPIRLID |
| | LGVRVFERAEVPKTGDSLAM |
| | ARRLARSVRRLTRRRAHRLL |
| | RARRLLKREGVLQDADFDEN |
| | GLVKSLPNTPWQLRAAALDR |
| | KLTCLEWSAVLLHLVKHRGY |
| | LSQRKNEGETADKELGALLK |
| | GVADNAHALQTGDFRTPAEL |
| | ALNKFEKESGHIRNQRGDYS |
| | HTFSRKDLQAELNLLFEKQK |
| | EFGNPHVSDGLKEDIETLLM |
| | AQRPALSGDAVQKMLGHCTF |
| | EPAEPKAAKNTYTAERFIWL |
| | TKLNNLRILEQGSERPLTDT |
| | ERATLMDEPYRKSKLTYAQA |
| | RKLLGLEDTAFFKGLRYGKD |
| | NAEASTLMEMKAYHAISRAL |
| | EKEGLKDKKSPLNLSTELQD |
| | EIGTAFSLFKTDKDITGRLK |
| | DRVQPEILEALLKHISFDKF |
| | VQISLKALRRIVPLMEQGKR |
| | YDEACAEIYGDHYCKKNAEE |
| | KIYLPPIPADEIRNPVVLRA |
| | LSQARKVINCVVRRYGSPAR |
| | IHIETAREVGKSFKDRKEIE |
| | KRQEENRKDREKAAAKFREY |
| | FPNFVGEPKSKDILKLRLYE |
| | QQHGKCLYSGKEINLVRLNE |
| | KGYVEIDHALPFSRTWDDSF |
| | NNKVLVLGSENQNKGNQTPY |
| | EYFNGKDNSREWQEFKARVE |
| | TSRFPRSKKQRILLQKFDEE |
| | GFKERNLNDTRYVNRFLCQF |
| | VADHILLTGKGKRRVFASNG |
| | QITNLLRGFWGLRKVRIEND |
| | RHHALDAVVVACSTVAMQQK |
| | ITRFVRYKEMNAFDGKTIDK |
| | ETGEVLHQKAHFPQPWEFFA |
| | QEVMIRVFGKPDGKPEFEEA |
| | DTPEKLRTLLAEKLSSRPEA |
| | VHEYVTPLFVSRAPNRKMSG |
| | QGHMETVKSAKRLDEGISVL |
| | RVPLTQLKLKGLEKMVNRER |
| | EPKLYDALKAQLETHKDDPA |
| | KAFAEPFYKYDKAGSRTQQV |
| | KAVRIEQVQKTGVWVRNHNG |
| | IADNATMVRVDVFEKGGKYY |
| | LVPIYSWQVAKGILPDRAVV |
| | AFKDEEDWTVMDDSFEFRFV |
| | LYANDLIKLTAKKNEFLGYF |
| | VSLNRATGAIDIRTHDTDST |
| | KGKNGIFQSVGVKTALSFQK |
| | NQIDELGKEIRPCRLKKRPP |
| | VR |
| | (SEQ ID NO: 19) |
| N. meningitides | MAAFKPNPINYILGLDIGIA |
| Cas9 | SVGWAMVEIDEDENPICLID |
| | LGVRVFERAEVPKTGDSLAM |
| | ARRLARSVRRLTRRRAHRLL |
| | RARRLLKREGVLQAADFDEN |
| | GLIKSLPNTPWQLRAAALDR |
| | KLTPLEWSAVLLHLIKHRGY |
| | LSQRKNEGETADKELGALLK |
| | GVADNAHALQTGDFRTPAEL |
| | ALNKFEKESGHIRNQRGDYS |
| | HTFSRKDLQAELILLFEKQK |
| | EFGNPHVSGGLKEGIETLLM |
| | TQRPALSGDAVQKMLGHCTF |
| | EPAEPKAAKNTYTAERFIWL |
| | TKLNNLRILEQGSERPLTDT |
| | ERATLMDEPYRKSKLTYAQA |
| | RKLLGLEDTAFFKGLRYGKD |
| | NAEASTLMEMKAYHAISRAL |
| | EKEGLKDKKSPLNLSPELQD |
| | EIGTAFSLFKTDEDITGRLK |
| | DRIQPEILEALLKHISFDKF |
| | VQISLKALRRIVPLMEQGKR |
| | YDEACAEIYGDHYGKKNT |
| | EEKIYLPPIPADEIRNPVVL |
| | RALSQARKVINGVVRRYGSP |
| | ARIHIETAREVGKSFKDRKE |
| | IEKRQEENRKDREKAAAKFR |
| | EYFPNFVGEPKSKDILKLRL |
| | YEQQHGKCLYSGKEINLGRL |
| | NEKGYVEIDHALPFSRTWDD |
| | SFNNKVLVLGSENQNKGNQT |
| | PYEYFNGKDNSREWQEFKAR |
| | VETSRFPRSKKQRILLQKFD |
| | EDGFKERNLNDTRYVNRFLC |
| | QFVADRMRLTGKGKKRVFAS |
| | NGQITNLLRGFWGLRKVRAE |
| | NDRHHALDAVVVACSTVAMQ |
| | QKITRFVRYKEMNAFDGKTI |
| | DKETGEVLHQKTHFPQPWEF |
| | FAQEVMIRVFGKPDGKPEFE |
| | EADTPEKLRTLLAEKLSSRP |
| | EAVHEYVTPLFVSRAPNRKM |
| | SGQGHMETVKSAKRLDEGVS |
| | VLRVPLTQLKLKDLEKMVNR |
| | EREPKLYEALKARLEAHKDD |
| | PAKAFAEPFYKYDKAGNRTQ |
| | QVKAVRVEQVQKTGVWVRNH |
| | NGIADNATMVRVDVFEKGDK |
| | YYLVPIYSWQVAKGILPDRA |
| | VVQGKDEEDWQLIDDSFNFK |
| | FSLHPNDLVEVITKKARMFG |
| | YFASCHRGTGNINIRIHDLD |
| | HKIGKNGILEGIGVKTALSF |
| | QKYQIDELGKEIRPCRLKKR |
| | PPVR |
| | (SEQ ID NO: 20) |
| B. longum | MLSRQLLGASHLARPVSYSY |
| Cas9 | NVQDNDVHCSYGERCFMRGK |
| | RYRIGIDVGLNSVGLAAVEV |
| | SDENSPVRLLNAQSVIHDGG |
| | VDPQKNKEAITRKNMSGVAR |
| | RTRRMRRRKRERLHKLDMLL |
| | GKFGYPVIEPESLDKPFEEW |
| | HVRAELATRYIEDDELRRES |
| | ISIALRHMARHRGWRNPYRQ |
| | VDSLISDNPYSKQYGELKEK |
| | AKAYNDDATAAEEESTPAQL |
| | VVAMLDAGYAEAPRLRWRTG |
| | SKKPDAEGYLPVRLMQEDNA |
| | NELKQIFRVQRVPADEWKPL |
| | FRSVFYAVSPKGSAEQRVGQ |
| | DPLAPEQARALKASLAFQEY |
| | RIANVITNLRIKDASAELRK |
| | LTVDEKQSIYDQLVSPSSED |
| | ITWSDLCDFLGFKRSQLKGV |
| | GSLTEDGEERISSRPPRLTS |
| | VQRIYESDNKIRKPLVAWWK |
| | SASDNEHEAMIRLLSNTVDI |
| | DKVREDVAYASAIEFIDGLD |
| | DDALTKLDSVDLPSGRAAYS |
| | VETLQKLTRQMLTTDDDLHE |
| | ARKTLFNVTDSWRPPADPIG |
| | EPLGNPSVDRVLKNVNRYLM |
| | NCQQRWGNPVSVNIEHVRSS |
| | FSSVAFARKDKREYEKNNEK |
| | RSIFRSSLSEQLRADEQMEK |
| | VRESDLRRLEAIQRQNGQCL |
| | YCGRTITFRTCEMDHIVPRK |
| | GVGSTNTRTNFAAVCAECNR |
| | MKSNTPFAIWARSEDAQTRG |
| | VSLAEAKKRVTMFTFNPKSY |
| | APREVKAFKQAVIARLQQTE |
| | DDAAIDNRSIESVAWMADEL |
| | HRRIDWYFNAKQYVNSASID |
| | DAEAETMKTTVSVFQGRVTA |
| | SARRAAGIEGKIHFIGQQSK |
| | TRLDRRHHAVDASVIAMMNT |
| | AAAQTLMERESLRESQRLIG |
| | LMPGERSWKEYPYEGTSRYE |
| | SFHLWLDNMDVLLELLNDAL |
| | DNDRIAVMQSQRYVLGNSIA |
| | HDATIHPLEKVPLGSAMSAD |
| | LIRRASTPALWCALTRLPDY |
| | DEKEGLPEDSHREIRVHDTR |
| | YSADDEMGFFASQAAQIAVQ |
| | EGSADIGSAIHHARVYRCWK |
| | TNAKGVRKYFYGMIRVFQTD |
| | LLRACHDDLFTVPLPPQSIS |
| | MRYGEPRVVQALQSGNAQYL |
| | GSLVVGDEIEMDFSSLDVDG |
| | QIGEYLQFFSQFSGGNLAWK |
| | HWVVDGFFNQTQLRIRPRYL |
| | AAEGLAKAFSDDVVPDGVQK |
| | IVTKQGWLPPVNTASKTAVR |
| | IVRRNAFGEPRLSSAHHMPC |
| | SWQWRHE |
| | (SEQ ID NO: 21) |
| A. muciniphila | MSRSLTFSFDIGYASIGWAV |
| Cas9 | IASASHDDADPSVCGCGTVL |
| | FPKDDCQAFKRREYRRLRRN |
| | IRSRRVRIERIGRLLVQAQI |
| | ITPEMKETSGHPAPFYLASE |
| | ALKGHRTLAPIELWHVLRWY |
| | AHNRGYDNNASWSNSLSEDG |
| | GNGEDTERVKHAQDLMDKHG |
| | TATMAETICRELKLEEGKAD |
| | APMEVSTPAYKNLNTAFPRL |
| | IVEKEVRRILELSAPLIPGL |
| | TAEIIELIAQHHPLTTEQ |
| | RGVLLQHGIKLARRYRGS |
| | LLFGQLIPRFDNRIISRCPV |
| | TWAQVYEAELKKGNSEQSAR |
| | ERAEKLSKVPTANCPEFYEY |
| | RMARILCNIRADGEPLSAEI |
| | RRELMNQARQEGKLTKASLE |
| | KAISSRLGKETETNVSNYFT |
| | LHPDSEEALYLNPAVEVLQR |
| | SGIGQILSPSVYRIAANRLR |
| | RGKSVTPNYLLNLLKSRGES |
| | GEALEKKIEKESKKKEADYA |
| | DTPLKPKYATGRAPYARTVL |
| | KKVVEEILDGEDPTRPARGE |
| | AHPDGELKAHDGCLYCLLDT |
| | DSSVNQHQKERRLDTMTNNH |
| | LVRHRMLILDRLLKDLIQDF |
| | ADGQKDRISRVCVEVGKELT |
| | TFSAMDSKKIQRELTLRQKS |
| | HTDAVNRLKRKLPGKALSAN |
| | LIRKCRIAMDMNWTCPFTGA |
| | TYGDHELENLELEHIVPHSF |
| | RQSNALSSLVLTWPGVNRMK |
| | GQRTGYDFVEQEQENPVPDK |
| | PNLHICSLNNYRELVEKLDD |
| | KKGHEDDRRRKKKRKALLMV |
| | RGLSHKHQSQNHEAMKEIGM |
| | TEGMMTQSSHLMKLACKSIK |
| | TSLPDAHIDMIPGAVTAEVR |
| | KAWDVFGVFKELCPEAADPD |
| | SGKILKENLRSLTHLHHALD |
| | ACVLGLIPYIIPAHHNGLLR |
| | RVLAMRRIPEKLIPQVRPVA |
| | NQRHYVLNDDGRMMLRDLSA |
| | SLKENIREQLMEQRVIQHVP |
| | ADMGGALLKETMQRVLSVDG |
| | SGEDAMVSLSKKKDGKKEKN |
| | QVKASKLVGVFPEGPSKLKA |
| | LKAAIEIDGNYGVALDPKPV |
| | VIRHIKVFKRIMALKEQNGG |
| | KPVRILKKGMLIHLTSSKDP |
| | KHAGVWRIESIQDSKGGVKL |
| | DLQRAHCAVPKNKTHECNWR |
| | EVDLISLLKKYQMKRYPTSY |
| | TGTPR |
| | (SEQ ID NO: 22) |
| O. laneus Cas9 | METTLGIDLGTNSIGLALVD |
| | QEEHQILYSGVRIFPEGINK |
| | DTIGLGEKEESRNATRRAKR |
| | QMRRQYFRKKLRKAKLLELL |
| | IAYDMCPLKPEDVRRWKNWD |
| | KQQKSTVRQFPDTPAFREWL |
| | KQNPYELRKQAVTEDVTRPE |
| | LGRILYQMIQRRGFLSSRKG |
| | KEEGKIFTGKDRMVGIDETR |
| | KNLQKQTLGAYLYDIAPKNG |
| | EKYRFRTERVRARYTLRDMY |
| | IREFEIIWQRQAGHLGLAHE |
| | QATRKKNIFLEGSATNVRNS |
| | KLITHLQAKYGRGHVLIEDT |
| | RITVTFQLPLKEVLGGKIEI |
| | EEEQLKFKSNESVLFWQRPL |
| | RSQKSLLSKCVFEGRNFYDP |
| | VHQKWIIAGPTPAPLSHPEF |
| | EEFRAYQFINNIIYGKNEHL |
| | TAIQREAVFELMCTESKDFN |
| | FEKIPKHLKLFEKFNFDDTT |
| | KVPACTTISQLRKLFPHPVW |
| | EEKREEIWHCFYFYDDNTLL |
| | FEKLQKDYALQTNDLEKIKK |
| | IRLSESYGNVSLKAIRRINP |
| | YLKKGYAYSTAVLLGGIRNS |
| | FGKRFEYFKEYEPEIEKAVC |
| | RILKEKNAEGEVIRKIKDYL |
| | VHNRFGFAKNDRAFQKLYHH |
| | SQAITTQAQKERLPETGNLR |
| | NPIVQQGLNELRRTVNKLLA |
| | TCREKYGPSFKFDHIHVEMG |
| | RELRSSKTEREKQSRQIREN |
| | EKKNEAAK |
| | VKLAEYGLKAYRDNIQKYLL |
| | YKEIEEKGGTVCCPYTGKTL |
| | NISHTLGSDNSVQIEHIIPY |
| | SISLDDSLANKTLCDATFNR |
| | EKGELTPYDFYQKDPSPEKW |
| | GASSWEEIEDRAFRLLPYAK |
| | AQRFIRRKPQESNEFISRQL |
| | NDTRYISKKAVEYLSAICSD |
| | VKAFPGQLTAELRHLWGLNN |
| | ILQSAPDITFPLPVSATENH |
| | REYYVITNEQNEVIRLFPKQ |
| | GETPRTEKGELLLTGEVERK |
| | VFRCKGMQEFQTDVSDGKYW |
| | RRIKLSSSVTWSPLFAPKPI |
| | SADGQIVLKGRIEKGVFVCN |
| | QLKQKLKTGLPDGSYWISLP |
| | VISQTFKEGESVNNSKLTSQ |
| | QVQLFGRVREGIFRCHNYQC |
| | PASGADGNFWCTLDTDTAQP |
| | AFTPIKNAPPGVGGGQIILT |
| | GDVDDKGIFHADDDLHYELP |
| | ASLPKGKYYGIFTVESCDPT |
| | LIPIELSAPKTSKGENLIEG |
| | NIWVDEHTGEVRFDPKKNRE |
| | DQRHHAIDAIVIALSSQSLF |
| | QRLSTYNARRENKKRGLDST |
| | EHFPSPWPGFAQDVRQSVVP |
| | LLVSYKQNPKTLCKISKTLY |
| | KDGKKIHSCGNAVRGQLHKE |
| | TVYGQRTAPGAIEKSYHIRK |
| | DIRELKTSKHIGKVVDITIR |
| | QMLLKHLQENYHIDITQEFN |
| | IPSNAFFKEGVYRIFLPNKH |
| | GEPVPIKKIRMKEELGNAER |
| | LKDNINQYVNPRNNHHVMIY |
| | QDADGNLKEEIVSFWSVIER |
| | QNQGQPIYQLPREGRNIVSI |
| | LQINDTFLIGLKEEEPEVYR |
| | NDLSTLSKHLYRVQKLSGMY |
| | YTFRHHLASTLNNEREEFRI |
| | QSLEAWKRANPVKVQIDEIG |
| | RITFLNGPLC |
| | (SEQ ID NO: 23) |

Nuclease inactivated S. pyogenes Cas9 proteins may comprise a substitution of an Alanine (A) for an Aspartic Acid (D) at position 10 and an alanine (A) for a Histidine (H) at position 840. Exemplary nuclease inactivated S. pyogenes Cas9 proteins of the disclosure may comprise or consist of the amino acid sequence (D10A and H840A bolded and underlined):

(SEQ ID NO: 24)

| | |
|---|---|
| 1 | MDKKYSIGLA IGTNSVGWAV ITDEYKVPSK |
| | KFKVLGNTDR HSIKKNLIGA LLFDSGETAE |
| 61 | ATRLKRTARR RYTRRKNRIC YLQEIFSNEM |
| | AKVDDSFFHR LEESFLVEED KKHERHPIFG |
| 121 | NIVDEVAYHE KYPTIYHLRK KLVDSTDKAD |
| | LRLIYLALAH MIKFRGHFLI EGDLNPDNSD |
| 181 | VDKLFIQLVQ TYNQLFEENP INASGVDAKA |
| | ILSARLSKSR RLENLIAQLP GEKKNGLFGN |
| 241 | LIALSLGLTP NFKSNFDLAE DAKLQLSKDT |
| | YDDDLDNLLA QIGDQYADLF LAAKNLSDAI |
| 301 | LLSDILRVNT EITKAPLSAS MIKRYDEHHQ |
| | DLTLLKALVR QQLPEKYKEI FFDQSKNGYA |
| 361 | GYIDGGASQE EFYKFIKPIL EKMDGIEELL |
| | VKLNREDLLR KQRTFDNGSI PHQIHLGELH |
| 421 | AILRRQEDFY PFLKDNREKI EKILTFRIPY |
| | YVGPLARGNS RFAWMTRKSE ETITPWNFEE |
| 481 | VVDKGASAQS FIERMTNFDK NLPNEKVLPK |
| | HSLLYEYFTV YNELTKVKYV IEGMRKPAFL |
| 541 | SGEQKKAIVD LLFKTNRKVT VKQLKEDYFK |
| | KIECFDSVEI SGVEDRFNAS LGTYHDLLKI |
| 601 | IKDKDFLDNE ENEDILEDIV LTLTLFEDRE |
| | MIEERLKTYA HLFDDKVMKQ LKRRRYTGWG |
| 661 | RLSRKLINGI RDKQSGKTIL DFLKSDGFAN |
| | RNFMQLIHDD SLTFKEDIQK AQVSGQGDSL |
| 721 | HEHIANLAGS PAIKKGILQT VKVVDELVKV |
| | MGRHKPENIV IEMARENQTT QKGQKNSRER |
| 781 | MKRIEEGIKE LGSQILKEHP VENTQLQNEK |
| | LYLYYLQNGR DMYVDQELDI NRLSDYDVDA |
| 841 | IVPQSFLKDD SIDNKVLTRS DKNRGKSDNV |
| | PSEEVVKKMK NYWRQLLNAK LITQRKFDNL |
| 901 | TKAERGGLSE LDKAGFIKRQ LVETRQITKH |
| | VAQILDSRMN TKYDENDKLI REVKVITLKS |
| 961 | KLVSDFRKDF QFYKVREINN YHHAHDAYLN |
| | AVVGTALIKK YPKLESEFVY GDYKVYDVRK |
| 1021 | MIAKSEQEIG KATAKYFFYS NIMNFFKIEI |
| | TLANGEIRKR PLIETNGETG EIVWDKGRDF |
| 1081 | ATVRKVLSMP QVNIVKKTEV QTGGFSKESI |
| | LPKRNSDKLI ARKKDWDPKK YGGFDSPTVA |
| 1141 | YSVLVVAKVE KGKSKKLKSV KELLGITIME |
| | RSSFEKNPID FLEAKGYKEV KKDLIIKLPK |
| 1201 | YSLFELENGR KRMLASAGEL QKGNELALPS |
| | KYVNFLYLAS HYEKLKGSPE DNEQKQLFVE |
| 1261 | QHKHYLDEII EQISEFSKRV ILADANLDKV |
| | LSAYNKHRDK PIREQAENII HLFTLTNLGA |
| 1321 | PAAFKYFDTT IDRKRYTSTK EVLDATLIHQ |
| | SITGLYETRI DLSQLGGD. |

Exemplary wild type Francisella tularensis subsp. Novicida Cpf1 (FnCpf1) proteins of the disclosure may comprise or consist of the amino acid sequence:

(SEQ ID NO: 25)

| | |
|---|---|
| 1 | MSIYQEFVNK YSLSKTLRFE LIPQGKTLEN |
| | IKARGLILDD EKRAKDYKKA KQIIDKYHQF |
| 61 | FIEEILSSVC ISEDLLQNYS DVYFKLKKSD |
| | DDNLQKDFKS AKDTIKKQIS EYIKDSEKFK |
| 121 | NLFNQNLIDA KKGQESDLIL WLKQSKDNGI |
| | ELFKANSDIT DIDEALEIIK SFKGWTTYFK |
| 181 | GFHENRKNVY SSNDIPTSII YRIVDDNLPK |
| | FLENKAKYES LKDKAPEAIN YEQIKKDLAE |
| 241 | ELTFDIDYKT SEVNQRVFSL DEVFEIANFN |
| | NYLNQSGITK FNTIIGGKFV NGENTKRKGI |
| 301 | NEYINLYSQQ INDKTLKKYK MSVLFKQILS |
| | DTESKSFVID KLEDDSDVVT TMQSFYEQIA |
| 361 | AFKTVEEKSI KETLSLLFDD LKAQKLDLSK |
| | IYFKNDKSLT DLSQQVFDDY SVIGTAVLEY |
| 421 | ITQQIAPKNL DNPSKKEQEL IAKKTEKAKY |
| | LSLETIKLAL EEFNKHRDID KQCRFEEILA |
| 481 | NFAAIPMIFD EIAQNKDNLA QISIKYQNQG |
| | KKDLLQASAE DDVKAIKDLL DQTNNLLHKL |
| 541 | KIFHISQSED KANILDKDEH FYLVFEECYF |
| | ELANIVPLYN KIRNYITQKP YSDEKFKLNF |
| 601 | ENSTLANGWD KNKEPDNTAI LFIKDDKYYL |
| | GVMNKKNNKI FDDKAIKENK GEGYKKIVYK |
| 661 | LLPGANKMLP KVFFSAKSIK FYNPSEDILR |
| | IRNHSTHTKN GSPQKGYEKF EFNIEDCRKF |
| 721 | IDFYKQSISK HPEWKDFGFR FSDTQRYNSI |
| | DEFYREVENQ GYKLTFENIS ESYIDSVVNQ |
| 781 | GKLYLFQIYN KDFSAYSKGR PNLHTLYWKA |
| | LFDERNLQDV VYKLNGEAEL FYRKQSIPKK |
| 841 | ITHPAKEAIA NKNKDNPKKE SVFEYDLIKD |
| | KRFTEDKFFF HCPITINFKS SGANKFNDEI |
| 901 | NLLLKEKAND VHILSIDRGE RHLAYYTLVD |
| | GKGNIIKQDT FNIIGNDRMK TNYHDKLAAI |
| 961 | EKDRDSARKD WKKINNIKEM KEGYLSQVVH |
| | EIAKLVIEYN AIVVFEDLNF GFKRGRFKVE |
| 1021 | KQVYQKLEKM LIEKLNYLVF KDNEFDKTGG |
| | VLRAYQLTAP FETFKKMGKQ TGIIYYVPAG |
| 1081 | FTSKICPVTG FVNQLYPKYE SVSKSQEFFS |
| | KFDKICYNLD KGYFEFSFDY KNFGDKAAKG |
| 1141 | KWTIASFGSR LINFRNSDKN HNWDTREVYP |
| | TKELEKLLKD YSIEYGHGEC IKAAICGESD |
| 1201 | KKFFAKLTSV LNTILQMRNS KTGTELDYLI |
| | SPVADVNGNF FDSRQAPKNM PQDADANGAY |
| 1261 | HIGLKGLMLL GRIKNNQEGK KLNLVIKNEE |
| | YFEFVQNRNN |

Exemplary wild type Lachnospiraceae bacterium sp. ND2006 Cpf1 (LbCpf1) proteins of the disclosure may comprise or consist of the amino acid sequence:

(SEQ ID NO: 26)

| | |
|---|---|
| 1 | AASKLEKFTN CYSLSKTLRF KAIPVGKTQE |
| | NIDNKRLLVE DEKRAEDYKG VKKLLDRYYL |
| 61 | SFINDVLHSI KLKNLNNYIS LFRKKTRTEK |
| | ENKELENLEI NLRKEIAKAF KGAAGYKSLF |
| 121 | KKDIIETILP EAADDKDEIA LVNSENGETT |
| | AFTGFFDNRE NMFSEEAKST SIAFRCINEN |
| 181 | LTRYISNMDI FEKVDAIFDK HEVQEIKEKI |
| | LNSDYDVEDF FEGEFFNFVL TQEGIDVYNA |
| 241 | IIGGFVTESG EKIKGLNEYI NLYNAKTKQA |
| | LPKFKPLYKQ VLSDRESLSF YGEGYTSDEE |
| 301 | VLEVFRNTLN KNSEIFSSIK KLEKLFKNFD |
| | EYSSAGIFVK NGPAISTISK DIFGEWNLIR |
| 361 | DKWNAEYDDI HLKKKAVVTE KYEDDRRKSF |
| | KKIGSFSLEQ LQEYADADLS VVEKLKEIII |
| 421 | QKVDEIYKVY GSSEKLFDAD FVLEKSLKKN |
| | DAVVAIMKDL LDSVKSFENY IKAFFGEGKE |
| 481 | TNRDESFYGD FVLAYDILLK VDHIYDAIRN |
| | YVTQKPYSKD KFKLYFQNPQ FMGGWDKDKE |
| 541 | TDYRATILRY GSKYYLAIMD KKYAKCLQKI |
| | DKDDVNGNYE KINYKLLPGP NKMLPKVFFS |
| 601 | KKWMAYYNPS EDIQKIYKNG TFKKGDMFNL |
| | NDCHKLIDFF KDSISRYPKW SNAYDFNFSE |
| 661 | TEKYKDIAGF YREVEEQGYK VSFESASKKE |
| | VDKLVEEGKL YMFQIYNKDF SDKSHGTPNL |
| 721 | HTMYFKLLFD ENNHGQIRLS GGAELFMRRA |
| | SLKKEELVVH PANSPIANKN PDNPKKTTTL |
| 781 | SYDVYKDKRF SEDQYELHIP IAINKCPKNI |
| | FKINTEVRVL LKHDDNPYVI GIDRGERNLL |
| 841 | YIVVVDGKGN IVEQYSLNEI INNFNGIRIK |
| | TDYHSLLDKK EKERFEARQN WTSIENIKEL |
| 901 | KAGYISQVVH KICELVEKYD AVIALEDLNS |
| | GFKNSRVKVE KQVYQKFEKM LIDKLNYMVD |
| 961 | KKSNPCATGG ALKGYQITNK FESFKSMSTQ |
| | NGFIFYIPAW LTSKIDPSTG FVNLLKTKYT |
| 1021 | SIADSKKFIS SFDRIMYVPE EDLFEFALDY |
| | KNFSRTDADY IKKWKLYSYG NRIRIFAAAK |
| 1081 | KNNVFAWEEV CLTSAYKELF NKYGINYQQG |
| | DIRALLCEQS DKAFYSSFMA LMSLMLQMRN |
| 1141 | SITGRTDVDF LISPVKNSDG IFYDSRNYEA |
| | QENAILPKNA DANGAYNIAR KVLWAIGQFK |
| 1201 | KAEDEKLDKV KIAISNKEWL EYAQTSVK |

Exemplary wild type Acidaminococcus sp. BV3L6 Cpf1 (AsCpf1) proteins of the disclosure may comprise or consist of the amino acid sequence:

(SEQ ID NO: 27)

| | |
|---|---|
| 1 | MTQFEGFTNL YQVSKTLRFE LIPQGKTLKH |
| | IQEQGFIEED KARNDHYKEL KPIIDRIYKT |
| 61 | YADQCLQLVQ LDWENLSAAI DSYRKEKTEE |
| | TRNALIEEQA TYRNAIHDYF IGRIDNLIDA |
| 121 | INKRHAEIYK GLFKAELENG KVLKQLGTVT |
| | TTEHENALLR SFDKFTTYFS GFYENRKNVF |
| 181 | SAEDISTAIP HRIVQDNFPK FKENCHIFTR |
| | LITAVPSLRE HFENVKKAIG IFVSTSIEEV |
| 241 | FSFPFYNQLL TQTQIDLYNQ LLGGISREAG |
| | TEKIKGLNEV LNLAIQKNDE TAHIIASLPH |
| 301 | RFIPLFKQIL SDRNTLSFIL EEFKSDEEVI |
| | QSFCKYKTLL RNENVLETAE ALFNELNSID |
| 361 | LTHIFISHKK LETISSALCD HWDTLRNALY |
| | ERRISELTGK ITKSAKEKVQ RSLKHEDINL |
| 421 | QEIISAAGKE LSEAFKQKTS EILSHAHAAL |
| | DQPLPTTLKK QEEKEILKSQ LDSLLGLYHL |
| 481 | LDWFAVDESN EVDPEFSARL TGIKLEMEPS |
| | LSFYNKARNY ATKKPYSVEK FKLNFQMPTL |
| 541 | ASGWDVNKEK NNGAILFVKN GLYYLGIMPK |
| | QKGRYKALSF EPTEKTSEGF DKMYYDYFPD |
| 601 | AAKMIPKCST QLKAVTAHFQ THTTPILLSN |
| | NFIEPLEITK EIYDLNNPEK EPKKFQTAYA |
| 661 | KKTGDQKGYR EALCKWIDFT RDFLSKYTKT |
| | TSIDLSSLRP SSQYKDLGEY YAELNPLLYH |
| 721 | ISFQRIAEKE IMDAVETGKL YLFQIYNKDF |
| | AKGHHGKPNL HTLYWTGLFS PENLAKTSIK |
| 781 | LNGQAELFYR PKSRMKRMAH RLGEKMLNKK |
| | LKDQKTPIPD TLYQELYDYV NHRLSHDLSD |
| 841 | EARALLPNVI TKEVSHEIIK DRRFTSDKFF |
| | FHVPITLNYQ AANSPSKFNQ RVNAYLKEHP |
| 901 | ETPIIGIDRG ERNLIYITVI DSTGKILEQR |
| | SLNTIQQFDY QKKLDNREKE RVAARQAWSV |
| 961 | VGTIKDLKQG YLSQVIHEIV DLMIHYQAVV |
| | VLENLNFGFK SKRTGIAEKA VYQQFEKMLI |
| 1021 | DKLNCLVLKD YPAEKVGGVL NPYQLTDQFT |
| | SFAKMGTQSG FLFYVPAPYT SKIDPLTGFV |
| 1081 | DPFVWKTIKN HESRKHFLEG FDFLHYDVKT |
| | GDFILHFKMN RNLSFQRGLP GFMPAWDIVF |
| 1141 | EKNETQFDAK GTPFIAGKRI VPVIENHRFT |
| | GRYRDLYPAN ELIALLEEKG IVFRDGSNIL |
| 1201 | PKLLENDDSH AIDTMVALIR SVLQMRNSNA |
| | ATGEDYINSP VRDLNGVCFD SRFQNPEWPM |
| 1261 | DADANGAYHI ALKGQLLLNH LKESKDLKLQ |
| | NGISNQDWLA YIQELRN |

In some embodiments of the compositions of the disclosure, the sequence encoding the RNA binding protein comprises a sequence isolated or derived from a CRISPR Cas protein or RNA-binding portion thereof. In some embodiments, the CRISPR Cas protein comprises a Type VI CRISPR Cas protein. In some embodiments, the Type VI CRISPR Cas protein comprises a Cas13 protein. Exemplary Cas13 proteins of the disclosure may be isolated or derived from any species, including, but not limited to, a bacteria or an archaea. Exemplary Cas13 proteins of the disclosure may be isolated or derived from any species, including, but not limited to, Leptotrichia wadei, Listeria seeligeri serovar 1/2b (strain ATCC 35967/DSM 20751/CIP 100100/SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d, and orthologs thereof. Exemplary Cas13b proteins of the disclosure include, but are not limited to, subtypes 1 and 2 referred to herein as Csx27 and Csx28, respectively.

Exemplary Cas13a proteins include, but are not limited to:

| | Cas13a | | | Direct |
|---|---|---|---|---|
| Cas13a | abbrevia- | Organism | Accession | Repeat |
| number | tion | name | number | sequence |
| Cas13a1 | LshCas13a | Leptotrichia | WP_018451595.1 | CCAC |
| | | shahii | (SEQ ID NO: | CCCA |
| | | | 194) | ATAT |
| | | | | CGAA |
| | | | | GGGG |
| | | | | ACTA |
| | | | | AAAC |
| | | | | (SEQ |
| | | | | ID |
| | | | | NO: |
| | | | | 28) |
| Cas13a2 | LwaCas13a | Leptotrichia | WP_021746774.1 | GATT |
| | | wadei | (SEQ ID NO: | TAGA |
| | | | 195) | CTAC |
| | | | | CCCA |
| | | | | AAAA |
| | | | | CGAA |
| | | | | GGGG |
| | | | | ACTA |
| | | | | AAAC |
| | | | | (SEQ |
| | | | | ID |
| | | | | NO: |
| | | | | 29) |
| Cas13a3 | LseCas13a | Listeria | WP_012985477.1 | GTAA |
| | | seeligeri | (SEQ ID NO: | GAGA |
| | | | 196) | CTAC |
| | | | | CTCT |
| | | | | ATAT |
| | | | | GAAA |
| | | | | GAGG |
| | | | | ACTA |
| | | | | AAAC |
| | | | | (SEQ |
| | | | | ID |
| | | | | NO: |
| | | | | 30) |
| Cas13a4 | LbmCas13a | Lachnospiraceae | WP_044921188.1 | GTAT |
| | | bacterium | (SEQ ID NO: | TGAG |
| | | MA2020 | 197) | AAAA |
| | | | | GCCA |
| | | | | GATA |
| | | | | TAGT |
| | | | | TGGC |
| | | | | AATA |
| | | | | GAC |
| | | | | (SEQ |
| | | | | ID |
| | | | | NO: |
| | | | | 31) |
| Cas13a5 | LbnCas13a | Lachnospiraceae | WP_022785443.1 | GTTG |
| | | bacterium | (SEQ ID NO: | ATGA |
| | | NK4A179 | 198) | GAAG |
| | | | | AGCC |
| | | | | CAAG |
| | | | | ATAG |
| | | | | AGGG |
| | | | | CAAT |
| | | | | AAC |
| | | | | (SEQ |
| | | | | ID |
| | | | | NO: |
| | | | | 32) |
| Cas13a6 | CamCas13a | [Clostridium] | WP_031473346.1 | GTCT |
| | | aminophilum | (SEQ ID NO: | ATTG |
| | | DSM 10710 | 199) | CCCT |
| | | | | CTAT |
| | | | | ATCG |
| | | | | GGCT |
| | | | | GTTC |
| | | | | TCCA |
| | | | | AAC |
| | | | | (SEQ |
| | | | | ID |
| | | | | NO: |
| | | | | 33) |
| Cas13a7 | CgaCas13a | Carnobacterium | WP_034560163.1 | ATTA |
| | | gallinarum | (SEQ ID NO: | AAGA |
| | | DSM 4847 | 200) | CTAC |
| | | | | CTCT |
| | | | | AAAT |
| | | | | GTAA |
| | | | | GAGG |
| | | | | ACTA |
| | | | | TAAC |
| | | | | (SEQ |
| | | | | ID |
| | | | | NO: |
| | | | | 34) |
| Cas13a8 | Cga2Cas13a | Carnobacterium | WP_034563842.1 | AATA |
| | | gallinarum | (SEQ ID NO: | TAAA |
| | | DSM 4847 | 201) | CTAC |
| | | | | CTCT |
| | | | | AAAT |
| | | | | GTAA |
| | | | | GAGG |
| | | | | ACTA |
| | | | | TAAC |
| | | | | (SEQ |
| | | | | ID |
| | | | | NO: |
| | | | | 35) |
| Cas13a9 | Pprcas13a | Paludibacter | WP_013443710.1 | CTTG |
| | | propionicigenes | (SEQ ID NO: | TGGA |
| | | WB4 | 202) | TTAT |
| | | | | CCCA |
| | | | | AAAT |
| | | | | TGAA |
| | | | | GGGA |
| | | | | ACTA |
| | | | | CAAC |
| | | | | (SEQ |
| | | | | ID |
| | | | | NO: |
| | | | | 36) |
| Cas13a10 | LweCas13a | Listeria | WP_036059185.1 | GATT |
| | | weihenstephanensis | (SEQ ID NO: | TAGA |
| | | FSL R9-0317 | 203) | GTAC |
| | | | | CTCA |
| | | | | AAAT |
| | | | | AGAA |
| | | | | GAGG |
| | | | | TCTA |
| | | | | AAAC |
| | | | | (SEQ |
| | | | | ID |
| | | | | NO: |
| | | | | 37) |
| Cas13a11 | LbfCas13a | Listeriaceae | WP_036091002.1 | GATT |
| | | bacterium FSL | (SEQ ID NO: | TAGA |
| | | M6-0635 | 204) | GTAC |
| | | (Listeria | | CTCA |
| | | newyorkensis) | | AAAC |
| | | | | AAAA |
| | | | | GAGG |
| | | | | ACTA |
| | | | | AAAC |
| | | | | (SEQ |
| | | | | ID |
| | | | | NO: |
| | | | | 38) |
| Cas13a12 | Lwa2cas13a | Leptotrichia | WP_021746774. | GATA |
| | | wadei F0279 | 1 (SEQ ID NO: | TAGA |
| | | | 205) | TAAC |
| | | | | CCCA |
| | | | | AAAA |
| | | | | CGAA |
| | | | | GGGA |
| | | | | TCTA |
| | | | | AAAC |
| | | | | (SEQ |
| | | | | ID |
| | | | | NO: |
| | | | | 39) |
| Cas13a13 | RcsCas13a | Rhodobacter | WP_013067728.1 | GCCT |
| | | capsulatus SB | (SEQ ID NO: | CACA |
| | | 1003 | 206) | TCAC |
| | | | | CGCC |
| | | | | AAGA |
| | | | | CGAC |
| | | | | GGCG |
| | | | | GACT |
| | | | | GAAC |
| | | | | (SEQ |
| | | | | ID |
| | | | | NO: |
| | | | | 40) |
| Cas13a14 | RcrCas13a | Rhodobacter | WP_023911507.1 | GCCT |
| | | capsulatus | (SEQ ID NO: | CACA |
| | | R121 | 207) | TCAC |
| | | | | CGCC |
| | | | | AAGA |
| | | | | CGAC |
| | | | | GGCG |
| | | | | GACT |
| | | | | GAAC |
| | | | | (SEQ |
| | | | | ID |
| | | | | NO: |
| | | | | 41) |
| Cas13a15 | RcdCas13a | Rhodobacter | WP_023911507.1 | GCCT |
| | | capsulatus | (SEQ ID NO: | CACA |
| | | DE442 | 208) | TCAC |
| | | | | CGCC |
| | | | | AAGA |
| | | | | CGAC |
| | | | | GGCG |
| | | | | GACT |
| | | | | GAAC |
| | | | | (SEQ |
| | | | | ID |
| | | | | NO: |
| | | | | 42) |

Exemplary wild type Cas13a proteins of the disclosure may comprise or consist of the amino acid sequence:

(SEQ ID NO: 43)

| | |
|---|---|
| 1 | MGNLFGHKRW YEVRDKKDFK IKRKVKVKRN |
| | YDGNKYILNI NENNNKEKID NNKFIRKYIN |
| 61 | YKKNDNILKE FTRKFHAGNI LFKLKGKEGI |
| | IRIENNDDFL ETEEVVLYIE AYGKSEKLKA |
| 121 | LGITKKKIID EAIRQGITKD DKKIEIKRQE |
| | NEEEIEIDIR DEYTNKTLND CSIILRIIEN |
| 181 | DELETKKSIY EIFKNINMSL YKIIEKIIEN |
| | ETEKVFENRY YEEHLREKLL KDDKIDVILT |
| 241 | NFMEIREKIK SNLEILGFVK FYLNVGGDKK |
| | KSKNKKMLVE KILNINVDLT VEDIADFVIK |
| 301 | ELEFWNITKR IEKVKKVNNE FLEKRRNRTY |
| | IKSYVLLDKH EKFKIERENK KDKIVKFFVE |
| 361 | NIKNNSIKEK IEKILAEFKI DELIKKLEKE |
| | LKKGNCDTEI FGIFKKHYKV NFDSKKFSKK |
| 421 | SDEEKELYKI IYRYLKGRIE KILVNEQKVR |
| | LKKMEKIEIE KILNESILSE KILKRVKQYT |
| 481 | LEHIMYLGKL RHNDIDMITV NTDDFSRLHA |
| | KEELDLELIT FFASTNMELN KIFSRENINN |
| 541 | DENIDFFGGD REKNYVLDKK ILNSKIKIIR |
| | DLDFIDNKNN ITNNFIRKFT KIGTNERNRI |
| 601 | LHAISKERDL QGTQDDYNKV INIIQNLKIS |
| | DEEVSKALNL DVVFKDKKNI ITKINDIKIS |
| 661 | EENNNDIKYL PSFSKVLPEI LNLYRNNPKN |
| | EPFDTIETEK IVLNALIYVN KELYKKLILE |
| 721 | DDLEENESKN IFLQELKKTL GNIDEIDENI |
| | IENYYKNAQI SASKGNNKAI KKYQKKVIEC |
| 781 | YIGYLRKNYE ELFDFSDFKM NIQEIKKQIK |
| | DINDNKTYER ITVKISDKTI VINDDFEYII |
| 841 | SIFALLNSNA VINKIRNRFF ATSVWLNTSE |
| | YQNIIDILDE IMQLNTLRNE CITENWNLNL |
| 901 | EEFIQKMKEI EKDFDDFKIQ TKKEIFNNYY |
| | EDIKNNILTE FKDDINGCDV LEKKLEKIVI |
| 961 | FDDETKFEID KKSNILQDEQ RKLSNINKKD |
| | LKKKVDQYIK DKDQEIKSKI LCRIIFNSDF |
| 1021 | LKKYKKEIDN LIEDMESENE NKFQEIYYPK |
| | ERKNELYIYK KNLFLNIGNP NFDKIYGLIS |
| 1081 | NDIKMADAKF LFNIDGKNIR KNKISEIDAI |
| | LKNLNDKLNG YSKEYKEKYI KKLKENDDFF |
| 1141 | AKNIQNKNYK SFEKDYNRVS EYKKIRDLVE |
| | FNYLNKIESY LIDINWKLAI QMARFERDMH |
| 1201 | YIVNGLRELG IIKLSGYNTG ISRAYPKRNG |
| | SDGFYTTTAY YKFFDEESYK KFEKICYGFG |
| 1261 | IDLSENSEIN KPENESIRNY ISHFYIVRNP |
| | FADYSIAEQI DRVSNLLSYS TRYNNSTYAS |
| 1321 | VFEVFKKDVN LDYDELKKKF KLIGNNDILE |
| | RLMKPKKVSV LELESYNSDY IKNLIIELLT |
| 1381 | KIENINDIL |

Exemplary Cas13b proteins include, but are not limited to:

| | | Cas13b |
|---|---|---|
| Species | Cas13b Accession | Size (aa) |
| Paludibacter propionicigenes WB4 | WP_013446107.1 | 1155 |
| | (SEQ ID NO: 209) | |
| Prevotella sp. P5-60 | WP_044074780.1 | 1091 |
| | (SEQ ID NO: 210) | |
| Prevotella sp. P4-76 | WP_044072147.1 | 1091 |
| | (SEQ ID NO: 211) | |
| Prevotella sp. P5-125 | WP_044065294.1 | 1091 |
| | (SEQ ID NO: 212) | |
| Prevotella sp. P5-119 | WP_042518169.1 | 1091 |
| | (SEQ ID NO: 213) | |
| Capnocytophaga canimorsus Cc5 | WP_013997271.1 | 1200 |
| | (SEQ ID NO: 214) | |
| Phaeodactylibacter xiamenensis | WP_044218239.1 | 1132 |
| | (SEQ ID NO: 215) | |
| Porphyromonas gingivalis W83 | WP_005873511.1 | 1136 |
| | (SEQ ID NO: 216) | |
| Porphyromonas gingivalis F0570 | WP_021665475.1 | 1136 |
| | (SEQ ID NO: 217) | |
| Porphyromonas gingivalis ATCC | WP_012458151.1 | 1136 |
| 33277 | (SEQ ID NO: 218) | |
| Porphyromonas gingivalis F0185 | ERJ81987.1 | 1136 |
| | (SEQ ID NO: 219) | |
| Porphyromonas gingivalis F0185 | WP_021677657.1 | 1136 |
| | (SEQ ID NO: 220) | |
| Porphyromonas gingivalis SJD2 | WP_023846767.1 | 1136 |
| | (SEQ ID NO: 221) | |
| Porphyromonas gingivalis F0568 | ERJ65637.1 | 1136 |
| | (SEQ ID NO: 222) | |
| Porphyromonas gingivalis W4087 | ERJ87335.1 | 1136 |
| | (SEQ ID NO: 223) | |
| Porphyromonas gingivalis W4087 | WP_021680012.1 | 1136 |
| | (SEQ ID NO: 224) | |
| Porphyromonas gingivalis F0568 | WP_021663197.1 | 1136 |
| | (SEQ ID NO: 225) | |
| Porphyromonas gingivalis | WP_061156637.1 | 1136 |
| | (SEQ ID NO: 226) | |
| Porphyromonas gulae | WP_039445055.1 | 1136 |
| | (SEQ ID NO: 227) | |
| Bacteroides pyogenes F0041 | ERI81700.1 | 1116 |
| | (SEQ ID NO: 228) | |
| Bacteroides pyogenes JCM 10003 | WP_034542281.1 | 1116 |
| | (SEQ ID NO: 229) | |
| Alistipes sp. ZOR0009 | WP_047447901.1 | 954 |
| | (SEQ ID NO: 230) | |
| Flavobacterium branchiophilum | WP_014084666.1 | 1151 |
| FL-15 | (SEQ ID NO: 231) | |
| Prevotella sp. MA2016 | WP_036929175.1 | 1323 |
| | (SEQ ID NO: 232) | |
| Myroides odoratimimus CCUG | EHO06562.1 | 1160 |
| 10230 | (SEQ ID NO: 233) | |
| Myroides odoratimimus CCUG | EKB06014.1 | 1158 |
| 3837 | (SEQ ID NO: 234) | |
| Myroides odoratimimus CCUG | WP_006265509.1 | 1158 |
| 3837 | (SEQ ID NO: 235) | |
| Myroides odoratimimus CCUG | WP_006261414.1 | 1158 |
| 12901 | (SEQ ID NO: 236) | |
| Myroides odoratimimus CCUG | EHO08761.1 | 1158 |
| 12901 | (SEQ ID NO: 237) | |
| Myroides odoratimimus | WP_058700060.1 | 1160 |
| (NZ_CP013690.1) | (SEQ ID NO: 238) | |
| Bergeyella zoohelcum ATCC | EKB54193.1 | 1225 |
| 43767 | (SEQ ID NO: 239) | |
| Capnocytophaga cynodegmi | WP_041989581.1 | 1219 |
| | (SEQ ID NO: 240) | |
| Bergeyella zoohelcum ATCC | WP_002664492.1 | 1225 |
| 43767 | (SEQ ID NO: 241) | |
| Flavobacterium sp. 316 | WP_045968377.1 | 1156 |
| | (SEQ ID NO: 242) | |
| Psychroflexus torquis ATCC | WP_015024765.1 | 1146 |
| 700755 | (SEQ ID NO: 243) | |
| Flavobacterium columnare ATCC | WP_014165541.1 | 1180 |
| 49512 | (SEQ ID NO: 244) | |
| Flavobacterium columnare | WP_060381855.1 | 1214 |
| | (SEQ ID NO: 245) | |
| Flavobacterium columnare | WP_063744070.1 | 1214 |
| | (SEQ ID NO: 246) | |
| Flavobacterium columnare | WP_065213424.1 | 1215 |
| | (SEQ ID NO: 247) | |
| Chryseobacterium sp. YR477 | WP_047431796.1 | 1146 |
| | (SEQ ID NO: 248) | |
| Riemerella anatipestifer ATCC | WP_004919755.1 | 1096 |
| 11845 = DSM 15868 | (SEQ ID NO: 249) | |
| Riemerella anatipestifer RA-CH-2 | WP_015345620.1 | 949 |
| Riemerella anatipestifer | WP_049354263.1 | 949 |
| | (SEQ ID NO: 250) | |
| Riemerella anatipestifer | WP_061710138.1 | 951 |
| | (SEQ ID NO: 251) | |
| Riemerella anatipestifer | WP_064970887.1 | 1096 |
| | (SEQ ID NO: 252) | |
| Prevotella saccharolytica F0055 | EKY00089.1 | 1151 |
| | (SEQ ID NO: 253) | |
| Prevotella saccharolytica JCM | WP_051522484.1 | 1152 |
| 17484 | | |
| Prevotella buccae ATCC 33574 | EFU31981.1 | 1128 |
| | (SEQ ID NO: 254) | |
| Prevotella buccae ATCC 33574 | WP_004343973.1 | 1128 |
| | (SEQ ID NO: 255) | |
| Prevotella buccae D17 | WP_004343581.1 | 1128 |
| | (SEQ ID NO: 256) | |
| Prevotella sp. MSX73 | WP_007412163.1 | 1128 |
| | (SEQ ID NO: 257) | |
| Prevotella pallens ATCC 700821 | EGQ18444.1 | 1126 |
| | (SEQ ID NO: 258) | |
| Prevotella pallens ATCC 700821 | WP_006044833.1 | 1126 |
| | (SEQ ID NO: 259) | |
| Prevotella intermedia ATCC | WP_036860899.1 | 1127 |
| 25611 = DSM 20706 | (SEQ ID NO: 260) | |
| Prevotella intermedia | WP_061868553.1 | 1121 |
| | (SEQ ID NO: 261) | |
| Prevotella intermedia 17 | AFJ07523.1 | 1135 |
| | (SEQ ID NO: 262) | |
| Prevotella intermedia | WP_050955369.1 | 1133 |
| Prevotella intermedia | BAU18623.1 | 1134 |
| | (SEQ ID NO: 263) | |
| Prevotella intermedia ZT | KJJ86756.1 | 1126 |
| | (SEQ ID NO: 264) | |
| Prevotella aurantiaca JCM 15754 | WP_025000926.1 | 1125 |
| | (SEQ ID NO: 265) | |
| Prevotella pleuritidis F0068 | WP_021584635.1 | 1140 |
| Prevotella pleuritidis JCM 14110 | WP_036931485.1 | 1117 |
| Prevotella falsenii DSM 22864 = | WP_036884929.1 | 1134 |
| JCM 15124 | (SEQ ID NO: 266) | |
| Porphyromonas gulae | WP_039418912.1 | 1176 |
| | (SEQ ID NO: 267) | |
| Porphyromonas sp. COT-052 | WP_039428968.1 | 1176 |
| OH4946 | (SEQ ID NO: 268) | |
| Porphyromonas gulae | WP_039442171.1 | 1175 |
| | (SEQ ID NO: 269) | |
| Porphyromonas gulae | WP_039431778.1 | 1176 |
| | (SEQ ID NO: 270) | |
| Porphyromonas gulae | WP_046201018.1 | 1176 |
| | (SEQ ID NO: 271) | |
| Porphyromonas gulae | WP_039434803.1 | 1176 |
| | (SEQ ID NO: 272) | |
| Porphyromonas gulae | WP_039419792.1 | 1120 |
| | (SEQ ID NO: 273) | |
| Porphyromonas gulae | WP_039426176.1 | 1120 |
| | (SEQ ID NO: 274) | |
| Porphyromonas gulae | WP_039437199.1 | 1120 |
| | (SEQ ID NO: 275) | |
| Porphyromonas gingivalis TDC60 | WP_013816155.1 | 1120 |
| | (SEQ ID NO: 276) | |
| Porphyromonas gingivalis ATCC | WP_012458414.1 | 1120 |
| 33277 | (SEQ ID NO: 277) | |
| Porphyromonas gingivalis | WP_058019250.1 | 1176 |
| A7A1-28 | (SEQ ID NO: 278) | |
| Porphyromonas gingivalis JCVI | EOA10535.1 | 1176 |
| SC001 | (SEQ ID NO: 279) | |
| Porphyromonas gingivalis W50 | WP_005874195.1 | 1176 |
| | (SEQ ID NO: 280) | |
| Porphyromonas gingivalis | WP_052912312.1 | 1176 |
| | (SEQ ID NO: 281) | |
| Porphyromonas gingivalis AJW4 | WP_053444417.1 | 1120 |
| | (SEQ ID NO: 282) | |
| Porphyromonas gingivalis | WP_039417390.1 | 1120 |
| | (SEQ ID NO: 283) | |
| Porphyromonas gingivalis | WP_061156470.1 | 1120 |
| | (SEQ ID NO: 284) | |

Exemplary wild type Bergeyella zoohelcum ATCC 43767 Cas13b (BzCas13b) proteins of the disclosure may comprise or consist of the amino acid sequence:

(SEQ ID NO: 44)

| | |
|---|---|
| 1 | menktslgnn iyynpfkpqd ksyfagyfna |
| | amentdsvfr elgkrlkgke ytsenffdai |
| 61 | fkenislvey eryvkllsdy fpmarlldkk |
| | evpikerken fkknfkgiik avrdlrnfyt |
| 121 | hkehgeveit deifgvldem lkstvltvkk |
| | kkvktdktke ilkksiekql dilcqkkley |
| 181 | lrdtarkiee krrnqrerge kelvapfkys |
| | dkrddliaai yndafdvyid kkkdslkess |
| 241 | kakyntksdp qqeegdlkip iskngvvfll |
| | slfltkqeih afkskiagfk atvideatvs |
| 301 | eatvshgkns icfmatheif shlaykklkr |
| | kvrtaeinyg eaenaeqlsv yaketlmmqm |
| 361 | ldelskvpdv vyqn1sedvg ktfiedwney |
| | lkenngdvgt meeeqvihpv irkryedkfn |
| 421 | yfairfldef aqfptlrfqv hlgnylhdsr |
| | pkenlisdrr ikekitvfgr lselehkkal |
| 481 | fikntetned rehyweifpn pnydfpkeni |
| | svndkdfpia gsildrekqp vagkigikvk |
| 541 | llnqqyvsev dkavkahqlk grkaskpsig |
| | niieeivpin esnpkeaivf ggqptaylsm |
| 601 | ndihsilyef fdkwekkkek lekkgekelr |
| | keigkelekk ivgkigagiq qiidkdtnak |
| 661 | ilkpyqdgns taidkeklik dlkqegnilq |
| | klkdeqtvre keyndfiayq dknreinkvr |
| 721 | drnhkqylkd nlkrkypeap arkevlyyre |
| | kgkvavwlan dikrfmptdf knewkgeqhs |
| 781 | llqkslayye qckeelknll pekvfqhlpf |
| | klggyfqqky lyqfytcyld krleyisglv |
| 841 | qqaenfksen kvfkkvenec fkflkkqnyt |
| | hkeldarvqs ilgypifler gfmdekptii |
| 901 | kgktfkgnea lfadwfryyk eyqnfqtfyd |
| | tenyplvele kkqadrkrkt kiyqqkkndv |
| 961 | ftllmakhif ksvfkqdsid qfsledlyqs |
| | reerlgnger arqtgerntn yiwnktvdlk |
| 1021 | lcdgkitven vklknvgdfi kyeydgrvqa |
| | flkyeeniew qaflikeske eenypyvver |
| 1081 | eiegyekvrr eellkevhli eeyilekvkd |
| | keilkkgdnq nfkyyilngl lkqlknedve |
| 1141 | sykvfnlnte pedvninqlk geatdlegka |
| | fvltyirnkf ahnqlpkkef wdycqekygk |
| 1201 | iekektyaey faevfkkeke alik |

Exemplary wild type Cas13d proteins of the disclosure may comprise or consist of the amino acid sequences:

| | |
|---|---|
| Cas13d | IEKKKSFAKGMGVKS |
| (Ruminococcus | TLVSGSKVYMTTFAE |
| flavefaciens | GSDARLEKIVEGDSI |
| XPD3002) | RSVNEGEAFSAEMAD |
| | KNAGYKIGNAKFSHP |
| | KGYAVVANNPLYTGP |
| | VQQDMLGLKETLEKR |
| | YFGESADGNDNICIQ |
| | VIHNILDIEKILAEY |
| | ITNAAYAVNNISGLD |
| | KDIIGFGKFSTVYTY |
| | DEFKDPEHHRAAFNN |
| | NDKLINAIKAQYDEF |
| | DNFLDNPRLGYFGQA |
| | FFSKEGRNYIINYGN |
| | ECYDILALLSGLAHW |
| | VVANNEEESRISRTW |
| | LYNLDKNLDNEYIST |
| | LNYLYDRITNELTNS |
| | FSKNSAANVNYIAET |
| | LGINPAEFAEQYFRF |
| | SIMKEQKNLGFNITK |
| | LREVMLDRKDMSEIR |
| | KNHKVFDSIRTKVYT |
| | MMDFVIYRYYIEEDA |
| | KVAAANKSLPDNEKS |
| | LSEKDIFVINLRGSF |
| | NDDQKDALYYDEANR |
| | IWRKLENIMHNIKEF |
| | RGNKTREYKKKDAPR |
| | LPRILPAGRDVSAFS |
| | KLMYALTMFLDGKEI |
| | NDLLTTLINKFDNIQ |
| | SFLKVMPLIGVNAKF |
| | VEEYAFFKDSAKIAD |
| | ELRLIKSFARMGEPI |
| | ADARRAMYIDAIRIL |
| | GTNLSYDELKALADT |
| | FSLDENGNKLKKGKH |
| | GMRNFIINNVISNKR |
| | FHYLIRYGDPAHLHE |
| | IAKNEAVVKFVLGRI |
| | ADIQKKQGQNGKNQI |
| | DRYYETCIGKDKGKS |
| | VSEKVDALTKIITGM |
| | NYDQFDKKRSVIEDT |
| | GRENAEREKFKKIIS |
| | LYLTVIYHILKNIVN |
| | INARYVIGFHCVERD |
| | AQLYKEKGYDINLKK |
| | LEEKGFSSVTKLCAG |
| | IDETAPDKRKDVEKE |
| | MAERAKESIDSLESA |
| | NPKLYANYIKYSDEK |
| | KAEEFTRQINREKAK |
| | TALNAYLRNTKWNVI |
| | IREDLLRIDNKTCTL |
| | FANKAVALEVARYVH |
| | AYINDIAEVNSYFQL |
| | YHYIMQRIIMNERYE |
| | KSSGKVSEYFDAVND |
| | EKKYNDRLLKLLCVP |
| | FGYCIPRFKNLSIEA |
| | LFDRNEAAKFDKEKK |
| | SGNS |
| | (SEQ ID NO: 45) |
| Cas13d | MKRQKTFAKRIGIKS |
| (contig e- | TVAYGQGKYAITTFG |
| k87_11092736) | KGSKAEIAVRSADPP |
| | EETLPTESDATLSIH |
| | AKFAKAGRDGREFKC |
| | GDVDETRIHTSRSEY |
| | ESLISNPAESPREDY |
| | LGLKGTLERKFFGDE |
| | YPKDNLRIQIIYSIL |
| | DIQKILGLYVEDILH |
| | FVDGLQDEPEDLVGL |
| | GLGDEKMQKLLSKAL |
| | PYMGFFGSTDVFKVT |
| | KKREERAAADEHNAK |
| | VFRALGAIRQKLAHF |
| | KWKESLAIFGANANM |
| | PIRFFQGATGGRQLW |
| | NDVIAPLWKKRIERV |
| | RKSFLSNSAKNLWVL |
| | YQVFKDDTDEKKKAR |
| | ARQYYHFSVLKEGKN |
| | LGFNLTKTREYFLDK |
| | FFPIFHSSAPDVKRK |
| | VDTFRSKFYAILDFI |
| | IYEASVSVANSGQMG |
| | KVAPWKGAIDNALVK |
| | LREAPDEEAKEKIYN |
| | VLAASIRNDSLFLRL |
| | KSACDKFGAEQNRPV |
| | FPNELRNNRDIRNVR |
| | SEWLEATQDVDAAAF |
| | VQLIAFLCNFLEGKE |
| | INELVTALIKKFEGI |
| | QALIDLLRNLEGVDS |
| | IRFENEFALFNDDKG |
| | NMAGRIARQLRLLAS |
| | VGKMKPDMTDAKRVL |
| | YKSALEILGAPPDEV |
| | SDEWLAENILLDKSN |
| | NDYQKAKKTVNPFRN |
| | YIAKNVITSRSFYYL |
| | VRYAKPTAVRKLMSN |
| | PKIVRYVLKRLPEKQ |
| | VASYYSAIWTQSESN |
| | SNEMVKLIEMIDRLT |
| | TEIAGFSFAVLKDKK |
| | DSIVSASRESRAVNL |
| | EVERLKKLTTLYMSI |
| | AYIAVKSLVKVNARY |
| | FIAYSALERDLYFFN |
| | EKYGEEFRLHFIPYE |
| | LNGKTCQFEYLAILK |
| | YYLARDEETLKRKCE |
| | ICEEIKVGCEKHKKN |
| | ANPPYEYDQEWIDKK |
| | KALNSERKACERRLH |
| | FSTHWAQYATKRDEN |
| | MAKHPQKWYDILASH |
| | YDELLALQATGWLAT |
| | QARNDAEHLNPVNEF |
| | DVYIEDLRRYPEGTP |
| | KNKDYHIGSYFEIYH |
| | YIRQRAYLEEVLAKR |
| | KEYRDSGSFTDEQLD |
| | KLQKILDDIRARGSY |
| | DKNLLKLEYLPFAYN |
| | LPRYKNLTTEALFDD |
| | DSVSGKKRVAEWRER |
| | EKTREAEREQRRQR |
| | (SEQ ID NO: 46) |
| Cas13d | GTGAGAAGTCTCCTT |
| (contig e- | ATGGGGAGATGCTAC |
| k87_11092736) | (SEQ ID NO:47) |
| Direct | |
| Repeat Sequence | |
| Cas13d | MKNSVTFKLIQAQEN |
| (160582958_ | KEAARKKAKDIAEQA |
| gene4983 | RIAKRNGVVKKEENR |
| 4) | INRIQIEIQTQKKSN |
| | TQNAYHLKSLAKAAG |
| | VKSVFAIGNDLLMTG |
| | FGPGNDATIEKRVFQ |
| | NRAIETLSSPEQYSA |
| | EFQNKQFKIKGNIKV |
| | LNHSTQKMEEIQTEL |
| | QDNYNRPHFDLLGCK |
| | NVLEQKYFGRTFSDN |
| | IHVQIAYNIMDIEKL |
| | LTPYINNIIYTLNEL |
| | MRDNSKDDFFGCDSH |
| | FSVAYLYDELKAGYS |
| | DRLKTKPNLSKNIDR |
| | IWNNFCNYMNSDSGN |
| | TEARLAYFGELFYKP |
| | KETGDAKSDYKTHLS |
| | NNQKEEWELKSDKEV |
| | YNIFAILCDLRHFCT |
| | HGESITPSGKPFPYN |
| | LEKNLFPEAKQVLNS |
| | LFEEKAESLGAEAFG |
| | KTAGKTDVSILLKVF |
| | EKEQASQKEQQALLK |
| | EYYDFKVQKTYKNMG |
| | FSIKKLREAIMEIPD |
| | AAKFKDDLYSSLRHK |
| | LYGLFDFILVKHFLD |
| | TSDSENLQNNDIFRQ |
| | LRACRCEEEKDQVYR |
| | SIAVKVWEKVKKKEL |
| | NMFKQVVVIPSLSKD |
| | ELKQMEMTKNTELLS |
| | SIETISTQASLFSEM |
| | IFMMTYLLDGKEINL |
| | LCTSLIEKFENIASF |
| | NEVLKSPQIGYETKY |
| | TEGYAFFKNADKTAK |
| | ELRQVNNMARMTKPL |
| | GGVNTKCVMYNEAAK |
| | ILGAKPMSKAELESV |
| | FNLDNHDYTYSPSGK |
| | KIPNKNFRNFIINNV |
| | ITSRRFLYLIRYGNP |
| | EKIRKIAINPSIISF |
| | VLKQIPDEQIKRYYP |
| | PCIGKRTDDVTLMRD |
| | ELGKMLQSVNFEQFS |
| | RVNNKQNAKQNPNGE |
| | KARLQACVRLYLTVP |
| | YLFIKNMVNINARYV |
| | LAFHCLERDHALCFN |
| | SRKLNDDSYNEMANK |
| | FQMVRKAKKEQYEKE |
| | YKCKKQETGTAHTKK |
| | IEKLNQQIAYIDKDI |
| | KNMHSYTCRNYRNLV |
| | AHLNVVSKLQNYVSE |
| | LPNDYQITSYFSFYH |
| | YCMQLGLMEKVSSKN |
| | IPLVESLKNEANDAQ |
| | SYSAKKTLEYFDLIE |
| | KNRTYCKDFLKALNA |
| | PFSYNLPRFKNLSIE |
| | ALFDKNIVYEQADLK |
| | KE (SEQ ID NO:48) |
| Cas13d | GAACTACACCCCTCT |
| (160582958_ | GTTCTTGTAGGGGTC |
| gene4983 | TAACAC |
| 4) Direct Repeat | (SEQ IDNO: 49) |
| Sequence | |
| Cas13d (contig | MKKQKSKKTVSKTSG |
| tpg \|DJXD01000002.1\|; | LKEALSVQGTVIMTS |
| uncultivated | FGKGNMANLSYKIPS |
| Ruminococcus | SQKPQNLNSSAGLKN |
| assembly, UBA7013, | VEVSGKKIKFQGRHP |
| from sheep gut | KIATTDNPLFKPQPG |
| metagenome) | MDLLCLKDKLEMHYF |
| | GKTFDDNIHIQLIYQ |
| | ILDIEKILAVHVNNI |
| | VFTLDNVLHPQKEEL |
| | TEDFIGAGGWRINLD |
| | YQTLRGQTNKYDRFK |
| | NYIKRKELLYFGEAF |
| | YHENERRYEEDIFAI |
| | LTLLSALRQFCFHSD |
| | LSSDESDHVNSFWLY |
| | QLEDQLSDEFKETLS |
| | ILWEEVTERIDSEFL |
| | KTNTVNLHILCHVFP |
| | KESKETIVRAYYEFL |
| | IKKSFKNMGFSIKKL |
| | REIMLEQSDLKSFKE |
| | DKYNSVRAKLYKLFD |
| | FIITYYYDHHAFEKE |
| | ALVSSLRSSLTEENK |
| | EEIYIKTARTLASAL |
| | GADFKKAAADVNAKN |
| | IRDYQKKANDYRISF |
| | EDIKIGNTGIGYFSE |
| | LIYMLTLLLDGKEIN |
| | DLLTTLINKFDNIIS |
| | FIDILKKLNLEFKFK |
| | PEYADFFNMTNCRYT |
| | LEELRVINSIARMQK |
| | PSADARKIMYRDALR |
| | ILGMDNRPDEEIDRE |
| | LERTMPVGADGKFIK |
| | GKQGFRNFIASNVIE |
| | SSRFHYLVRYNNPHK |
| | TRTLVKNPNVVKFVL |
| | EGIPETQIKRYFDVC |
| | KGQEIPPTSDKSAQI |
| | DVLARIISSVDYKIF |
| | EDVPQSAKINKDDPS |
| | RNFSDALKKQRYQAI |
| | VSLYLTVMYLITKNL |
| | VYVNSRYVIAFHCLE |
| | RDAFLHGVTLPKMNK |
| | KIVYSQLTTHLLTDK |
| | NYTTYGHLKNQKGHR |
| | KWYVLVKNNLQNSDI |
| | TAVSSFRNIVAHISV |
| | VRNSNEYISGIGELH |
| | SYFELYHYLVQSMIA |
| | KNNWYDTSHQPKTAE |
| | YLNNLKKHHTYCKDF |
| | VKAYCIPFGYVVPRY |
| | KNLTINELFDRNNPN |
| | PEPKEEV |
| | (SEQ ID NO: 50) |
| Cas13d (contig | CAACTACAACCCCGT |
| tpg | AAAAATACGGGGTTC |
| \|DJXD01000002.1\|; | TGAAAC |
| uncultivated | (SEQ ID NO: 51) |
| Ruminococcus | |
| assembly, | |
| UBA7013, | |
| from sheep gut | |
| metagenome) | |
| Cas13d | SEQ ID NO: 286 |
| (Gut_metagenome_ | |
| contig6049000251) | |
| Cas13d | SEQ ID NO: 287 |
| (Gut_metagenome_ | |
| contig546000275) | |
| Cas13d | SEQ ID NO: 288 |
| (Gut_metagenome_ | |
| contig4114000374) | |
| Cas13d | SEQ ID NO: 289 |
| (Gut_metagenome_ | |
| contig721000619) | |
| Cas13d | SEQ ID NO: 290 |
| (Gut_metagenome_ | |
| contig2002000411) | |
| Cas13d | SEQ ID NO: 291 |
| (Gut_metagenome_co | |
| ntigl3552000311) | |
| Cas13d | SEQ ID NO: 292 |
| (Gut_metagenome_co | |
| ntigl0037000527) | |
| Cas13d(293.Cas13d | SEQ ID NO: 293 |
| from | |
| Gut_metagenom e_con | |
| tig238000329) | |
| Cas13d | SEQ ID NO: 294 |
| (Gut_metagenome_co | |
| ntig2643000492) | |
| Cas13d | SEQ ID NO: 295 |
| (Gut_metagenome_ | |
| contig874000057) | |
| Cas13d | SEQ ID NO: 296 |
| (Gut_metagenome_ | |
| contig4781000489) | |
| Cas13d | SEQ ID NO: 297 |
| (Gut_metagenome_ | |
| contigl2144000352) | |
| Cas13d | SEQ ID NO: 298 |
| (Gut_metagenome_ | |
| contig5590000448) | |
| Cas13d | SEQ ID NO: 299 |
| (Gut_metagenome_ | |
| contig525000349) | |
| Cas13d | SEQ ID NO: 300 |
| (Gut_metagenome_ | |
| contig7229000302) | |
| Cas13d | SEQ ID NO: 301 |
| (Gut_metagenome_ | |
| contig3227000343) | |
| Cas13d | SEQ ID NO: 302 |
| (Gut_metagenome_ | |
| contig7030000469) | |
| Cas13d | SEQ ID NO: 303 |
| (gut_metagenome_P1 | |
| 7E0k2120140920,_c87 | |
| 000043) | |
| Cas13d (Metagenomic | SEQ ID NO: 304 |
| hit (no protein | |
| accession): contig | |
| emb | |
| \|OBVH01003037.1, | |
| human gut | |
| metagenome sequence | |
| (also found in WGS | |
| contigs | |
| emb\|OBXZ01000094.1 | |
| \|and | |
| emb\|OBJF01000033.1\|)) | |
| Cas13d (Metagenomic | SEQ ID NO: 305 |
| hit (no protein | |
| accession): contig | |
| OGZC01000639.1 | |
| (human gut | |
| metagenome | |
| assembly)) | |
| Cas13d (Metagenomic | SEQ ID NO: 306 |
| hit (no protein | |
| accession): contig | |
| emb\|OHBM01000764.1 | |
| (human gut | |
| metagenome | |
| assembly)) | |
| Cas13d (Metagenomic | SEQ ID NO: 307 |
| hit (no protein | |
| accession): contig | |
| emb\|OHCP01000044.1 | |
| (human gut | |
| metagenome | |
| assembly)) | |
| Cas13d (Metagenomic | SEQ ID NO: 308 |
| hit (no protein | |
| accession): contig | |
| emb | |
| \|OGDF01008514.1\| | |
| (human gut | |
| metagenome | |
| assembly)) | |
| Cas13d (Metagenomic | SEQ ID NO: 309 |
| hit (no protein | |
| accession): contig | |
| emb\|OGPN01002610.1 | |
| (human gut | |
| metagenome | |
| assembly)) | |
| Cas13d (Metagenomic | SEQ ID NO: 310 |
| hit (no protein | |
| accession): | |
| from contig | |
| emb\|OBLI01020244 | |
| and | |
| emb\|OBLI01038679 | |
| (from pig gut | |
| metagenome)) | |
| Cas13d (Metagenomic | SEQ ID NO: 311 |
| hit (no protein | |
| accession): contig | |
| OIZX01000427.1) | |
| Cas13d (Metagenomic | SEQ ID NO: 312 |
| hit (no protein | |
| accession): contig | |
| OCTW011587266.1) | |
| Cas13d (Metagenomic | SEQ ID NO: 313 |
| hit (no protein | |
| accession): contig | |
| emb | |
| \|OGNF01009141.1) | |
| Cas13d (Metagenomic | SEQ ID NO: 314 |
| hit (no protein | |
| accession): | |
| contig | |
| emb | |
| \|OIEN01002196.11) | |
| Cas13d | SEQ ID NO: 315 |
| (Ga0129306_1000735) | |
| Cas13d | SEQ ID NO: 316 |
| (Ga0129317_1008067) | |
| Cas13d | SEQ ID NO: 317 |
| (Ga0224415_10048792) | |
| Cas13d | SEQ ID NO: 318 |
| (250twins_35838_ | |
| GL0110300) | |
| Cas13d | SEQ ID NO: 319 |
| (250twins_36050_ | |
| GL0158985) | |

The term “cell” as used herein may refer to either a prokaryotic or eukaryotic cell, optionally obtained from a subject or a commercially available source.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guide RNA or a combination of a crRNA and a tracrRNA. A CRISPR system can be used to cause double stranded or single stranded breaks in a target polynucleotide such as DNA or RNA. A CRISPR system can also be used to recruit proteins or label a target polynucleotide. In some aspects, CRISPR-mediated gene editing utilizes the pathways of non-homologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “gRNA target sequences” as used herein refers to the use of guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

In some embodiments of the compositions of the disclosure, a target sequence of an RNA molecule comprises a sequence motif corresponding to the RNA binding protein and/or the RNA binding proteins and/or fusion protein thereof.

In some embodiments of the compositions and methods of the disclosure, the sequence motif is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A sequence motif of the disclosure may comprise or consist of a mutation in an endogenous sequence that causes a disease or disorder. The mutation may comprise or consist of a sequence substitution, inversion, deletion, insertion, transposition, or any combination thereof.

A sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A sequence motif of the disclosure may comprise or consist of a secondary, tertiary, or quaternary structure. The secondary, tertiary, or quaternary structure may be endogenous or naturally occurring. The secondary, tertiary, or quaternary structure may be induced or non-naturally occurring. The secondary, tertiary, or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

In some embodiments of the compositions and methods of the disclosure, an RNA molecule of the disclosure comprises a target sequence. In some embodiments, the RNA molecule of the disclosure comprises at least one target sequence. In some embodiments, the RNA molecule of the disclosure comprises one or more target sequence(s). In some embodiments, the RNA molecule of the disclosure comprises two or more target sequences.

In some embodiments of the compositions and methods of the disclosure, an RNA molecule of the disclosure is a naturally occurring RNA molecule. In some embodiments, the RNA molecule of the disclosure is a non-naturally occurring molecule. Exemplary non-naturally occurring RNA molecules may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, an RNA molecule of the disclosure comprises or consists of a sequence isolated or derived from a virus.

In some embodiments of the compositions and methods of the disclosure, an RNA molecule of the disclosure comprises or consists of a sequence isolated or derived from a prokaryotic organism. In some embodiments, an RNA molecule of the disclosure comprises or consists of a sequence isolated or derived from a species or strain of archaea or a species or strain of bacteria.

In some embodiments of the compositions and methods of the disclosure, the RNA molecule of the disclosure comprises or consists of a sequence isolated or derived from a eukaryotic organism. In some embodiments, an RNA molecule of the disclosure comprises or consists of a sequence isolated or derived from a species of protozoa, parasite, protist, algae, fungi, yeast, amoeba, worm, microorganism, invertebrate, vertebrate, insect, rodent, mouse, rat, mammal, or a primate. In some embodiments, an RNA molecule of the disclosure comprises or consists of a sequence isolated or derived from a human.

In some embodiments of the compositions and methods of the disclosure, the RNA molecule of the disclosure comprises or consists of a sequence derived from a coding sequence from a genome of an organism or a virus. In some embodiments, the RNA molecule of the disclosure comprises or consists of a primary RNA transcript, a precursor messenger RNA (pre-mRNA) or messenger RNA (mRNA). In some embodiments, the RNA molecule of the disclosure comprises or consists of a gene product that has not been processed (e.g. a transcript). In some embodiments, the RNA molecule of the disclosure comprises or consists of a gene product that has been subject to post-transcriptional processing (e.g. a transcript comprising a 5′cap and a 3′ polyadenylation signal). In some embodiments, the RNA molecule of the disclosure comprises or consists of a gene product that has been subject to alternative splicing (e.g. a splice variant). In some embodiments, the RNA molecule of the disclosure comprises or consists of a gene product that has been subject to removal of non-coding and/or intronic sequences (e.g. a messenger RNA (mRNA)).

In some embodiments of the compositions and methods of the disclosure, the RNA molecule of the disclosure comprises or consists of a sequence derived from a non-coding sequence (e.g. a non-coding RNA (ncRNA)). In some embodiments, the RNA molecule of the disclosure comprises or consists of a ribosomal RNA. In some embodiments, the RNA molecule of the disclosure comprises or consists of a small ncRNA molecule. Exemplary small RNA molecules of the disclosure include, but are not limited to, microRNAs (miRNAs), small interfering (siRNAs), piwi-interacting RNAs (piRNAs), small nucleolar RNAs (snoRNAs), small nuclear RNAs (snRNAs), extracellular or exosomal RNAs (exRNAs), and small Cajal body-specific RNAs (scaRNAs). In some embodiments, the RNA molecule of the disclosure comprises or consists of a long ncRNA molecule. Exemplary long RNA molecules of the disclosure include, but are not limited to, X-inactive specific transcript (Xist) and HOX transcript antisense RNA (HOTAIR).

In some embodiments of the compositions and methods of the disclosure, the RNA molecule of the disclosure contacted by a composition of the disclosure in an intracellular space. In some embodiments, the RNA molecule of the disclosure contacted by a composition of the disclosure in a cytosolic space. In some embodiments, the RNA molecule of the disclosure contacted by a composition of the disclosure in a nucleus. In some embodiments, the RNA molecule of the disclosure contacted by a composition of the disclosure in a vesicle, membrane-bound compartment of a cell, or an organelle.

In some embodiments of the compositions and methods of the disclosure, the RNA molecule of the disclosure contacted by a composition of the disclosure in an extracellular space.

In some embodiments, the RNA molecule of the disclosure contacted by a composition of the disclosure in an exosome. In some embodiments, the RNA molecule of the disclosure contacted by a composition of the disclosure in a liposome, a polymersome, a micelle or a nanoparticle. In some embodiments, the RNA molecule of the disclosure contacted by a composition of the disclosure in an extracellular matrix. In some embodiments, the RNA molecule of the disclosure contacted by a composition of the disclosure in a droplet. In some embodiments, the RNA molecule of the disclosure contacted by a composition of the disclosure in a microfluidic droplet.

In some embodiments of the compositions and methods of the disclosure, a RNA molecule of the disclosure comprises or consists of a single-stranded sequence. In some embodiments, the RNA molecule of the disclosure comprises or consists of a double-stranded sequence. In some embodiments, the double-stranded sequence comprises two RNA molecules. In some embodiments, the double-stranded sequence comprises one RNA molecule and one DNA molecule. In some embodiments, including those wherein the double-stranded sequence comprises one RNA molecule and one DNA molecule, compositions of the disclosure selectively bind and, optionally, selectively cut the RNA molecule.

The term “intein” refers to a class of protein that is able to excise itself and join the remaining portion(s) of the protein via protein splicing. A “split intein” comes from two genes. A non-limiting example of a “split-intein” are the C-intein and N-intein sequences originally derived from N. punctiforme.

The term “isolated” as used herein refers to molecules or biologicals or cellular materials being substantially free from other materials.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nβ2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “IRES” refers to an internal ribosome entry site or portion thereof of viral, prokaryotic, or eukaryotic origin. In some embodiments, an IRES is an RNA element that allows for translation initiation in a cap-independent manner. Common structural features of IRES elements are described in Gritsenko A., et al. (2017) PLoS Comput Biol 13(9): e1005734, incorporated herein by reference. “IRES-like sequences” of the fusion RNAs disclosed herein refers to sequences of synthetic origin that function in a manner of an IRES or portion thereof to control translation of a target nucleic acid in a cell. In some embodiments, the IRES is one or more of the IRES or IRES-like sequences disclosed herein. In some embodiments, the IRES is having at least 65%, at least 70%, at least 75%, at least 78%, at least 80%, at least 83%, at least 85%, at least 88%, at least 90%, at least 92%, at least 95%, at least 96%, at least 97%, at least 98%, at least 99%, or 100% sequence identity to one or more of the IRES or IRES-like sequences disclosed herein.

The term “self-cleaving peptides” or “sequences encoding self-cleaving peptides” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

The term “protein”, “peptide”, and “polypeptide” are used interchangeably and in their broadest sense to refer to a compound of two or more subunits of amino acids, amino acid analogs, or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “PAMmer” refers to an oligonucleotide comprising a PAM sequence that is capable of interacting with a guide nucleotide sequence-programmable RNA binding protein. Non-limiting examples of PAMmers are described in O'Connell et al. Nature 516, pages 263-266 (2014), incorporated herein by reference. A PAM sequence refers to a protospacer adjacent motif comprising about 2 to about 10 nucleotides. PAM sequences are specific to the guide nucleotide sequence-programmable RNA binding protein with which they interact and are known in the art. For example, Streptococcus pyogenes PAM has the sequence 5′-NGG-3′, where “N” is any nucleobase followed by two guanine (“G”) nucleobases. Cas9 of Francisella novicida recognizes the canonical PAM sequence 5′-NGG-3′, but has been engineered to recognize the PAM 5′-YG-3′ (where “Y” is a pyrimidine), thus adding to the range of possible Cas9 targets. The Cpf1 nuclease of Francisella novicida recognizes the PAM 5′-TTTN-3′ or 5′-YTN-3′.

As used herein, the term “recombinant expression system” refers to a genetic construct for the expression of certain genetic material formed by recombination.

As used herein, the term “RNA-binding protein” or “RBP” includes an RNA-binding protein, polypeptide, or domain thereof including without limitation, an RNA-binding portion or portions of the RNA-binding protein or polypeptide or domain. In some embodiments, an RNA-binding protein of the disclosure is a guide nucleotide sequence-programmable RNA binding protein disclosed herein. In other embodiments, an RNA-binding protein of the disclosure is a Pumilio and FBF (PUF) protein or RNA-binding portion thereof. In some embodiments, the RNA-binding protein comprises a Pumilio-based assembly (PUMBY) protein or RNA-binding portion thereof. In some embodiments, the RNA-binding protein comprises a Pentatricopeptide Repeat (PPR) motif or motifs or RNA-binding portion thereof. In some embodiments, the RNA-binding protein does not require multimerization for RNA-binding activity. In some embodiments, the RNA-binding protein is not a monomer of a multimer complex. In some embodiments, a multimer protein complex does not comprise the RNA binding protein. In some embodiments, the RNA-binding protein selectively binds to a target sequence within the RNA molecule. In some embodiments, the RNA-binding protein does not comprise an affinity for a second sequence within the RNA molecule. In some embodiments, the RNA-binding protein does not comprise a high affinity for or selectively bind a second sequence within the RNA molecule. In some embodiments, the RNA-binding protein comprises between 2 and 1300 amino acids, inclusive of the endpoints. In some embodiments, the sequence encoding the RNA-binding protein further comprises a sequence encoding a nuclear localization signal (NLS). In some embodiments, the sequence encoding a nuclear localization signal (NLS) is positioned 3′ to the sequence encoding the RNA binding protein. In some embodiments, the RNA-binding protein comprises an NLS at a C-terminus of the protein. In some embodiments, the sequence encoding the RNA-binding protein further comprises a first sequence encoding a first NLS and a second sequence encoding a second NLS. In some embodiments, the sequence encoding the first NLS or the second NLS is positioned 3′ to the sequence encoding the RNA-binding protein. In some embodiments, the RNA-binding protein comprises the first NLS or the second NLS at a C-terminus of the protein. In some embodiments, the RNA-binding protein further comprises an NES (nuclear export signal) or other peptide tag or secretory signal. In some embodiments, a fusion protein disclosed herein comprises the RNA-binding protein as a first RNA-binding protein together with a second RNA-binding protein comprising or consisting of a nuclease domain.

As used herein, the term “subject” is intended to mean any eukaryotic organism such as a plant or an animal. In some embodiments, the subject may be a mammal; in further embodiments, the subject may be a bovine, equine, feline, murine, porcine, canine, human, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “vector” intends a recombinant vector that retains the ability to infect and transduce non-dividing and/or slowly-dividing cells and integrate into the target cell's genome. A vector can be a viral vector, bacteriophage, bacterial artificial chromosome or yeast artificial chromosome. A vector can be a DNA or RNA vector. A vector can be a self-replicating extrachromosomal vector. A vector can be a DNA plasmid. The vector may be derived from or based on a wild-type virus. Aspects of this disclosure relate to an adeno-associated virus vector, an adenovirus vector, and a lentivirus vector.

The term “translation modifier protein” refers to a protein that is able to modify translation. In some embodiments, the translation modifier protein represses translation. In some embodiments, the translation modifier protein enhances translation. In some embodiments, the translation modifier protein represses translation by 1%, 2%, 5%, 10%, 15%, 20%, 25%, 35%, 50%, 75%, 80%, 85%, 90%, 95%, 97%, 98%, 99%, or 100% as compared to a control. In some embodiments, the translation modifier protein enhances translation by 1%, 2%, 5%, 10%, 15%, 20%, 25%, 35%, 50%, 75%, 80%, 85%, 90%, 95%, 97%, 98%, 99%, or 100% as compared to a control.

As used in some embodiments herein “kinase phosphorylation domain” refers to an area within a molecule, typically but not always an amino acid, that is susceptible to the chemical addition of one or more phosphate groups by a kinase enzyme. Kinases are known to regulate a number of cellular and signal transduction pathways. Sometimes, the kinase phosphorylation domain is mutated, wherein the mutation effects the functioning of the molecule.

As used in some embodiments herein “selectable marker” refers to a component of a vector. In some embodiments, a selectable marker is a type of reporter gene used to indicate the success of a transfection. There are positive selectable markers, wherein the marker provides an advantage to the host organism. There are also negative selectable markers that eliminate or stunt growth of the host organism. There are also positive and negative selectable markers that can either advantage or inhibit growth depending on the condition. Non-limiting examples or types of markers are drug-resistance markers and auxotrophic markers.

As used in some embodiments herein, “post-transcriptionally” refers to events that occur after transcription of a gene. In some embodiments, post-transcriptional modification is when an RNA primary transcript is chemically altered following transcription from a gene to produce a functional RNA molecule. Non-limiting examples of post-transcriptional modification include addition of a cap to the 5′ end of an RNA molecule, addition of a polyadenylated tail to the 3′ end of an RNA molecule, and splicing. Additional, non-limiting examples of post-transcriptional modifications include 2′-O-Methylation (2′-OMe) (2′-O-methylation occurs on the oxygen of the free 2′-OH of the ribose moiety), N6-methyladenosine (m6A), and 5-methylcytosine (m5C). In some embodiments, gene expression may be post-transcriptionally increased or up-regulated by the implementation of the compositions and methods described herein. In some embodiments, gene expression by be post-transcriptionally decreased or down-regulated by the implementation of the compositions and methods described herein.

As used herein, the term “2-component RNA targeting system” is a nucleic acid molecule encoding a 2-component RNA targeting system comprises (a) nucleic acid sequence encoding a RNA-targeted CRISPR/Cas protein or translation modifier protein fusion; and (b) a single guide RNA (sgRNA) sequence comprising: on its 5′ end, an RNA sequence (or spacer sequence) that hybridizes to or binds to a target RNA sequence; and on its 3′ end, an RNA sequence (or scaffold sequence) capable of binding to or associating with the CRISPR/Cas protein; and wherein the 2-component RNA targeting system recognizes and alters the target RNA in a cell in the absence of a PAMmer. In some embodiments, the sequences of the 2-component system are in a single vector. In some embodiments, the spacer sequence of the 2-component system is a repeat sequence selected from the group consisting of CUG, CCUG, CAG, and GGGGCC.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Provided herein are the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., SEQ ID NO: 46 (Cas13d), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, an Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W. H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Fusion RNAs

In some aspects, provided herein are are fusion RNAs comprising, consisting of, or consisting essentially of: (i) a guide nucleotide sequence-programmable RNA; and (ii) one or more internal ribosome entry sites (IRES) or portion thereof. In some embodiments, the fusion RNA comprises a guide RNA and one or more IRES-like sequences which function as an IRES as disclosed herein to control translation of the target nucleic acid. In some embodiments, the guide nucleotide sequence-programmable RNA is a guide RNA (gRNA, such as a single gRNA (sgRNA) or a crisprRNA (crRNA). In some embodiments of the fusion RNA, the guide nucleotide sequence-programmable RNA is derived from a guide RNA scaffold from Steptococcus pyogenes, Staphylococcus aureus, Francisella novicida, Neisseria meningitidis, Streptococcus thermophilus, or Brevibacillus laterosporus. In some embodiments, the guide nucleotide sequence-programmable RNA scaffold is derived from the same bacterial species as the guide nucleotide sequence-programmable RNA binding protein.

In some embodiments of the fusion RNA, the guide nucleotide sequence-programmable RNA comprises a nucleotide sequence complementary to a target nucleic acid. In some embodiments, the target nucleic acid is an RNA, messenger RNA (mRNA), transfer RNA (tRNA), or ribosomal RNA (rRNA). In particular embodiments, the target nucleic acid is an mRNA.

In some embodiments, the sequence that is complementary and/or homologous to a target nucleic acid is about 8 to about 100, about 10 to about 50, about 15 to about 40, about 15 to about 30, or about 20 to about 30 nucleotides in length. In some embodiments, the sequence that is complementary and/or homologous to a target nucleic acid is about 20 nucleotides in length. In some embodiments, the sequence is about 50%, about 60%, about 70%, about 75%, about 80%, about 85%, about 90%, about 95%, about 97%, about 98%, about 99%, or about 100% homologous to the target nucleic acid. In particular embodiments, the sequence is about 90-100% homologous to the target nucleic acid. In some embodiments, the sequence that is complementary and/or homologous to a target nucleic acid in the fusion RNA is a spacer sequence.

In some embodiments of the fusion RNA, the IRES is a type I or a type II IRES. In some embodiments, the IRES is a viral IRES or a eukaryotic IRES. In some embodiments, the IRES is selected from a Poliovirus IRES, Rhinovirus IRES, Encephalomyocarditis virus IRES (EMCV-IRES), Picornavirus IRES, Foot-and-mouth disease virus IRES (FMDV-IRES), Aphthovirus IRES, Kaposi's sarcoma-associated herpesvirus IRES (KSHV-IRES), Hepatitis A IRES, Hepatitis C IRES, Classical swine fever virus IRES, Pestivirus IRES, Bovine viral diarrhea virus IRES, Friend murine leukemia IRES, Moloney murine leukemia IRES (MMLV-IRES), Rous sarcoma virus IRES, Human immunodeficiency virus IRES (HIV-IRES), Plautia stali intestine virus IRES, Cripavirus IRES, Cricket paralysis virus IRES, Triatoma virus IRES, Rhopalosiphum padi virus IRES, Marek's disease virus IRES, Fibroblast growth factor (FGF-1 IRES and FGF-2 IRES), Platelet-derived growth factor B (PDGF/c-sis IRES), Vascular endothelial growth factor (VEGF IRES), and an Insulin-like growth factor 2 (IGF-II IRES). In some embodiments, the IRES or IRES-like sequence is a portion of an IRES or IRES-like sequence.

In some embodiments of the fusion RNA, the fusion RNA further comprises a linker sequence located between the guide nucleotide sequence-programmable RNA and the IRES. In some embodiments, the fusion RNA comprises the structure 5′-[guide nucleotide sequence-programmable RNA]-[linker sequence]-[IRES]-3′. In some embodiments, the fusion RNA comprises the structure 5′-[IRES]-[linker sequence]-[guide nucleotide sequence-programmable RNA]-3′. In some embodiments, the linker sequence is about 1 to about 3, about 1 to about 5, about 1 to about 10, about 5 to about 20, about 10 to about 50, or about 50 to about 200 nucleobases in length. In some embodiments, the linker sequence RNA is not complementary to the target nucleic acid.

### Fusion Proteins

In some aspects, provided herein are compositions comprising one or more polynucleotides encoding: (i) a guide nucleotide sequence-programmable RNA binding protein; and (ii) a translation modifier protein or a biological equivalent thereof.

In some aspects, provided herein are fusion proteins comprising, consisting of, or consisting essentially of: (i) a guide nucleotide sequence-programmable RNA binding protein; and (ii) a translation modifier protein or a biological equivalent thereof.

In some embodiments, the translation modifier protein is at least one of eukaryotic translation initiation factor 4E (EIF4E) (SEQ ID NO 52-59), eukaryotic translation initiation factor 4E-binding protein (EIF4E-BP1) (SEQ ID NO 61-22), ubiquitin-associated protein 2-like (UBAP2L) (SEQ ID NO 64-71), and a biological equivalent of each thereof.

In some embodiments, the translation modifier protein is encoded by at least one of the polynucleotides in Table 2.

TABLE 2

| Protein Name | Accession |
|---|---|
| Homo sapiens RNA pseudouridylate synthase | BC032135.2 (SEQ ID NO: 94) |
| domain containing 3 (RPUSD3) | |
| Homo sapiens La ribonucleoprotein domain | BC131630.1 (SEQ ID NO: 95) |
| family, member 5 (LARP4B) | |
| Homo sapiens CDC-like kinase 1 (CLK1) | BC031549.1 (SEQ ID NO: 96) |
| Homo sapiens paternally expressed 10 | BC050659.2 (SEQ ID NO: 285) |
| (PEG10) | |
| Homo sapiens nucleolar and spindle | BC010838.1 (SEQ ID NO: 97) |
| associated protein 1 (NUSAP1) | |
| Homo sapiens BRCA2 and CDKN1A | BC009771.1 (SEQ ID NO: 98) |
| interacting protein (BCCIP) | |
| Homo sapiens La ribonucleoprotein domain | BC001460.2 (SEQ ID NO: 99) |
| family, member 1 (LARP1) | |
| Homo sapiens ribosomal protein L10a | BC006791.1 (SEQ ID NO: 100), BC011366.1 |
| (RPL10A) | (SEQ ID NO: 101) |
| Homo sapiens annexin A2 (ANXA2) | BC009564.1 (SEQ ID NO: 102) |
| Homo sapiens CDC-like kinase 2 (CLK2) | BC014067.2 (SEQ ID NO: 103) |
| Homo sapiens small inducible cytokine | BC014051.2 (SEQ ID NOD: 104) |
| subfamily E, member 1 (AIMP1) | |
| Homo sapiens THO complex 1 (THOC1) | BC010381 (SEQ ID NO: 105) |
| Homo sapiens KIAA1324 (KIAA1324) | BC125208.1 (SEQ ID NO: 106) |
| Homo sapiens decorin (DCN) | BC005322.1 (SEQ ID NO: 107) |
| Homo sapiens annexin A2 (ANXA2) | BC023990.1 (SEQ ID NO: 108) |
| Homo sapiens PRP3 pre-mRNA processing | BC001954.1 (SEQ ID NO: 109) |
| factor 3 homolog (S. cerevisiae) (PRPF3) | |
| Homo sapiens heat shock 27 kDa protein 1 | BC073768 (SEQ ID NO: 110), BC000510 |
| (HSPB1) | (SEQ ID NO: 111) |
| Synthetic construct Homo sapiens clone | HQ44869 (SEQ ID NO: 112) |
| (SFRS13A) | |
| Homo sapiens zinc finger, RAN-binding | BC039814.1 (SEQ ID NO: 113) |
| domain containing 2 (ZRANB2) | |
| Homo sapiens mitochondrial ribosomal | BC004896.2 (SEQ ID NO: 114) |
| protein L39 (MRPL39) | |
| Homo sapiens microtubule-associated protein | BC008715.2 (SEQ ID NO: 115), BC012794.2 |
| 4 (MAP4) | (SEQ ID NO: 116) |
| Homo sapiens RNA binding motif protein 4 | BC021120.1 (SEQ ID NO: 117) |
| (RBM4) | |
| Homo sapiens RNA binding motif protein 33 | BC011923.2 (SEQ ID NO: 118) |
| (RBM33) | |
| Homo sapiens nucleosome assembly protein | BC034954 (SEQ ID NO: 119) |
| 1-like 3 (NAP1L3) | |
| Homo sapiens mRNA; cDNA | AL832026 (SEQ ID NO: 120) |
| DKFZp451K134 (from clone | |
| DKFZp451K134) (ZCCHC6) | |
| Homo sapiens poly(A)-specific ribonuclease | BC112246.1 (SEQ ID NO: 121) |
| (PARN)-like domain containing 1 (PNLDC1) | |
| Homo sapiens RNA binding motif protein 38 | BC018711 (SEQ ID NO: 122) |
| (RBM38) | |
| Homo sapiens ELAV (embryonic lethal, | BC030692.1 (SEQ ID NO: 123) |
| abnormal vision, Drosophila)-like 2 (Hu | |
| antigen B) (ELAVL2) | |
| Homo sapiens heat shock protein 90 kDa | BC121062.2 (SEQ ID NO: 124) |
| alpha (cytosolic), class A member 1 | |
| (HSP90AA1) | |
| Homo sapiens CDC-like kinase 3 (CLK3) | BC019881.1 (SEQ ID NO: 125) |
| Homo sapiens metadherin (MTDH) | BC045642.1 (SEQ ID NO: 126) |
| Homo sapiens ribosomal protein S4, X-linked | BC100903.1 (SEQ ID NO: 127) |
| (RPS4X) | |
| Homo sapiens peptidylprolyl isomerase B | BC00112 (SEQ ID NO: 128) |
| (cyclophilin B) (PPIB) | |
| Homo sapiens interferon-induced protein with | BC032839.2 (SEQ ID NO: 129) |
| tetratricopeptide repeats 2 (IFIT2) | |
| Homo sapiens DiGeorge syndrome critical | BC009323.2 (SEQ ID NO: 130) |
| region gene 8 (DGCR8) | |
| Homo sapiens bol, boule-like (Drosophila) | BC033674.1 (SEQ ID NO: 131) |
| (BOLL) | |
| Homo sapiens exportin 5 (XPO5) | BC000129.1 (SEQ ID NO: 132) |
| Homo sapiens deleted in azoospermia 4 | BC047480.1 (SEQ ID NO: 133), BC047617.1 |
| (DAZ4) | (SEQ ID NO: 134) |
| Homo sapiens fragile X mental retardation, | BC020090.1 (SEQ ID NO: 135) |
| autosomal homolog 2 (FXR2) | |
| Homo sapiens KRR1, small subunit (SSU) | BC016778.1 (SEQ ID NO: 136) |
| processome component, homolog (yeast) | |
| (KRR1) | |
| Homo sapiens viral DNA polymerase- | BC018736.1 (SEQ ID NO: 137) |
| transactivated protein 6 (SPATS2L) | |
| Homo sapiens NIN1/RPN12 binding protein | BC064630.1 (SEQ ID NO: 138) |
| 1 homolog (S. cerevisiae) (NOB1) | |
| Homo sapiens GTPase activating protein | BC011731.2 (SEQ ID NO: 139) |
| (SH3 domain) binding protein 2 (G3BP2) | |
| Homo sapiens piwi-like 2 (Drosophila) | BC025995.1 (SEQ ID NO: 140) |
| (PIWIL2) | |
| Homo sapiens DEAD (Asp-Glu-Ala-Asp, | BC032128.2 (SEQ ID NO: 141) |
| (SEQ ID NO: 350)) box polypeptide 39 | |
| (DDX39A) | |
| Homo sapiens eukaryotic translation | BC126259.1 (SEQ ID NO: 142) |
| elongation factor 2 (EEF2) | |
| Homo sapiens piwi-like 1 (Drosophila) | BC028581.2 (SEQ ID NO: 143) |
| (PIWIL1) | |
| Homo sapiens bruno-like 5, RNA binding | BC028101.1 (SEQ ID NO: 144) |
| protein (Drosophila) (CELF5) | |
| Homo sapiens TNF receptor-associated | BC018950.2 (SEQ ID NO: 145) |
| protein 1 (TRAP1) | |
| Homo sapiens DEAD (Asp-Glu-Ala-Asp, | BC005162.2 (SEQ ID NO: 146), BC006544.2 |
| (SEQ ID NO: 350)) box polypeptide 19A | (SEQ ID NO: 147) |
| (DDX19A) | |
| Homo sapiens superkiller viralicidic activity | BC015758 (SEQ ID NO: 148) |
| 2-like (SKIV2L) | |
| Homo sapiens tripartite motif-containing 39 | BC007661.2 (SEQ ID NO: 149) |
| (TRIM39) | |
| Homo sapiens heterogeneous nuclear | BC002355.2 (SEQ ID NO: 150), BC009600.1 |
| ribonucleoprotein A1 (HNRNPA) | (SEQ ID NO: 151), BC012158.1 (SEQ ID |
| | NO: 152), BC033714.1 (SEQ ID NO: 153) |
| Homo sapiens piwi-like 4 (Drosophila) | BC031060.1 (SEQ ID NO: 154) |
| (PIWIL4) | |
| Homo sapiens zinc finger CCCH-type | BC027607.2 (SEQ ID NO: 155) |
| containing 14 (ZC3H14) | |
| Homo sapiens C1D nuclear receptor co- | BC005235.1 (SEQ ID NO: 156) |
| repressor (C1D) | |
| Homo sapiens RNA binding motif protein, Y- | BC030018.2 (SEQ ID NO: 157) |
| linked, family 1, member F (RBMY1F) | |
| Homo sapiens RNA binding motif (RNP1, | BC006825.1 (SEQ ID NO: 158) |
| RRM) protein 3 (RBM3) | |
| Homo sapiens RNA binding motif protein 19 | BC004289.1 (SEQ ID NO: 159), BC006137.1 |
| (RBM19) | (SEQ ID NO: 160) |
| Homo sapiens CDGSH iron sulfur domain 2 | BC032300.1 (SEQ ID NO: 161) |
| (CISD2) | |
| Homo sapiens histone cluster 1, H1c | BC002649.1 (SEQ ID NO: 162) |
| (HIST1H1C) | |
| Homo sapiens methyl CpG binding protein 2 | BC011612.1 (SEQ ID NO: 163) |
| (Rett syndrome) (MECP2) | |
| Homo sapiens heat shock 60 kDa protein 1 | BC003030.1 (SEQ ID NO: 164) |
| (chaperonin) (HSPD1) | |
| Homo sapiens YTH domain family, member | BC052970.1 (SEQ ID NO: 165) |
| 3 (YTHDF3) | |
| Homo sapiens RNA binding motif protein 42 | BC004204.2 (SEQ ID NO: 166) |
| (RBM42) | |
| Homo sapiens cytoplasmic polyadenylation | BC117150 (SEQ ID NO: 167) |
| element binding protein 4 (CPEB4) | |
| Homo sapiens YTH domain family, member | BC050284.1 (SEQ ID NO: 168) |
| 1 (YTHDF1) | |
| Homo sapiens Ewing sarcoma breakpoint | BC004817 (SEQ ID NO: 169) |
| region 1 (EWSR1) | |
| Synthetic construct Homo sapiens clone | BC148673 (SEQ ID NO: 170) |
| (PABPN1L) | |
| Homo sapiens cytoplasmic polyadenylation | BC103939.1 (SEQ ID NO: 171) |
| element binding protein 2 (CPEB2) | |
| Homo sapiens YTH domain family, member | BC002559.2 (SEQ ID NO: 172) |
| 2 (YTHDF2) | |
| Homo sapiens protein associated with | BC065264.1 (SEQ ID NO: 173), BC109038.1 |
| topoisomerase II homolog 1 (yeast) (PATL1) | (SEQ ID NO: 174) |
| Homo sapiens RNA binding motif protein 7 | BC034381.1 (SEQ ID NO: 175) |
| (RBM7) | |
| Homo sapiens zinc finger protein 36, C3H | BC005010 (SEQ ID NO: 176) |
| type-like 2 (ZFP36L2) | |
| Homo sapiens zinc finger CCCH-type | BC050463.1 (SEQ ID NO: 177) |
| containing 18 (ZC3H18) | |
| Homo sapiens CCR4-NOT transcription | BC011826 (SEQ ID NO: 178) |
| complex, subunit 2 (CNOT2) | |
| Homo sapiens DEAD (Asp-Glu-Ala-Asp, | BC065007.1 (SEQ ID NO: 179) |
| (SEQ ID NO: 350)) box polypeptide 6 | |
| (DDX6) | |
| Homo sapiens CCR4-NOT transcription | BC060852.1 (SEQ ID NO: 180) |
| complex, subunit 7 (CNOT7) | |
| Homo sapiens zinc finger protein 36 | BC018340.1 (SEQ ID NO: 181) |
| (ZFP36L1) | |
| Homo sapiens CCR4-NOT transcription | BC035590.1 (SEQ ID NO: 182) |
| complex, subunit 4 (CNOT4) | |
| Homo sapiens zinc finger protein 36, C3H | BC009693 (SEQ ID NO: 183) |
| type, homolog (mouse) (ZFP36) | |
| Homo sapiens poly(A)-specific ribonuclease | BC050029.1 (SEQ ID NO: 184) |
| (deadenylation nuclease) (PARN) | |
| Synthetic construct Homo sapiens clone | BC156179 (SEQ ID NO: 185) |
| (NANOS1) | |
| Homo sapiens small nuclear | BC000405.2 (SEQ ID NO: 186), BC008290.1 |
| ribonucleoprotein polypeptide A (SNRPA) | (SEQ ID NO: 187) |
| Synthetic construct Homo sapiens clone | DQ893993 (SEQ ID NO: 188) |
| (TOB1) | |
| Homo sapiens nanos homolog 2 (Drosophila) | BC117484.1 (SEQ ID NO: 189), BC117486.1 |
| (NANOS2) | (SEQ ID NO: 190) |
| Homo sapiens nanos homolog 3 (Drosophila) | BC101209.2 (SEQ ID NO: 191) |
| (NANOS3) | |
| Homo sapiens B-cell translocation gene 1, | BC016759 (SEQ ID NO: 192) |
| anti-proliferative (BTG1) | |
| Homo sapiens transducer of ERBB2, 2 | BC038957 (SEQ ID NO: 193) |
| (TOB2) | |
| Homo sapiens pre-mRNA processing factor 3 | ENST00000324862.7 (SEQ ID NO: 320) |
| (PRPF3) | |
| Homo sapiens RNA-binding E3 ubiquitin- | ENST00000406189.4 (SEQ ID NO: 321), |
| protein ligase (MEX3C) | ENST00000592416.1 (SEQ ID NO: 322), |
| | ENST00000616921.1 (SEQ ID NO: 323) |
| Homo sapiens family with sequence | ENST00000238823.13 (SEQ ID NO: 324), |
| similarity 98 member A (FAM98A) | ENST00000403368.1 (SEQ ID NO: 325), |
| | ENST00000431950.1 (SEQ ID NO: 326) |
| Homo sapiens terminal nucleotidyltransferase | ENST00000423041.6 (SEQ ID NO: 327), |
| 2 (TUT2) | ENST00000504233.5 (SEQ ID NO: 328), |
| | ENST00000296783.7 (SEQ ID NO: 329), |
| | ENST00000428308.6 (SEQ ID NO: 330), |
| | ENST00000453514.5 (SEQ ID NO: 331) |
| Homo sapiens terminal nucleotidyltransferase | ENST00000436909.7 (SEQ ID NO: 332), |
| 4B (TUT3) | ENST00000561678.5 (SEQ ID NO: 333), |
| | ENST00000357464.4 (SEQ ID NO: 334) |
| Homo sapiens terminal uridylyl transferase 4 | ENST00000528457.5 (SEQ ID NO: 335), |
| (TUT4 (ZCCHC11)) | ENST00000527941.5 (SEQ ID NO: 336), |
| | ENST00000257177.9 (SEQ ID NO: 337), |
| | ENST00000371544.7 (SEQ ID NO: 338), |
| | ENST00000494469.5 (SEQ ID NO: 339), |
| | ENST00000471623.1 (SEQ ID NO: 340), |
| | ENST00000531722.5 (SEQ ID NO: 341), |
| | ENST00000474453.6 (SEQ ID NO: 342), |
| | ENST00000528642.5 (SEQ ID NO: 343), |
| | ENST00000484723.6 (SEQ ID NO: 344), |
| | ENST00000473856.5 (SEQ ID NO: 345), |
| | ENST00000355809.4 (SEQ ID NO: 346), |
| | ENST00000470626.1 (SEQ ID NO: 347), |
| | ENST00000524582.1 (SEQ ID NO: 348) |

In some embodiments, the translation modifier protein is at least one of eukaryotic translation initiation factor 4G (EIF4G), eukaryotic translation initiation factor 4A (EIF4A), eukaryotic translation initiation factor 4B (EIF4B), eukaryotic translation initiation factor 4H (EIF4H), eukaryotic translation initiation factor 3 (EIF3), polyadenylate-binding protein 1 (PABP1), and a biological equivalent of each thereof. EIF4G and EIF3 are eukaryotic translation initiation factors involved in stabilizing preinitiation complexes by targeting 5′UTRs. PABP1 is a eukaryotic polyadenylate-binding protein which enhances circularization of messenger RNAs and promotes ribosome recycling. EIF4A, EIF4B, and EIF4H are eukaryotic helicases that unwind 5′UTR secondary structure and help preinitiation complexes find target start codons.

In some aspects, provided herein are fusion proteins comprising, consisting of, or consisting essentially of: (i) a guide nucleotide sequence-programmable RNA binding protein; and (ii) a eukaryotic translation initiation factor 4E (EIF4E) protein or a biological equivalent thereof. EIF4E is a eukaryotic translation initiation factor involved in directing ribosomes to the cap structure of mRNAs. In some embodiments, it is a 24-kD polypeptide that exists as both a free form and as part of the EIF4F pre-initiation complex. Many cellular mRNA require EIF4E in order to be translated into protein. In some embodiments, the EIF4E polypeptide is the rate-limiting component of the eukaryotic translation apparatus and is involved in the mRNA-ribosome binding step of eukaryotic protein synthesis.

In some embodiments, the guide nucleotide sequence-programmable RNA binding protein is all or part of a protein selected from: Cas9, modified Cas9, Cpf1, Cas13a, Cas13b, CasM, CasRX/Cas13d, and a biological equivalent of each thereof. In some embodiments, the guide nucleotide sequence-programmable RNA binding protein is all or part of a protein selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus CRISPR 1 Cas9 (St1Cas9), Streptococcus thermophilus CRISPR 3 Cas9 (St3Cas9), and Brevibacillus laterosporus Cas9 (BlatCas9). In some embodiments, the guide nucleotide sequence-programmable RNA binding protein is modified to be nuclease inactive.

In some embodiments, the CasRX/Cas13d protein is an effector of the type VI-D CRISPR-Cas systems. In some embodiments, the CasRX/Case13d protein is an RNA-guided RNA endonuclease enzyme that can cut or bind RNA. In some embodiments, the CasRX/Cas13d protein can include one or more higher eukaryotes and prokaryotes nucleotide-binding (HEPN) domains. In some embodiments, the CasRX/Case13d protein can include either a wild-type or mutated HEPN domain. In some embodiments, the CasRX/Case13d protein includes a mutated HEPN domain that cannot cut RNA but can process guide RNA. In some embodiments, the CasRX/Cas13d protein does not require a protospacer flanking sequence. Also see WO Publication No. WO2019/040664 & US2019/0062724, which is incorporated herein by reference in its entirety, for further examples and sequences of CasRX/Cas13d protein, without limitation, specific reference is made to SEQ ID NOS: 54, 57, 61, 67, 69, 71, 72, 73, 74, 75, 76, 77, 78, 85, 86, 87, 88, 113, 147, 153, 154, 155, 158, 160, 162, 164, 170, 179, 183, 185, 187, 189, 190, 202, 204, 206, 208, 209, 210, and 212 reproduced herein. Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described CasRX/Cas13d proteins and both of which are incorporated by reference herein in their entireties. Also see WO Publication Nos. WO2018/183703 (CasM) and WO2019/006471 (Cas13d), which are incorporated herein by reference in their entirety.

In some embodiments of the fusion proteins of the disclosure, an RNA-binding protein or RNA-binding portion thereof which is a PUF (Pumilio and FBF homology family) can be used in place of the guide nucleotide sequence-programmable RNA binding protein. The unique RNA recognition mode of PUF proteins (named for Drosophila Pumilio and C. elegans fem-3 binding factor) that are involved in mediating mRNA stability and translation are well known in the art. The PUF domain of human Pumiliol, also known in the art, binds tightly to cognate RNA sequences and its specificity can be modified. It contains eight PUF repeats that recognize eight consecutive RNA bases with each repeat recognizing a single base. Since two amino acid side chains in each repeat recognize the Watson-Crick edge of the corresponding base and determine the specificity of that repeat, a PUF domain can be designed to specifically bind most 8-nt RNA. Wang et al., Nat Methods. 2009; 6(11): 825-830. See also WO2012/068627 which is incorporated by reference herein in its entirety.

In some embodiments of the fusion proteins of the disclosure, the RNA-binding protein or RNA-binding portion thereof which is a PUMBY (Pumilio-based assembly) protein can be used in the place of the guide nucleotide sequence-programmable RNA binding protein. RNA-binding protein PumHD (Pumilio homology domain, a member of the PUF family), which has been widely used in native and modified form for targeting RNA, has been engineered to yield a set of four canonical protein modules, each of which targets one RNA base. These modules (i.e., Pumby, for Pumilio-based assembly) can be concatenated in chains of varying composition and length, to bind desired target RNAs. The specificity of such Pumby—RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, the RNA-binding protein or RNA-binding portion thereof which is a PPR protein can be used in place of the guide nucleotide sequence-programmable RNA binding protein disclosed herein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the guide nucleotide sequence-programmable RNA binding protein is bound to the fusion RNA. In some embodiments, the nucleic acid sequences encoding the RNA binding protein and the fusion RNA sequence are comprised within a single vector. In some embodiments, the nucleic acid sequences encoding the RNA binding protein and the fusion RNA sequence are comprised within two vectors.

In some embodiments, the fusion protein further comprises, consists of, or consists essentially of a linker. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the fusion protein comprises, consists of, or consists essentially of the structure NH₂-[EIF4E]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[EIF4E]-COOH.

In some embodiments, the guide nucleotide sequence-programmable RNA binding protein is bound to a guide RNA (gRNA) such as a single gRNA (sgRNA), a crisprRNA (crRNA), and/or a trans-activating crRNA (tracrRNA). In some embodiments, the sequence encoding the guide nucleotide sequence-programmable RNA binding protein and the gRNA is a 2-component system. In some embodiments, the 2-component system is comprised within a single vector.

In some embodiments, the EIF4E protein is encoded by a polynucleotide having a sequence comprising, consisting of, or consisting essentially of all or part of a sequence selected from SEQ ID NO: 52, SEQ ID NO: 53, SEQ ID NO: 54, SEQ ID NO: 55, and a biological equivalent of each thereof.

| | |
|---|---|
| (NM_001130678) | GAGTATTGCCTTTGGCCCCCACCCCCACGGGTCCCCGCGCTCCGTCTTCCT |
| Homo sapiens | TCTGACTGGGGGACTCCGCGGGACGGCGTTCCCGGCGCGCACTGTACCCC |
| eukaryotic | TTGCCGCCCCTTCCCCTTCATGTTGGACCTGACCTCCCGCGGACAAGTGG |
| translation initiation | GGACGTCCCGGAGGATGGCCGAGGCGGCGTGTAGCGCACACTTTCTGGA |
| factor 4E (EIF4E), | AACCACCCCTACTCCTAATCCCCCGACTACAGAAGAGGAGAAAACGGAA |
| transcript variant 3 | TCTAATCAGGAGGTTGCTAACCCAGAACACTATATTAAACATCCCCTACA |
| | GAACAGATGGGCACTCTGGTTTTTTAAAAATGATAAAAGCAAAACTTGG |
| | CAAGCAAACCTGCGGCTGATCTCCAAGTTTGATACTGTTGAAGACTTTTG |
| | GGCTCTGTACAACCATATCCAGTTGTCTAGTAATTTAATGCCTGGCTGTG |
| | ACTACTCACTTTTTAAGGATGGTATTGAGCCTATGTGGGAAGATGAGAAA |
| | AACAAACGGGGAGGACGATGGCTAATTACATTGAACAAACAGCAGAGAC |
| | GAAGTGACCTCGATCGCTTTTGGCTAGAGACACTTCTGTGCCTTATTGGA |
| | GAATCTTTTGATGACTACAGTGATGATGTATGTGGCGCTGTTGTTAATGTT |
| | AGAGCTAAAGGTGATAAGATAGCAATATGGACTACTGAATGTGAAAACA |
| | GAGAAGCTGTTACACATATAGGGAGGGTATACAAGGAAAGGTTAGGACT |
| | TCCTCCAAAGATAGTGATTGGTTATCAGTCCCACGCAGACACAGCTACTA |
| | AGAGCGGCTCCACCACTAAAAATAGGTTTGTTGTTTAAGAAGACACCTTC |
| | TGAGTATTCTCATAGGAGACTGCGTCAAGCAATCGAGATTTGGGAGCTGA |
| | ACCAAAGCCTCTTCAAAAAGCAGAGTGGACTGCATTTAAATTTGATTTCC |
| | ATCTTAATGTTACTCAGATATAAGAGAAGTCTCATTCGCCTTTGTCTTGTA |
| | CTTCTGTGTTCATTTTTTTTTTTTTTTTTGGCTAGAGTTTCCACTATCCCAA |
| | TCAAAGAATTACAGTACACATCCCCAGAATCCATAAATGTGTTCCTGGCC |
| | CACTCTGTAATAGTTCAGTAGAATTACCATTAATTACATACAGATTTTAC |
| | CTATCCACAATAGTCAGAAAACAACTTGGCATTTCTATACTTTACAGGAA |
| | AAAAAATTCTGTTGTTCCATTTTATGCAGAAGCATATTTTGCTGGTTTGAA |
| | AGATTATGATGCATACAGTTTTCTAGCAATTTTCTTTGTTTCTTTTTACAG |
| | CATTGTCTTTGCTGTACTCTTGCTGATGGCTGCTAGATTTTAATTTATTTGT |
| | TTCCCTACTTGATAATATTAGTGATTCTGATTTCAGTTTTTCATTTGTTTTG |
| | CTTTTGTTTTTTTCCTCATGTAACATTGGTGAAGGATCCAGGAATATGACA |
| | CAAAGGTGGAATAAACATTAATTTTGTGCATTCTTTGGTAATTTTTTTTGT |
| | TTTTTGTAACTACAAAGCTTTGCTACAAATTTATGCATTTCATTCAAATCA |
| | GTGATCTATGTTTGTGTGATTTCCTAAACATAATTGTGGATTATAAAAAA |
| | TGTAACATCATAATTACATTCCTAACTAGAATTAGTATGTCTGTTTTTGTA |
| | TCTTTATGCTGTATTTTAACACTTTGTATTACTTAGGTTATTTTGCTTTGGT |
| | TAAAAATGGCTCAAGTAGAAAAGCAGTCCCATTCATATTAAGACAGTGT |
| | ACAAAACTGTAAATAAAATGTGTACAGTGAATTGTCTTTTAGACAACTAG |
| | ATTTGTCCTTTTATTTCTCCATCTTTATAGAAGGAATTTGTACTTCTTATTG |
| | CAAGGCAGTCTCTATATTATGTCTTCTTTTGTGGTGTCTTCCATGTGAACA |
| | GCATAAGTTTGGAGCACTAGTTTGATTATTATGTTTATTACAATTTTTAAT |
| | AAATTGAATAGGTAGTATCATATATATGGAATTAAATTGATGTGGCTATC |
| | TTTGTTTTTTTATAAAGTAAGGCACAGTCATTCAGTCTTAGGTAAATAATG |
| | TACTCTCTTAATATGTTAATACTCATGAGAATTGGGATCTGATGCATCAC |
| | CATTTGATTGGTAGCAACAGTGGTTGTAAAACTTGGTTGCTGAATTGAGT |
| | TGTTTCTATGTTAAGTGTCAAAATGATAGTGTAGGGAAAGTACAGGTGGT |
| | GGGGACATATGCATTAAGAATCTTGTTAGTGTTGCAATCTAAATAGAATG |
| | GAATAAACAGGTGTTAAGACATATTTATAGTGGTAAATTGTTGTAGTATG |
| | GTATTCTGTAAACTTGAAAACTTGATCTACTCTTTGTAGGTATCATTTGAA |
| | AGCAAACTTGAAAATGTTTTGTACATAGTACATACTTGTATAGTCCTGTG |
| | AGATGAAGTATGGCTATCAGACCAAAGGATAAGCCAAACTGTAGGTAGC |
| | AGAATGGAAATTATTATTTTGAGAGGAAAATTTGTCTTTGAATGGTGATT |
| | ATGACTTAATCATTTTAAAACTGATAAACTTGACAAAAACCCTGTATGAA |
| | ATAAACATGAAATTAATAGCACTGATTTCATTGTAAAATTTTAAAGCAGT |
| | TTAAAGGGTACCACAGGTTATCACAGTACTCTCAATGCCACAAACACCTC |
| | TTGTTCAGTATTCTAGAAATACTGAATCAGAATTCTGTGTTTATTATAATC |
| | TCAGCATACTGTACATAATATCTGCTAGTTAAACTTGGGTAATTGGTTAA |
| | GGTGACTTACTGTCTATGTCAATATGTATAGTTTTGAGTACTTCAAGAGTT |
| | TACTTAAAAGTGATGATGTTACTGGTATGTTGGCAGTGGGTGGGACTGAA |
| | GTAGTGTATCTATTATAAATTGATCTATTTTCTTAATTCTAAGATGAAGTC |
| | CAATTTTAAGCATCAGCTTTTAGGTGCAAAGGAGGAATTAACACATTAAA |
| | TGTATACAGTTCTAAATTTTTGAAATAACTGATGTGTAGCATTTGATTATT |
| | GGTATTACCATTTTAGAATCATGATGTTATTTTAAACCTTTTTCCTGGGGA |
| | CAAGAAAGGATAATAAATTACGCTGAATCACTTTTGGCAGTTGCCACTTA |
| | AATAGTACAGTGACTTGCAACTTTTATAACTTTATCAGCATCTTCTCTAAA |
| | TACAAAATTAGGCTATATGTTATTTTCCAACTTACTGTTTTCTCTCTGTTT |
| | AGCAGGATATTATAAATAGATTAAATAGATATATTTTCTTTTTTTTTTTTT |
| | TTTTTTGAGACGGAGTCTCGCTTTGTCTCCCAGGCTGGAGTGCAGTGGCG |
| | TGATCTCCCAGTAGCTGGGACTACAAGCACCTGCCACCATGCCCGGCTAA |
| | TTTTTTTTGTATTTTTAGTAGAGACGGGGTTTCACTGTGTTAGCCAAGATG |
| | GTCTCAATCTCCTGACTTTGTGATCTGCCTGCTTCTGCCTCCCAAAGTGCT |
| | GGGATTACAGGTGTGAGCCACCGTACCCAGCCCAAATAGATGTATTTTCA |
| | TAATAGAGAATTGAAATAGGCTTTAATGGGTGAATAGCAGTTTATTGTAG |
| | GCATGTGACATTTCATTTAATGAATTTAAAGTTTATTATCCCAATTCTACA |
| | GAAGGATTTAATGCATACTATGCAATTAAATAATTATAACACTACATAGT |
| | AATAATTTATGTGCCAGGCAGTAGTTCAGTCACTTTACATGCTTATTAAC |
| | CTGCAGAATAATCTTTTGAGATGTAGGTGCTGTTACTGAGAATTTAACTT |
| | TTGCTTGTAAATTGCAAAGGGTGGATTTGAATTCTGGAAATTTGGTTCCA |
| | GAGACAATAATTACATAACACTTTCTCCATAGGGTACAGCCTGTCTAATA |
| | GGCTATAGTAAATCACCTCAGCTTGTTATAGGTCGGGCATGCAAACATTT |
| | CTCCATTTTACTCCCTTTGGTAATGAATCTAGTAATAGATGGAAATTTTCC |
| | CTAGATTCACTGTGTTAGTCAGTTGGGGAAGTTTGGAGGCAAAGATACAG |
| | GAGTTTATGGGGAGGTAGTGTACATAAATATAATCATATGCTATATAAGG |
| | AAGTTTTGGTCAGCAGCAGACCATATATAGGATGGTGGGCCAGTAACATT |
| | GTAACACTGTATTTTTACTGTATCTTTTCCATGTTTTGTTATGTTTAGATAC |
| | ACAAATAACATTATGGAGTATTCAGTATGGTAACATGCCATACAGGTTTG |
| | TAGCCTAGGAGCAGTAATAGACTGTTCCCTGAAACCTATATTTGTGGTAG |
| | GTTTATACCATTCAGGTTTGTGTAAGTACACAACGAAATCATCTAATGGC |
| | CCATTTCTCAAAACATATTCCCATCATTAATCAATGCATGGTCATGTTTTC |
| | GTATACATTTTAAGCTTCTGTATTCTAATCTAATATAAATGGCAAAATATT |
| | CAAACTGATAGGCATTGAGATTCTTAAATGCTAAAGTTGCATTCAAAAGG |
| | ATAATTTTAGGCGTTGTGACAAAGCAGTGTTATATTTTAAAGTTAGTGAC |
| | AAGGCTATGCACCTTTTATCTCTAATTGTTTCTTACAGAATGTTTTTATTA |
| | TTGAGTAGTAAAACAATAAATGTCAGATCCTTTATACAAATTCAAGATTG |
| | ACATTGATAAACAAAACTTCAGCATATCACTCAAGGTCAGCGTAGAAATT |
| | GTGTGTCTGGAAACTTCTATAGTAATTTTATATTACTGTGACATTAGTATG |
| | TGATCACTTTTCTAGTAATGTTTTAAAAAAATATATCTTACAGGCCAGGC |
| | ATAGTGCTTTATGCCTGTAATCCCAGCACTTTGGGAGGCCAAGGTGGCAG |
| | GATTGCATGAGTCCAGGAGTTCAACACCAGTCTGGGCAATAAAGTGAGA |
| | CCCCATCGCTACAAACAAATTAAAAAATATTTATGTATGTGTGTAATATA |
| | TATAATATATAACAAAACACATATGTATGTGTATATATAGTATGTCTGGC |
| | AGAGTACAATTTAGGGGTTAAGACTGGTCCCTCACATATGGTGTGAGAA |
| | ACACTGTTCACAGGTTGCTTTCCCCATTAGCCCAGGGCAACTCATTTGCC |
| | CATCATTTCCTAGAACAACCGGGTCTGTTACGTCTACAGTTTTCATCTTCA |
| | TGCAGTTATGGATTTGGTGTCAAAAACTTTGGTCCTGTTCTCTACCATCTT |
| | AAAATAAACTCTTGTGTCCTGCTTTACTATGAATTGCAAAGTAGGCATTA |
| | GGTAGCCTTCCTACTACCATAGTTTAGAGTTCAATATTCTTATGACCATTC |
| | TACTGGTAGAAGCAAAAAATGAACTTGTAGGCATGTGATCACATGTGCCT |
| | ATGGTGCTGTCTTTTCCAGTACAGGGGAACTAATTTTCATATTTTAATCTT |
| | GCAGCTTTTTGTTTACTTCATGCATTGTGATTTCTCATAGTTTTGCACAGA |
| | ACTCACTTCCCTACCTTTTCTGAAACAAAAGTATGTATACACACATACAT |
| | ATGTATTGAGCACCTCTATTTACTGTGTTCCACGTGCTGGGCATATAGCA |
| | AGAACAGAATGGTCTGGGGCCCTGCTCTAAAGAAGATTTAAAAGCAAAC |
| | ATATATTAAAAATGCGTGAGTCTGGCCAGGAGCAGTGGCTCATGCCTGCA |
| | ACCCCAGCACTTTGGGAGGCTGAAGCGGGTGAATCACCTGAGATCAGGA |
| | GTTGAGACCAGCCTGGCCAACATGGTGAAACCCCGTCTCTACTAAAAATA |
| | CAAAAATTAGCCTGGCATGGTGGCATGCGCCTGTAATTCCAGCTACTTTG |
| | GAGACTGAGACAGGAGAATCACTTGAGCCCAGGAAGTGGAGGTTGCAGT |
| | GAGCTGAGATCGCGCCACTGCACTCCAGCCTGGGTGACAGAGCAAGACT |
| | CCATCTCAAAAAAAAAAAAAAAACGTGTCTGTTCATAAGGTTCTACAAA |
| | TAGCTGTTGTTACAGAAAATAGGACTAGGGTTTTATTACTGGGGATTATG |
| | CAGTCGAGGTAAATATAAAATGAGGTTGTTTCTTCTTTTTTTTATTTGAGA |
| | CAGAGTCTTGTTTGCCAGGCTGGAGTGCAGTGGCGTGCTCTTAGCTCCGC |
| | CTCCCGGGATCAAACGATTCTCTTGCCTCAGCCTCTCGAGTAGCTGGGAC |
| | TACAGGCGCGTGCCACCACACCCAGCTGATTTTTGTATTTTTAGTAGAGA |
| | TGGGATTTCACCATGATGGCCAGGATGGTCTCGATCTTTTGACCTCATGA |
| | TCCACCCACCTCGGCCTCCCAAAGTGCTGGGATTACAGGCGTGAGCCACT |
| | GCACCCGGCCCTGGTTCTAAATACTCTTCCAGCTCTAAAACATTGATTCT |
| | AAACAGATCACATTCCAGGAGGACTCTAGCAAACCAGCATATGCAAATT |
| | ATAGCTTTTGCAAGACCGTTTCTACTTTTATATTACTGAACTCTATATGAT |
| | TGTCCAAGTAAAGTTTTGTGTCTCTTTGATTATTTGATTGTACTTTAAAAT |
| | TTTTTCACCATTCATTTAACATTTTTTACCATACTGTAGTATTTTTAATGCA |
| | ATTGTGTTTGCATTGGTGTGGCTTTAGAGGCTTCTCCAACCACCTTCCCAA |
| | AATACTGATCTGTGATTTTTTTCTTTAATGTTTGGCCAAACATAATACATG |
| | CTTATTTTATTTTTCATCCCTACAGAAAGGTAGAAGATGAGAATTCTGTCT |
| | CCTACTGTTGTTTTTCAAGGTGCCACTCAAATTTCTTGTACGTGTCTAGAA |
| | ACTTGTGCATACAATAGAAGTACACTGTGGCTGGGCATGGTGGCTCATGC |
| | CTGTAATCCCAGCACTTTGTGAAGCTGAGGTGGGTGGATCACCTGAGGTC |
| | AGGGAGTTTGAGACCAGCCTGGCCAACATGATGAAACCCCGTCTTTACTA |
| | AAATTAGAAAAAATTAGCCAGGCGTGGTGGTGTGCGCCTGTAATCCCAG |
| | CTACTCGGGAGGCTGAGGCATGAGAATCACTTGAACTCGGGTGGCAGAG |
| | GCTGCAGTGAGCTGAGATCATGCCACTGCATTCCAGCCTGCGCAGCTGAG |
| | CCAGACTCCATCTCCAAAAAAAAAAAAAAAAAAAAAAAAAGATGTCCA |
| | ATATATGTAACTTTTTTCTTTGGACACAAAAATTCCATTAGCTTTGTTTTC |
| | TCATTTTTACTTGTCATGATGTATGTCGAACACATTATTTTTAGTGTCTGG |
| | TGTTCCATTATACAGATGTCTCTCTTGTTGGTTGAATTTTTGCATTCACAG |
| | ACCCTCAAGTTGGATTCATATCTTTTTACACTAAGCATAAAGAAGACGGA |
| | TTGGGGTCGGGTATGGTGGCTCACGCCTGTAATCCCAGCACTTCGGGAGG |
| | CTGAGGTGGGCGGATCACGAGGTCAGGAGTTCGAGACCAGCCTGGCCAA |
| | CATACTGAAACCCCGTCTCTAAAAATATGAAAAAAAAATTAGCCAGGCG |
| | TGGTGGTGCGCAGCTGTAGCCCCAGCTACTTGGGAGGCTGAGGCAGGAG |
| | AATCACTTGAACCCAGGAGGTGGAGGTTGCAGTGAGCATATTGCACCATT |
| | GCATTCCAGCCTGCACGACAGAGGAAGACGCCATTTCAAAAAAAAAAAA |
| | AAAAAGACGGATTGATTTTCTTCAGCAGTATCAAGTGGCACTTACCATCC |
| | ACCCCTTTAACACCCAAACCATTCTAATCCATGTATACAGACCATCATCT |
| | GTTTCGTTATTGTGTTCTATAATGCAGCTTTGCGAGTTATGCAGTTTTGGT |
| | CCTCATGATATTTTTCTGATTGGTTTTTAATGTATTTTGTTCTAACGAGCC |
| | AACATTGTTATACATGTAATTTGTTTATTTGCTAGATTGTACTCTTTCCCA |
| | AAGATGGGCTATGAACTAGCATCTACTTTCATTTTACCTATTTACCTGTAA |
| | ACATTGAAAAAACTGAATCAAATGCAGTGATATCGGACCTAGTTTTATTG |
| | TTATGCCTTATAATGAATTTAACTTCACAGTTTTCTAAATGAGAGCATTTC |
| | CCAAAGACATCTTTATGGTCATAACCAGTTTCCCTTGGCATTTGATTTATT |
| | TTTATTTTTATTTATTTATCTTTTTGAGAAGGAGTTTCGCTCTTGTTGCCCA |
| | GGCTAGAGTGCAATGGCGTGATCTCGGGTCACTGCAACCTCTGCCTCCCG |
| | GGTTCAAGCGATTCTCCTGCCTCAGCCTCCAAGTAGCTGGGATTACAGGC |
| | ATGCACCACCACGCCCGACTAATTTGTATTTTTAGAGACGGGGTTTCTCC |
| | GTGTTGGTCAGGCTGGTCTCAAACTTCCAACCTCAGGTGATCCGCCCGCC |
| | GTGGCCTCCCAAAGTGTTGGGATTACAGGCGTGAGCCACAGTGTCTGGCC |
| | TGATTTGTTTTTAAGAGCATTATTTTTCTGCTTTATTTTGTGACTTCAACAT |
| | TTGACACAATTTTGGGTGAATGGTTTGTGCATGGTGCCTGACATCGTGTTT |
| | TGATGTGTAGTATATGCCATAGGACATGTGAGACAAGATATGTCCCAACT |
| | TGACCTTGTTTTGTATTGTTTATGTCAAGGTGTTGAGTGTATTAGATATAC |
| | TGTTGGGGCTCTGTGTTCTAGCTCTGCCTTTTAGATAATAACCATGGTTAA |
| | ATATTGCAATGTCCTGCATGTCTCCACACATGGGTTTTGTAACTGAGTCA |
| | GAATGATAAGTGATTAGTAAGCACATTTTTTCTCCTTTCAGGAAACCACT |
| | ATTTTCCTTTTCTACATGCTGTTTTGTAAGTAGTACTTTTATGAAGGTTGT |
| | CTTCAAATGTTCGCATCTTCCATTTCTACTGCCCTTGGGTTATCCATCCTG |
| | TCATTTTGTGCCAATCACTTTTTTTTTTAACTTTTAAGTTCAGGGATGAAA |
| | GTGTAGATTTGTTACATAGGTAAACTTGTGTCATGGGGTTGTTGTACAGA |
| | TTATTTCATCACCCAGGTGTTAAGCCTAGTATGCACTAGTTGTTTTTCCTG |
| | ATCCTCTCCCTGCTCCCACCCTCCACCCTCTGATAGGCCCCAGTGTGTGTT |
| | GTTCCCCTCTGTGTGTCCATGTGTTCTCATCATTTAGCTCCCACTTACAAG |
| | TGAGAATGTGGTGTTTGGTTTTCTGTTCCTGCATTAGTTTGCTAAGGATAA |
| | TGGCCTCGAGCTCCATCCAAGTCCCTGTAAAGGACATGATCTTGTACTTT |
| | TTTATGGCTGCATAGTATTCTGTGGTGTATATGTACCACATTTTCTTTATC |
| | CAGTCTATCATTAGGCATTTAGGTTGATTCCATGTTTTTGCTATTGTGAAT |
| | AGTGCTGCAATGAACATACATGTGCATGTCTTTTTATAATAGAATGATTT |
| | ATATTCTGTTGTGTATATACCCAGTAATGGGATTGCTGAGTTGAATGCTA |
| | TTTCTGCCTTTAGGTCTTTGATGAATTGCCACACTGTCTTCCACAATGGTT |
| | GAACTAATTTACACTCCCACCAACGGTGAATAAGTGTTCACTTTTCTCCA |
| | CAACCTTGTCAGCATCTATTATTTTTTGACTTTTTAGTAATAGCCATTCTG |
| | ACTGCTCACATCTATTTTGTAAATAAAGTTTTATTGAAACATGGCCTTACC |
| | CATTTGTTTACATATATTCATGGCTGTTTTTGTGCCACAATGTCAGAGTTG |
| | TCTTAAAGTAGACAGAGACTATCTGGCTGTAAAGCCTGAGATATATACTA |
| | ACTGGTTCTTTATGTAAAAAGTTTGCTGACCACCTACTCTAAACGTTTTGC |
| | AGTGATGGTAGTGTTGGCAAAAAACCAAATAGCTTACCCTCTTTAAATTT |
| | CCCTTTTACTTCTTACAAACTCCTAACACCATTTACGACTTTGTCATCAAT |
| | ATGGTCAACTAAGCTTGGTTTGCATGGCTCTACTTCCTTTCACCTTCCACT |
| | TAGGCAGTGTCTCCAAGTCCACTGCAGTTTCTATTTGTCTCCTGACTGTTA |
| | CTGTATCAGTTCTTACCTAAATAACATAACAACTGATCTCCCTACTTTTTG |
| | CCTATGCCCTCAAATGTGCTCATTGTTGATCTATCTCCCTGTTAGGTGTTC |
| | TTTTTCTCCTCTTTAGAAAGCAGCCAAGGAAACCAGGGTTCTCTCAAAGT |
| | GGAAAATACTGGAACTTATGTACTGTTATCATAATGATAGTTGGTGTTTT |
| | GAATTATAAGAATGATTCCAGGTGGTTTCTAAATCATCCAATAAAGCTGT |
| | ATTCACTCTGTAAAAAAAAAAA (SEQ ID NO: 52) |
| (NM_001130679) | AGGCACAGGCAGCCTGCATACACTCCTTTTCCTGGTGTCAACATTATTTA |
| Homo sapiens | AAAGCATGGGAAATAGTAATGAGACAGTGTCTTCTTCATTAGAACCTTAG |
| eukaryotic | GAGTCTACTAGATTTCTTCATCTCTATTTGTTGTTATTAGTAGCCAAACTG |
| translation initiation | TGCAAAAAACACGGTCTTGAGAAATGACAGCACAGTATCTTAGAGGGAA |
| factor 4E (EIF4E), | AGGAAATGTAGGATGCCAGTGTGGGGACAAATTTCTGATTGCCAGTGATT |
| transcript variant 2 | GTTGTGAGCATAACAATAATTTCATGAACATTAAAGCCTCTATTGAGGGC |
| | AGCTGCAGTTGTAAAGGAAAAAAAATGGTCCTGAACATTTAAAACTACA |
| | CTGGTGTACATCATAATCAAACAAAGTAAACAGAAAAAAATTTAAACTT |
| | TGCTAAAAAAAAAAAGCAGAAGCACTTGATCTTTAGGAAGGCACGCAGT |
| | TGCTTATTATGAATCATTTCTAGAGTCCGATGCATTTTCAAAGCCGGTTAC |
| | AGTCATTACGAAGCACACCCTTGTGAGGTAAGTGTATCATCACCTTTGGT |
| | TCATAAATAAAAAAGCTGAGACGCCGAGCGATTAAGTCACTCGCCTAAG |
| | GAGAATGAGTCAACGTCAAGAGTCATAGTTGACCCGGCCTAAAGACTCC |
| | AGACCATCAGTCCAGGGCTTAGTCAGCGGGGCCCGGAGTGGCTTCCCTG |
| | GCTGGCATCTGGACTTAGGCTATTTCCGTGCACGTAAAAGCGGAATATTG |
| | GAACGGTTGCACAGAACTTCCAAATAATTTTTACCGCCACGCAAGATTTA |
| | GCCCTGAGGTCTTAATCTCAGGATTTGGGACAGTAAAAGCTGTCGTCCCT |
| | CCCCCTCGTCCAGCCGGTGGCAAGCGGGTACTGCGGGCGGTTCCGTCCGT |
| | CCCCTTTCGCAGAAATGGCAACGAATGACCACCAGCATTAGCTGAGCCA |
| | GGGGACGTGGGAGGGTTGATTGCCTAAACGACTCTGCATCGCCGCCTCTT |
| | TTTGAAACTAAGAGAAAATGGTGGGAGATCAAAAGAAAACTAAATAAAC |
| | ACACAGGCAACTTGTCCTGGGACCTCAACTAAGCAAATGAAGCCTTATTG |
| | TGTGTGCTGAGCCTGCAGTTCCCAACCTTCCGGGGAAGATGGGAGGACA |
| | GGGCGACAAAGGGCACAGTAGGCTTGCCTGGCAGTAAGTGTGACCGCAG |
| | CTATCCAGGCGGAAGAGCAGAGGACTGAAACCACCCTCCAGCAAGCGAG |
| | TGTCCGCCGCGTTGAGAACCGCGCACCCTACCCATCGGCCACGTGACCAG |
| | TCCTTTTTAAAAAAAATTTCTTTACCTTAAAAAAAAAAAAAAAAAAAAG |
| | GTGGGGGAGAGACTCCACTTCCCAGAAGCCTCTCGTTACTCACGCAGCCG |
| | CAGTCTTGCGCAGGTGCCGCCAGGGCCAAACGGACATATCCGTCACGTG |
| | GCCAGAAGCTGGCCAATCCGGTTTGAATCTCATTTTTTTCCTCTTACCCCC |
| | CCTTCTGGAGCGGTTGTGCGATCAGATCGATCTAAGATGGCGACTGTCGA |
| | ACCGGAAACCACCCCTACTCCTAATCCCCCGACTACAGAAGAGGAGAAA |
| | ACGGAATCTAATCAGGAGGTTGCTAACCCAGAACACTATATTAAACATCC |
| | CCTACAGAACAGATGGGCACTCTGGTTTTTTAAAAATGATAAAAGCAAA |
| | ACTTGGCAAGCAAACCTGCGGCTGATCTCCAAGTTTGATACTGTTGAAGA |
| | CTTTTGGGCTCTGTACAACCATATCCAGTTGTCTAGTAATTTAATGCCTGG |
| | CTGTGACTACTCACTTTTTAAGGATGGTATTGAGCCTATGTGGGAAGATG |
| | AGAAAAACAAACGGGGAGGACGATGGCTAATTACATTGAACAAACAGC |
| | AGAGACGAAGTGACCTCGATCGCTTTTGGCTAGAGACAAGATGGGATCT |
| | TGCTATGTTGCCCAGGTTGGTCTCAAACTTCTGGCCTCAAGTGATCCTCCC |
| | ACTTCAGCCTCCCAAAGTGCTGGAATTACAGCTTCTGTGCCTTATTGGAG |
| | AATCTTTTGATGACTACAGTGATGATGTATGTGGCGCTGTTGTTAATGTTA |
| | GAGCTAAAGGTGATAAGATAGCAATATGGACTACTGAATGTGAAAACAG |
| | AGAAGCTGTTACACATATAGGGAGGGTATACAAGGAAAGGTTAGGACTT |
| | CCTCCAAAGATAGTGATTGGTTATCAGTCCCACGCAGACACAGCTACTAA |
| | GAGCGGCTCCACCACTAAAAATAGGTTTGTTGTTTAAGAAGACACCTTCT |
| | GAGTATTCTCATAGGAGACTGCGTCAAGCAATCGAGATTTGGGAGCTGA |
| | ACCAAAGCCTCTTCAAAAAGCAGAGTGGACTGCATTTAAATTTGATTTCC |
| | ATCTTAATGTTACTCAGATATAAGAGAAGTCTCATTCGCCTTTGTCTTGTA |
| | CTTCTGTGTTCATTTTTTTTTTTTTTTTTGGCTAGAGTTTCCACTATCCCAA |
| | TCAAAGAATTACAGTACACATCCCCAGAATCCATAAATGTGTTCCTGGCC |
| | CACTCTGTAATAGTTCAGTAGAATTACCATTAATTACATACAGATTTTAC |
| | CTATCCACAATAGTCAGAAAACAACTTGGCATTTCTATACTTTACAGGAA |
| | AAAAAATTCTGTTGTTCCATTTTATGCAGAAGCATATTTTGCTGGTTTGAA |
| | AGATTATGATGCATACAGTTTTCTAGCAATTTTCTTTGTTTCTTTTTACAG |
| | CATTGTCTTTGCTGTACTCTTGCTGATGGCTGCTAGATTTTAATTTATTTGT |
| | TTCCCTACTTGATAATATTAGTGATTCTGATTTCAGTTTTTCATTTGTTTTG |
| | CTTTTGTTTTTTTCCTCATGTAACATTGGTGAAGGATCCAGGAATATGACA |
| | CAAAGGTGGAATAAACATTAATTTTGTGCATTCTTTGGTAATTTTTTTTGT |
| | TTTTTGTAACTACAAAGCTTTGCTACAAATTTATGCATTTCATTCAAATCA |
| | GTGATCTATGTTTGTGTGATTTCCTAAACATAATTGTGGATTATAAAAAA |
| | TGTAACATCATAATTACATTCCTAACTAGAATTAGTATGTCTGTTTTTGTA |
| | TCTTTATGCTGTATTTTAACACTTTGTATTACTTAGGTTATTTTGCTTTGGT |
| | TAAAAATGGCTCAAGTAGAAAAGCAGTCCCATTCATATTAAGACAGTGT |
| | ACAAAACTGTAAATAAAATGTGTACAGTGAATTGTCTTTTAGACAACTAG |
| | ATTTGTCCTTTTATTTCTCCATCTTTATAGAAGGAATTTGTACTTCTTATTG |
| | CAAGGCAGTCTCTATATTATGTCTTCTTTTGTGGTGTCTTCCATGTGAACA |
| | GCATAAGTTTGGAGCACTAGTTTGATTATTATGTTTATTACAATTTTTAAT |
| | AAATTGAATAGGTAGTATCATATATATGGAATTAAATTGATGTGGCTATC |
| | TTTGTTTTTTTATAAAGTAAGGCACAGTCATTCAGTCTTAGGTAAATAATG |
| | TACTCTCTTAATATGTTAATACTCATGAGAATTGGGATCTGATGCATCAC |
| | CATTTGATTGGTAGCAACAGTGGTTGTAAAACTTGGTTGCTGAATTGAGT |
| | TGTTTCTATGTTAAGTGTCAAAATGATAGTGTAGGGAAAGTACAGGTGGT |
| | GGGGACATATGCATTAAGAATCTTGTTAGTGTTGCAATCTAAATAGAATG |
| | GAATAAACAGGTGTTAAGACATATTTATAGTGGTAAATTGTTGTAGTATG |
| | GTATTCTGTAAACTTGAAAACTTGATCTACTCTTTGTAGGTATCATTTGAA |
| | AGCAAACTTGAAAATGTTTTGTACATAGTACATACTTGTATAGTCCTGTG |
| | AGATGAAGTATGGCTATCAGACCAAAGGATAAGCCAAACTGTAGGTAGC |
| | AGAATGGAAATTATTATTTTGAGAGGAAAATTTGTCTTTGAATGGTGATT |
| | ATGACTTAATCATTTTAAAACTGATAAACTTGACAAAAACCCTGTATGAA |
| | ATAAACATGAAATTAATAGCACTGATTTCATTGTAAAATTTTAAAGCAGT |
| | TTAAAGGGTACCACAGGTTATCACAGTACTCTCAATGCCACAAACACCTC |
| | TTGTTCAGTATTCTAGAAATACTGAATCAGAATTCTGTGTTTATTATAATC |
| | TCAGCATACTGTACATAATATCTGCTAGTTAAACTTGGGTAATTGGTTAA |
| | GGTGACTTACTGTCTATGTCAATATGTATAGTTTTGAGTACTTCAAGAGTT |
| | TACTTAAAAGTGATGATGTTACTGGTATGTTGGCAGTGGGTGGGACTGAA |
| | GTAGTGTATCTATTATAAATTGATCTATTTTCTTAATTCTAAGATGAAGTC |
| | CAATTTTAAGCATCAGCTTTTAGGTGCAAAGGAGGAATTAACACATTAAA |
| | TGTATACAGTTCTAAATTTTTGAAATAACTGATGTGTAGCATTTGATTATT |
| | GGTATTACCATTTTAGAATCATGATGTTATTTTAAACCTTTTTCCTGGGGA |
| | CAAGAAAGGATAATAAATTACGCTGAATCACTTTTGGCAGTTGCCACTTA |
| | AATAGTACAGTGACTTGCAACTTTTATAACTTTATCAGCATCTTCTCTAAA |
| | TACAAAATTAGGCTATATGTTATTTTCCAACTTACTGTTTTCTCTCTGTTT |
| | AGCAGGATATTATAAATAGATTAAATAGATATATTTTCTTTTTTTTTTTTT |
| | TTTTTTGAGACGGAGTCTCGCTTTGTCTCCCAGGCTGGAGTGCAGTGGCG |
| | TGATCTCCCAGTAGCTGGGACTACAAGCACCTGCCACCATGCCCGGCTAA |
| | TTTTTTTTGTATTTTTAGTAGAGACGGGGTTTCACTGTGTTAGCCAAGATG |
| | GTCTCAATCTCCTGACTTTGTGATCTGCCTGCTTCTGCCTCCCAAAGTGCT |
| | GGGATTACAGGTGTGAGCCACCGTACCCAGCCCAAATAGATGTATTTTCA |
| | TAATAGAGAATTGAAATAGGCTTTAATGGGTGAATAGCAGTTTATTGTAG |
| | GCATGTGACATTTCATTTAATGAATTTAAAGTTTATTATCCCAATTCTACA |
| | GAAGGATTTAATGCATACTATGCAATTAAATAATTATAACACTACATAGT |
| | AATAATTTATGTGCCAGGCAGTAGTTCAGTCACTTTACATGCTTATTAAC |
| | CTGCAGAATAATCTTTTGAGATGTAGGTGCTGTTACTGAGAATTTAACTT |
| | TTGCTTGTAAATTGCAAAGGGTGGATTTGAATTCTGGAAATTTGGTTCCA |
| | GAGACAATAATTACATAACACTTTCTCCATAGGGTACAGCCTGTCTAATA |
| | GGCTATAGTAAATCACCTCAGCTTGTTATAGGTCGGGCATGCAAACATTT |
| | CTCCATTTTACTCCCTTTGGTAATGAATCTAGTAATAGATGGAAATTTTCC |
| | CTAGATTCACTGTGTTAGTCAGTTGGGGAAGTTTGGAGGCAAAGATACAG |
| | GAGTTTATGGGGAGGTAGTGTACATAAATATAATCATATGCTATATAAGG |
| | AAGTTTTGGTCAGCAGCAGACCATATATAGGATGGTGGGCCAGTAACATT |
| | GTAACACTGTATTTTTACTGTATCTTTTCCATGTTTTGTTATGTTTAGATAC |
| | ACAAATAACATTATGGAGTATTCAGTATGGTAACATGCCATACAGGTTTG |
| | TAGCCTAGGAGCAGTAATAGACTGTTCCCTGAAACCTATATTTGTGGTAG |
| | GTTTATACCATTCAGGTTTGTGTAAGTACACAACGAAATCATCTAATGGC |
| | CCATTTCTCAAAACATATTCCCATCATTAATCAATGCATGGTCATGTTTTC |
| | GTATACATTTTAAGCTTCTGTATTCTAATCTAATATAAATGGCAAAATATT |
| | CAAACTGATAGGCATTGAGATTCTTAAATGCTAAAGTTGCATTCAAAAGG |
| | ATAATTTTAGGCGTTGTGACAAAGCAGTGTTATATTTTAAAGTTAGTGAC |
| | AAGGCTATGCACCTTTTATCTCTAATTGTTTCTTACAGAATGTTTTTATTA |
| | TTGAGTAGTAAAACAATAAATGTCAGATCCTTTATACAAATTCAAGATTG |
| | ACATTGATAAACAAAACTTCAGCATATCACTCAAGGTCAGCGTAGAAATT |
| | GTGTGTCTGGAAACTTCTATAGTAATTTTATATTACTGTGACATTAGTATG |
| | TGATCACTTTTCTAGTAATGTTTTAAAAAAATATATCTTACAGGCCAGGC |
| | ATAGTGCTTTATGCCTGTAATCCCAGCACTTTGGGAGGCCAAGGTGGCAG |
| | GATTGCATGAGTCCAGGAGTTCAACACCAGTCTGGGCAATAAAGTGAGA |
| | CCCCATCGCTACAAACAAATTAAAAAATATTTATGTATGTGTGTAATATA |
| | TATAATATATAACAAAACACATATGTATGTGTATATATAGTATGTCTGGC |
| | AGAGTACAATTTAGGGGTTAAGACTGGTCCCTCACATATGGTGTGAGAA |
| | ACACTGTTCACAGGTTGCTTTCCCCATTAGCCCAGGGCAACTCATTTGCC |
| | CATCATTTCCTAGAACAACCGGGTCTGTTACGTCTACAGTTTTCATCTTCA |
| | TGCAGTTATGGATTTGGTGTCAAAAACTTTGGTCCTGTTCTCTACCATCTT |
| | AAAATAAACTCTTGTGTCCTGCTTTACTATGAATTGCAAAGTAGGCATTA |
| | GGTAGCCTTCCTACTACCATAGTTTAGAGTTCAATATTCTTATGACCATTC |
| | TACTGGTAGAAGCAAAAAATGAACTTGTAGGCATGTGATCACATGTGCCT |
| | ATGGTGCTGTCTTTTCCAGTACAGGGGAACTAATTTTCATATTTTAATCTT |
| | GCAGCTTTTTGTTTACTTCATGCATTGTGATTTCTCATAGTTTTGCACAGA |
| | ACTCACTTCCCTACCTTTTCTGAAACAAAAGTATGTATACACACATACAT |
| | ATGTATTGAGCACCTCTATTTACTGTGTTCCACGTGCTGGGCATATAGCA |
| | AGAACAGAATGGTCTGGGGCCCTGCTCTAAAGAAGATTTAAAAGCAAAC |
| | ATATATTAAAAATGCGTGAGTCTGGCCAGGAGCAGTGGCTCATGCCTGCA |
| | ACCCCAGCACTTTGGGAGGCTGAAGCGGGTGAATCACCTGAGATCAGGA |
| | GTTGAGACCAGCCTGGCCAACATGGTGAAACCCCGTCTCTACTAAAAATA |
| | CAAAAATTAGCCTGGCATGGTGGCATGCGCCTGTAATTCCAGCTACTTTG |
| | GAGACTGAGACAGGAGAATCACTTGAGCCCAGGAAGTGGAGGTTGCAGT |
| | GAGCTGAGATCGCGCCACTGCACTCCAGCCTGGGTGACAGAGCAAGACT |
| | CCATCTCAAAAAAAAAAAAAAAACGTGTCTGTTCATAAGGTTCTACAAA |
| | TAGCTGTTGTTACAGAAAATAGGACTAGGGTTTTATTACTGGGGATTATG |
| | CAGTCGAGGTAAATATAAAATGAGGTTGTTTCTTCTTTTTTTTATTTGAGA |
| | CAGAGTCTTGTTTGCCAGGCTGGAGTGCAGTGGCGTGCTCTTAGCTCCGC |
| | CTCCCGGGATCAAACGATTCTCTTGCCTCAGCCTCTCGAGTAGCTGGGAC |
| | TACAGGCGCGTGCCACCACACCCAGCTGATTTTTGTATTTTTAGTAGAGA |
| | TGGGATTTCACCATGATGGCCAGGATGGTCTCGATCTTTTGACCTCATGA |
| | TCCACCCACCTCGGCCTCCCAAAGTGCTGGGATTACAGGCGTGAGCCACT |
| | GCACCCGGCCCTGGTTCTAAATACTCTTCCAGCTCTAAAACATTGATTCT |
| | AAACAGATCACATTCCAGGAGGACTCTAGCAAACCAGCATATGCAAATT |
| | ATAGCTTTTGCAAGACCGTTTCTACTTTTATATTACTGAACTCTATATGAT |
| | TGTCCAAGTAAAGTTTTGTGTCTCTTTGATTATTTGATTGTACTTTAAAAT |
| | TTTTTCACCATTCATTTAACATTTTTTACCATACTGTAGTATTTTTAATGCA |
| | ATTGTGTTTGCATTGGTGTGGCTTTAGAGGCTTCTCCAACCACCTTCCCAA |
| | AATACTGATCTGTGATTTTTTTCTTTAATGTTTGGCCAAACATAATACATG |
| | CTTATTTTATTTTTCATCCCTACAGAAAGGTAGAAGATGAGAATTCTGTCT |
| | CCTACTGTTGTTTTTCAAGGTGCCACTCAAATTTCTTGTACGTGTCTAGAA |
| | ACTTGTGCATACAATAGAAGTACACTGTGGCTGGGCATGGTGGCTCATGC |
| | CTGTAATCCCAGCACTTTGTGAAGCTGAGGTGGGTGGATCACCTGAGGTC |
| | AGGGAGTTTGAGACCAGCCTGGCCAACATGATGAAACCCCGTCTTTACTA |
| | AAATTAGAAAAAATTAGCCAGGCGTGGTGGTGTGCGCCTGTAATCCCAG |
| | CTACTCGGGAGGCTGAGGCATGAGAATCACTTGAACTCGGGTGGCAGAG |
| | GCTGCAGTGAGCTGAGATCATGCCACTGCATTCCAGCCTGCGCAGCTGAG |
| | CCAGACTCCATCTCCAAAAAAAAAAAAAAAAAAAAAAAAAGATGTCCA |
| | ATATATGTAACTTTTTTCTTTGGACACAAAAATTCCATTAGCTTTGTTTTC |
| | TCATTTTTACTTGTCATGATGTATGTCGAACACATTATTTTTAGTGTCTGG |
| | TGTTCCATTATACAGATGTCTCTCTTGTTGGTTGAATTTTTGCATTCACAG |
| | ACCCTCAAGTTGGATTCATATCTTTTTACACTAAGCATAAAGAAGACGGA |
| | TTGGGGTCGGGTATGGTGGCTCACGCCTGTAATCCCAGCACTTCGGGAGG |
| | CTGAGGTGGGCGGATCACGAGGTCAGGAGTTCGAGACCAGCCTGGCCAA |
| | CATACTGAAACCCCGTCTCTAAAAATATGAAAAAAAAATTAGCCAGGCG |
| | TGGTGGTGCGCAGCTGTAGCCCCAGCTACTTGGGAGGCTGAGGCAGGAG |
| | AATCACTTGAACCCAGGAGGTGGAGGTTGCAGTGAGCATATTGCACCATT |
| | GCATTCCAGCCTGCACGACAGAGGAAGACGCCATTTCAAAAAAAAAAAA |
| | AAAAAGACGGATTGATTTTCTTCAGCAGTATCAAGTGGCACTTACCATCC |
| | ACCCCTTTAACACCCAAACCATTCTAATCCATGTATACAGACCATCATCT |
| | GTTTCGTTATTGTGTTCTATAATGCAGCTTTGCGAGTTATGCAGTTTTGGT |
| | CCTCATGATATTTTTCTGATTGGTTTTTAATGTATTTTGTTCTAACGAGCC |
| | AACATTGTTATACATGTAATTTGTTTATTTGCTAGATTGTACTCTTTCCCA |
| | AAGATGGGCTATGAACTAGCATCTACTTTCATTTTACCTATTTACCTGTAA |
| | ACATTGAAAAAACTGAATCAAATGCAGTGATATCGGACCTAGTTTTATTG |
| | TTATGCCTTATAATGAATTTAACTTCACAGTTTTCTAAATGAGAGCATTTC |
| | CCAAAGACATCTTTATGGTCATAACCAGTTTCCCTTGGCATTTGATTTATT |
| | TTTATTTTTATTTATTTATCTTTTTGAGAAGGAGTTTCGCTCTTGTTGCCCA |
| | GGCTAGAGTGCAATGGCGTGATCTCGGGTCACTGCAACCTCTGCCTCCCG |
| | GGTTCAAGCGATTCTCCTGCCTCAGCCTCCAAGTAGCTGGGATTACAGGC |
| | ATGCACCACCACGCCCGACTAATTTGTATTTTTAGAGACGGGGTTTCTCC |
| | GTGTTGGTCAGGCTGGTCTCAAACTTCCAACCTCAGGTGATCCGCCCGCC |
| | GTGGCCTCCCAAAGTGTTGGGATTACAGGCGTGAGCCACAGTGTCTGGCC |
| | TGATTTGTTTTTAAGAGCATTATTTTTCTGCTTTATTTTGTGACTTCAACAT |
| | TTGACACAATTTTGGGTGAATGGTTTGTGCATGGTGCCTGACATCGTGTTT |
| | TGATGTGTAGTATATGCCATAGGACATGTGAGACAAGATATGTCCCAACT |
| | TGACCTTGTTTTGTATTGTTTATGTCAAGGTGTTGAGTGTATTAGATATAC |
| | TGTTGGGGCTCTGTGTTCTAGCTCTGCCTTTTAGATAATAACCATGGTTAA |
| | ATATTGCAATGTCCTGCATGTCTCCACACATGGGTTTTGTAACTGAGTCA |
| | GAATGATAAGTGATTAGTAAGCACATTTTTTCTCCTTTCAGGAAACCACT |
| | ATTTTCCTTTTCTACATGCTGTTTTGTAAGTAGTACTTTTATGAAGGTTGT |
| | CTTCAAATGTTCGCATCTTCCATTTCTACTGCCCTTGGGTTATCCATCCTG |
| | TCATTTTGTGCCAATCACTTTTTTTTTTAACTTTTAAGTTCAGGGATGAAA |
| | GTGTAGATTTGTTACATAGGTAAACTTGTGTCATGGGGTTGTTGTACAGA |
| | TTATTTCATCACCCAGGTGTTAAGCCTAGTATGCACTAGTTGTTTTTCCTG |
| | ATCCTCTCCCTGCTCCCACCCTCCACCCTCTGATAGGCCCCAGTGTGTGTT |
| | GTTCCCCTCTGTGTGTCCATGTGTTCTCATCATTTAGCTCCCACTTACAAG |
| | TGAGAATGTGGTGTTTGGTTTTCTGTTCCTGCATTAGTTTGCTAAGGATAA |
| | TGGCCTCGAGCTCCATCCAAGTCCCTGTAAAGGACATGATCTTGTACTTT |
| | TTTATGGCTGCATAGTATTCTGTGGTGTATATGTACCACATTTTCTTTATC |
| | CAGTCTATCATTAGGCATTTAGGTTGATTCCATGTTTTTGCTATTGTGAAT |
| | AGTGCTGCAATGAACATACATGTGCATGTCTTTTTATAATAGAATGATTT |
| | ATATTCTGTTGTGTATATACCCAGTAATGGGATTGCTGAGTTGAATGCTA |
| | TTTCTGCCTTTAGGTCTTTGATGAATTGCCACACTGTCTTCCACAATGGTT |
| | GAACTAATTTACACTCCCACCAACGGTGAATAAGTGTTCACTTTTCTCCA |
| | CAACCTTGTCAGCATCTATTATTTTTTGACTTTTTAGTAATAGCCATTCTG |
| | ACTGCTCACATCTATTTTGTAAATAAAGTTTTATTGAAACATGGCCTTACC |
| | CATTTGTTTACATATATTCATGGCTGTTTTTGTGCCACAATGTCAGAGTTG |
| | TCTTAAAGTAGACAGAGACTATCTGGCTGTAAAGCCTGAGATATATACTA |
| | ACTGGTTCTTTATGTAAAAAGTTTGCTGACCACCTACTCTAAACGTTTTGC |
| | AGTGATGGTAGTGTTGGCAAAAAACCAAATAGCTTACCCTCTTTAAATTT |
| | CCCTTTTACTTCTTACAAACTCCTAACACCATTTACGACTTTGTCATCAAT |
| | ATGGTCAACTAAGCTTGGTTTGCATGGCTCTACTTCCTTTCACCTTCCACT |
| | TAGGCAGTGTCTCCAAGTCCACTGCAGTTTCTATTTGTCTCCTGACTGTTA |
| | CTGTATCAGTTCTTACCTAAATAACATAACAACTGATCTCCCTACTTTTTG |
| | CCTATGCCCTCAAATGTGCTCATTGTTGATCTATCTCCCTGTTAGGTGTTC |
| | TTTTTCTCCTCTTTAGAAAGCAGCCAAGGAAACCAGGGTTCTCTCAAAGT |
| | GGAAAATACTGGAACTTATGTACTGTTATCATAATGATAGTTGGTGTTTT |
| | GAATTATAAGAATGATTCCAGGTGGTTTCTAAATCATCCAATAAAGCTGT |
| | ATTCACTCTGTAAAAAAAAAAA (SEQ ID NO: 53) |
| (NM_001968) | AGGCACAGGCAGCCTGCATACACTCCTTTTCCTGGTGTCAACATTATTTA |
| Homo sapiens | AAAGCATGGGAAATAGTAATGAGACAGTGTCTTCTTCATTAGAACCTTAG |
| eukaryotic | GAGTCTACTAGATTTCTTCATCTCTATTTGTTGTTATTAGTAGCCAAACTG |
| translation initiation | TGCAAAAAACACGGTCTTGAGAAATGACAGCACAGTATCTTAGAGGGAA |
| factor 4E (EIF4E), | AGGAAATGTAGGATGCCAGTGTGGGGACAAATTTCTGATTGCCAGTGATT |
| transcript variant 1 | GTTGTGAGCATAACAATAATTTCATGAACATTAAAGCCTCTATTGAGGGC |
| | AGCTGCAGTTGTAAAGGAAAAAAAATGGTCCTGAACATTTAAAACTACA |
| | CTGGTGTACATCATAATCAAACAAAGTAAACAGAAAAAAATTTAAACTT |
| | TGCTAAAAAAAAAAAGCAGAAGCACTTGATCTTTAGGAAGGCACGCAGT |
| | TGCTTATTATGAATCATTTCTAGAGTCCGATGCATTTTCAAAGCCGGTTAC |
| | AGTCATTACGAAGCACACCCTTGTGAGGTAAGTGTATCATCACCTTTGGT |
| | TCATAAATAAAAAAGCTGAGACGCCGAGCGATTAAGTCACTCGCCTAAG |
| | GAGAATGAGTCAACGTCAAGAGTCATAGTTGACCCGGCCTAAAGACTCC |
| | AGACCATCAGTCCAGGGCTTAGTCAGCGGGGCCCGGAGTGGCTTCCCTG |
| | GCTGGCATCTGGACTTAGGCTATTTCCGTGCACGTAAAAGCGGAATATTG |
| | GAACGGTTGCACAGAACTTCCAAATAATTTTTACCGCCACGCAAGATTTA |
| | GCCCTGAGGTCTTAATCTCAGGATTTGGGACAGTAAAAGCTGTCGTCCCT |
| | CCCCCTCGTCCAGCCGGTGGCAAGCGGGTACTGCGGGCGGTTCCGTCCGT |
| | CCCCTTTCGCAGAAATGGCAACGAATGACCACCAGCATTAGCTGAGCCA |
| | GGGGACGTGGGAGGGTTGATTGCCTAAACGACTCTGCATCGCCGCCTCTT |
| | TTTGAAACTAAGAGAAAATGGTGGGAGATCAAAAGAAAACTAAATAAAC |
| | ACACAGGCAACTTGTCCTGGGACCTCAACTAAGCAAATGAAGCCTTATTG |
| | TGTGTGCTGAGCCTGCAGTTCCCAACCTTCCGGGGAAGATGGGAGGACA |
| | GGGCGACAAAGGGCACAGTAGGCTTGCCTGGCAGTAAGTGTGACCGCAG |
| | CTATCCAGGCGGAAGAGCAGAGGACTGAAACCACCCTCCAGCAAGCGAG |
| | TGTCCGCCGCGTTGAGAACCGCGCACCCTACCCATCGGCCACGTGACCAG |
| | TCCTTTTTAAAAAAAATTTCTTTACCTTAAAAAAAAAAAAAAAAAAAAG |
| | GTGGGGGAGAGACTCCACTTCCCAGAAGCCTCTCGTTACTCACGCAGCCG |
| | CAGTCTTGCGCAGGTGCCGCCAGGGCCAAACGGACATATCCGTCACGTG |
| | GCCAGAAGCTGGCCAATCCGGTTTGAATCTCATTTTTTTCCTCTTACCCCC |
| | CCTTCTGGAGCGGTTGTGCGATCAGATCGATCTAAGATGGCGACTGTCGA |
| | ACCGGAAACCACCCCTACTCCTAATCCCCCGACTACAGAAGAGGAGAAA |
| | ACGGAATCTAATCAGGAGGTTGCTAACCCAGAACACTATATTAAACATCC |
| | CCTACAGAACAGATGGGCACTCTGGTTTTTTAAAAATGATAAAAGCAAA |
| | ACTTGGCAAGCAAACCTGCGGCTGATCTCCAAGTTTGATACTGTTGAAGA |
| | CTTTTGGGCTCTGTACAACCATATCCAGTTGTCTAGTAATTTAATGCCTGG |
| | CTGTGACTACTCACTTTTTAAGGATGGTATTGAGCCTATGTGGGAAGATG |
| | AGAAAAACAAACGGGGAGGACGATGGCTAATTACATTGAACAAACAGC |
| | AGAGACGAAGTGACCTCGATCGCTTTTGGCTAGAGACACTTCTGTGCCTT |
| | ATTGGAGAATCTTTTGATGACTACAGTGATGATGTATGTGGCGCTGTTGT |
| | TAATGTTAGAGCTAAAGGTGATAAGATAGCAATATGGACTACTGAATGT |
| | GAAAACAGAGAAGCTGTTACACATATAGGGAGGGTATACAAGGAAAGGT |
| | TAGGACTTCCTCCAAAGATAGTGATTGGTTATCAGTCCCACGCAGACACA |
| | GCTACTAAGAGCGGCTCCACCACTAAAAATAGGTTTGTTGTTTAAGAAGA |
| | CACCTTCTGAGTATTCTCATAGGAGACTGCGTCAAGCAATCGAGATTTGG |
| | GAGCTGAACCAAAGCCTCTTCAAAAAGCAGAGTGGACTGCATTTAAATTT |
| | GATTTCCATCTTAATGTTACTCAGATATAAGAGAAGTCTCATTCGCCTTTG |
| | TCTTGTACTTCTGTGTTCATTTTTTTTTTTTTTTTTGGCTAGAGTTTCCACT |
| | ATCCCAATCAAAGAATTACAGTACACATCCCCAGAATCCATAAATGTGTT |
| | CCTGGCCCACTCTGTAATAGTTCAGTAGAATTACCATTAATTACATACAG |
| | ATTTTACCTATCCACAATAGTCAGAAAACAACTTGGCATTTCTATACTTTA |
| | CAGGAAAAAAAATTCTGTTGTTCCATTTTATGCAGAAGCATATTTTGCTG |
| | GTTTGAAAGATTATGATGCATACAGTTTTCTAGCAATTTTCTTTGTTTCTT |
| | TTTACAGCATTGTCTTTGCTGTACTCTTGCTGATGGCTGCTAGATTTTAAT |
| | TTATTTGTTTCCCTACTTGATAATATTAGTGATTCTGATTTCAGTTTTTCAT |
| | TTGTTTTGCTTTTGTTTTTTTCCTCATGTAACATTGGTGAAGGATCCAGGA |
| | ATATGACACAAAGGTGGAATAAACATTAATTTTGTGCATTCTTTGGTAAT |
| | TTTTTTTGTTTTTTGTAACTACAAAGCTTTGCTACAAATTTATGCATTTCAT |
| | TCAAATCAGTGATCTATGTTTGTGTGATTTCCTAAACATAATTGTGGATTA |
| | TAAAAAATGTAACATCATAATTACATTCCTAACTAGAATTAGTATGTCTG |
| | TTTTTGTATCTTTATGCTGTATTTTAACACTTTGTATTACTTAGGTTATTTT |
| | GCTTTGGTTAAAAATGGCTCAAGTAGAAAAGCAGTCCCATTCATATTAAG |
| | ACAGTGTACAAAACTGTAAATAAAATGTGTACAGTGAATTGTCTTTTAGA |
| | CAACTAGATTTGTCCTTTTATTTCTCCATCTTTATAGAAGGAATTTGTACT |
| | TCTTATTGCAAGGCAGTCTCTATATTATGTCTTCTTTTGTGGTGTCTTCCAT |
| | GTGAACAGCATAAGTTTGGAGCACTAGTTTGATTATTATGTTTATTACAA |
| | TTTTTAATAAATTGAATAGGTAGTATCATATATATGGAATTAAATTGATG |
| | TGGCTATCTTTGTTTTTTTATAAAGTAAGGCACAGTCATTCAGTCTTAGGT |
| | AAATAATGTACTCTCTTAATATGTTAATACTCATGAGAATTGGGATCTGA |
| | TGCATCACCATTTGATTGGTAGCAACAGTGGTTGTAAAACTTGGTTGCTG |
| | AATTGAGTTGTTTCTATGTTAAGTGTCAAAATGATAGTGTAGGGAAAGTA |
| | CAGGTGGTGGGGACATATGCATTAAGAATCTTGTTAGTGTTGCAATCTAA |
| | ATAGAATGGAATAAACAGGTGTTAAGACATATTTATAGTGGTAAATTGTT |
| | GTAGTATGGTATTCTGTAAACTTGAAAACTTGATCTACTCTTTGTAGGTAT |
| | CATTTGAAAGCAAACTTGAAAATGTTTTGTACATAGTACATACTTGTATA |
| | GTCCTGTGAGATGAAGTATGGCTATCAGACCAAAGGATAAGCCAAACTG |
| | TAGGTAGCAGAATGGAAATTATTATTTTGAGAGGAAAATTTGTCTTTGAA |
| | TGGTGATTATGACTTAATCATTTTAAAACTGATAAACTTGACAAAAACCC |
| | TGTATGAAATAAACATGAAATTAATAGCACTGATTTCATTGTAAAATTTT |
| | AAAGCAGTTTAAAGGGTACCACAGGTTATCACAGTACTCTCAATGCCACA |
| | AACACCTCTTGTTCAGTATTCTAGAAATACTGAATCAGAATTCTGTGTTTA |
| | TTATAATCTCAGCATACTGTACATAATATCTGCTAGTTAAACTTGGGTAA |
| | TTGGTTAAGGTGACTTACTGTCTATGTCAATATGTATAGTTTTGAGTACTT |
| | CAAGAGTTTACTTAAAAGTGATGATGTTACTGGTATGTTGGCAGTGGGTG |
| | GGACTGAAGTAGTGTATCTATTATAAATTGATCTATTTTCTTAATTCTAAG |
| | ATGAAGTCCAATTTTAAGCATCAGCTTTTAGGTGCAAAGGAGGAATTAAC |
| | ACATTAAATGTATACAGTTCTAAATTTTTGAAATAACTGATGTGTAGCAT |
| | TTGATTATTGGTATTACCATTTTAGAATCATGATGTTATTTTAAACCTTTT |
| | TCCTGGGGACAAGAAAGGATAATAAATTACGCTGAATCACTTTTGGCAGT |
| | TGCCACTTAAATAGTACAGTGACTTGCAACTTTTATAACTTTATCAGCATC |
| | TTCTCTAAATACAAAATTAGGCTATATGTTATTTTCCAACTTACTGTTTTC |
| | TCTCTGTTTAGCAGGATATTATAAATAGATTAAATAGATATATTTTCTTTT |
| | TTTTTTTTTTTTTTTGAGACGGAGTCTCGCTTTGTCTCCCAGGCTGGAGTG |
| | CAGTGGCGTGATCTCCCAGTAGCTGGGACTACAAGCACCTGCCACCATGC |
| | CCGGCTAATTTTTTTTGTATTTTTAGTAGAGACGGGGTTTCACTGTGTTAG |
| | CCAAGATGGTCTCAATCTCCTGACTTTGTGATCTGCCTGCTTCTGCCTCCC |
| | AAAGTGCTGGGATTACAGGTGTGAGCCACCGTACCCAGCCCAAATAGAT |
| | GTATTTTCATAATAGAGAATTGAAATAGGCTTTAATGGGTGAATAGCAGT |
| | TTATTGTAGGCATGTGACATTTCATTTAATGAATTTAAAGTTTATTATCCC |
| | AATTCTACAGAAGGATTTAATGCATACTATGCAATTAAATAATTATAACA |
| | CTACATAGTAATAATTTATGTGCCAGGCAGTAGTTCAGTCACTTTACATG |
| | CTTATTAACCTGCAGAATAATCTTTTGAGATGTAGGTGCTGTTACTGAGA |
| | ATTTAACTTTTGCTTGTAAATTGCAAAGGGTGGATTTGAATTCTGGAAAT |
| | TTGGTTCCAGAGACAATAATTACATAACACTTTCTCCATAGGGTACAGCC |
| | TGTCTAATAGGCTATAGTAAATCACCTCAGCTTGTTATAGGTCGGGCATG |
| | CAAACATTTCTCCATTTTACTCCCTTTGGTAATGAATCTAGTAATAGATGG |
| | AAATTTTCCCTAGATTCACTGTGTTAGTCAGTTGGGGAAGTTTGGAGGCA |
| | AAGATACAGGAGTTTATGGGGAGGTAGTGTACATAAATATAATCATATG |
| | CTATATAAGGAAGTTTTGGTCAGCAGCAGACCATATATAGGATGGTGGG |
| | CCAGTAACATTGTAACACTGTATTTTTACTGTATCTTTTCCATGTTTTGTT |
| | ATGTTTAGATACACAAATAACATTATGGAGTATTCAGTATGGTAACATGC |
| | CATACAGGTTTGTAGCCTAGGAGCAGTAATAGACTGTTCCCTGAAACCTA |
| | TATTTGTGGTAGGTTTATACCATTCAGGTTTGTGTAAGTACACAACGAAA |
| | TCATCTAATGGCCCATTTCTCAAAACATATTCCCATCATTAATCAATGCAT |
| | GGTCATGTTTTCGTATACATTTTAAGCTTCTGTATTCTAATCTAATATAAA |
| | TGGCAAAATATTCAAACTGATAGGCATTGAGATTCTTAAATGCTAAAGTT |
| | GCATTCAAAAGGATAATTTTAGGCGTTGTGACAAAGCAGTGTTATATTTT |
| | AAAGTTAGTGACAAGGCTATGCACCTTTTATCTCTAATTGTTTCTTACAGA |
| | ATGTTTTTATTATTGAGTAGTAAAACAATAAATGTCAGATCCTTTATACA |
| | AATTCAAGATTGACATTGATAAACAAAACTTCAGCATATCACTCAAGGTC |
| | AGCGTAGAAATTGTGTGTCTGGAAACTTCTATAGTAATTTTATATTACTGT |
| | GACATTAGTATGTGATCACTTTTCTAGTAATGTTTTAAAAAAATATATCTT |
| | ACAGGCCAGGCATAGTGCTTTATGCCTGTAATCCCAGCACTTTGGGAGGC |
| | CAAGGTGGCAGGATTGCATGAGTCCAGGAGTTCAACACCAGTCTGGGCA |
| | ATAAAGTGAGACCCCATCGCTACAAACAAATTAAAAAATATTTATGTATG |
| | TGTGTAATATATATAATATATAACAAAACACATATGTATGTGTATATATA |
| | GTATGTCTGGCAGAGTACAATTTAGGGGTTAAGACTGGTCCCTCACATAT |
| | GGTGTGAGAAACACTGTTCACAGGTTGCTTTCCCCATTAGCCCAGGGCAA |
| | CTCATTTGCCCATCATTTCCTAGAACAACCGGGTCTGTTACGTCTACAGTT |
| | TTCATCTTCATGCAGTTATGGATTTGGTGTCAAAAACTTTGGTCCTGTTCT |
| | CTACCATCTTAAAATAAACTCTTGTGTCCTGCTTTACTATGAATTGCAAAG |
| | TAGGCATTAGGTAGCCTTCCTACTACCATAGTTTAGAGTTCAATATTCTTA |
| | TGACCATTCTACTGGTAGAAGCAAAAAATGAACTTGTAGGCATGTGATCA |
| | CATGTGCCTATGGTGCTGTCTTTTCCAGTACAGGGGAACTAATTTTCATAT |
| | TTTAATCTTGCAGCTTTTTGTTTACTTCATGCATTGTGATTTCTCATAGTTT |
| | TGCACAGAACTCACTTCCCTACCTTTTCTGAAACAAAAGTATGTATACAC |
| | ACATACATATGTATTGAGCACCTCTATTTACTGTGTTCCACGTGCTGGGC |
| | ATATAGCAAGAACAGAATGGTCTGGGGCCCTGCTCTAAAGAAGATTTAA |
| | AAGCAAACATATATTAAAAATGCGTGAGTCTGGCCAGGAGCAGTGGCTC |
| | ATGCCTGCAACCCCAGCACTTTGGGAGGCTGAAGCGGGTGAATCACCTG |
| | AGATCAGGAGTTGAGACCAGCCTGGCCAACATGGTGAAACCCCGTCTCT |
| | ACTAAAAATACAAAAATTAGCCTGGCATGGTGGCATGCGCCTGTAATTCC |
| | AGCTACTTTGGAGACTGAGACAGGAGAATCACTTGAGCCCAGGAAGTGG |
| | AGGTTGCAGTGAGCTGAGATCGCGCCACTGCACTCCAGCCTGGGTGACA |
| | GAGCAAGACTCCATCTCAAAAAAAAAAAAAAAACGTGTCTGTTCATAAG |
| | GTTCTACAAATAGCTGTTGTTACAGAAAATAGGACTAGGGTTTTATTACT |
| | GGGGATTATGCAGTCGAGGTAAATATAAAATGAGGTTGTTTCTTCTTTTT |
| | TTTATTTGAGACAGAGTCTTGTTTGCCAGGCTGGAGTGCAGTGGCGTGCT |
| | CTTAGCTCCGCCTCCCGGGATCAAACGATTCTCTTGCCTCAGCCTCTCGA |
| | GTAGCTGGGACTACAGGCGCGTGCCACCACACCCAGCTGATTTTTGTATT |
| | TTTAGTAGAGATGGGATTTCACCATGATGGCCAGGATGGTCTCGATCTTT |
| | TGACCTCATGATCCACCCACCTCGGCCTCCCAAAGTGCTGGGATTACAGG |
| | CGTGAGCCACTGCACCCGGCCCTGGTTCTAAATACTCTTCCAGCTCTAAA |
| | ACATTGATTCTAAACAGATCACATTCCAGGAGGACTCTAGCAAACCAGC |
| | ATATGCAAATTATAGCTTTTGCAAGACCGTTTCTACTTTTATATTACTGAA |
| | CTCTATATGATTGTCCAAGTAAAGTTTTGTGTCTCTTTGATTATTTGATTG |
| | TACTTTAAAATTTTTTCACCATTCATTTAACATTTTTTACCATACTGTAGT |
| | ATTTTTAATGCAATTGTGTTTGCATTGGTGTGGCTTTAGAGGCTTCTCCAA |
| | CCACCTTCCCAAAATACTGATCTGTGATTTTTTTCTTTAATGTTTGGCCAA |
| | ACATAATACATGCTTATTTTATTTTTCATCCCTACAGAAAGGTAGAAGAT |
| | GAGAATTCTGTCTCCTACTGTTGTTTTTCAAGGTGCCACTCAAATTTCTTG |
| | TACGTGTCTAGAAACTTGTGCATACAATAGAAGTACACTGTGGCTGGGCA |
| | TGGTGGCTCATGCCTGTAATCCCAGCACTTTGTGAAGCTGAGGTGGGTGG |
| | ATCACCTGAGGTCAGGGAGTTTGAGACCAGCCTGGCCAACATGATGAAA |
| | CCCCGTCTTTACTAAAATTAGAAAAAATTAGCCAGGCGTGGTGGTGTGCG |
| | CCTGTAATCCCAGCTACTCGGGAGGCTGAGGCATGAGAATCACTTGAACT |
| | CGGGTGGCAGAGGCTGCAGTGAGCTGAGATCATGCCACTGCATTCCAGC |
| | CTGCGCAGCTGAGCCAGACTCCATCTCCAAAAAAAAAAAAAAAAAAAAA |
| | AAAAGATGTCCAATATATGTAACTTTTTTCTTTGGACACAAAAATTCCAT |
| | TAGCTTTGTTTTCTCATTTTTACTTGTCATGATGTATGTCGAACACATTATT |
| | TTTAGTGTCTGGTGTTCCATTATACAGATGTCTCTCTTGTTGGTTGAATTT |
| | TTGCATTCACAGACCCTCAAGTTGGATTCATATCTTTTTACACTAAGCATA |
| | AAGAAGACGGATTGGGGTCGGGTATGGTGGCTCACGCCTGTAATCCCAG |
| | CACTTCGGGAGGCTGAGGTGGGCGGATCACGAGGTCAGGAGTTCGAGAC |
| | CAGCCTGGCCAACATACTGAAACCCCGTCTCTAAAAATATGAAAAAAAA |
| | ATTAGCCAGGCGTGGTGGTGCGCAGCTGTAGCCCCAGCTACTTGGGAGG |
| | CTGAGGCAGGAGAATCACTTGAACCCAGGAGGTGGAGGTTGCAGTGAGC |
| | ATATTGCACCATTGCATTCCAGCCTGCACGACAGAGGAAGACGCCATTTC |
| | AAAAAAAAAAAAAAAAAGACGGATTGATTTTCTTCAGCAGTATCAAGTG |
| | GCACTTACCATCCACCCCTTTAACACCCAAACCATTCTAATCCATGTATA |
| | CAGACCATCATCTGTTTCGTTATTGTGTTCTATAATGCAGCTTTGCGAGTT |
| | ATGCAGTTTTGGTCCTCATGATATTTTTCTGATTGGTTTTTAATGTATTTTG |
| | TTCTAACGAGCCAACATTGTTATACATGTAATTTGTTTATTTGCTAGATTG |
| | TACTCTTTCCCAAAGATGGGCTATGAACTAGCATCTACTTTCATTTTACCT |
| | ATTTACCTGTAAACATTGAAAAAACTGAATCAAATGCAGTGATATCGGAC |
| | CTAGTTTTATTGTTATGCCTTATAATGAATTTAACTTCACAGTTTTCTAAA |
| | TGAGAGCATTTCCCAAAGACATCTTTATGGTCATAACCAGTTTCCCTTGG |
| | CATTTGATTTATTTTTATTTTTATTTATTTATCTTTTTGAGAAGGAGTTTCG |
| | CTCTTGTTGCCCAGGCTAGAGTGCAATGGCGTGATCTCGGGTCACTGCAA |
| | CCTCTGCCTCCCGGGTTCAAGCGATTCTCCTGCCTCAGCCTCCAAGTAGCT |
| | GGGATTACAGGCATGCACCACCACGCCCGACTAATTTGTATTTTTAGAGA |
| | CGGGGTTTCTCCGTGTTGGTCAGGCTGGTCTCAAACTTCCAACCTCAGGT |
| | GATCCGCCCGCCGTGGCCTCCCAAAGTGTTGGGATTACAGGCGTGAGCCA |
| | CAGTGTCTGGCCTGATTTGTTTTTAAGAGCATTATTTTTCTGCTTTATTTTG |
| | TGACTTCAACATTTGACACAATTTTGGGTGAATGGTTTGTGCATGGTGCC |
| | TGACATCGTGTTTTGATGTGTAGTATATGCCATAGGACATGTGAGACAAG |
| | ATATGTCCCAACTTGACCTTGTTTTGTATTGTTTATGTCAAGGTGTTGAGT |
| | GTATTAGATATACTGTTGGGGCTCTGTGTTCTAGCTCTGCCTTTTAGATAA |
| | TAACCATGGTTAAATATTGCAATGTCCTGCATGTCTCCACACATGGGTTTT |
| | GTAACTGAGTCAGAATGATAAGTGATTAGTAAGCACATTTTTTCTCCTTT |
| | CAGGAAACCACTATTTTCCTTTTCTACATGCTGTTTTGTAAGTAGTACTTT |
| | TATGAAGGTTGTCTTCAAATGTTCGCATCTTCCATTTCTACTGCCCTTGGG |
| | TTATCCATCCTGTCATTTTGTGCCAATCACTTTTTTTTTTAACTTTTAAGTT |
| | CAGGGATGAAAGTGTAGATTTGTTACATAGGTAAACTTGTGTCATGGGGT |
| | TGTTGTACAGATTATTTCATCACCCAGGTGTTAAGCCTAGTATGCACTAG |
| | TTGTTTTTCCTGATCCTCTCCCTGCTCCCACCCTCCACCCTCTGATAGGCC |
| | CCAGTGTGTGTTGTTCCCCTCTGTGTGTCCATGTGTTCTCATCATTTAGCT |
| | CCCACTTACAAGTGAGAATGTGGTGTTTGGTTTTCTGTTCCTGCATTAGTT |
| | TGCTAAGGATAATGGCCTCGAGCTCCATCCAAGTCCCTGTAAAGGACATG |
| | ATCTTGTACTTTTTTATGGCTGCATAGTATTCTGTGGTGTATATGTACCAC |
| | ATTTTCTTTATCCAGTCTATCATTAGGCATTTAGGTTGATTCCATGTTTTTG |
| | CTATTGTGAATAGTGCTGCAATGAACATACATGTGCATGTCTTTTTATAAT |
| | AGAATGATTTATATTCTGTTGTGTATATACCCAGTAATGGGATTGCTGAG |
| | TTGAATGCTATTTCTGCCTTTAGGTCTTTGATGAATTGCCACACTGTCTTC |
| | CACAATGGTTGAACTAATTTACACTCCCACCAACGGTGAATAAGTGTTCA |
| | CTTTTCTCCACAACCTTGTCAGCATCTATTATTTTTTGACTTTTTAGTAATA |
| | GCCATTCTGACTGCTCACATCTATTTTGTAAATAAAGTTTTATTGAAACAT |
| | GGCCTTACCCATTTGTTTACATATATTCATGGCTGTTTTTGTGCCACAATG |
| | TCAGAGTTGTCTTAAAGTAGACAGAGACTATCTGGCTGTAAAGCCTGAGA |
| | TATATACTAACTGGTTCTTTATGTAAAAAGTTTGCTGACCACCTACTCTAA |
| | ACGTTTTGCAGTGATGGTAGTGTTGGCAAAAAACCAAATAGCTTACCCTC |
| | TTTAAATTTCCCTTTTACTTCTTACAAACTCCTAACACCATTTACGACTTT |
| | GTCATCAATATGGTCAACTAAGCTTGGTTTGCATGGCTCTACTTCCTTTCA |
| | CCTTCCACTTAGGCAGTGTCTCCAAGTCCACTGCAGTTTCTATTTGTCTCC |
| | TGACTGTTACTGTATCAGTTCTTACCTAAATAACATAACAACTGATCTCCC |
| | TACTTTTTGCCTATGCCCTCAAATGTGCTCATTGTTGATCTATCTCCCTGTT |
| | AGGTGTTCTTTTTCTCCTCTTTAGAAAGCAGCCAAGGAAACCAGGGTTCT |
| | CTCAAAGTGGAAAATACTGGAACTTATGTACTGTTATCATAATGATAGTT |
| | GGTGTTTTGAATTATAAGAATGATTCCAGGTGGTTTCTAAATCATCCAAT |
| | AAAGCTGTATTCACTCTGTAAAAAAAAAAA (SEQ ID NO: 54) |
| | AGGCACAGGCAGCCTGCATACACTCCTTTTCCTGGTGTCAACATTATTTA |
| (NM_001331017) | AAAGCATGGGAAATAGTAATGAGACAGTGTCTTCTTCATTAGAACCTTAG |
| Homo sapiens | GAGTCTACTAGATTTCTTCATCTCTATTTGTTGTTATTAGTAGCCAAACTG |
| eukaryotic | TGCAAAAAACACGGTCTTGAGAAATGACAGCACAGTATCTTAGAGGGAA |
| translation initiation | AGGAAATGTAGGATGCCAGTGTGGGGACAAATTTCTGATTGCCAGTGATT |
| factor 4E (EIF4E), | GTTGTGAGCATAACAATAATTTCATGAACATTAAAGCCTCTATTGAGGGC |
| transcript variant 4 | AGCTGCAGTTGTAAAGGAAAAAAAATGGTCCTGAACATTTAAAACTACA |
| | CTGGTGTACATCATAATCAAACAAAGTAAACAGAAAAAAATTTAAACTT |
| | TGCTAAAAAAAAAAAGCAGAAGCACTTGATCTTTAGGAAGGCACGCAGT |
| | TGCTTATTATGAATCATTTCTAGAGTCCGATGCATTTTCAAAGCCGGTTAC |
| | AGTCATTACGAAGCACACCCTTGTGAGGTAAGTGTATCATCACCTTTGGT |
| | TCATAAATAAAAAAGCTGAGACGCCGAGCGATTAAGTCACTCGCCTAAG |
| | GAGAATGAGTCAACGTCAAGAGTCATAGTTGACCCGGCCTAAAGACTCC |
| | AGACCATCAGTCCAGGGCTTAGTCAGCGGGGCCCGGAGTGGCTTCCCTG |
| | GCTGGCATCTGGACTTAGGCTATTTCCGTGCACGTAAAAGCGGAATATTG |
| | GAACGGTTGCACAGAACTTCCAAATAATTTTTACCGCCACGCAAGATTTA |
| | GCCCTGAGGTCTTAATCTCAGGATTTGGGACAGTAAAAGCTGTCGTCCCT |
| | CCCCCTCGTCCAGCCGGTGGCAAGCGGGTACTGCGGGCGGTTCCGTCCGT |
| | CCCCTTTCGCAGAAATGGCAACGAATGACCACCAGCATTAGCTGAGCCA |
| | GGGGACGTGGGAGGGTTGATTGCCTAAACGACTCTGCATCGCCGCCTCTT |
| | TTTGAAACTAAGAGAAAATGGTGGGAGATCAAAAGAAAACTAAATAAAC |
| | ACACAGGCAACTTGTCCTGGGACCTCAACTAAGCAAATGAAGCCTTATTG |
| | TGTGTGCTGAGCCTGCAGTTCCCAACCTTCCGGGGAAGATGGGAGGACA |
| | GGGCGACAAAGGGCACAGTAGGCTTGCCTGGCAGTAAGTGTGACCGCAG |
| | CTATCCAGGCGGAAGAGCAGAGGACTGAAACCACCCTCCAGCAAGCGAG |
| | TGTCCGCCGCGTTGAGAACCGCGCACCCTACCCATCGGCCACGTGACCAG |
| | TCCTTTTTAAAAAAAATTTCTTTACCTTAAAAAAAAAAAAAAAAAAAAG |
| | GTGGGGGAGAGACTCCACTTCCCAGAAGCCTCTCGTTACTCACGCAGCCG |
| | CAGTCTTGCGCAGGTGCCGCCAGGGCCAAACGGACATATCCGTCACGTG |
| | GCCAGAAGCTGGCCAATCCGGTTTGAATCTCATTTTTTTCCTCTTACCCCC |
| | CCTTCTGGAGCGGTTGTGCGATCAGATCGATCTAAGATGGCGACTGTCGA |
| | ACCGGGTGCTGTGAGGAAATGCATGGTTTGTTGAAAAGAACTGAGGACT |
| | GACAGCCGCCTCCTCCATGATTACCCTGGCTCTCTGTCCCACCCTTCTCAT |
| | GGCGCTTGGGGGACCATGGCTGATCCTGTCCTGAGACAAATTAAGACTCC |
| | TGTGGTGAAGCAGTTGGTCGAGAAAAAGTGATGCATGAAAAAGAAGCAA |
| | AACAAGAAGGAAAGAAAAAAAATGAGAGCTGAAGATGGTGAAAATGAT |
| | GCCATTAAAAAGCAGGCAGAAAGTCTGCGAGAATCCCAGGAAACCACCC |
| | CTACTCCTAATCCCCCGACTACAGAAGAGGAGAAAACGGAATCTAATCA |
| | GGAGGTTGCTAACCCAGAACACTATATTAAACATCCCCTACAGAACAGA |
| | TGGGCACTCTGGTTTTTTAAAAATGATAAAAGCAAAACTTGGCAAGCAA |
| | ACCTGCGGCTGATCTCCAAGTTTGATACTGTTGAAGACTTTTGGGCTCTGT |
| | ACAACCATATCCAGTTGTCTAGTAATTTAATGCCTGGCTGTGACTACTCA |
| | CTTTTTAAGGATGGTATTGAGCCTATGTGGGAAGATGAGAAAAACAAAC |
| | GGGGAGGACGATGGCTAATTACATTGAACAAACAGCAGAGACGAAGTGA |
| | CCTCGATCGCTTTTGGCTAGAGACACTTCTGTGCCTTATTGGAGAATCTTT |
| | TGATGACTACAGTGATGATGTATGTGGCGCTGTTGTTAATGTTAGAGCTA |
| | AAGGTGATAAGATAGCAATATGGACTACTGAATGTGAAAACAGAGAAGC |
| | TGTTACACATATAGGGAGGGTATACAAGGAAAGGTTAGGACTTCCTCCA |
| | AAGATAGTGATTGGTTATCAGTCCCACGCAGACACAGCTACTAAGAGCG |
| | GCTCCACCACTAAAAATAGGTTTGTTGTTTAAGAAGACACCTTCTGAGTA |
| | TTCTCATAGGAGACTGCGTCAAGCAATCGAGATTTGGGAGCTGAACCAA |
| | AGCCTCTTCAAAAAGCAGAGTGGACTGCATTTAAATTTGATTTCCATCTT |
| | AATGTTACTCAGATATAAGAGAAGTCTCATTCGCCTTTGTCTTGTACTTCT |
| | GTGTTCATTTTTTTTTTTTTTTTTGGCTAGAGTTTCCACTATCCCAATCAAA |
| | GAATTACAGTACACATCCCCAGAATCCATAAATGTGTTCCTGGCCCACTC |
| | TGTAATAGTTCAGTAGAATTACCATTAATTACATACAGATTTTACCTATCC |
| | ACAATAGTCAGAAAACAACTTGGCATTTCTATACTTTACAGGAAAAAAA |
| | ATTCTGTTGTTCCATTTTATGCAGAAGCATATTTTGCTGGTTTGAAAGATT |
| | ATGATGCATACAGTTTTCTAGCAATTTTCTTTGTTTCTTTTTACAGCATTGT |
| | CTTTGCTGTACTCTTGCTGATGGCTGCTAGATTTTAATTTATTTGTTTCCCT |
| | ACTTGATAATATTAGTGATTCTGATTTCAGTTTTTCATTTGTTTTGCTTTTG |
| | TTTTTTTCCTCATGTAACATTGGTGAAGGATCCAGGAATATGACACAAAG |
| | GTGGAATAAACATTAATTTTGTGCATTCTTTGGTAATTTTTTTTGTTTTTTG |
| | TAACTACAAAGCTTTGCTACAAATTTATGCATTTCATTCAAATCAGTGAT |
| | CTATGTTTGTGTGATTTCCTAAACATAATTGTGGATTATAAAAAATGTAA |
| | CATCATAATTACATTCCTAACTAGAATTAGTATGTCTGTTTTTGTATCTTT |
| | ATGCTGTATTTTAACACTTTGTATTACTTAGGTTATTTTGCTTTGGTTAAA |
| | AATGGCTCAAGTAGAAAAGCAGTCCCATTCATATTAAGACAGTGTACAA |
| | AACTGTAAATAAAATGTGTACAGTGAATTGTCTTTTAGACAACTAGATTT |
| | GTCCTTTTATTTCTCCATCTTTATAGAAGGAATTTGTACTTCTTATTGCAA |
| | GGCAGTCTCTATATTATGTCTTCTTTTGTGGTGTCTTCCATGTGAACAGCA |
| | TAAGTTTGGAGCACTAGTTTGATTATTATGTTTATTACAATTTTTAATAAA |
| | TTGAATAGGTAGTATCATATATATGGAATTAAATTGATGTGGCTATCTTT |
| | GTTTTTTTATAAAGTAAGGCACAGTCATTCAGTCTTAGGTAAATAATGTA |
| | CTCTCTTAATATGTTAATACTCATGAGAATTGGGATCTGATGCATCACCA |
| | TTTGATTGGTAGCAACAGTGGTTGTAAAACTTGGTTGCTGAATTGAGTTG |
| | TTTCTATGTTAAGTGTCAAAATGATAGTGTAGGGAAAGTACAGGTGGTGG |
| | GGACATATGCATTAAGAATCTTGTTAGTGTTGCAATCTAAATAGAATGGA |
| | ATAAACAGGTGTTAAGACATATTTATAGTGGTAAATTGTTGTAGTATGGT |
| | ATTCTGTAAACTTGAAAACTTGATCTACTCTTTGTAGGTATCATTTGAAAG |
| | CAAACTTGAAAATGTTTTGTACATAGTACATACTTGTATAGTCCTGTGAG |
| | ATGAAGTATGGCTATCAGACCAAAGGATAAGCCAAACTGTAGGTAGCAG |
| | AATGGAAATTATTATTTTGAGAGGAAAATTTGTCTTTGAATGGTGATTAT |
| | GACTTAATCATTTTAAAACTGATAAACTTGACAAAAACCCTGTATGAAAT |
| | AAACATGAAATTAATAGCACTGATTTCATTGTAAAATTTTAAAGCAGTTT |
| | AAAGGGTACCACAGGTTATCACAGTACTCTCAATGCCACAAACACCTCTT |
| | GTTCAGTATTCTAGAAATACTGAATCAGAATTCTGTGTTTATTATAATCTC |
| | AGCATACTGTACATAATATCTGCTAGTTAAACTTGGGTAATTGGTTAAGG |
| | TGACTTACTGTCTATGTCAATATGTATAGTTTTGAGTACTTCAAGAGTTTA |
| | CTTAAAAGTGATGATGTTACTGGTATGTTGGCAGTGGGTGGGACTGAAGT |
| | AGTGTATCTATTATAAATTGATCTATTTTCTTAATTCTAAGATGAAGTCCA |
| | ATTTTAAGCATCAGCTTTTAGGTGCAAAGGAGGAATTAACACATTAAATG |
| | TATACAGTTCTAAATTTTTGAAATAACTGATGTGTAGCATTTGATTATTGG |
| | TATTACCATTTTAGAATCATGATGTTATTTTAAACCTTTTTCCTGGGGACA |
| | AGAAAGGATAATAAATTACGCTGAATCACTTTTGGCAGTTGCCACTTAAA |
| | TAGTACAGTGACTTGCAACTTTTATAACTTTATCAGCATCTTCTCTAAATA |
| | CAAAATTAGGCTATATGTTATTTTCCAACTTACTGTTTTCTCTCTGTTTAG |
| | CAGGATATTATAAATAGATTAAATAGATATATTTTCTTTTTTTTTTTTTTTT |
| | TTTGAGACGGAGTCTCGCTTTGTCTCCCAGGCTGGAGTGCAGTGGCGTGA |
| | TCTCCCAGTAGCTGGGACTACAAGCACCTGCCACCATGCCCGGCTAATTT |
| | TTTTTGTATTTTTAGTAGAGACGGGGTTTCACTGTGTTAGCCAAGATGGTC |
| | TCAATCTCCTGACTTTGTGATCTGCCTGCTTCTGCCTCCCAAAGTGCTGGG |
| | ATTACAGGTGTGAGCCACCGTACCCAGCCCAAATAGATGTATTTTCATAA |
| | TAGAGAATTGAAATAGGCTTTAATGGGTGAATAGCAGTTTATTGTAGGCA |
| | TGTGACATTTCATTTAATGAATTTAAAGTTTATTATCCCAATTCTACAGAA |
| | GGATTTAATGCATACTATGCAATTAAATAATTATAACACTACATAGTAAT |
| | AATTTATGTGCCAGGCAGTAGTTCAGTCACTTTACATGCTTATTAACCTGC |
| | AGAATAATCTTTTGAGATGTAGGTGCTGTTACTGAGAATTTAACTTTTGCT |
| | TGTAAATTGCAAAGGGTGGATTTGAATTCTGGAAATTTGGTTCCAGAGAC |
| | AATAATTACATAACACTTTCTCCATAGGGTACAGCCTGTCTAATAGGCTA |
| | TAGTAAATCACCTCAGCTTGTTATAGGTCGGGCATGCAAACATTTCTCCA |
| | TTTTACTCCCTTTGGTAATGAATCTAGTAATAGATGGAAATTTTCCCTAGA |
| | TTCACTGTGTTAGTCAGTTGGGGAAGTTTGGAGGCAAAGATACAGGAGTT |
| | TATGGGGAGGTAGTGTACATAAATATAATCATATGCTATATAAGGAAGTT |
| | TTGGTCAGCAGCAGACCATATATAGGATGGTGGGCCAGTAACATTGTAA |
| | CACTGTATTTTTACTGTATCTTTTCCATGTTTTGTTATGTTTAGATACACAA |
| | ATAACATTATGGAGTATTCAGTATGGTAACATGCCATACAGGTTTGTAGC |
| | CTAGGAGCAGTAATAGACTGTTCCCTGAAACCTATATTTGTGGTAGGTTT |
| | ATACCATTCAGGTTTGTGTAAGTACACAACGAAATCATCTAATGGCCCAT |
| | TTCTCAAAACATATTCCCATCATTAATCAATGCATGGTCATGTTTTCGTAT |
| | ACATTTTAAGCTTCTGTATTCTAATCTAATATAAATGGCAAAATATTCAA |
| | ACTGATAGGCATTGAGATTCTTAAATGCTAAAGTTGCATTCAAAAGGATA |
| | ATTTTAGGCGTTGTGACAAAGCAGTGTTATATTTTAAAGTTAGTGACAAG |
| | GCTATGCACCTTTTATCTCTAATTGTTTCTTACAGAATGTTTTTATTATTGA |
| | GTAGTAAAACAATAAATGTCAGATCCTTTATACAAATTCAAGATTGACAT |
| | TGATAAACAAAACTTCAGCATATCACTCAAGGTCAGCGTAGAAATTGTGT |
| | GTCTGGAAACTTCTATAGTAATTTTATATTACTGTGACATTAGTATGTGAT |
| | CACTTTTCTAGTAATGTTTTAAAAAAATATATCTTACAGGCCAGGCATAG |
| | TGCTTTATGCCTGTAATCCCAGCACTTTGGGAGGCCAAGGTGGCAGGATT |
| | GCATGAGTCCAGGAGTTCAACACCAGTCTGGGCAATAAAGTGAGACCCC |
| | ATCGCTACAAACAAATTAAAAAATATTTATGTATGTGTGTAATATATATA |
| | ATATATAACAAAACACATATGTATGTGTATATATAGTATGTCTGGCAGAG |
| | TACAATTTAGGGGTTAAGACTGGTCCCTCACATATGGTGTGAGAAACACT |
| | GTTCACAGGTTGCTTTCCCCATTAGCCCAGGGCAACTCATTTGCCCATCAT |
| | TTCCTAGAACAACCGGGTCTGTTACGTCTACAGTTTTCATCTTCATGCAGT |
| | TATGGATTTGGTGTCAAAAACTTTGGTCCTGTTCTCTACCATCTTAAAATA |
| | AACTCTTGTGTCCTGCTTTACTATGAATTGCAAAGTAGGCATTAGGTAGC |
| | CTTCCTACTACCATAGTTTAGAGTTCAATATTCTTATGACCATTCTACTGG |
| | TAGAAGCAAAAAATGAACTTGTAGGCATGTGATCACATGTGCCTATGGT |
| | GCTGTCTTTTCCAGTACAGGGGAACTAATTTTCATATTTTAATCTTGCAGC |
| | TTTTTGTTTACTTCATGCATTGTGATTTCTCATAGTTTTGCACAGAACTCA |
| | CTTCCCTACCTTTTCTGAAACAAAAGTATGTATACACACATACATATGTA |
| | TTGAGCACCTCTATTTACTGTGTTCCACGTGCTGGGCATATAGCAAGAAC |
| | AGAATGGTCTGGGGCCCTGCTCTAAAGAAGATTTAAAAGCAAACATATA |
| | TTAAAAATGCGTGAGTCTGGCCAGGAGCAGTGGCTCATGCCTGCAACCCC |
| | AGCACTTTGGGAGGCTGAAGCGGGTGAATCACCTGAGATCAGGAGTTGA |
| | GACCAGCCTGGCCAACATGGTGAAACCCCGTCTCTACTAAAAATACAAA |
| | AATTAGCCTGGCATGGTGGCATGCGCCTGTAATTCCAGCTACTTTGGAGA |
| | CTGAGACAGGAGAATCACTTGAGCCCAGGAAGTGGAGGTTGCAGTGAGC |
| | TGAGATCGCGCCACTGCACTCCAGCCTGGGTGACAGAGCAAGACTCCAT |
| | CTCAAAAAAAAAAAAAAAACGTGTCTGTTCATAAGGTTCTACAAATAGC |
| | TGTTGTTACAGAAAATAGGACTAGGGTTTTATTACTGGGGATTATGCAGT |
| | CGAGGTAAATATAAAATGAGGTTGTTTCTTCTTTTTTTTATTTGAGACAGA |
| | GTCTTGTTTGCCAGGCTGGAGTGCAGTGGCGTGCTCTTAGCTCCGCCTCC |
| | CGGGATCAAACGATTCTCTTGCCTCAGCCTCTCGAGTAGCTGGGACTACA |
| | GGCGCGTGCCACCACACCCAGCTGATTTTTGTATTTTTAGTAGAGATGGG |
| | ATTTCACCATGATGGCCAGGATGGTCTCGATCTTTTGACCTCATGATCCA |
| | CCCACCTCGGCCTCCCAAAGTGCTGGGATTACAGGCGTGAGCCACTGCAC |
| | CCGGCCCTGGTTCTAAATACTCTTCCAGCTCTAAAACATTGATTCTAAAC |
| | AGATCACATTCCAGGAGGACTCTAGCAAACCAGCATATGCAAATTATAG |
| | CTTTTGCAAGACCGTTTCTACTTTTATATTACTGAACTCTATATGATTGTC |
| | CAAGTAAAGTTTTGTGTCTCTTTGATTATTTGATTGTACTTTAAAATTTTTT |
| | CACCATTCATTTAACATTTTTTACCATACTGTAGTATTTTTAATGCAATTG |
| | TGTTTGCATTGGTGTGGCTTTAGAGGCTTCTCCAACCACCTTCCCAAAATA |
| | CTGATCTGTGATTTTTTTCTTTAATGTTTGGCCAAACATAATACATGCTTA |
| | TTTTATTTTTCATCCCTACAGAAAGGTAGAAGATGAGAATTCTGTCTCCTA |
| | CTGTTGTTTTTCAAGGTGCCACTCAAATTTCTTGTACGTGTCTAGAAACTT |
| | GTGCATACAATAGAAGTACACTGTGGCTGGGCATGGTGGCTCATGCCTGT |
| | AATCCCAGCACTTTGTGAAGCTGAGGTGGGTGGATCACCTGAGGTCAGG |
| | GAGTTTGAGACCAGCCTGGCCAACATGATGAAACCCCGTCTTTACTAAAA |
| | TTAGAAAAAATTAGCCAGGCGTGGTGGTGTGCGCCTGTAATCCCAGCTAC |
| | TCGGGAGGCTGAGGCATGAGAATCACTTGAACTCGGGTGGCAGAGGCTG |
| | CAGTGAGCTGAGATCATGCCACTGCATTCCAGCCTGCGCAGCTGAGCCAG |
| | ACTCCATCTCCAAAAAAAAAAAAAAAAAAAAAAAAAGATGTCCAATATA |
| | TGTAACTTTTTTCTTTGGACACAAAAATTCCATTAGCTTTGTTTTCTCATTT |
| | TTACTTGTCATGATGTATGTCGAACACATTATTTTTAGTGTCTGGTGTTCC |
| | ATTATACAGATGTCTCTCTTGTTGGTTGAATTTTTGCATTCACAGACCCTC |
| | AAGTTGGATTCATATCTTTTTACACTAAGCATAAAGAAGACGGATTGGGG |
| | TCGGGTATGGTGGCTCACGCCTGTAATCCCAGCACTTCGGGAGGCTGAGG |
| | TGGGCGGATCACGAGGTCAGGAGTTCGAGACCAGCCTGGCCAACATACT |
| | GAAACCCCGTCTCTAAAAATATGAAAAAAAAATTAGCCAGGCGTGGTGG |
| | TGCGCAGCTGTAGCCCCAGCTACTTGGGAGGCTGAGGCAGGAGAATCAC |
| | TTGAACCCAGGAGGTGGAGGTTGCAGTGAGCATATTGCACCATTGCATTC |
| | CAGCCTGCACGACAGAGGAAGACGCCATTTCAAAAAAAAAAAAAAAAA |
| | GACGGATTGATTTTCTTCAGCAGTATCAAGTGGCACTTACCATCCACCCC |
| | TTTAACACCCAAACCATTCTAATCCATGTATACAGACCATCATCTGTTTCG |
| | TTATTGTGTTCTATAATGCAGCTTTGCGAGTTATGCAGTTTTGGTCCTCAT |
| | GATATTTTTCTGATTGGTTTTTAATGTATTTTGTTCTAACGAGCCAACATT |
| | GTTATACATGTAATTTGTTTATTTGCTAGATTGTACTCTTTCCCAAAGATG |
| | GGCTATGAACTAGCATCTACTTTCATTTTACCTATTTACCTGTAAACATTG |
| | AAAAAACTGAATCAAATGCAGTGATATCGGACCTAGTTTTATTGTTATGC |
| | CTTATAATGAATTTAACTTCACAGTTTTCTAAATGAGAGCATTTCCCAAA |
| | GACATCTTTATGGTCATAACCAGTTTCCCTTGGCATTTGATTTATTTTTAT |
| | TTTTATTTATTTATCTTTTTGAGAAGGAGTTTCGCTCTTGTTGCCCAGGCT |
| | AGAGTGCAATGGCGTGATCTCGGGTCACTGCAACCTCTGCCTCCCGGGTT |
| | CAAGCGATTCTCCTGCCTCAGCCTCCAAGTAGCTGGGATTACAGGCATGC |
| | ACCACCACGCCCGACTAATTTGTATTTTTAGAGACGGGGTTTCTCCGTGTT |
| | GGTCAGGCTGGTCTCAAACTTCCAACCTCAGGTGATCCGCCCGCCGTGGC |
| | CTCCCAAAGTGTTGGGATTACAGGCGTGAGCCACAGTGTCTGGCCTGATT |
| | TGTTTTTAAGAGCATTATTTTTCTGCTTTATTTTGTGACTTCAACATTTGAC |
| | ACAATTTTGGGTGAATGGTTTGTGCATGGTGCCTGACATCGTGTTTTGAT |
| | GTGTAGTATATGCCATAGGACATGTGAGACAAGATATGTCCCAACTTGAC |
| | CTTGTTTTGTATTGTTTATGTCAAGGTGTTGAGTGTATTAGATATACTGTT |
| | GGGGCTCTGTGTTCTAGCTCTGCCTTTTAGATAATAACCATGGTTAAATAT |
| | TGCAATGTCCTGCATGTCTCCACACATGGGTTTTGTAACTGAGTCAGAAT |
| | GATAAGTGATTAGTAAGCACATTTTTTCTCCTTTCAGGAAACCACTATTTT |
| | CCTTTTCTACATGCTGTTTTGTAAGTAGTACTTTTATGAAGGTTGTCTTCA |
| | AATGTTCGCATCTTCCATTTCTACTGCCCTTGGGTTATCCATCCTGTCATT |
| | TTGTGCCAATCACTTTTTTTTTTAACTTTTAAGTTCAGGGATGAAAGTGTA |
| | GATTTGTTACATAGGTAAACTTGTGTCATGGGGTTGTTGTACAGATTATTT |
| | CATCACCCAGGTGTTAAGCCTAGTATGCACTAGTTGTTTTTCCTGATCCTC |
| | TCCCTGCTCCCACCCTCCACCCTCTGATAGGCCCCAGTGTGTGTTGTTCCC |
| | CTCTGTGTGTCCATGTGTTCTCATCATTTAGCTCCCACTTACAAGTGAGAA |
| | TGTGGTGTTTGGTTTTCTGTTCCTGCATTAGTTTGCTAAGGATAATGGCCT |
| | CGAGCTCCATCCAAGTCCCTGTAAAGGACATGATCTTGTACTTTTTTATG |
| | GCTGCATAGTATTCTGTGGTGTATATGTACCACATTTTCTTTATCCAGTCT |
| | ATCATTAGGCATTTAGGTTGATTCCATGTTTTTGCTATTGTGAATAGTGCT |
| | GCAATGAACATACATGTGCATGTCTTTTTATAATAGAATGATTTATATTCT |
| | GTTGTGTATATACCCAGTAATGGGATTGCTGAGTTGAATGCTATTTCTGC |
| | CTTTAGGTCTTTGATGAATTGCCACACTGTCTTCCACAATGGTTGAACTAA |
| | TTTACACTCCCACCAACGGTGAATAAGTGTTCACTTTTCTCCACAACCTTG |
| | TCAGCATCTATTATTTTTTGACTTTTTAGTAATAGCCATTCTGACTGCTCA |
| | CATCTATTTTGTAAATAAAGTTTTATTGAAACATGGCCTTACCCATTTGTT |
| | TACATATATTCATGGCTGTTTTTGTGCCACAATGTCAGAGTTGTCTTAAAG |
| | TAGACAGAGACTATCTGGCTGTAAAGCCTGAGATATATACTAACTGGTTC |
| | TTTATGTAAAAAGTTTGCTGACCACCTACTCTAAACGTTTTGCAGTGATG |
| | GTAGTGTTGGCAAAAAACCAAATAGCTTACCCTCTTTAAATTTCCCTTTTA |
| | CTTCTTACAAACTCCTAACACCATTTACGACTTTGTCATCAATATGGTCAA |
| | CTAAGCTTGGTTTGCATGGCTCTACTTCCTTTCACCTTCCACTTAGGCAGT |
| | GTCTCCAAGTCCACTGCAGTTTCTATTTGTCTCCTGACTGTTACTGTATCA |
| | GTTCTTACCTAAATAACATAACAACTGATCTCCCTACTTTTTGCCTATGCC |
| | CTCAAATGTGCTCATTGTTGATCTATCTCCCTGTTAGGTGTTCTTTTTCTCC |
| | TCTTTAGAAAGCAGCCAAGGAAACCAGGGTTCTCTCAAAGTGGAAAATA |
| | CTGGAACTTATGTACTGTTATCATAATGATAGTTGGTGTTTTGAATTATAA |
| | GAATGATTCCAGGTGGTTTCTAAATCATCCAATAAAGCTGTATTCACTCT |
| | GTAAAAAAAAAAA (SEQ ID NO: 55) |

In some embodiments, the EIF4E is an ortholog of human EIF4E. For example, in some embodiments, the EIF4E is a plant ortholog such as the protein described in German-Retana, S. et al. J. Virol. (2008) vol. 82 no. 15 7601-7612 (incorporated herein by reference).

In some embodiments, the EIF4E protein has an amino acid sequence comprising, consisting of, or consisting essentially of all or part of a sequence selected from SEQ ID NO: 56, SEQ ID NO: 57, SEQ ID NO: 58, SEQ ID NO: 59, and a biological equivalent of each thereof.

| | |
|---|---|
| (NP_001124150) | MLDLTSRGQVGTSRRMAEAACSAHFLETTPTPNPPTTEEEKTES |
| | NQEVANPEHYIKHPLQNRWALWFFKNDKSKTWQANLRLISKF |
| | DTVEDFWALYNHIQLSSNLMPGCDYSLFKDGIEPMWEDEKNK |
| | RGGRWLITLNKQQRRSDLDRFWLETLLCLIGESFDDYSDDVCG |
| | AVVNVRAKGDKIAIWTTECENREAVTHIGRVYKERLGLPPKIVI |
| | GYQSHADTATKSGSTTKNRFVV (SEQ ID NO: 56) |
| (NP_001124151) | MATVEPETTPTPNPPTTEEEKTESNQEVANPEHYIKHPLQNRWA |
| | LWFFKNDKSKTWQANLRLISKFDTVEDFWALYNHIQLSSNLMP |
| | GCDYSLFKDGIEPMWEDEKNKRGGRWLITLNKQQRRSDLDRF |
| | WLETRWDLAMLPRLVSNFWPQVILPLQPPKVLELQLLCLIGESF |
| | DDYSDDVCGAVVNVRAKGDKIAIWTTECENREAVTHIGRVYK |
| | ERLGLPPKIVIGYQSHADTATKSGSTTKNRFVV (SEQ ID NO: 57) |
| (NP_001317946) | MKKKQNKKERKKMRAEDGENDAIKKQAESLRESQETTPTPNPP |
| | TTEEEKTESNQEVANPEHYIKHPLQNRWALWFFKNDKSKTWQ |
| | ANLRLISKFDTVEDFWALYNHIQLSSNLMPGCDYSLFKDGIEPM |
| | WEDEKNKRGGRWLITLNKQQRRSDLDRFWLETLLCLIGESFDD |
| | YSDDVCGAVVNVRAKGDKIAIWTTECENREAVTHIGRVYKERL |
| | GLPPKIVIGYQSHADTATKSGSTTKNRFVV (SEQ ID NO: 58) |
| (NP_001959) | MATVEPETTPTPNPPTTEEEKTESNQEVANPEHYIKHPLQNRWA |
| | LWFFKNDKSKTWQANLRLISKFDTVEDFWALYNHIQLSSNLMP |
| | GCDYSLFKDGIEPMWEDEKNKRGGRWLITLNKQQRRSDLDRF |
| | WLETLLCLIGESFDDYSDDVCGAVVNVRAKGDKIAIWTTECEN |
| | REAVTHIGRVYKERLGLPPKIVIGYQSHADTATKSGSTTKNRFV |
| | V (SEQ ID NO: 59) |

In some embodiments, one or more kinase phosphorylation domains of the EIF4E protein are mutated. In some embodiments, all kinase phosphorylation domains of the EIF4E protein are mutated. In some embodiments, the mutation replaces the amino acid of the phosphorylation domain with a negatively charged amino acid such as aspartic acid or glutamic acid. In other embodiments, the mutation replaces the amino acid of the phosphorylation domain with an uncharged residue such alanine or glycine. In some embodiments, EIF4E comprises one or more phosphomimetic mutations and/or mutations to reduce EIF4E′s interaction with EIF4G. In some embodiments, the EIF4E protein comprises one or more mutations selected from the group consisting of: S209D, H37R, V69A, and W73F. In some embodiments, the mutated EIF4E is constitutively active.

| Mutation | Type |
|---|---|
| S209D | phosphomimetic |
| H37R | reduce EIF4G interaction |
| V69A | reduce EIF4G interaction |
| W73F | reduce EIF4G interaction |

In some embodiments, the fusion protein is a dCas9-EIF4E fusion protein encoded by a nucleic acid comprising the following nucleic acid sequence:

(SEQ ID NO: 60)
ATGGGTAAGCCTATCCCTAACCCTCTCCTCGGTCTCGATTCTACGGCTAGCATGGAC
AAGAAGTACAGCATCGGCCTGGCCATCGGCACCAACTCTGTGGGCTGGGCCGTGAT
CACCGACGAGTACAAGGTGCCCAGCAAGAAATTCAAGGTGCTGGGCAACACCGACC
GGCACAGCATCAAGAAGAACCTGATCGGCGCCCTGCTGTTCGACAGCGGAGAAACA
GCCGAGGCCACCCGGCTGAAGAGAACCGCCAGAAGAAGATACACCAGACGGAAGA
ACCGGATCTGCTATCTGCAAGAGATCTTCAGCAACGAGATGGCCAAGGTGGACGAC
AGCTTCTTCCACAGACTGGAAGAGTCCTTCCTGGTGGAAGAGGATAAGAAGCACGA
GCGGCACCCCATCTTCGGCAACATCGTGGACGAGGTGGCCTACCACGAGAAGTACC
CCACCATCTACCACCTGAGAAAGAAACTGGTGGACAGCACCGACAAGGCCGACCTG
CGGCTGATCTATCTGGCCCTGGCCCACATGATCAAGTTCCGGGGCCACTTCCTGATC
GAGGGCGACCTGAACCCCGACAACAGCGACGTGGACAAGCTGTTCATCCAGCTGGT
GCAGACCTACAACCAGCTGTTCGAGGAAAACCCCATCAACGCCAGCGGCGTGGACG
CCAAGGCCATCCTGTCTGCCAGACTGAGCAAGAGCAGACGGCTGGAAAATCTGATC
GCCCAGCTGCCCGGCGAGAAGAAGAATGGCCTGTTCGGCAACCTGATTGCCCTGAG
CCTGGGCCTGACCCCCAACTTCAAGAGCAACTTCGACCTGGCCGAGGATGCCAAAC
TGCAGCTGAGCAAGGACACCTACGACGACGACCTGGACAACCTGCTGGCCCAGATC
GGCGACCAGTACGCCGACCTGTTTCTGGCCGCCAAGAACCTGTCCGACGCCATCCTG
CTGAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCTCG
TGCGGCAGCAGCTGCCTGAGAAGTACAAAGAGATTTTCTTCGACCAGAGCAAGAAC
GGCTACGCCGGCTACATCGATGGCGGAGCCAGCCAGGAAGAGTTCTACAAGTTCAT
CAAGCCCATCCTGGAAAAGATGGACGGCACCGAGGAACTGCTCGTGAAGCTGAACA
GAGAGGACCTGCTGCGGAAGCAGCGGACCTTCGACAACGGCAGCATCCCCCACCAG
ATCCACCTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCCTACTA
CGTGGGCCCTCTGGCCAGGGGAAACAGCAGATTCGCCTGGATGACCAGAAAGAGCG
AGGAAACCATCACCCCCTGGAACTTCGAGGAAGTGGTGGACAAGGGCGCCAGCGCC
CAGAGCTTCATCGAGCGGATGACCAACTTCGATAAGAACCTGCCCAACGAGAAGGT
GCTGCCCAAGCACAGCCTGCTGTACGAGTACTTCACCGTGTACAACGAGCTGACCA
AAGTGAAATACGTGACCGAGGGAATGAGAAAGCCCGCCTTCCTGAGCGGCGAGCAG
AAAAAAGCCATCGTGGACCTGCTGTTCAAGACCAACCGGAAAGTGACCGTGAAGCA
GCTGAAAGAGGACTACTTCAAGAAAATCGAGTGCTTCGACTCCGTGGAAATCTCCG
GCGTGGAAGATCGGTTCAACGCCTCCCTGGGCACATACCACGATCTGCTGAAAATTA
TCAAGGACAAGGACTTCCTGGACAATGAGGAAAACGAGGACATTCTGGAAGATATC
GTGCTGACCCTGACACTGTTTGAGGACAGAGAGATGATCGAGGAACGGCTGAAAAC
CTATGCCCACCTGTTCGACGACAAAGTGATGAAGCAGCTGAAGCGGCGGAGATACA
CCGGCTGGGGCAGGCTGAGCCGGAAGCTGATCAACGGCATCCGGGACAAGCAGTCC
GGCAAGACAATCCTGGATTTCCTGAAGTCCGACGGCTTCGCCAACAGAAACTTCATG
CAGCTGATCCACGACGACAGCCTGACCTTTAAAGAGGACATCCAGAAAGCCCAGGT
GTCCGGCCAGGGCGATAGCCTGCACGAGCACATTGCCAATCTGGCCGGCAGCCCCG
CCATTAAGAAGGGCATCCTGCAGACAGTGAAGGTGGTGGACGAGCTCGTGAAAGTG
ATGGGCCGGCACAAGCCCGAGAACATCGTGATCGAAATGGCCAGAGAGAACCAGA
CCACCCAGAAGGGACAGAAGAACAGCCGCGAGAGAATGAAGCGGATCGAAGAGGG
CATCAAAGAGCTGGGCAGCCAGATCCTGAAAGAACACCCCGTGGAAAACACCCAGC
TGCAGAACGAGAAGCTGTACCTGTACTACCTGCAGAATGGGCGGGATATGTACGTG
GACCAGGAACTGGACATCAACCGGCTGTCCGACTACGATGTGGACGCTATCGTGCC
TCAGAGCTTTCTGAAGGACGACTCCATCGATAACAAAGTGCTGACTCGGAGCGACA
AGAACCGGGGCAAGAGCGACAACGTGCCCTCCGAAGAGGTCGTGAAGAAGATGAA
GAACTACTGGCGCCAGCTGCTGAATGCCAAGCTGATTACCCAGAGGAAGTTCGACA
ATCTGACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCCTGG
ACTCCCGGATGAACACTAAGTACGACGAGAACGACAAACTGATCCGGGAAGTGAAA
GTGATCACCCTGAAGTCCAAGCTGGTGTCCGATTTCCGGAAGGATTTCCAGTTTTAC
AAAGTGCGCGAGATCAACAACTACCACCACGCCCACGACGCCTACCTGAACGCCGT
CGTGGGAACCGCCCTGATCAAAAAGTACCCTAAGCTGGAAAGCGAGTTCGTGTACG
GCGACTACAAGGTGTACGACGTGCGGAAGATGATCGCCAAGAGCGAGCAGGAAATC
GGCAAGGCTACCGCCAAGTACTTCTTCTACAGCAACATCATGAACTTTTTCAAGACC
GAGATTACCCTGGCCAACGGCGAGATCCGGAAGCGGCCTCTGATCGAGACAAACGG
CGAAACAGGCGAGATCGTGTGGGATAAGGGCCGGGACTTTGCCACCGTGCGGAAAG
TGCTGTCTATGCCCCAAGTGAATATCGTGAAAAAGACCGAGGTGCAGACAGGCGGC
TTCAGCAAAGAGTCTATCCTGCCCAAGAGGAACAGCGACAAGCTGATCGCCAGAAA
GAAGGACTGGGACCCTAAGAAGTACGGCGGCTTCGACAGCCCCACCGTGGCCTATT
CTGTGCTGGTGGTGGCCAAAGTGGAAAAGGGCAAGTCCAAGAAACTGAAGAGTGTG
AAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCTCTG
CCGGCGAACTGCAGAAGGGAAACGAACTGGCCCTGCCCTCCAAATATGTGAACTTC
CTGTACCTGGCCAGCCACTATGAGAAGCTGAAGGGCTCCCCCGAGGATAATGAGCA
GAAACAGCTGTTTGTGGAACAGCACAAACACTACCTGGACGAGATCATCGAGCAGA
TCAGCGAGTTCTCCAAGAGAGTGATCCTGGCCGACGCTAATCTGGACAAGGTGCTG
AGCGCCTACAACAAGCACAGAGACAAGCCTATCAGAGAGCAGGCCGAGAATATCAT
CCACCTGTTTACCCTGACCAATCTGGGAGCCCCTGCCGCCTTCAAGTACTTTGACAC
CACCATCGACCGGAAGAGGTACACCAGCACCAAAGAGGTGCTGGACGCCACCCTGA
TCCACCAGAGCATCACCGGCCTGTACGAGACACGGATCGACCTGTCTCAGCTGGGA
GGCGACCTCGAGGGCGGATCCGGTGGTTCCGGAGGAGCTGTCGACATGGCGACTGT
CGAACCGGAAACCACCCCTACTCCTAATCCCCCGACTACAGAAGAGGAGAAAACGG
AATCTAATCAGGAGGTTGCTAACCCAGAACACTATATTAAACGGCCCCTACAGAAC
AGATGGGCACTCTGGTTTTTTAAAAATGATAAAAGCAAAACTTGGCAAGCAAACCT
GCGGCTGATCTCCAAGTTTGATACTGCTGAAGACTTTTTTGCTCTGTACAACCATATC
CAGTTGTCTAGTAATTTAATGCCTGGCTGTGACTACTCACTTTTTAAGGATGGTATTG
AGCCTATGTGGGAAGATGAGAAAAACAAACGGGGAGGACGATGGCTAATTACATTG
AACAAACAGCAGAGACGAAGTGACCTCGATCGCTTTTGGCTAGAGACACTTCTGTG
CCTTATTGGAGAATCTTTTGATGACTACAGTGATGATGTATGTGGCGCTGTTGTTAAT
GTTAGAGCTAAAGGTGATAAGATAGCAATATGGACTACTGAATGTGAAAACAGAGA
AGCTGTTACACATATAGGGAGGGTATACAAGGAAAGGTTAGGACTTCCTCCAAAGA
TAGTGATTGGTTATCAGTCCCACGCAGACACAGCTACTAAGAGCGGCGACACCACT
AAAAATAGGTTTGTTGTTTCTAGACTTAAGTAA

In other aspects, provided herein are fusion proteins comprising, consisting of, or consisting essentially of: (i) a guide nucleotide sequence-programmable RNA binding protein;

and (ii) a eukaryotic translation initiation factor 4E-binding protein 1 (EIF4E-BP1) protein. EIF4E-BP1 is part of a family of translation repressor proteins. In some embodiments, EIF4E-BP1 directly interacts with endogenous or exogenous EIF4E. Without being bound by theory, it is believed that the interaction of EIF4E-BP1 protein with EIF4E inhibits complex assembly and represses translation.

In some embodiments, the guide nucleotide sequence-programmable RNA binding protein is all or part of a protein selected from: Cas9, modified Cas9, Cas13a, Cas13b, CasRX/Cas13d, and a biological equivalent of each thereof. In some embodiments, the guide nucleotide sequence-programmable RNA binding protein is all or part of a protein selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus CRISPR 1 Cas9 (St1Cas9), Streptococcus thermophilus CRISPR 3 Cas9 (St3Cas9), and Brevibacillus laterosporus Cas9 (BlatCas9). In some embodiments, the guide nucleotide sequence-programmable RNA binding protein is nuclease inactive.

In some embodiments, the fusion protein further comprises, consists of, or consists essentially of a linker. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the fusion protein comprises, consists of, or consists essentially of the structure NH₂-[EIF4E-BP1]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[EIF4E-BP1]-COOH.

In some embodiments, the guide nucleotide sequence-programmable RNA binding protein is bound to a guide RNA (gRNA), a crisprRNA (crRNA), and/or a trans-activating crRNA (tracrRNA).

In some embodiments, the EIF4E-BP1 protein is encoded by a polynucleotide having a sequence comprising all or part of SEQ ID NO: 61 or a biological equivalent thereof. In some embodiments, the EIF4E-BP1 protein has an amino acid sequence comprising all or part of SEQ ID NO: 62 or a biological equivalent thereof.

(NM_004095)
(SEQ ID NO: 61)
GGGGCGAGGCGGAGCGAGGCTGGAGGCGCGGGAGGGCAGCGAGAGGTTCG
CGGGTGCAGCGCACAGGAGACCATGTCCGGGGGCAGCAGCTGCAGCCAGA
CCCCAAGCCGGGCCATCCCCGCCACTCGCCGGGTGGTGCTCGGCGACGGC
GTGCAGCTCCCGCCCGGGGACTACAGCACGACCCCCGGCGGCACGCTCTT
CAGCACCACCCCGGGAGGTACCAGGATCATCTATGACCGGAAATTCCTGA
TGGAGTGTCGGAACTCACCTGTGACCAAAACACCCCCAAGGGATCTGCCC
ACCATTCCGGGGGTCACCAGCCCTTCCAGTGATGAGCCCCCCATGGAAGC
CAGCCAGAGCCACCTGCGCAATAGCCCAGAAGATAAGCGGGCGGGCGGTG
AAGAGTCACAGTTTGAGATGGACATTTAAAGCACCAGCCATCGTGTGGAG
CACTACCAAGGGGCCCCTCAGGGCCTTCCTGGGAGGAGTCCCACCAGCCA
GGCCTTATGAAAGTGATCATACTGGGCAGGCGTTGGCGTGGGGTCGGACA
CCCCAGCCCTTTCTCCCTCACTCAGGGCACCTGCCCCCTCCTCTTCGTGA
ACACCAGCAGATACCTCCTTGTGCCTCCACTGATGCAGGAGCTGCCACCC
CAAGGGGAGTGACCCCTGCCAGCACACCCTGCAGCCAAGGGCCAGGAAGT
GGACAAGAACGAACCCTTCCTTCCGAATGATCAGCAGTTCCAGCCCCTCG
CTGCTGGGGGCGCAACCACCCCTTCCTTAGGTTGATGTGCTTGGGAAAGC
TCCCTCCCCCTCCTTCCCCAAGAGAGGAAATAAAAGCCACCTTCGCCCTA
GGGCCAAGAAAAAAAAAAAAAAAAAAA
(NP_004086)
(SEQ ID NO: 62)
MSGGSSCSQTPSRAIPATRRVVLGDGVQLPPGDYSTTPGGTLFSTTPGGT
RIIYDRKFLMECRNSPVTKTPPRDLPTIPGVTSPSSDEPPMEASQSHLRN
SPEDKRAGGEESQFEMDI

Wild type EIF4E-BP1can be phosphorylated in response to various signals including UV irradiation and insulin signaling, resulting in its dissociation from EIF4E and activation of cap-dependent mRNA translation. In some embodiments, one or more kinase phosphorylation domains of the EIF4E-BP1 protein are mutated. In some embodiments, all kinase phosphorylation domains of the EIF4E-BP1 protein are mutated. In some embodiments, the mutation replaces the amino acid of the phosphorylation domain with a negatively charged amino acid such as aspartic acid or glutamic acid. In other embodiments, the mutation replaces the amino acid of the phosphorylation domain with an uncharged residue such alanine or glycine. In some embodiments, EIF4E-BP1 comprises one or more phosphomimetic mutations and/or mutations to reduce EIF4E-BP1′s interaction with mTOR kinase. In some embodiments, the EIF4E-BP1 protein comprises one or more mutations selected from the group consisting of: mutant FEMDI motif, mutant RAIP motif, mutant caspase site at residue 25, MT37A, T46A, S65A and T70A. In some embodiments, the mutated EIF4E-BP1 is constitutively active.

| Mutation | Type |
|---|---|
| Mutant FEMDI | Inhibits interaction with mTOR kinase |
| Mutant RAIP | Inhibits interaction with mTOR kinase |
| Mutant caspase | Caspase site regulation |
| site at residue 25 | |
| MT37A | Decouples EIF4E-BP1 regulation from |
| T46A | kinase signaling |
| S65A | |
| T70A | |

In some embodiments, the fusion protein is a dCas9-EIF4E-BP1 fusion protein encoded by a nucleic acid comprising the following nucleic acid sequence:

(SEQ ID NO: 63)
ATGGGTAAGCCTATCCCTAACCCTCTCCTCGGTCTCGATTCTACGGCTAGCATGGAC
AAGAAGTACAGCATCGGCCTGGCCATCGGCACCAACTCTGTGGGCTGGGCCGTGAT
CACCGACGAGTACAAGGTGCCCAGCAAGAAATTCAAGGTGCTGGGCAACACCGACC
GGCACAGCATCAAGAAGAACCTGATCGGCGCCCTGCTGTTCGACAGCGGAGAAACA
GCCGAGGCCACCCGGCTGAAGAGAACCGCCAGAAGAAGATACACCAGACGGAAGA
ACCGGATCTGCTATCTGCAAGAGATCTTCAGCAACGAGATGGCCAAGGTGGACGAC
AGCTTCTTCCACAGACTGGAAGAGTCCTTCCTGGTGGAAGAGGATAAGAAGCACGA
GCGGCACCCCATCTTCGGCAACATCGTGGACGAGGTGGCCTACCACGAGAAGTACC
CCACCATCTACCACCTGAGAAAGAAACTGGTGGACAGCACCGACAAGGCCGACCTG
CGGCTGATCTATCTGGCCCTGGCCCACATGATCAAGTTCCGGGGCCACTTCCTGATC
GAGGGCGACCTGAACCCCGACAACAGCGACGTGGACAAGCTGTTCATCCAGCTGGT
GCAGACCTACAACCAGCTGTTCGAGGAAAACCCCATCAACGCCAGCGGCGTGGACG
CCAAGGCCATCCTGTCTGCCAGACTGAGCAAGAGCAGACGGCTGGAAAATCTGATC
GCCCAGCTGCCCGGCGAGAAGAAGAATGGCCTGTTCGGCAACCTGATTGCCCTGAG
CCTGGGCCTGACCCCCAACTTCAAGAGCAACTTCGACCTGGCCGAGGATGCCAAAC
TGCAGCTGAGCAAGGACACCTACGACGACGACCTGGACAACCTGCTGGCCCAGATC
GGCGACCAGTACGCCGACCTGTTTCTGGCCGCCAAGAACCTGTCCGACGCCATCCTG
CTGAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCTCG
TGCGGCAGCAGCTGCCTGAGAAGTACAAAGAGATTTTCTTCGACCAGAGCAAGAAC
GGCTACGCCGGCTACATCGATGGCGGAGCCAGCCAGGAAGAGTTCTACAAGTTCAT
CAAGCCCATCCTGGAAAAGATGGACGGCACCGAGGAACTGCTCGTGAAGCTGAACA
GAGAGGACCTGCTGCGGAAGCAGCGGACCTTCGACAACGGCAGCATCCCCCACCAG
ATCCACCTGGGAGAGCTGCACGCCATTCTGCGGCGGCAGGAAGATTTTTACCCATTC
CTGAAGGACAACCGGGAAAAGATCGAGAAGATCCTGACCTTCCGCATCCCCTACTA
CGTGGGCCCTCTGGCCAGGGGAAACAGCAGATTCGCCTGGATGACCAGAAAGAGCG
AGGAAACCATCACCCCCTGGAACTTCGAGGAAGTGGTGGACAAGGGCGCCAGCGCC
CAGAGCTTCATCGAGCGGATGACCAACTTCGATAAGAACCTGCCCAACGAGAAGGT
GCTGCCCAAGCACAGCCTGCTGTACGAGTACTTCACCGTGTACAACGAGCTGACCA
AAGTGAAATACGTGACCGAGGGAATGAGAAAGCCCGCCTTCCTGAGCGGCGAGCAG
AAAAAAGCCATCGTGGACCTGCTGTTCAAGACCAACCGGAAAGTGACCGTGAAGCA
GCTGAAAGAGGACTACTTCAAGAAAATCGAGTGCTTCGACTCCGTGGAAATCTCCG
GCGTGGAAGATCGGTTCAACGCCTCCCTGGGCACATACCACGATCTGCTGAAAATTA
TCAAGGACAAGGACTTCCTGGACAATGAGGAAAACGAGGACATTCTGGAAGATATC
GTGCTGACCCTGACACTGTTTGAGGACAGAGAGATGATCGAGGAACGGCTGAAAAC
CTATGCCCACCTGTTCGACGACAAAGTGATGAAGCAGCTGAAGCGGCGGAGATACA
CCGGCTGGGGCAGGCTGAGCCGGAAGCTGATCAACGGCATCCGGGACAAGCAGTCC
GGCAAGACAATCCTGGATTTCCTGAAGTCCGACGGCTTCGCCAACAGAAACTTCATG
CAGCTGATCCACGACGACAGCCTGACCTTTAAAGAGGACATCCAGAAAGCCCAGGT
GTCCGGCCAGGGCGATAGCCTGCACGAGCACATTGCCAATCTGGCCGGCAGCCCCG
CCATTAAGAAGGGCATCCTGCAGACAGTGAAGGTGGTGGACGAGCTCGTGAAAGTG
ATGGGCCGGCACAAGCCCGAGAACATCGTGATCGAAATGGCCAGAGAGAACCAGA
CCACCCAGAAGGGACAGAAGAACAGCCGCGAGAGAATGAAGCGGATCGAAGAGGG
CATCAAAGAGCTGGGCAGCCAGATCCTGAAAGAACACCCCGTGGAAAACACCCAGC
TGCAGAACGAGAAGCTGTACCTGTACTACCTGCAGAATGGGCGGGATATGTACGTG
GACCAGGAACTGGACATCAACCGGCTGTCCGACTACGATGTGGACGCTATCGTGCC
TCAGAGCTTTCTGAAGGACGACTCCATCGATAACAAAGTGCTGACTCGGAGCGACA
AGAACCGGGGCAAGAGCGACAACGTGCCCTCCGAAGAGGTCGTGAAGAAGATGAA
GAACTACTGGCGCCAGCTGCTGAATGCCAAGCTGATTACCCAGAGGAAGTTCGACA
ATCTGACCAAGGCCGAGAGAGGCGGCCTGAGCGAACTGGATAAGGCCGGCTTCATC
AAGAGACAGCTGGTGGAAACCCGGCAGATCACAAAGCACGTGGCACAGATCCTGG
ACTCCCGGATGAACACTAAGTACGACGAGAACGACAAACTGATCCGGGAAGTGAAA
GTGATCACCCTGAAGTCCAAGCTGGTGTCCGATTTCCGGAAGGATTTCCAGTTTTAC
AAAGTGCGCGAGATCAACAACTACCACCACGCCCACGACGCCTACCTGAACGCCGT
CGTGGGAACCGCCCTGATCAAAAAGTACCCTAAGCTGGAAAGCGAGTTCGTGTACG
GCGACTACAAGGTGTACGACGTGCGGAAGATGATCGCCAAGAGCGAGCAGGAAATC
GGCAAGGCTACCGCCAAGTACTTCTTCTACAGCAACATCATGAACTTTTTCAAGACC
GAGATTACCCTGGCCAACGGCGAGATCCGGAAGCGGCCTCTGATCGAGACAAACGG
CGAAACAGGCGAGATCGTGTGGGATAAGGGCCGGGACTTTGCCACCGTGCGGAAAG
TGCTGTCTATGCCCCAAGTGAATATCGTGAAAAAGACCGAGGTGCAGACAGGCGGC
TTCAGCAAAGAGTCTATCCTGCCCAAGAGGAACAGCGACAAGCTGATCGCCAGAAA
GAAGGACTGGGACCCTAAGAAGTACGGCGGCTTCGACAGCCCCACCGTGGCCTATT
CTGTGCTGGTGGTGGCCAAAGTGGAAAAGGGCAAGTCCAAGAAACTGAAGAGTGTG
AAAGAGCTGCTGGGGATCACCATCATGGAAAGAAGCAGCTTCGAGAAGAATCCCAT
CGACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCTCTG
CCGGCGAACTGCAGAAGGGAAACGAACTGGCCCTGCCCTCCAAATATGTGAACTTC
CTGTACCTGGCCAGCCACTATGAGAAGCTGAAGGGCTCCCCCGAGGATAATGAGCA
GAAACAGCTGTTTGTGGAACAGCACAAACACTACCTGGACGAGATCATCGAGCAGA
TCAGCGAGTTCTCCAAGAGAGTGATCCTGGCCGACGCTAATCTGGACAAGGTGCTG
AGCGCCTACAACAAGCACAGAGACAAGCCTATCAGAGAGCAGGCCGAGAATATCAT
CCACCTGTTTACCCTGACCAATCTGGGAGCCCCTGCCGCCTTCAAGTACTTTGACAC
CACCATCGACCGGAAGAGGTACACCAGCACCAAAGAGGTGCTGGACGCCACCCTGA
TCCACCAGAGCATCACCGGCCTGTACGAGACACGGATCGACCTGTCTCAGCTGGGA
GGCGACCTCGAGGGCGGATCCGGTGGTTCCGGAGGAGCTGTCGACATGTCCGGGGG
CAGCAGCTGCAGCCAGACCCCAAGCGCTGCCGCAGCCGCCACTCGCCGGGTGGTGC
TCGGCGCCGGCGTGCAGCTCCCGCCCGGGGACTACAGCACGGCCCCCGGCGGCACG
CTCTTCAGCACCGCCCCGGGAGGTACCAGGATCATCTATGACCGGAAATTCCTGATG
GAGTGTCGGAACGCACCTGTGACCAAAGCACCCCCAAGGGATCTGCCCACCATTCC
GGGGGTCACCAGCCCTTCCAGTGATGAGCCCCCCATGGAAGCCAGCCAGAGCCACC
TGCGCAATAGCCCAGAAGATAAGCGGGCGGGCGGTGAAGAGTCACAGGCTGAGAT
GGACATTTCTAGACTTAAG

In other aspects, provided herein are fusion proteins comprising, consisting of, or consisting essentially of: (i) a guide nucleotide sequence-programmable RNA binding protein; and (ii) a ubiquitin-associated protein 2-like (UBAP2L) protein.

In some embodiments, the guide nucleotide sequence-programmable RNA binding protein is all or part of a protein selected from: Cas9, modified Cas9, Cas13a, Cas13b, CasRX/Cas13d, and a biological equivalent of each thereof. In some embodiments, the guide nucleotide sequence-programmable RNA binding protein is all or part of a protein selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus CRISPR 1 Cas9 (St1Cas9), Streptococcus thermophilus CRISPR 3 Cas9 (St3Cas9), and Brevibacillus laterosporus Cas9 (BlatCas9). In some embodiments, the guide nucleotide sequence-programmable RNA binding protein is nuclease inactive.

In some embodiments, the fusion protein further comprises, consists of, or consists essentially of a linker. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the fusion protein comprises, consists of, or consists essentially of the structure NH₂-[UBAP2L]-[linker]-[guide nucleotide sequence-programmable RNA binding protein]-COOH. In other embodiments, the fusion protein comprises, consists of, or consists essentially of the structure NH₂-[guide nucleotide sequence-programmable RNA binding protein]-[linker]-[UBAP2L]-COOH.

In some embodiments, the guide nucleotide sequence-programmable RNA binding protein is bound to a guide RNA (gRNA), a crisprRNA (crRNA), and/or a trans-activating crRNA (tracrRNA).

In some embodiments, the UBAP2L protein is encoded by a polynucleotide having a sequence comprising all or part of a sequence selected from SEQ ID NO: 64, SEQ ID NO: 65, SEQ ID NO: 66, SEQ ID NO: 67 and a biological equivalent thereof.

(NM_014847.4) Homo sapiens ubiquitin associated
protein 2 like (UBAP2L), transcript variant 1
(SEQ ID NO: 64)
GTCAGTGTGGAGGAGACTGAGTATTCTACCTTGTAAATACT
GTTATTTGTATATACTGTAAATGATGACATCGGTGGGCACT
AACCGAGCCCGGGGAAACTGGGAACAACCTCAAAACCAAAA
CCAGACACAGCACAAGCAGCGGCCACAGGCCACTGCAGAAC
AAATTAGACTTGCACAGATGATTTCGGACCATAATGATGCT
GACTTTGAGGAGAAGGTGAAACAATTGATTGAGATAACAGG
CAAGAACCAGGATGAATGTGTGATTGCTTTGCATGACTGCA
ATGGAGATGTCAACAGAGCTATCAATGTTCTTCTGGAAGGA
AACCCAGACACGCATTCCTGGGAGATGGTCGGGAAGAAGAA
GGGAGTCTCAGGCCAGAAGGATGGTGGCCAGACGGAATCCA
ATGAGGAAGGCAAAGAAAATCGAGACCGGGACAGAGACTAT
AGTCGGCGACGTGGTGGGCCACCAAGACGGGGGAGAGGTGC
CAGCCGTGGACGAGAGTTTCGAGGTCAGGAAAATGGATTGG
ATGGCACCAAGAGTGGAGGGCCTTCTGGAAGAGGAACAGAA
AGAGGCAGAAGGGGCCGTGGCCGAGGCAGAGGTGGCTCTGG
TAGGCGAGGAGGAAGGTTTTCTGCTCAAGGAATGGGAACCT
TTAACCCAGCTGATTATGCAGAGCCAGCCAATACTGATGAT
AACTATGGCAATAGCAGCGGCAATACGTGGAACAACACTGG
CCACTTTGAACCAGATGATGGGACGAGTGCATGGAGGACTG
CAACAGAGGAGTGGGGGACTGAAGATTGGAATGAAGATCTT
TCTGAGACCAAGATCTTCACTGCCTCTAATGTGTCTTCAGT
GCCTCTGCCTGCGGAGAATGTGACAATCACTGCTGGTCAGA
GAATTGACCTTGCTGTTCTGCTGGGGAAGACACCATCTACA
ATGGAGAATGATTCATCTAATCTGGATCCGTCTCAGGCTCC
TTCTCTGGCCCAGCCTCTGGTGTTCAGTAATTCGAAGCAGA
CTGCCATATCACAGCCTGCTTCAGGGAACACATTTTCTCAT
CACAGTATGGTGAGCATGTTAGGGAAAGGATTTGGTGATGT
CGGTGAAGCTAAAGGCGGCAGTACTACAGGCTCCCAGTTCT
TGGAGCAATTCAAGACTGCCCAAGCCCTGGCTCAGTTGGCA
GCTCAGCATTCTCAGTCTGGAAGCACCACCACCTCCTCTTG
GGACATGGGCTCGACGACACAATCCCCATCACTGGTGCAGT
ATGATTTGAAGAACCCAAGTGATTCAGCAGTGCACAGCCCC
TTTACAAAGCGCCAGGCTTTTACCCCATCTTCAACCATGAT
GGAGGTGTTCCTTCAGGAGAAGTCACCTGCAGTGGCTACCT
CCACAGCTGCACCTCCACCTCCGTCTTCTCCTCTGCCAAGC
AAATCCACATCGGCTCCACAGATGTCGCCTGGATCTTCAGA
CAACCAGTCCTCTAGCCCTCAGCCGGCTCAGCAGAAACTGA
AACAGCAGAAGAAAAAAGCCTCCTTGACTTCTAAGATTCCT
GCTCTGGCTGTGGAGATGCCTGGCTCAGCAGATATCTCAGG
GCTAAACCTGCAGTTTGGGGCATTGCAGTTTGGGTCAGAGC
CTGTCCTTTCTGATTATGAGTCCACCCCCACCACGAGCGCC
TCTTCAAGCCAGGCTCCAAGTAGCCTGTATACCAGCACGGC
CAGTGAATCATCCTCTACAATTTCATCTAACCAGAGTCAGG
AGTCTGGTTATCAGAGCGGCCCAATTCAGTCGACAACCTAT
ACCTCCCAAAATAATGCTCAGGGCCCTCTTTATGAACAGAG
ATCCACACAGACTCGGCGGTACCCCAGCTCCATCTCTTCAT
CACCCCAAAAGGACCTGACTCAGGCAAAGAATGGCTTCAGT
TCTGTGCAGGCCACGCAGTTACAGACCACACAATCTGTTGA
AGGTGCTACAGGCTCTGCAGTGAAATCTGATTCACCTTCCA
CTTCTAGCATCCCCCCTCTCAATGAAACGGTATCTGCAGCT
TCCTTACTGACGACAACCAATCAGCATTCATCCTCCTTGGG
TGGCTTGAGCCACAGTGAGGAGATTCCAAATACTACCACCA
CACAACACAGCAGCACGTTATCTACGCAGCAGAATACCCTT
TCATCATCAACATCTTCTGGGCGCACTTCGACATCCACTCT
TTTGCACACAAGTGTGGAGAGTGAGGCGAATCTCCATTCTT
CCTCCAGCACTTTTTCCACCACATCCAGCACAGTCTCTGCA
CCTCCCCCAGTGGTCAGTGTCTCCTCCAGTCTCAATAGTGG
CAGTAGCCTGGGCCTCAGCCTAGGCAGCAACTCCACTGTCA
CAGCCTCGACTCGAAGCTCAGTTGCTACGACTTCAGGAAAA
GCTCCTCCCAACCTCCCTCCTGGGGTCCCGCCGTTGTTGCC
TAATCCGTATATTATGGCTCCAGGGCTGTTACATGCCTACC
CGCCACAAGTATATGGTTATGATGACTTGCAGATGCTTCAG
ACAAGATTTCCATTGGATTACTACAGCATCCCATTTCCCAC
ACCCACTACTCCGCTGACTGGGAGGGATGGTAGCCTGGCCA
GCAACCCTTATTCTGGTGACCTCACAAAGTTCGGCCGTGGG
GATGCCTCCTCCCCAGCCCCGGCCACAACCTTGGCCCAACC
CCAACAGAACCAGACGCAGACTCACCATACCACGCAGCAGA
CATTCCTGAACCCGGCGCTGCCTCCTGGCTACAGTTACACC
AGCCTGCCATACTATACAGGGGTCCCGGGCCTCCCCAGCAC
CTTCCAGTATGGGCCTGCTGTGTTCCCTGTGGCTCCTACCT
CTTCCAAGCAGCATGGTGTGAATGTCAGTGTGAATGCATCG
GCCACCCCTTTCCAACAGCCGAGTGGATATGGGTCTCATGG
ATACAACACTGGTGTTTCAGTCACCTCCAGTAACACGGGCG
TGCCAGATATCTCGGGTTCTGTGTACTCCAAAACCCAGCAG
TCCTTTGAGAAACAAGGTTTTCATTCCGGTACTCCTGCTGC
TTCCTTCAACTTGCCTTCAGCCCTAGGAAGTGGGGGCCCCA
TCAATCCGGCCACAGCTGCTGCCTACCCACCTGCCCCCTTT
ATGCACATTCTGACCCCCCATCAGCAGCCGCATTCTCAGAT
CCTTCACCATCACCTGCAGCAGGATGGCCAGACGGGCAGCG
GGCAACGTAGCCAGACCAGCTCCATCCCGCAGAAGCCCCAG
ACCAACAAGTCTGCCTACAACAGCTACAGCTGGGGGGCCAA
CTGAGGCCCTGACCCTCTTCTCCCGGTCCCATCTTCTGAGA
GGGCTTCTCAGCCTGGAAACTATGGAAACAGCATCAAAGAG
AAAGGAATGTGGGGGGTTTCCGCTGCCCCCCACCCCCAGCG
GCCCACCCCATGCCTCAGCTTCATGTCTGTCCCATTCCTAT
ACCATCCCCACCCTGTTGTATGTATTATAGGATTTGTATTT
TCTCCTTTTTTTTCCCCCTTCCATTCCTTCTCCCCTCTTGC
ATTCAAGATTATGAAACTTTGCTATGGGCCCTGCACTTCCT
TTGCTTCCTCCTGTTCACCCTGGTGGTGTACGGATGAGGCG
GGGAGGTGGGACCCCCAAACATATATCAGCCCAACAGCCCT
AAGTCTCCTTCTTTATTATTAGGAAAACAACAACAACAACA
AACAAAAAAATGGCGTCATGAATATGAACAGCATTGTCAGA
TGAATTAGTTGAAGTGGTTTTTTTTTTGTTTTTTTTTTTTT
TTTGTACTGTGTCCTCAAATTTAATGGATTAATGTGTCTTG
TATATATAAAAAGAAAACCTCTA
(NM_001127320.2) Homo sapiens ubiquitin associated
protein 2 like (UBAP2L), transcript variant 2
(SEQ ID NO: 65)
GGGTCGGCCCGACTAAGTGACTTAAACTCCCACCTACTCCT
GGAATAAGGAGTCAAAGCCCGGATAGGCGCAGTATTCTACC
TTGTAAATACTGTTATTTGTATATACTGTAAATGATGACAT
CGGTGGGCACTAACCGAGCCCGGGGAAACTGGGAACAACCT
CAAAACCAAAACCAGACACAGCACAAGCAGCGGCCACAGGC
CACTGCAGAACAAATTAGACTTGCACAGATGATTTCGGACC
ATAATGATGCTGACTTTGAGGAGAAGGTGAAACAATTGATT
GATATTACAGGCAAGAACCAGGATGAATGTGTGATTGCTTT
GCATGACTGCAATGGAGATGTCAACAGAGCTATCAATGTTC
TTCTGGAAGGAAACCCAGACACGCATTCCTGGGAGATGGTC
GGGAAGAAGAAGGGAGTCTCAGGCCAGAAGGATGGTGGCCA
GACGGAATCCAATGAGGAAGGCAAAGAAAATCGAGACCGGG
ACAGAGACTATAGTCGGCGACGTGGTGGGCCACCAAGACGG
GGGAGAGGTGCCAGCCGTGGACGAGAGTTTCGAGGTCAGGA
AAATGGATTGGATGGCACCAAGAGTGGAGGGCCTTCTGGAA
GAGGAACAGAAAGAGGCAGAAGGGGCCGTGGCCGAGGCAGA
GGTGGCTCTGGTAGGCGAGGAGGAAGGTTTTCTGCTCAAGG
AATGGGAACCTTTAACCCAGCTGATTATGCAGAGCCAGCCA
ATACTGATGATAACTATGGCAATAGCAGCGGCAATACGTGG
AACAACACTGGCCACTTTGAACCAGATGATGGGACGAGTGC
ATGGAGGACTGCAACAGAGGAGTGGGGGACTGAAGATTGGA
ATGAAGATCTCTTTGAGACCAAGATCTTCACTGCCTCTAAT
GTGTCTTCAGTGCCTCTGCCTGCGGAGAATGTGACAATCAC
TGCTGGTCAGAGAATTGACCTTGCTGTTCTGCTGGGGAAGA
CACCATCTACAATGGAGAATGATTCATCTAATCTGGATCCG
TCTCAGGCTCCTTCTCTGGCCCAGCCTCTGGTGTTCAGTAA
TTCGAAGCAGACTGCCATATCACAGCCTGCTTCAGGGAACA
CATTTTCTCATCACAGTATGGTGAGCATGTTAGGGAAAGGA
TTTGGTGATGTCGGTGAAGCTAAAGGCGGCAGTACTACAGG
CTCCCAGTTCTTGGAGCAATTCAAGACTGCCCAAGCCCTGG
CTCAGTTGGCAGCTCAGCATTCTCAGTCTGGAAGCACCACC
ACCTCCTCTTGGGACATGGGCTCGACGACACAATCCCCATC
ACTGGTGCAGTATGATTTGAAGAACCCAAGTGATTCAGCAG
TGCACAGCCCCTTTACAAAGCGCCAGGCTTTTACCCCATCT
TCAACCATGATGGAGGTGTTCCTTCAGGAGAAGTCACCTGC
AGTGGCTACCTCCACAGCTGCACCTCCACCTCCGTCTTCTC
CTCTGCCAAGCAAATCCACATCGGCTCCACAGATGTCGCCT
GGATCTTCAGACAACCAGTCCTCTAGCCCTCAGCCGGCTCA
GCAGAAACTGAAACAGCAGAAGAAAAAAGCCTCCTTGACTT
CTAAGATTCCTGCTCTGGCTGTGGAGATGCCTGGCTCAGCA
GATATCTCAGGGCTAAACCTGCAGTTTGGGGCATTGCAGTT
TGGGTCAGAGCCTGTCCTTTCTGATTATGAGTCCACCCCCA
CCACGAGCGCCTCTTCAAGCCAGGCTCCAAGTAGCCTGTAT
ACCAGCACGGCCAGTGAATCATCCTCTACAATTTCATCTAA
CCAGAGTCAGGAGTCTGGTTATCAGAGCGGCCCAATTCAGT
CGACAACCTATACCTCCCAAAATAATGCTCAGGGCCCTCTT
TATGAACAGAGATCCACACAGACTCGGCGGTACCCCAGCTC
CATCTCTTCATCACCCCAAAAGGACCTGACTCAGGCAAAGA
ATGGCTTCAGTTCTGTGCAGGCCACGCAGTTACAGACCACA
CAATCTGTTGAAGGTGCTACAGGCTCTGCAGTGAAATCTGA
TTCACCTTCCACTTCTAGCATCCCCCCTCTCAATGAAACGG
TATCTGCAGCTTCCTTACTGACGACAACCAATCAGCATTCA
TCCTCCTTGGGTGGCTTGAGCCACAGTGAGGAGATTCCAAA
TACTACCACCACACAACACAGCAGCACGTTATCTACGCAGC
AGAATACCCTTTCATCATCAACATCTTCTGGGCGCACTTCG
ACATCCACTCTTTTGCACACAAGTGTGGAGAGTGAGGCGAA
TCTCCATTCTTCCTCCAGCACTTTTTCCACCACATCCAGCA
CAGTCTCTGCACCTCCCCCAGTGGTCAGTGTCTCCTCCAGT
CTCAATAGTGGCAGTAGCCTGGGCCTCAGCCTAGGCAGCAA
CTCCACTGTCACAGCCTCGACTCGAAGCTCAGTTGCTACGA
CTTCAGGAAAAGCTCCTCCCAACCTCCCTCCTGGGGTCCCG
CCGTTGTTGCCTAATCCGTATATTATGGCTCCAGGGCTGTT
ACATGCCTACCCGCCACAAGTATATGGTTATGATGACTTGC
AGATGCTTCAGACAAGATTTCCATTGGATTACTACAGCATC
CCATTTCCCACACCCACTACTCCGCTGACTGGGAGGGATGG
TAGCCTGGCCAGCAACCCTTATTCTGGTGACCTCACAAAGT
TCGGCCGTGGGGATGCCTCCTCCCCAGCCCCGGCCACAACC
TTGGCCCAACCCCAACAGAACCAGACGCAGACTCACCATAC
CACGCAGCAGACATTCCTGAACCCGGCGCTGCCTCCTGGCT
ACAGTTACACCAGCCTGCCATACTATACAGGGGTCCCGGGC
CTCCCCAGCACCTTCCAGTATGGGCCTGCTGTGTTCCCTGT
GGCTCCTACCTCTTCCAAGCAGCATGGTGTGAATGTCAGTG
TGAATGCATCGGCCACCCCTTTCCAACAGCCGAGTGGATAT
GGGTCTCATGGATACAACACTGGAAGAAAATATCCACCCCC
TTACAAGCATTTCTGGACGGCTGAGAGCTAATTTGGCCCAA
GGCTGGGGGCTGTGTTTTGTGTGTGTGTATAAATTTGCACT
GAAGTCTTGTTTCAGAAACCAGACCACTGAGGAGAGCCTGC
TGAGCTGAGGCCATGGCCTGCGTGGCTTGGGGAAATGAGTT
GGTGGATACCTTCTGGGCTTTTGAACTTGCCCCTCCCCCAT
TTCCCTCTCCCCCATGTGTCTGACCCTGTCTTACCCATTTC
AAGTTCAAGCGGTGCAGCACCTTCGAAGCATCAATGCACAC
ACCTGCTGTTGCTTTTGATTTCTGGAAGGCATGTAGTTTCA
ACGTAACAAAAATATGTAGTCCAATAAACTGTGGT
ATTTCTTTAGCTAAC
(NM_001287815.1) Homo sapiens ubiquitin associated
protein 2 like (UBAP2L), transcript variant 3
(SEQ ID NO: 66)
AAGTGGGCGGGGGAAGGCGCGAGAGCGAGCGCGAGAGGGAA
AAGGAGGGAGGGGGTGGGGAAGAGGGAATCTTATATCACGT
GACAGGGGCGGCGCGGCCCGGGGTGTCAGTGTGGAGGAGAC
TGAGTATTCTACCTTGTAAATACTGTTATTTGTATATACTG
TAAATGATGACATCGGTGGGCACTAACCGAGCCCGGGGAAA
CTGGGAACAACCTCAAAACCAAAACCAGACACAGCACAAGC
AGCGGCCACAGGCCACTGCAGAACAAATTAGACTTGCACAG
ATGATTTCGGACCATAATGATGCTGACTTTGAGGAGAAGGT
GAAACAATTGATTGATATTACAGGCAAGAACCAGGATGAAT
GTGTGATTGCTTTGCATGACTGCAATGGAGATGTCAACAGA
GCTATCAATGTTCTTCTGGAAGGAAACCCAGACACGCATTC
CTGGGAGATGGTCGGGAAGAAGAAGGGAGTCTCAGGCCAGA
AGGATGGTGGCCAGACGGAATCCAATGAGGAAGGCAAAGAA
AATCGAGACCGGGACAGAGACTATAGTCGGCGACGTGGTGG
GCCACCAAGACGGGGGAGAGTTCGAGGTCAGGAAAATGGAT
TGGATGGCACCAAGAGTGGAGGGCCTTCTGGAAGAGGAACA
GAAAGAGGCAGAAGGGGCCGTGGCCGAGGCAGAGGTGGCTC
TGGTAGGCGAGGAGGAAGGTTTTCTGCTCAAGGAATGGGAA
CCTTTAACCCAGCTGATTATGCAGAGCCAGCCAATACTGAT
GATAACTATGGCAATAGCAGCGGCAATACGTGGAACAACAC
TGGCCACTTTGAACCAGATGATGGGACGAGTGCATGGAGGA
CTGCAACAGAGGAGTGGGGGACTGAAGATTGGAATGAAGAT
CTTTCTGAGACCAAGATCTTCACTGCCTCTAATGTGTCTTC
AGTGCCTCTGCCTGCGGAGAATGTGACAATCACTGCTGGTC
AGAGAATTGACCTTGCTGTTCTGCTGGGGAAGACACCATCT
ACAATGGAGAATGATTCATCTAATCTGGATCCGTCTCAGGC
TCCTTCTCTGGCCCAGCCTCTGGTGTTCAGTAATTCGAAGC
AGACTGCCATATCACAGCCTGCTTCAGGGAACACATTTTCT
CATCACAGTATGGTGAGCATGTTAGGGAAAGGATTTGGTGA
TGTCGGTGAAGCTAAAGGCGGCAGTACTACAGGCTCCCAGT
TCTTGGAGCAATTCAAGACTGCCCAAGCCCTGGCTCAGTTG
GCAGCTCAGCATTCTCAGTCTGGAAGCACCACCACCTCCTC
TTGGGACATGGGCTCGACGACACAATCCCCATCACTGGTGC
AGTATGATTTGAAGAACCCAAGTGATTCAGCAGTGCACAGC
CCCTTTACAAAGCGCCAGGCTTTTACCCCATCTTCAACCAT
GATGGAGGTGTTCCTTCAGGAGAAGTCACCTGCAGTGGCTA
CCTCCACAGCTGCACCTCCACCTCCGTCTTCTCCTCTGCCA
AGCAAATCCACATCGGCTCCACAGATGTCGCCTGGATCTTC
AGACAACCAGTCCTCTAGCCCTCAGCCGGCTCAGCAGAAAC
TGAAACAGCAGAAGAAAAAAGCCTCCTTGACTTCTAAGATT
CCTGCTCTGGCTGTGGAGATGCCTGGCTCAGCAGATATCTC
AGGGCTAAACCTGCAGTTTGGGGCATTGCAGTTTGGGTCAG
AGCCTGTCCTTTCTGATTATGAGTCCACCCCCACCACGAGC
GCCTCTTCAAGCCAGGCTCCAAGTAGCCTGTATACCAGCAC
GGCCAGTGAATCATCCTCTACAATTTCATCTAACCAGAGTC
AGGAGTCTGGTTATCAGAGCGGCCCAATTCAGTCGACAACC
TATACCTCCCAAAATAATGCTCAGGGCCCTCTTTATGAACA
GAGATCCACACAGACTCGGCGGTACCCCAGCTCCATCTCTT
CATCACCCCAAAAGGACCTGACTCAGGCAAAGAATGGCTTC
AGTTCTGTGCAGGCCACGCAGTTACAGACCACACAATCTGT
TGAAGGTGCTACAGGCTCTGCAGTGAAATCTGATTCACCTT
CCACTTCTAGCATCCCCCCTCTCAATGAAACGGTATCTGCA
GCTTCCTTACTGACGACAACCAATCAGCATTCATCCTCCTT
GGGTGGCTTGAGCCACAGTGAGGAGATTCCAAATACTACCA
CCACACAACACAGCAGCACGTTATCTACGCAGCAGAATACC
CTTTCATCATCAACATCTTCTGGGCGCACTTCGACATCCAC
TCTTTTGCACACAAGTGTGGAGAGTGAGGCGAATCTCCATT
CTTCCTCCAGCACTTTTTCCACCACATCCAGCACAGTCTCT
GCACCTCCCCCAGTGGTCAGTGTCTCCTCCAGTCTCAATAG
TGGCAGTAGCCTGGGCCTCAGCCTAGGCAGCAACTCCACTG
TCACAGCCTCGACTCGAAGCTCAGTTGCTACGACTTCAGGA
AAAGCTCCTCCCAACCTCCCTCCTGGGGTCCCGCCGTTGTT
GCCTAATCCGTATATTATGGCTCCAGGGCTGTTACATGCCT
ACCCGCCACAAGTATATGGTTATGATGACTTGCAGATGCTT
CAGACAAGATTTCCATTGGATTACTACAGCATCCCATTTCC
CACACCCACTACTCCGCTGACTGGGAGGGATGGTAGCCTGG
CCAGCAACCCTTATTCTGGTGACCTCACAAAGTTCGGCCGT
GGGGATGCCTCCTCCCCAGCCCCGGCCACAACCTTGGCCCA
ACCCCAACAGAACCAGACGCAGACTCACCATACCACGCAGC
AGACATTCCTGAACCCGGCGCTGCCTCCTGGCTACAGTTAC
ACCAGCCTGCCATACTATACAGGGGTCCCGGGCCTCCCCAG
CACCTTCCAGTATGGGCCTGCTGTGTTCCCTGTGGCTCCTA
CCTCTTCCAAGCAGCATGGTGTGAATGTCAGTGTGAATGCA
TCGGCCACCCCTTTCCAACAGCCGAGTGGATATGGGTCTCA
TGGATACAACACTGGAAGAAAATATCCACCCCCTTACAAGC
ATTTCTGGACGGCTGAGAGCTAATTTGGCCCAAGGCTGGGG
GCTGTGTTTTGTGTGTGTGTATAAATTTGCACTGAAGTCTT
GTTTCAGAAACCAGACCACTGAGGAGAGCCTGCTGAGCTGA
GGCCATGGCCTGCGTGGCTTGGGGAAATGAGTTGGTGGATA
CCTTCTGGGCTTTTGAACTTGCCCCTCCCCCATTTCCCTCT
CCCCCATGTGTCTGACCCTGTCTTACCCATTTCAAGTTCAA
GCGGTGCAGCACCTTCGAAGCATCAATGCACACACCTGCTG
TTGCTTTTGATTTCTGGAAGGCATGTAGTTTCAACTTGTAA
CAAAAATATTTGTAGTCTTCAATAAACTGTGGTATTTCTTT
AGCTAAC
(NM_001287816.1) Homo sapiens ubiquitin associated
protein 2 like (UBAP2L), transcript variant 4
(SEQ ID NO: 67)
AAGTGGGCGGGGGAAGGCGCGAGAGCGAGCGCGAGAGGGAA
AAGGAGGGAGGGGGTGGGGAAGAGGGAATCTTATATCACGT
GACAGGGGCGGCGCGGCCCGGGGTGTCAGTGTGGAGGAGAC
TGAGTATTCTACTTCGTAAATACTGTTATTTGTATATACTG
TAAATGATGACATCGGTGGGCACTAACCGAGCCCGGGGAAA
CTGGGAACAACCTCAAAACCAAAACCAGACACAGCACAAGC
AGCGGCCACAGGCCACTGCAGAACAAATTAGACTTGCACAG
ATGATTTCGGACCATAATGATGCTGACTTTGAGGAGAAGGT
GAAACAATTGATTGATATTACAGGCAAGAACCAGGATGAAT
GTGTGATTGCTTTGCATGACTGCAATGGAGATGTCAACAGA
GCTATCAATGTTCTTCTGGAAGGAAACCCAGACACGCATTC
CTGGGAGATGGTCGGGAAGAAGAAGGGAGTCTCAGGCCAGA
AGGATGGTGGCCAGACGGAATCCAATGAGGAAGGCAAAGAA
AATCGAGACCGGGACAGAGACTATAGTCGGCGACGTGGTGG
GCCACCAAGACGGGGGAGAGGTGCCAGCCGTGGACGAGAGT
GTATGCATGGGGCTTTATCAAAACCAGCTGTGGTTCGAGGT
CAGGAAAATGGATTGGATGGCACCAAGAGTGGAGGGCCTTC
TGGAAGAGGAACAGAAAGAGGCAGAAGGGGCCGTGGCCGAG
GCAGAGGTGGCTCTGGTAGGCGAGGAGGAAGGTTTTCTGCT
CAAGGAATGGGAACCTTTAACCCAGCTGATTATGCAGAGCC
AGCCAATACTGATGATAACTATGGCAATAGCAGCGGCAATA
CGTGGAACAACACTGGCCACTTTGAACCAGATGATGGGACG
AGTGCATGGAGGACTGCAACAGAGGAGTGGGGGACTGAAGA
TTGGAATGAAGATCTTTCTGAGACCAAGATCTTCACTGCCT
CTAATGTGTCTTCAGTGCCTCTGCCTGCGGAGAATGTGACA
ATCACTGCTGGTCAGAGAATTGACCTTGCTGTTCTGCTGGG
GAAGACACCATCTACAATGGAGAATGATTCATCTAATCTGG
ATCCGTCTCAGGCTCCTTCTCTGGCCCAGCCTCTGGTGTTC
AGTAATTCGAAGCAGACTGCCATATCACAGCCTGCTTCAGG
GAACACATTTTCTCATCACAGTATGGTGAGCATGTTAGGGA
AAGGATTTGGTGATGTCGGTGAAGCTAAAGGCGGCAGTACT
ACAGGCTCCCAGTTCTTGGAGCAATTCAAGACTGCCCAAGC
CCTGGCTCAGTTGGCAGCTCAGCATTCTCAGTCTGGAAGCA
CCACCACCTCCTCTTGGGACATGGGCTCGACGACACAATCC
CCATCACTGGTGCAGTATGATTTGAAGAACCCAAGTGATTC
AGCAGTGCACAGCCCCTTTACAAAGCGCCAGGCTTTTACCC
CATCTTCAACCATGATGGAGGTGTTCCTTCAGGAGAAGTCA
CCTGCAGTGGCTACCTCCACAGCTGCACCTCCACCTCCGTC
TTCTCCTCTGCCAAGCAAATCCACATCGGCTCCACAGATGT
CGCCTGGATCTTCAGACAACCAGTCCTCTAGCCCTCAGCCG
GCTCAGCAGAAACTGAAACAGCAGAAGAAAAAAGCCTCCTT
GACTTCTAAGATTCCTGCTCTGGCTGTGGAGATGCCTGGCT
CAGCAGATATCTCAGGGCTAAACCTGCAGTTTGGGGCATTG
CAGTTTGGGTCAGAGCCTGTCCTTTCTGATTATGAGTCCAC
CCCCACCACGAGCGCCTCTTCAAGCCAGGCTCCAAGTAGCC
TGTATACCAGCACGGCCAGTGAATCATCCTCTACAATTTCA
TCTAACCAGAGTCAGGAGTCTGGTTATCAGAGCGGCCCAAT
TCAGTCGACAACCTATACCTCCCAAAATAATGCTCAGGGCC
CTCTTTATGAACAGAGATCCACACAGACTCGGCGGTACCCC
AGCTCCATCTCTTCATCACCCCAAAAGGACCTGACTCAGGC
AAAGAATGGCTTCAGTTCTGTGCAGGCCACGCAGTTACAGA
CCACACAATCTGTTGAAGGTGCTACAGGCTCTGCAGTGAAA
TCTGATTCACCTTCCACTTCTAGCATCCCCCCTCTCAATGA
AACGGTATCTGCAGCTTCCTTACTGACGACAACCAATCAGC
ATTCATCCTCCTTGGGTGGCTTGAGCCACAGTGAGGAGATT
CCAAATACTACCACCACACAACACAGCAGCACGTTATCTAC
GCAGCAGAATACCCTTTCATCATCAACATCTTCTGGGCGCA
CTTCGACATCCACTCTTTTGCACACAAGTGTGGAGAGTGAG
GCGAATCTCCATTCTTCCTCCAGCACTTTTTCCACCACATC
CAGCACAGTCTCTGCACCTCCCCCAGTGGTCAGTGTCTCCT
CCAGTCTCAATAGTGGCAGTAGCCTGGGCCTCAGCCTAGGC
AGCAACTCCACTGTCACAGCCTCGACTCGAAGCTCAGTTGC
TACGACTTCAGGAAAAGCTCCTCCCAACCTCCCTCCTGGGG
TCCCGCCGTTGTTGCCTAATCCGTATATTATGGCTCCAGGG
CTGTTACATGCCTACCCGCCACAAGTATATGGTTATGATGA
CTTGCAGATGCTTCAGACAAGATTTCCATTGGATTACTACA
GCATCCCATTTCCCACACCCACTACTCCGCTGACTGGGAGG
GATGGTAGCCTGGCCAGCAACCCTTATTCTGGTGACCTCAC
AAAGTTCGGCCGTGGGGATGCCTCCTCCCCAGCCCCGGCCA
CAACCTTGGCCCAACCCCAACAGAACCAGACGCAGACTCAC
CATACCACGCAGCAGACATTCCTGAACCCGGCGCTGCCTCC
TGGCTACAGTTACACCAGCCTGCCATACTATACAGGGGTCC
CGGGCCTCCCCAGCACCTTCCAGTATGGGCCTGCTGTGTTC
CCTGTGGCTCCTACCTCTTCCAAGCAGCATGGTGTGAATGT
CAGTGTGAATGCATCGGCCACCCCTTTCCAACAGCCGAGTG
GATATGGGTCTCATGGATACAACACTGGTGTTTCAGTCACC
TCCAGTAACACGGGCGTGCCAGATATCTCGGGTTCTGTGTA
CTCCAAAACCCAGTCCTTTGAGAAACAAGGTTTTCATTCCG
GTACTCCTGCTGCTTCCTTCAACTTGCCTTCAGCCCTAGGA
AGTGGGGGCCCCATCAATCCGGCCACAGCTGCTGCCTACCC
ACCTGCCCCCTTTATGCACATTCTGACCCCCCATCAGCAGC
CGCATTCTCAGATCCTTCACCATCACCTGCAGCAGGATGGC
CAGGACATCCTCAATTTCGTCGATGACCAGCTTGGTGAATA
AGTATTACTGTACCAACTGGGCCTCCTCTAGCAGGCCCCTG
AAGGCAGTGGAATAAAATGAAATCTTCGCCCTTTAAGAACT
CCTGACCTTAATGTGGTAGTAGTATCTTGTCCTTGAGGGGA
TTTCCTTCCCCTCACCCCTAAGACTTTCACAACCTGGTGAC
TGGAAAGAACCACCACAAATCTTCATTTCCTCCAGAAACTG
CTACATCTACAGCCGATTTCAGGCAGTAAAGGGAGAGGGAT
AGAGGAGATTGGGTGGAAAATGGAGAGGATCAAGAAGGAGC
TGAGACCATTTCAAAGAAAAAAAATGCTTTATAGAGTTTTA
AGTATGACTTAGATGGGTCCAGGCAAATAAACTAAAAAGAA
GTGAAGGCAACATGTATCGTCTGGCAGAACTAAATCTTGGA
GTGGGGTGAGGGATGAAAGACTACATATTGGGTACAGTGTA
CACTGCTCGGGTGATGGGTGCGCTAAAGTCGCAGAAATCAC
TAAAGAACTCATCCATGTAACCAAACACCACCTGTACCCCA
AAAACGAAATAAAAAAACAAAACCTTGGGGCCCATTCCCCC
TAGGAATGGACTACTGTAAAAA

In some embodiments, the UBAP2L protein has an amino acid sequence comprising, consisting of, or consisting essentially of all or part of a sequence selected from SEQ ID NO: 68, SEQ ID NO: 69, SEQ ID NO: 70, SEQ ID NO: 71 and a biological equivalent of each thereof.

(NP_0556623)
(SEQ ID NO: 68)
MMTSVGTNRARGNWEQPQNQNQTQHKQRPQATAEQIRLAQMISDHNDAD
FEEKVKQLIDITGKNQDECVIALHDCNGDVNRAINVLLEGNPDTHSWEM
VGKKKGVSGQKDGGQTESNEEGKENRDRDRDYSRRRGGPPRRGRGASRG
REFRGQENGLDGTKSGGPSGRGTERGRRGRGRGRGGSGRRGGRFSAQGM
GTFNPADYAEPANTDDNYGNSSGNTWNNTGHFEPDDGTSAWRTATEEWG
TEDWNEDLSETKIFTASNVSSVPLPAENVTITAGQRIDLAVLLGKTPST
MENDSSNLDPSQAPSLAQPLVFSNSKQTAISQPASGNTFSHHSMVSMLG
KGFGDVGEAKGGSTTGSQFLEQFKTAQALAQLAAQHSQSGSTTTSSWDM
GSTTQSPSLVQYDLKNPSDSAVHSPFTKRQAFTPSSTMMEVFLQEKSPA
VATSTAAPPPPSSPLPSKSTSAPQMSPGSSDNQSSSPQPAQQKLKQQKK
KASLTSKIPALAVEMPGSADISGLNLQFGALQFGSEPVLSDYESTPTTS
ASSSQAPSSLYTSTASESSSTISSNQSQESGYQSGPIQSTTYTSQNNAQ
GPLYEQRSTQTRRYPSSISSSPQKDLTQAKNGFSSVQATQLQTTQSVEG
ATGSAVKSDSPSTSSIPPLNETVSAASLLTTTNQHSSSLGGLSHSEEIP
NTTTTQHSSTLSTQQNTLSSSTSSGRTSTSTLLHTSVESEANLHSSSST
FSTTSSTVSAPPPVVSVSSSLNSGSSLGLSLGSNSTVTASTRSSVATTS
GKAPPNLPPGVPPLLPNPYIMAPGLLHAYPPQVYGYDDLQMLQTRFPLD
YYSIPFPTPTTPLTGRDGSLASNPYSGDLTKFGRGDASSPAPATTLAQP
QQNQTQTHHTTQQTFLNPALPPGYSYTSLPYYTGVPGLPSTFQYGPAVF
PVAPTSSKQHGVNVSVNASATPFQQPSGYGSHGYNTGVSVTSSNTGVPD
ISGSVYSKTQQSFEKQGFHSGTPAASFNLPSALGSGGPINPATAAAYPP
APFMHILTPHQQPHSQILHHHLQQDGQTGSGQRSQTSSIPQKPQTNKSA
YNSYSWGAN
(NP_001120792.1)
(SEQ ID NO: 69)
MMTSVGTNRARGNWEQPQNQNQTQHKQRPQATAEQIRLAQMISDHNDAD
FEEKVKQLIDITGKNQDECVIALHDCNGDVNRAINVLLEGNPDTHSWEM
VGKKKGVSGQKDGGQTESNEEGKENRDRDRDYSRRRGGPPRRGRGASRG
REFRGQENGLDGTKSGGPSGRGTERGRRGRGRGRGGSGRRGGRFSAQGM
GTFNPADYAEPANTDDNYGNSSGNTWNNTGHFEPDDGTSAWRTATEEWG
TEDWNEDLSETKIFTASNVSSVPLPAENVTITAGQRIDLAVLLGKTPST
MENDSSNLDPSQAPSLAQPLVFSNSKQTAISQPASGNTFSHHSMVSMLG
KGFGDVGEAKGGSTTGSQFLEQFKTAQALAQLAAQHSQSGSTTTSSWDM
GSTTQSPSLVQYDLKNPSDSAVHSPFTKRQAFTPSSTMMEVFLQEKSPA
VATSTAAPPPPSSPLPSKSTSAPQMSPGSSDNQSSSPQPAQQKLKQQKK
KASLTSKIPALAVEMPGSADISGLNLQFGALQFGSEPVLSDYESTPTTS
ASSSQAPSSLYTSTASESSSTISSNQSQESGYQSGPIQSTTYTSQNNAQ
GPLYEQRSTQTRRYPSSISSSPQKDLTQAKNGFSSVQATQLQTTQSVEG
ATGSAVKSDSPSTSSIPPLNETVSAASLLTTTNQHSSSLGGLSHSEEIP
NTTTTQHSSTLSTQQNTLSSSTSSGRTSTSTLLHTSVESEANLHSSSST
FSTTSSTVSAPPPVVSVSSSLNSGSSLGLSLGSNSTVTASTRSSVATTS
GKAPPNLPPGVPPLLPNPYIMAPGLLHAYPPQVYGYDDLQMLQTRFPLD
YYSIPFPTPTTPLTGRDGSLASNPYSGDLTKFGRGDASSPAPATTLAQP
QQNQTQTHHTTQQTFLNPALPPGYSYTSLPYYTGVPGLPSTFQYGPAVF
PVAPTSSKQHGVNVSVNASATPFQQPSGYGSHGYNTGRKYPPPYKHFWT
AES
(NP_001274744.1)
(SEQ ID NO: 70)
MMTSVGTNRARGNWEQPQNQNQTQHKQRPQATAEQIRLAQMISDHNDAD
FEEKVKQLIDITGKNQDECVIALHDCNGDVNRAINVLLEGNPDTHSWEM
VGKKKGVSGQKDGGQTESNEEGKENRDRDRDYSRRRGGPPRRGRVRGQE
NGLDGTKSGGPSGRGTERGRRGRGRGRGGSGRRGGRFSAQGMGTFNPAD
YAEPANTDDNYGNSSGNTWNNTGHFEPDDGTSAWRTATEEWGTEDWNED
LSETKIFTASNVSSVPLPAENVTITAGQRIDLAVLLGKTPSTMENDSSN
LDPSQAPSLAQPLVFSNSKQTAISQPASGNTFSHHSMVSMLGKGFGDVG
EAKGGSTTGSQFLEQFKTAQALAQLAAQHSQSGSTTTSSWDMGSTTQSP
SLVQYDLKNPSDSAVHSPFTKRQAFTPSSTMMEVFLQEKSPAVATSTAA
PPPPSSPLPSKSTSAPQMSPGSSDNQSSSPQPAQQKLKQQKKKASLTSK
IPALAVEMPGSADISGLNLQFGALQFGSEPVLSDYESTPTTSASSSQAP
SSLYTSTASESSSTISSNQSQESGYQSGPIQSTTYTSQNNAQGPLYEQR
STQTRRYPSSISSSPQKDLTQAKNGFSSVQATQLQTTQSVEGATGSAVK
SDSPSTSSIPPLNETVSAASLLTTTNQHSSSLGGLSHSEEIPNTTTTQH
SSTLSTQQNTLSSSTSSGRTSTSTLLHTSVESEANLHSSSSTFSTTSST
VSAPPPVVSVSSSLNSGSSLGLSLGSNSTVTASTRSSVATTSGKAPPNL
PPGVPPLLPNPYIMAPGLLHAYPPQVYGYDDLQMLQTRFPLDYYSIPFP
TPTTPLTGRDGSLASNPYSGDLTKFGRGDASSPAPATTLAQPQQNQTQT
HHTTQQTFLNPALPPGYSYTSLPYYTGVPGLPSTFQYGPAVFPVAPTSS
KQHGVNVSVNASATPFQQPSGYGSHGYNTGRKYPPPYKHFWTAES
(NP_001274745.1)
(SEQ ID NO: 71
MMTSVGTNRARGNWEQPQNQNQTQHKQRPQATAEQIRLAQMISDHNDAD
FEEKVKQLIDITGKNQDECVIALHDCNGDVNRAINVLLEGNPDTHSWEM
VGKKKGVSGQKDGGQTESNEEGKENRDRDRDYSRRRGGPPRRGRGASRG
RECMHGALSKPAVVRGQENGLDGTKSGGPSGRGTERGRRGRGRGRGGSG
RRGGRFSAQGMGTFNPADYAEPANTDDNYGNSSGNTWNNTGHFEPDDGT
SAWRTATEEWGTEDWNEDLSETKIFTASNVSSVPLPAENVTITAGQRID
LAVLLGKTPSTMENDSSNLDPSQAPSLAQPLVFSNSKQTAISQPASGNT
FSHHSMVSMLGKGFGDVGEAKGGSTTGSQFLEQFKTAQALAQLAAQHSQ
SGSTTTSSWDMGSTTQSPSLVQYDLKNPSDSAVHSPFTKRQAFTPSSTM
MEVFLQEKSPAVATSTAAPPPPSSPLPSKSTSAPQMSPGSSDNQSSSPQ
PAQQKLKQQKKKASLTSKIPALAVEMPGSADISGLNLQFGALQFGSEPV
LSDYESTPTTSASSSQAPSSLYTSTASESSSTISSNQSQESGYQSGPIQ
STTYTSQNNAQGPLYEQRSTQTRRYPSSISSSPQKDLTQAKNGFSSVQA
TQLQTTQSVEGATGSAVKSDSPSTSSIPPLNETVSAASLLTTTNQHSSS
LGGLSHSEEIPNTTTTQHSSTLSTQQNTLSSSTSSGRTSTSTLLHTSVE
SEANLHSSSSTFSTTSSTVSAPPPVVSVSSSLNSGSSLGLSLGSNSTVT
ASTRSSVATTSGKAPPNLPPGVPPLLPNPYIMAPGLLHAYPPQVYGYDD
LQMLQTRFPLDYYSIPFPTPTTPLTGRDGSLASNPYSGDLTKFGRGDAS
SPAPATTLAQPQQNQTQTHHTTQQTFLNPALPPGYSYTSLPYYTGVPGL
PSTFQYGPAVFPVAPTSSKQHGVNVSVNASATPFQQPSGYGSHGYNTGV
SVTSSNTGVPDISGSVYSKTQSFEKQGFHSGTPAASFNLPSALGSGGPI
NPATAAAYPPAPFMHILTPHQQPHSQILHHHLQQDGQDILNFVDDQLGE

In some embodiments, the fusion protein is a dCas9-UBAP2L fusion protein encoded by a nucleic acid comprising the following nucleic acid sequence:

(SEQ ID NO: 72)
ATGGGTAAGCCTATCCCTAACCCTCTCCTCGGTCTCGATTCTACGGCTAGCATGGACAAGAAGT
ACAGCATCGGCCTGGCCATCGGCACCAACTCTGTGGGCTGGGCCGTGATCACCGACGAGTACAA
GGTGCCCAGCAAGAAATTCAAGGTGCTGGGCAACACCGACCGGCACAGCATCAAGAAGAACCTG
ATCGGCGCCCTGCTGTTCGACAGCGGAGAAACAGCCGAGGCCACCCGGCTGAAGAGAACCGCCA
GAAGAAGATACACCAGACGGAAGAACCGGATCTGCTATCTGCAAGAGATCTTCAGCAACGAGAT
GGCCAAGGTGGACGACAGCTTCTTCCACAGACTGGAAGAGTCCTTCCTGGTGGAAGAGGATAAG
AAGCACGAGCGGCACCCCATCTTCGGCAACATCGTGGACGAGGTGGCCTACCACGAGAAGTACC
CCACCATCTACCACCTGAGAAAGAAACTGGTGGACAGCACCGACAAGGCCGACCTGCGGCTGAT
CTATCTGGCCCTGGCCCACATGATCAAGTTCCGGGGCCACTTCCTGATCGAGGGCGACCTGAAC
CCCGACAACAGCGACGTGGACAAGCTGTTCATCCAGCTGGTGCAGACCTACAACCAGCTGTTCG
AGGAAAACCCCATCAACGCCAGCGGCGTGGACGCCAAGGCCATCCTGTCTGCCAGACTGAGCAA
GAGCAGACGGCTGGAAAATCTGATCGCCCAGCTGCCCGGCGAGAAGAAGAATGGCCTGTTCGGC
AACCTGATTGCCCTGAGCCTGGGCCTGACCCCCAACTTCAAGAGCAACTTCGACCTGGCCGAGG
ATGCCAAACTGCAGCTGAGCAAGGACACCTACGACGACGACCTGGACAACCTGCTGGCCCAGAT
CGGCGACCAGTACGCCGACCTGTTTCTGGCCGCCAAGAACCTGTCCGACGCCATCCTGCTGAGC
GACATCCTGAGAGTGAACACCGAGATCACCAAGGCCCCCCTGAGCGCCTCTATGATCAAGAGAT
ACGACGAGCACCACCAGGACCTGACCCTGCTGAAAGCTCTCGTGCGGCAGCAGCTGCCTGAGAA
GTACAAAGAGATTTTCTTCGACCAGAGCAAGAACGGCTACGCCGGCTACATCGATGGCGGAGCC
AGCCAGGAAGAGTTCTACAAGTTCATCAAGCCCATCCTGGAAAAGATGGACGGCACCGAGGAAC
TGCTCGTGAAGCTGAACAGAGAGGACCTGCTGCGGAAGCAGCGGACCTTCGACAACGGCAGCAT
CCCCCACCAGATCCACCTGGGAGAGCTGCACGCCATTCTGCGGCGGCAGGAAGATTTTTACCCA
TTCCTGAAGGACAACCGGGAAAAGATCGAGAAGATCCTGACCTTCCGCATCCCCTACTACGTGG
GCCCTCTGGCCAGGGGAAACAGCAGATTCGCCTGGATGACCAGAAAGAGCGAGGAAACCATCAC
CCCCTGGAACTTCGAGGAAGTGGTGGACAAGGGCGCCAGCGCCCAGAGCTTCATCGAGCGGATG
ACCAACTTCGATAAGAACCTGCCCAACGAGAAGGTGCTGCCCAAGCACAGCCTGCTGTACGAGT
ACTTCACCGTGTACAACGAGCTGACCAAAGTGAAATACGTGACCGAGGGAATGAGAAAGCCCGC
CTTCCTGAGCGGCGAGCAGAAAAAAGCCATCGTGGACCTGCTGTTCAAGACCAACCGGAAAGTG
ACCGTGAAGCAGCTGAAAGAGGACTACTTCAAGAAAATCGAGTGCTTCGACTCCGTGGAAATCT
CCGGCGTGGAAGATCGGTTCAACGCCTCCCTGGGCACATACCACGATCTGCTGAAAATTATCAA
GGACAAGGACTTCCTGGACAATGAGGAAAACGAGGACATTCTGGAAGATATCGTGCTGACCCTG
ACACTGTTTGAGGACAGAGAGATGATCGAGGAACGGCTGAAAACCTATGCCCACCTGTTCGACG
ACAAAGTGATGAAGCAGCTGAAGCGGCGGAGATACACCGGCTGGGGCAGGCTGAGCCGGAAGCT
GATCAACGGCATCCGGGACAAGCAGTCCGGCAAGACAATCCTGGATTTCCTGAAGTCCGACGGC
TTCGCCAACAGAAACTTCATGCAGCTGATCCACGACGACAGCCTGACCTTTAAAGAGGACATCC
AGAAAGCCCAGGTGTCCGGCCAGGGCGATAGCCTGCACGAGCACATTGCCAATCTGGCCGGCAG
CCCCGCCATTAAGAAGGGCATCCTGCAGACAGTGAAGGTGGTGGACGAGCTCGTGAAAGTGATG
GGCCGGCACAAGCCCGAGAACATCGTGATCGAAATGGCCAGAGAGAACCAGACCACCCAGAAGG
GACAGAAGAACAGCCGCGAGAGAATGAAGCGGATCGAAGAGGGCATCAAAGAGCTGGGCAGCCA
GATCCTGAAAGAACACCCCGTGGAAAACACCCAGCTGCAGAACGAGAAGCTGTACCTGTACTAC
CTGCAGAATGGGCGGGATATGTACGTGGACCAGGAACTGGACATCAACCGGCTGTCCGACTACG
ATGTGGACGCTATCGTGCCTCAGAGCTTTCTGAAGGACGACTCCATCGATAACAAAGTGCTGAC
TCGGAGCGACAAGAACCGGGGCAAGAGCGACAACGTGCCCTCCGAAGAGGTCGTGAAGAAGATG
AAGAACTACTGGCGCCAGCTGCTGAATGCCAAGCTGATTACCCAGAGGAAGTTCGACAATCTGA
CCAAGGCCGAGAGAGGCGGCCTGAGCGAACTGGATAAGGCCGGCTTCATCAAGAGACAGCTGGT
GGAAACCCGGCAGATCACAAAGCACGTGGCACAGATCCTGGACTCCCGGATGAACACTAAGTAC
GACGAGAACGACAAACTGATCCGGGAAGTGAAAGTGATCACCCTGAAGTCCAAGCTGGTGTCCG
ATTTCCGGAAGGATTTCCAGTTTTACAAAGTGCGCGAGATCAACAACTACCACCACGCCCACGA
CGCCTACCTGAACGCCGTCGTGGGAACCGCCCTGATCAAAAAGTACCCTAAGCTGGAAAGCGAG
TTCGTGTACGGCGACTACAAGGTGTACGACGTGCGGAAGATGATCGCCAAGAGCGAGCAGGAAA
TCGGCAAGGCTACCGCCAAGTACTTCTTCTACAGCAACATCATGAACTTTTTCAAGACCGAGAT
TACCCTGGCCAACGGCGAGATCCGGAAGCGGCCTCTGATCGAGACAAACGGCGAAACAGGCGAG
ATCGTGTGGGATAAGGGCCGGGACTTTGCCACCGTGCGGAAAGTGCTGTCTATGCCCCAAGTGA
ATATCGTGAAAAAGACCGAGGTGCAGACAGGCGGCTTCAGCAAAGAGTCTATCCTGCCCAAGAG
GAACAGCGACAAGCTGATCGCCAGAAAGAAGGACTGGGACCCTAAGAAGTACGGCGGCTTCGAC
AGCCCCACCGTGGCCTATTCTGTGCTGGTGGTGGCCAAAGTGGAAAAGGGCAAGTCCAAGAAAC
TGAAGAGTGTGAAAGAGCTGCTGGGGATCACCATCATGGAAAGAAGCAGCTTCGAGAAGAATCC
CATCGACTTTCTGGAAGCCAAGGGCTACAAAGAAGTGAAAAAGGACCTGATCATCAAGCTGCCT
AAGTACTCCCTGTTCGAGCTGGAAAACGGCCGGAAGAGAATGCTGGCCTCTGCCGGCGAACTGC
AGAAGGGAAACGAACTGGCCCTGCCCTCCAAATATGTGAACTTCCTGTACCTGGCCAGCCACTA
TGAGAAGCTGAAGGGCTCCCCCGAGGATAATGAGCAGAAACAGCTGTTTGTGGAACAGCACAAA
CACTACCTGGACGAGATCATCGAGCAGATCAGCGAGTTCTCCAAGAGAGTGATCCTGGCCGACG
CTAATCTGGACAAGGTGCTGAGCGCCTACAACAAGCACAGAGACAAGCCTATCAGAGAGCAGGC
CGAGAATATCATCCACCTGTTTACCCTGACCAATCTGGGAGCCCCTGCCGCCTTCAAGTACTTT
GACACCACCATCGACCGGAAGAGGTACACCAGCACCAAAGAGGTGCTGGACGCCACCCTGATCC
ACCAGAGCATCACCGGCCTGTACGAGACACGGATCGACCTGTCTCAGCTGGGAGGCGACCTCGA
GGGCGGATCCGGTGGTTCCGGAGGAGCTGTCGACACATCGGTGGGCACTAACCGAGCCCGGGGA
AACTGGGAACAACCTCAAAACCAAAACCAGACACAGCACAAGCAGCGGCCACAGGCCACTGCAG
AACAAATTAGACTTGCACAGATGATTTCGGACCATAATGATGCTGACTTTGAGGAGAAGGTGAA
ACAATTGATTGATATTACAGGCAAGAACCAGGATGAATGTGTGATTGCTTTGCATGACTGCAAT
GGAGATGTCAACAGAGCTATCAATGTTCTTCTGGAAGGAAACCCAGACACGCATTCCTGGGAGA
TGGTCGGGAAGAAGAAGGGAGTCTCAGGCCAGAAGGATGGTGGCCAGACGGAATCCAATGAGGA
AGGCAAAGAAAATCGAGACCGGGACAGAGACTATAGTCGGCGACGTGGTGGGCCACCAAGACGG
GGGAGAGGTGCCAGCCGTGGACGAGAGTTTCGAGGTCAGGAAAATGGATTGGATGGCACCAAGA
GTGGAGGGCCTTCTGGAAGAGGAACAGAAAGAGGCAGAAGGGGCCGTGGCCGAGGCAGAGGTGG
CTCTGGTAGGCGAGGAGGAAGGTTTTCTGCTCAAGGAATGGGAACCTTTAACCCAGCTGATTAT
GCAGAGCCAGCCAATACTGATGATAACTATGGCAATAGCAGCGGCAATACGTGGAACAACACTG
GCCACTTTGAACCAGATGATGGGACGAGTGCATGGAGGACTGCAACAGAGGAGTGGGGGACTGA
AGATTGGAATGAAGATCTTTCTGAGACCAAGATCTTCACTGCCTCTAATGTGTCTTCAGTGCCT
CTGCCTGCGGAGAATGTGACAATCACTGCTGGTCAGAGAATTGACCTTGCTGTTCTGCTGGGGA
AGACACCATCTACAATGGAGAATGATTCATCTAATCTGGATCCGTCTCAGGCTCCTTCTCTGGC
CCAGCCTCTGGTGTTCAGTAATTCGAAGCAGACTGCCATATCACAGCCTGCTTCAGGGAACACA
TTTTCTCATCACAGTATGGTGAGCATGTTAGGGAAAGGATTTGGTGATGTCGGTGAAGCTAAAG
GCGGCAGTACTACAGGCTCCCAGTTCTTGGAGCAATTCAAGACTGCCCAAGCCCTGGCTCAGTT
GGCAGCTCAGCATTCTCAGTCTGGAAGCACCACCACCTCCTCTTGGGACATGGGCTCGACGACA
CAATCCCCATCACTGGTGCAGTATGATTTGAAGAACCCAAGTGATTCAGCAGTGCACAGCCCCT
TTACAAAGCGCCAGGCTTTTACCCCATCTTCAACCATGATGGAGGTGTTCCTTCAGGAGAAGTC
ACCTGCAGTGGCTACCTCCACAGCTGCACCTCCACCTCCGTCTTCTCCTCTGCCAAGCAAATCC
ACATCGGCTCCACAGATGTCGCCTGGATCTTCAGACAACCAGTCCTCTAGCCCTCAGCCGGCTC
AGCAGAAACTGAAACAGCAGAAGAAAAAAGCCTCCTTGACTTCTAAGATTCCTGCTCTGGCTGT
GGAGATGCCTGGCTCAGCAGATATCTCAGGGCTAAACCTGCAGTTTGGGGCATTGCAGTTTGGG
TCAGAGCCTGTCCTTTCTGATTATGAGTCCACCCCCACCACGAGCGCCTCTTCAAGCCAGGCTC
CAAGTAGCCTGTATACCAGCACGGCCAGTGAATCATCCTCTACAATTTCATCTAACCAGAGTCA
GGAGTCTGGTTATCAGAGCGGCCCAATTCAGTCGACAACCTATACCTCCCAAAATAATGCTCAG
GGCCCTCTTTATGAACAGAGATCCACACAGACTCGGCGGTACCCCAGCTCCATCTCTTCATCAC
CCCAAAAGGACCTGACTCAGGCAAAGAATGGCTTCAGTTCTGTGCAGGCCACGCAGTTACAGAC
CACACAATCTGTTGAAGGTGCTACAGGCTCTGCAGTGAAATCTGATTCACCTTCCACTTCTAGC
ATCCCCCCTCTCAATGAAACGGTATCTGCAGCTTCCTTACTGACGACAACCAATCAGCATTCAT
CCTCCTTGGGTGGCTTGAGCCACAGTGAGGAGATTCCAAATACTACCACCACACAACACAGCAG
CACGTTATCTACGCAGCAGAATACCCTTTCATCATCAACATCTTCTGGGCGCACTTCGACATCC
ACTCTTTTGCACACAAGTGTGGAGAGTGAGGCGAATCTCCATTCTTCCTCCAGCACTTTTTCCA
CCACATCCAGCACAGTCTCTGCACCTCCCCCAGTGGTCAGTGTCTCCTCCAGTCTCAATAGTGG
CAGTAGCCTGGGCCTCAGCCTAGGCAGCAACTCCACTGTCACAGCCTCGACTCGAAGCTCAGTT
GCTACGACTTCAGGAAAAGCTCCTCCCAACCTCCCTCCTGGGGTCCCGCCGTTGTTGCCTAATC
CGTATATTATGGCTCCAGGGCTGTTACATGCCTACCCGCCACAAGTATATGGTTATGATGACTT
GCAGATGCTTCAGACAAGATTTCCATTGGATTACTACAGCATCCCATTTCCCACACCCACTACT
CCGCTGACTGGGAGGGATGGTAGCCTGGCCAGCAACCCTTATTCTGGTGACCTCACAAAGTTCG
GCCGTGGGGATGCCTCCTCCCCAGCCCCGGCCACAACCTTGGCCCAACCCCAACAGAACCAGAC
GCAGACTCACCATACCACGCAGCAGACATTCCTGAACCCGGCGCTGCCTCCTGGCTACAGTTAC
ACCAGCCTGCCATACTATACAGGGGTCCCGGGCCTCCCCAGCACCTTCCAGTATGGGCCTGCTG
TGTTCCCTGTGGCTCCTACCTCTTCCAAGCAGCATGGTGTGAATGTCAGTGTGAATGCATCGGC
CACCCCTTTCCAACAGCCGAGTGGATATGGGTCTCATGGATACAACACTGGTGTTTCAGTCACC
TCCAGTAACACGGGCGTGCCAGATATCTCGGGTTCTGTGTACTCCAAAACCCAGCAGTCCTTTG
AGAAACAAGGTTTTCATTCCGGTACTCCTGCTGCTTCCTTCAACTTGCCTTCAGCCCTAGGAAG
TGGGGGCCCCATCAATCCGGCCACAGCTGCTGCCTACCCACCTGCCCCCTTTATGCACATTCTG
ACCCCCCATCAGCAGCCGCATTCTCAGATCCTTCACCATCACCTGCAGCAGGATGGCCAGACGG
GCAGCGGGCAACGTAGCCAGACCAGCTCCATCCCGCAGAAGCCCCAGACCAACAAGTCTGCCTA
CAACAGCTACAGCTGGGGGGCCAACTCTAGACTTAAG

### Polynucleotides and Vectors

In some aspects, provided herein are polynucleotides encoding a fusion protein comprising, consisting of, or consisting essentially of: (i) a guide nucleotide sequence-programmable RNA binding protein; and (ii) an EIF4E protein. In other aspects, provided herein are polynucleotides encoding a fusion protein comprising, consisting of, or consisting essentially of: (i) a guide nucleotide sequence-programmable RNA binding protein; and (ii) an EIF4E-BP1 protein. In some embodiments, the polynucleotides further comprise a nucleic acid sequence encoding a linker peptide.

In some aspects, provided herein are vectors comprising, consisting of, or consisting essentially of a polynucleotide encoding a fusion protein comprising, consisting of, or consisting essentially of: (i) a guide nucleotide sequence-programmable RNA binding protein; and (ii) an EIF4E protein. In some embodiments, the guide nucleotide sequence-programmable RNA binding protein and the EIF4E protein are encoded in a single vector. In other aspects, provided herein are vectors comprising, consisting of, or consisting essentially of a polynucleotide encoding a fusion protein comprising, consisting of, or consisting essentially of: (i) a guide nucleotide sequence-programmable RNA binding protein; and (ii) an EIF4E-BP1 protein. In some embodiments, the polynucleotides further comprise a nucleic acid sequence encoding a linker peptide. In some embodiments, the guide nucleotide sequence-programmable RNA binding protein and the EIF4-BP1 protein are encoded in a single vector.

In some aspects, provided herein are polynucleotides encoding a fusion RNA comprising, consisting of, or consisting essentially of: (i) a guide nucleotide sequence-programmable RNA; and (ii) one or more internal ribosome entry sites (IRES). In some embodiments, the polynucleotides further comprise a nucleic acid sequence encoding a spacer RNA.

In some aspects, provided herein are vectors comprising, consisting of, or consisting essentially of a polynucleotide encoding a fusion RNA comprising, consisting of, or consisting essentially of: (i) a guide nucleotide sequence-programmable RNA; and (ii) one or more internal ribosome entry sites (IRES), optionally wherein the vector is an adenoviral vector, an adeno-associated viral vector, or a lentiviral vector. In some embodiments, the vector further comprises an expression control element. In some embodiments, the vector further comprises a selectable marker. In some embodiments, the vector further comprises a polynucleotide encoding a tracrRNA and/or a PAMmer. In some embodiments, the guide nucleotide sequence-programmable RNA and one or more internal ribosome binding sites (IRES) are encoded in a single vector.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments, the vector further comprises, consists of, or consists essentially of a polynucleotide encoding either (i) a gRNA, or (ii) a crRNA and a tracrRNA. In some embodiments, the gRNA or the crRNA comprises a nucleotide sequence complementary to a target RNA. In some embodiments, the guide nucleotide sequence-programmable RNA binding protein and the EIF4E protein are encoded in a single vector further comprising, consisting of, or consisting essentially of a polynucleotide encoding either (i) a gRNA, or (ii) a crRNA and a tracrRNA. In some embodiments, the guide nucleotide sequence-programmable RNA binding protein and the EIF4E-BP1 protein are encoded in a single vector further comprising, consisting of, or consisting essentially of a polynucleotide encoding either (i) a gRNA, or (ii) a crRNA and a tracrRNA

In some embodiments, the vector further comprises, consists of, or consists essentially of a polynucleotide encoding (i) a tracrRNA and/or (ii) a PAMmer oligonucleotide. In some embodiments, the fusion RNA comprises a nucleotide sequence complementary to a target RNA. In some embodiments, the guide nucleotide sequence-programmable RNA and one or more internal ribosome binding sites (IRES) are encoded in a single vector further comprising, consisting of, or consisting essentially of a polynucleotide encoding (i) a tracrRNA and/or (ii) a PAMmer oligonucleotide.

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In some embodiments, the vector further comprises a fusion protein of the disclosure. In some embodiments, the fusion protein comprises a first RNA binding protein and a second RNA binding protein.

In some embodiments of the compositions and methods of the disclosure, a first vector comprises a guide RNA of the disclosure and a second vector comprises a fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical. In some embodiments, the first vector and the second vector are not identical.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex, or a dendrimer.

### Cells

In other aspects, provided herein are cells comprising, consisting of, or consisting essentially of one or more vectors comprising, consisting of, or consisting essentially of a polynucleotide encoding a fusion protein comprising, consisting of, or consisting essentially of: (i) a guide nucleotide sequence-programmable RNA binding protein; and (ii) an EIF4E protein. In other aspects, provided herein are cells comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an EIF4E-BP1 protein. In some embodiments, the polynucleotides further comprise a nucleic acid sequence encoding a linker peptide.

In some aspects, provided herein are cells comprising, consisting of, or consisting essentially of a fusion protein comprising, consisting of, or consisting essentially of: (i) a guide nucleotide sequence-programmable RNA binding protein; and (ii) an EIF4E protein. In other aspects, provided herein are cells comprising, consisting of, or consisting essentially of a fusion protein comprising, consisting of, or consisting essentially of: (i) a guide nucleotide sequence-programmable RNA binding protein; and (ii) an EIF4E-BP1 protein.

In some aspects, provided herein are cells comprising, consisting of, or consisting essentially of a fusion RNA, a polynucleotide encoding the fusion RNA, a vector comprising the polynucleotide, or a viral particle comprising the fusion RNA, polynucleotide, or vector; wherein the fusion RNA comprises, consists of, or consists essentially of: (i) a guide nucleotide sequence-programmable RNA; and (ii) one or more internal ribosome entry sites (IRES). In some embodiments, the guide nucleotide sequence-programmable RNA is a guide RNA (gRNA) or a crisprRNA (crRNA). In some embodiments, the cell is a eukaryotic cell. In some embodiments, the cell is a prokaryotic cell. In some embodiments, the cell is a mammalian cell, optionally a bovine, murine, feline, equine, porcine, canine, simian, or human cell.

In some aspects, provided herein is a population of cells comprising, consisting of, or consisting essentially of a fusion RNA, a polynucleotide encoding the fusion RNA, a vector comprising the polynucleotide, or a viral particle comprising the fusion RNA, polynucleotide, or vector; wherein the fusion RNA comprises, consists of, or consists essentially of: (i) a guide nucleotide sequence-programmable RNA; and (ii) one or more internal ribosome entry sites (IRES). In some embodiments, the guide nucleotide sequence-programmable RNA is a guide RNA (gRNA) or a crisprRNA (crRNA). In some embodiments, the cell is a eukaryotic cell. In some embodiments, the cell is a prokaryotic cell. In some embodiments, the cell is a mammalian cell, optionally a bovine, murine, feline, equine, porcine, canine, simian, or human cell.

In some embodiments, the cell is a eukaryotic cell. In other embodiments, the cell is a prokaryotic cell. In some embodiments, the cell is a mammalian cell. In some embodiments, the cell is a bovine, murine, feline, equine, porcine, canine, simian, or human cell. In particular embodiments, the cell is a human cell. In some embodiments, the cell is isolated from a subject.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is an immune cell. In some embodiments, an immune cell of the disclosure is a lymphocyte. In some embodiments, an immune cell of the disclosure is a T lymphocyte (also referred to herein as a T-cell). Exemplary T-cells of the disclosure include, but are not limited to, naive T cells, effector T cells, helper T cells, memory T cells, regulatory T cells (Tregs), and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland, and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of the retina. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo, or in situ.

In some embodiments of the compositions and methods of the disclosure, a somatic cell of the disclosure is autologous or allogeneic.

### RNA-Targeted CRISPR Systems

In some aspects, provided herein are systems for post-transcriptional gene regulation, the systems comprising, consisting of, or consisting essentially of: (i) fusion protein comprising, consisting of, or consisting essentially of: (a) a guide nucleotide sequence-programmable RNA binding protein; and (b) an EIF4E protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In other aspects, provided herein are systems for post-transcriptional gene regulation, the systems comprising, consisting of, or consisting essentially of: (i) fusion protein comprising, consisting of, or consisting essentially of: (a) a guide nucleotide sequence-programmable RNA binding protein; and (b) an EIF4E-BP1 protein; and either (ii) a gRNA or (iii) a crRNA and a tracrRNA, wherein the gRNA or the crRNA comprises a sequence complementary to a target mRNA. In some embodiments, the complementary sequence is a spacer sequence. In some embodiments, the fusion protein disclosed herein is used with the fusion RNA disclosed herein.

In some aspects, provided herein are systems for upregulating or increasing translation of a target mRNA, the systems comprising, consisting of, or consisting essentially of: (i) fusion protein comprising, consisting of, or consisting essentially of: (a) a guide nucleotide sequence-programmable RNA binding protein; and (b) an EIF4E protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In some aspects, provided herein are systems for post-transcriptional gene regulation, the systems comprising, consisting of, or consisting essentially of: (a) a fusion RNA comprising, consisting of, or consisting essentially of: (i) a guide nucleotide sequence-programmable RNA; and (ii) one or more internal ribosome entry sites (IRES); and (b) a guide nucleotide sequence-programmable RNA binding protein, wherein the fusion RNA comprises a sequence complementary to a target mRNA. In some embodiments, the system further comprises a PAMmer. In some embodiments, the target mRNA does not comprise a PAM sequence or its complement.

In some aspects, provided herein are systems for increasing translation of a target mRNA, the systems comprising, consisting of, or consisting essentially of: (a) a fusion RNA comprising, consisting of, or consisting essentially of: (i) a guide nucleotide sequence-programmable RNA; and (ii) one or more internal ribosome entry sites (IRES); and (b) a guide nucleotide sequence-programmable RNA binding protein, wherein the fusion RNA comprises a sequence complementary to a target mRNA. In some embodiments, the system further comprises a PAMmer. In some embodiments, the target mRNA does not comprise a PAM sequence or its complement.

In some embodiments of the system, the guide nucleotide-sequence programmable RNA binding protein is selected from: Cas9, modified Cas9, Cpf1, Cas13a, Cas13b, CasRX/Cas13d, CasM and a biological equivalent of each thereof. In some embodiments, the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9). In some embodiments, the guide nucleotide sequence-programmable RNA binding protein is nuclease inactive.

In some embodiments, the CasRX/Cas13d protein is an effector of the type VI-D CRISPR-Cas systems. In some embodiments, the CasRX/Case13d protein is an RNA-guided RNA endonuclease enzyme that can cut or bind RNA. In some embodiments, the CasRX/Cas13d protein can include one or more higher eukaryotes and prokaryotes nucleotide-binding (HEPN) domains. In some embodiments, the CasRX/Case13d protein can include either a wild-type or mutated HEPN domain. In some embodiments, the CasRX/Case13d protein includes a mutated HEPN domain that cannot cut RNA but can process guide RNA. In some embodiments, the CasRX/Cas13d protein does not require a protospacer flanking sequence. Also see WO Publication No. WO2019/040664 & US2019/0062724, which is incorporated herein by reference in its entirety, for further examples and sequences of CasRX/Cas13d protein, without limitation, specific reference is made to SEQ ID NOS: 54, 57, 61, 67, 69, 71, 72, 73, 74, 75, 76, 77, 78, 85, 86, 87, 88, 113, 147, 153, 154, 155, 158, 160, 162, 164, 170, 179, 183, 185, 187, 189, 190, 202, 204, 206, 208, 209, 210, and 212 reproduced herein. Yan et al. (2018) Mol Cell. 70(2):327-339 (doi: 10.1016/j.molce1.2018.02.2018) and Konermann et al. (2018) Cell 173(3):665-676 (doi: 10.1016/j.ce11/2018.02.033) have described CasRX/Cas13d proteins and both of which are incorporated by reference herein in their entireties. Also see WO Publication Nos. WO2018/183703 (CasM) and WO2019/006471 (Cas13d), which are incorporated herein by reference in their entirety.

In some embodiments, increasing or upregulating translation refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In other aspects, provided herein are systems for decreasing or downregulating translation of a target mRNA, the systems comprising, consisting of, or consisting essentially of: (i) fusion protein comprising, consisting of, or consisting essentially of: (a) a guide nucleotide sequence-programmable RNA binding protein; and (b) an EIF4E-BP1 protein; and either (ii) a gRNA or (iii) a crRNA and a tracrRNA, wherein the gRNA or the crRNA comprises a sequence complementary to a target mRNA. In some embodiments, the complementary sequence is a spacer sequence.

In some embodiments, decreasing or downregulating translation refers to a decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some embodiments of the systems described herein, the target mRNA comprises a PAM sequence. In other embodiments, the target mRNA does not comprise a PAM sequence. In some embodiments, the system comprises a PAMmer oligonucleotide. In other embodiments, the system does not comprise a PAMmer oligonucleotide.

### Methods

In some aspects, provided herein are methods for post-transcriptionally increasing or upregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an EIF4E protein, wherein the guide nucleotide sequence-programmable RNA binding protein binds a gRNA or a crRNA that hybridizes to a region of the target RNA.

In some embodiments, increasing or upregulating gene expression refers to an increase in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some aspects, provided herein are methods for post-transcriptionally decreasing or downregulating gene expression, the methods comprising, consisting of, or consisting essentially of contacting a target mRNA with a fusion protein comprising, consisting of, or consisting essentially of: (a) a guide nucleotide sequence-programmable RNA binding protein; and (b) an EIF4E-BP1 protein, wherein the guide nucleotide sequence-programmable RNA binding protein binds a gRNA or a crRNA that hybridizes to a region of the target RNA.

In some embodiments, decreasing or downregulating gene expression refers to a decrease in the amount of peptide translated from the target mRNA as compared to a control. In some embodiments, the control comprises a level of peptide translated from the target mRNA in the absence of the fusion protein. In some embodiments, the control comprises the level of the peptide translated from the target mRNA prior to addition of the fusion protein. In some embodiments, translation is decreased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

The amount of peptide translated can be determined by any method known in the art. Non-limiting examples of suitable methods of detection include Western blots, ELISAs, mass spectrometry, immunohistochemistry, immunofluorescence, and use of a reporter gene such as a fluorescence reporter gene.

In some embodiments of the methods described herein, the target mRNA comprises a PAM sequence. In other embodiments, the target mRNA does not comprise a PAM sequence. In some embodiments, the method further comprises providing a PAMmer oligonucleotide. In other embodiments, the method does not comprise providing a PAMmer oligonucleotide.

In some embodiments, the target mRNA is in a cell. In some embodiments, the cell is a eukaryotic cell. In some embodiments, the cell is a prokaryotic cell. In some embodiments, the cell is a mammalian cell. In some embodiments, the cell is a bovine, murine, feline, equine, porcine, canine, simian, or human cell. In some embodiments, the cell is a plant cell. In some embodiments, the cell is in a subject. In some embodiments, the cell is in vivo, in vitro, ex vivo, or in situ. In some embodiments, the composition comprises a vector comprising composition comprising a guide RNA of the disclosure and a fusion protein of the disclosure. In some embodiments, the vector is an AAV.

In some aspects, the disclosure provides a method of treating a disease or disorder comprising administering to a subject a therapeutically effective amount of a composition of the disclosure. Also provided herein are methods for treating a disease or condition in a subject in need thereof, the methods comprising, consisting of, or consisting essentially of administering a fusion protein comprising, consisting of, or consisting essentially of: (a) a guide nucleotide sequence-programmable RNA binding protein; and (b) an EIF4E-BP1 protein, a polynucleotide encoding the fusion protein, a vector comprising the polynucleotide encoding the fusion protein, or viral particle comprising the vector to the subject, thereby decreasing or downregulating translation of a target mRNA in the subject. In some embodiments, the target mRNA is involved in the etiology of a disease or condition in the subject.

In some aspects, also provided herein are methods for treating a disease or condition in a subject in need thereof, the methods comprising, consisting of, or consisting essentially of administering a fusion protein comprising, consisting of, or consisting essentially of: (a) a guide nucleotide sequence-programmable RNA binding protein; and (b) an EIF4E protein, a polynucleotide encoding the fusion protein, a vector comprising the polynucleotide encoding the fusion protein, or viral particle comprising the vector to the subject, thereby increasing or upregulating translation of a target mRNA in the subject. In some embodiments, a deficiency in the target mRNA is related to the etiology of a disease or condition in the subject.

In some embodiments of the methods described herein, the subject is a plant or an animal. In some embodiments, the subject is a mammal. In some embodiments, the mammal is a bovine, equine, porcine, canine, feline, simian, murine, or human. In some embodiments, the subject is a human.

In some embodiments of the methods described herein, the subject is further administered (i) a gRNA complementary to the target mRNA, or (ii) a crRNA complementary to the target mRNA and a tracrRNA. In some embodiments, the complementary sequence is a spacer sequence.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the disclosure, a disease or disorder of the disclosure includes, but is not limited to, an inflammatory disease or disorder.

In some embodiments of the compositions and methods of the disclosure, a disease or disorder of the disclosure includes, but is not limited to, a metabolic disease or disorder.

In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer , Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma , Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma , Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5,6, 7, 8, 9, 10, 11, 12, 13, 14, 15, 16, 17, 18, 19, 20, 21, 22, 23, 24, 25, 26, 27,28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal, or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

### Viral Particles

In some aspects, provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an EIF4E protein. In other aspects, provided herein are viral particles comprising, consisting of, or consisting essentially of a vector comprising, consisting of, or consisting essentially of a polynucleotide encoding a fusion protein comprising, consisting of, or consisting essentially of: (i) a guide nucleotide sequence-programmable RNA binding protein; and (ii) an EIF4E-BP1 protein. In some embodiments, the polynucleotides further comprise a nucleic acid sequence encoding a linker peptide.

In general, methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell. Similar vector based systems may employ other vectors such as sleeping beauty vectors or transposon elements.

The resulting packaged expression systems may then be introduced via an appropriate route of administration, discussed in detail with respect to the method aspects disclosed herein.

### Compositions

Also provided by this invention is a composition comprising any one or more of the fusion proteins, or the nucleic acid sequences encoding the fusion proteins, and a carrier. In some embodiments, a composition can be one or more polynucleotides encoding a guide nucleotide sequence-programmable RNA binding protein and a translation modifier protein. In some embodiments, a composition can be any of the fusion proteins described herein. In some embodiments, a composition can be any polynucleotide described herein. In some embodiments, the carrier is a pharmaceutically acceptable carrier. In some embodiments, the composition is a pharmaceutical composition comprising one or more fusion proteins, or one or more nucleic acid sequences encoding the fusion proteins, and a pharmaceutically acceptable carrier. In some embodiments, the composition or pharmaceutical composition further comprises one or more gRNAs, crRNAs, and/or tracrRNAs.

Briefly, pharmaceutical compositions of the present invention may comprise an fusion proteins or a polynucleotide encoding said fusion protein, optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

### Kits

In some aspects, provided herein are kits comprising, consisting of, or consisting essentially of one or more fusion proteins, polynucleotides encoding a fusion protein, vectors comprising the polynucleotide, or viral particles comprising the vector, wherein the fusion protein comprises, consists of, or consists essentially of: (a) a guide nucleotide sequence-programmable RNA binding protein; and (b) an EIF4E protein; or wherein the fusion protein comprises, consists of, or consists essentially of: (a) a guide nucleotide sequence-programmable RNA binding protein; and (b) an EIF4E-BP1 protein. In some embodiments, the kits further comprise, consist of, or consist essentially of instructions for use.

In some embodiments of the kits described herein, the kits further comprise, consist of, or consist essentially of one or more nucleic acids selected from: (i) a gRNA; (ii) a crRNA and a tracrRNA; (iii) a PAMmer oligonucleotide; and (iv) a vector for expressing the nucleic acid of (i), (ii), or (iii).

In some embodiments, the kits further comprise, consist of, or consist essentially of one or more reagents for carrying out a method of the disclosure. Non-limiting examples of such reagents comprise viral packaging cells, viral vectors, vector backbones, gRNAs, transfection reagents, transduction reagents, viral particles, and PCR primers. Accordingly, other embodiments are within the scope of the following claims.

### Example Embodiments

Embodiment 1 is a composition comprising one or more polynucleotides encoding:
   (i) a guide nucleotide sequence-programmable RNA binding protein; and
   (ii) a translation modifier protein.
Embodiment 2 is the composition of embodiment 1, wherein the guide nucleotide sequence-programmable RNA binding protein comprises at least one of Cas9, modified Cas9, Cas13a, Cas13b, CasRX/Cas13d, CasM and a biological equivalent of each thereof.
Embodiment 3 is the composition of embodiment 2, wherein the guide nucleotide sequence-programmable RNA binding protein comprises at least one of Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 4 is the composition of embodiment 2 or 3, wherein the guide nucleotide sequence-programmable RNA binding protein is nuclease inactive.
Embodiment 5 is the composition of any one of the preceding embodiments, wherein the translation modifier protein is at least one of translation initiation factor 4E (EIF4E) (SEQ ID NO: 52-59), eukaryotic translation initiation factor 4E-binding protein (EIF4E-BP1) (SEQ ID NO: 61-62), ubiquitin-associated protein 2-like (UBAP2L) (SEQ ID NO: 64-71), and a biological equivalent of each thereof.
Embodiment 6 is the composition of any one of the preceding embodiments, wherein the translation modifier protein is encoded by a polynucleotide having a sequence comprising all or part of at least one of SEQ ID NO: 52-55, SEQ ID NO: 61, SEQ ID NO: 64-67, SEQ ID NO: 94-193, SEQ ID NO: 285, SEQ ID NO: 320-348, and a biological equivalent of each thereof.
Embodiment 7 is the composition of any one of the preceding embodiments, wherein the translation modifier protein has an amino acid sequence comprising all or part of at least one of SEQ ID NO: 56-59, SEQ ID NO: 62, SEQ ID NO: 68-71, and a biological equivalent of each thereof.
Embodiment 8 is the composition of any one of the previous embodiments, further comprising a linker.
Embodiment 9 is the composition of embodiment 8, wherein the linker is a peptide linker.
Embodiment 10 is the composition of embodiment 9, wherein the peptide linker comprises one or more repeats of the tri-peptide GGS.
Embodiment 11 is the composition of embodiment 8, wherein the linker is a non-peptide linker.
Embodiment 12 is the composition of embodiment 11, wherein the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.
Embodiment 13 is the composition of any one of the preceding embodiments, wherein the guide nucleotide sequence-programmable RNA binding protein is bound to a guide RNA (gRNA), a crisprRNA (crRNA), or a trans-activating crRNA (tracrRNA).
Embodiment 14 is the composition of any one of the preceding embodiments, wherein one or more kinase phosphorylation domains of the eukaryotic translation modifier protein is mutated.
Embodiment 15 is the composition of any one of the preceding embodiments, further comprising a vector.
Embodiment 16 is the vector of embodiment 15, wherein the vector is an adenoviral vector, an adeno-associated viral vector, or a lentiviral vector.
Embodiment 17 is the vector of embodiment 15 or 16, further comprising an expression control element.
Embodiment 18 is the vector of embodiments 15-17, further comprising a selectable marker.
Embodiment 19 is the vector of any one of embodiments 15-18, further comprising a polynucleotide encoding either (i) a gRNA, or (ii) a crRNA and a tracrRNA.
Embodiment 20 is the vector of embodiment 19, wherein the gRNA or the crRNA comprises a nucleotide sequence complementary to a target RNA.
Embodiment 21 is a system for post-transcriptional gene regulation, the system comprising:
   (i) a composition according to any one of embodiments 1-20; and
   (ii) a gRNA; or
   (iii) a crRNA and a tracrRNA;
   wherein the gRNA or the crRNA comprises a sequence complementary to a target mRNA.
Embodiment 22 is a method for post-transcriptionally regulating gene expression, the method comprising contacting a target mRNA with a composition according to any one of embodiments 1-20, wherein the guide nucleotide sequence-programmable RNA binding protein binds a gRNA or a crRNA that hybridizes to a region of the target RNA.
Embodiment 23 is a fusion protein comprising:
   (i) a RNA binding protein; and
   (ii) a translation modifier protein.
Embodiment 24 is the fusion protein of embodiment 23, wherein the RNA binding protein is selected from a Pumilio and FBF (PUF) protein, a Pumilio-based assembly (PUMBY) protein, a pentatricopeptide repeat (PPR) protein, and a biological equivalent of each thereof.
Embodiment 25 is the fusion protein of embodiment 23, wherein the RNA binding protein is a Pumilio and FBF (PUF) protein.
Embodiment 26 is the fusion protein of embodiment 23, wherein the RNA binding protein is a Pumilio-based assembly (PUMBY) protein.
Embodiment 27 is the fusion protein of embodiment 23, wherein the RNA binding protein is a pentatricopeptide repeat (PPR) protein.
Embodiment 28 is the composition of any one of embodiments 23-27, wherein the translation modifier protein is at least one of translation initiation factor 4E (EIF4E) (SEQ ID NO: 52-59), eukaryotic translation initiation factor 4E-binding protein (EIF4E-BP1) (SEQ ID NO: 61-62), ubiquitin-associated protein 2-like (UBAP2L) (SEQ ID NO: 64-71), and a biological equivalent of each thereof.
Embodiment 29 is the composition of any one of embodiments 23-28, wherein the translation modifier protein is encoded by a polynucleotide having a sequence comprising all or part of at least one of SEQ ID NO: 52-55, SEQ ID NO: 61, SEQ ID NO: 64-67, SEQ ID NO: 94-193, SEQ ID NO: 285, SEQ ID NO: 320-348, and a biological equivalent of each thereof.
Embodiment 30 is the composition of any one of embodiments 23-29, wherein the translation modifier protein has an amino acid sequence comprising all or part of at least one of SEQ ID NO: 56-59, SEQ ID NO: 62, SEQ ID NO: 68-71, and a biological equivalent of each thereof.
Embodiment 31 is the composition of any one of the preceding embodiments, wherein the translatin modifier protein is eukaryotic.
Embodiment 32 is the composition of any one of the preceding embodiments, wherein the translatin modifier protein is human.
Embodiment 33 is the composition of any one of the preceding embodiments, wherein the translatin modifier protein is prokaryotic.
Embodiment 34 is a fusion protein comprising:
   (i) a guide nucleotide sequence-programmable RNA binding protein; and
   (ii) a eukaryotic translation initiation factor 4E (EIF4E) protein.
Embodiment 35 is the fusion protein of embodiment 34, wherein the guide nucleotide sequence-programmable RNA binding protein is selected from: Cas9, modified Cas9, Cas13a, Cas13b, CasRX/Cas13d, and a biological equivalent of each thereof. Embodiment 36 is the fusion protein of embodiment 35, wherein the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 37 is the fusion protein of embodiment 35 or 36, wherein the guide nucleotide sequence-programmable RNA binding protein is nuclease inactive.
Embodiment 38 is the fusion protein of any one of embodiments 34-37, further comprising a linker.
Embodiment 39 is the fusion protein of embodiment 38, wherein the linker is a peptide linker.
Embodiment 40 is the fusion protein of embodiment 39, wherein the peptide linker comprises one or more repeats of the tri-peptide GGS.
Embodiment 41 is the fusion protein of embodiment 38, wherein the linker is a non-peptide linker.
Embodiment 42 is the fusion protein of embodiment 41, wherein the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.
Embodiment 43 is the fusion protein of any one of embodiments 38-42, wherein the fusion protein comprises the structure NH₂-[EIF4E]-[linker]-[guide nucleotide sequence-programmable RNA binding protein]-COOH.
Embodiment 44 is the fusion protein of any one of embodiments 38-42, wherein the fusion protein comprises the structure NH₂-[guide nucleotide sequence-programmable RNA binding protein]-[linker]-[EIF4E]-COOH.
Embodiment 45 is the fusion protein of any one of embodiments 34-44, wherein the guide nucleotide sequence-programmable RNA binding protein is bound to a guide RNA (gRNA), a crisprRNA (crRNA), or a trans-activating crRNA (tracrRNA).
Embodiment 46 is the fusion protein of any one of embodiments 34-45, wherein the EIF4E protein is encoded by a polynucleotide having a sequence comprising all or part of a sequence selected from SEQ ID NO: 52, SEQ ID NO: 53, SEQ ID NO: 54, SEQ ID NO: 55, and a biological equivalent of each thereof.
Embodiment 47 is the fusion protein of any one of embodiments 34-46, wherein the EIF4E protein has an amino acid sequence comprising all or part of a sequence selected from SEQ ID NO: 56, SEQ ID NO: 57, SEQ ID NO: 58, SEQ ID NO: 59, and a biological equivalent of each thereof.
Embodiment 48 is the fusion protein of any one of embodiments 34-47, wherein one or more kinase phosphorylation domains of the EIF4E is mutated.
Embodiment 49 is the fusion protein of embodiment 48, wherein the mutated EIF4E is constituitively active.
Embodiment 50 is a fusion protein comprising:
   (i) a guide nucleotide sequence-programmable RNA binding protein; and
   (ii) a eukaryotic translation initiation factor 4E-binding protein 1 (EIF4E-BP1) protein.
Embodiment 51 is the fusion protein of embodiment 50, wherein the guide nucleotide sequence-programmable RNA binding protein is selected from: Cas9, modified Cas9, Cas13a, Cas13b, CasRX/Cas13d, and a biological equivalent of each thereof.
Embodiment 52 is the fusion protein of embodiment 51, wherein the guide nucleotide sequence-programmable RNA binding protein is selected from: Streptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 53 is the fusion protein of embodiment 51 or 52, wherein the guide nucleotide sequence-programmable RNA binding protein is nuclease inactive.
Embodiment 54 is the fusion protein of any one of embodiments 50-53, further comprising a linker.
Embodiment 55 is the fusion protein of embodiment 54, wherein the linker is a peptide linker.
Embodiment 56 is the fusion protein of embodiment 55, wherein the peptide linker comprises one or more repeats of the tri-peptide GGS.
Embodiment 57 is the fusion protein of embodiment 54, wherein the linker is a non-peptide linker.
Embodiment 58 is the fusion protein of embodiment 57, wherein the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.
Embodiment 59 is the fusion protein of any one of embodiments 54-58, wherein the fusion protein comprises the structure NH₂-[EIF4E-BP1]-[linker]-[guide nucleotide sequence-programmable RNA binding protein]-COOH.
Embodiment 60 is the fusion protein of any one of embodiments 54-58, wherein the fusion protein comprises the structure NH₂-[guide nucleotide sequence-programmable RNA binding protein]-[linker]-[EIF4E-BP1]-COOH.
Embodiment 61 is the fusion protein of any one of embodiments 50-60, wherein the guide nucleotide sequence-programmable RNA binding protein is bound to a guide RNA (gRNA), a crisprRNA (crRNA), or a trans-activating crRNA (tracrRNA).
Embodiment 62 is the fusion protein of any one of embodiments 50-61, wherein the EIF4E-BP1 protein is encoded by a polynucleotide having a sequence comprising all or part of SEQ ID NO: 61 or a biological equivalent thereof.
Embodiment 63 is the fusion protein of any one of embodiments 50-62, wherein the EIF4E-BP1 protein has an amino acid sequence comprising all or part of SEQ ID NO: 62 or a biological equivalent thereof.
Embodiment 64 is the fusion protein any one of embodiments 50-63, wherein one or more kinase phosphorylation domains of the EIF4E-BP1 protein is mutated.
Embodiment 65 is the fusion protein of embodiment 64, wherein the mutated EIF4E-BP1 is constituitively active.
Embodiment 66 is a polynucleotide encoding the fusion protein of any one of embodiments 34-65.
Embodiment 67 is a vector comprising the polynucleotide of embodiment 66, optionally wherein the vector is an adenoviral vector, an adeno-associated viral vector, or a lentiviral vector.
Embodiment 68 is the vector of embodiment 67, further comprising an expression control element.
Embodiment 69 is the vector of embodiment 67 or 68, further comprising a selectable marker.
Embodiment 70 is the vector of any one of embodiments 67-69, further comprising a polynucleotide encoding either (i) a gRNA, or (ii) a crRNA and a tracrRNA.
Embodiment 71 is the vector of embodiment 70, wherein the gRNA or the crRNA comprises a nucleotide sequence complementary to a target RNA.
Embodiment 72 is a viral particle comprising the fusion protein of any one of embodiments 34-65, the polynucleotide of embodiment 66, or the vector of any one of embodiments 67-71.
Embodiment 73 is a cell comprising the fusion protein of any one of embodiments 34-65, the polynucleotide of embodiment 66, the vector of any one of embodiments 67-71, or the viral particle of embodiment 72.
Embodiment 74 is the cell of embodiment 73, wherein the cell is a eukaryotic cell.
Embodiment 75 is the cell of embodiment 73, wherein the cell is a prokaryotic cell.
Embodiment 76 is the cell of embodiment 74, wherein the cell is a mammalian cell, optionally a bovine, murine, feline, equine, porcine, canine, simian, or human cell.
Embodiment 77 is a system for post-transcriptional gene regulation, the system comprising:
   (i) a fusion protein according to any one of embodiments 34-65; and
   (ii) a gRNA; or
   (iii) a crRNA and a tracrRNA;
   wherein the gRNA or the crRNA comprises a sequence complementary to a target mRNA.
Embodiment 78 is a system for increasing translation of a target mRNA, the system comprising:
   (i) a fusion protein according to any one of embodiments 34-39; and
   (ii) a gRNA; or
   (iii) a crRNA and a tracrRNA;
   wherein the gRNA or the crRNA comprises a sequence complementary to a target mRNA.
Embodiment 79 is a system for decreasing translation of a target mRNA, the system comprising:
   (i) a fusion protein according to any one of embodiments 50-65; and
   (ii) a gRNA; or
   (iii) a crRNA and a tracrRNA;
   wherein the gRNA or the crRNA comprises a sequence complementary to a target mRNA.
Embodiment 80 is the system of any one of embodiments 77-79, further comprising a PAMmer.
Embodiment 81 is the system of any one of embodiments 77-79, wherein the target mRNA does not comprise a PAM sequence or complement thereof.
Embodiment 82 is a method for post-transcriptionally increasing gene expression, the method comprising contacting a target mRNA with a fusion protein according to any one of embodiments 34-49, wherein the guide nucleotide sequence-programmable RNA binding protein binds a gRNA or a crRNA that hybridizes to a region of the target RNA.
Embodiment 83 is a method for post-transcriptionally decreasing gene expression, the method comprising contacting a target mRNA with a fusion protein according to any one of embodiments 50-65, wherein the guide nucleotide sequence-programmable RNA binding protein binds a gRNA or a crRNA that hybridizes to a region of the target RNA.
Embodiment 84 is the method of embodiment 82 or 83, wherein the target mRNA comprises a PAM sequence or complement thereof.
Embodiment 85 is the method of embodiment 82 or 83, wherein the target mRNA does not comprise a PAM sequence or complement thereof.
Embodiment 86 is the method of any one of embodiments 82-85, wherein the target mRNA is in a cell.
Embodiment 87 is the method of embodiment 86, wherein the cell is a eukaryotic cell.
Embodiment 88 is the method of embodiment 86, wherein the cell is a prokaryotic cell.
Embodiment 89 is the method of embodiment 87, wherein the eukaryotic cell is a mammalian cell, optionally a bovine, murine, feline, equine, porcine, canine, simian, or human cell.
Embodiment 90 is the method of any one of embodiments 86-89, wherein the cell is in a subject.
Embodiment 91 is a method for treating a disease or condition in a subject in need thereof, the method comprising administering the fusion protein of any one of embodiments 34-65, the polynucleotide of embodiment 66, the vector of any one of embodiments 67-71, or the viral particle of embodiment 72 to the subject, thereby increasing or decreasing translation of a target mRNA in the subject.
Embodiment 92 is the method of embodiment 90 or 91, wherein the subject is a human.
Embodiment 93 is the method of embodiment 91, further comprising administering to the subject: (i) a gRNA complementary to the mRNA, or (ii) a crRNA complementary to the mRNA and a tracrRNA.
Embodiment 94 is the method of embodiment 93, further comprising administering to the subject a PAMmer.
Embodiment 95 is a kit comprising one or more of: the fusion protein of any one of embodiments 34-65, the polynucleotide of embodiment 66, the vector of any one of embodiments 67-71, or the viral particle of embodiment 72 to the subject, and optionally instructions for use.
Embodiment 96 is the kit embodiment 95, further comprising one or more nucleic acids selected from:
   (i) a gRNA;
   (ii) a crRNA and a tracrRNA;
   (iii) a PAMmer; and
   (iv) a vector for expressing the nucleic acid of (i), (ii), or (iii).
Embodiment 97 is a non-human transgenic animal comprising a fusion protein or viral vector as described herein.
Embodiment 98 is a fusion RNA comprising:
   (i) a guide nucleotide sequence-programmable RNA; and
   (ii) one or more internal ribosome entry sites (IRES).
Embodiment 99 is the fusion RNA of embodiment 98, wherein the guide nucleotide sequence-programmable RNA is a guide RNA (gRNA) or a crisprRNA (crRNA).
Embodiment 100 is the fusion RNA of embodiment 99, wherein the guide nucleotide sequence-programmable RNA is derived from a guide RNA scaffold from Steptococcus pyogenes, Staphilococcus aureus, Francisella novicida, Neisseria meningitidis, Streptococcus thermophilus, or Brevibacillus laterosporus.
Embodiment 101 is the fusion RNA of any one of embodiments 98-100, wherein the IRES is a type I or a type II IRES.
Embodiment 102 is the fusion RNA of any one of embodiments 98-101, wherein the IRES is a viral IRES or a eukaryotic IRES.
Embodiment 103 is the fusion RNA of any one of embodiments 98-102, wherein the IRES is selected from a Poliovirus IRES, Rhinovirus IRES, Encephalomyocarditis virus IRES (EMCV-IRES), Picornavirus IRES, Foot-and-mouth disease virus IRES (FMDV-IRES), Aphthovirus IRES, Kaposi's sarcoma-associated herpesvirus IRES (KSHV-IRES), Hepatitis A IRES, Hepatitis C IRES, Classical swine fever virus IRES, Pestivirus IRES, Bovine viral diarrhea virus IRES, Friend murine leukemia IRES, Moloney murine leukemia IRES (MMLV-IRES), Rous sarcoma virus IRES, Human immunodeficiency virus IRES (HIV-IRES), Plautia stali intestine virus IRES, Cripavirus IRES, Cricket paralysis virus IRES, Triatoma virus IRES, Rhopalosiphum padi virus IRES, Marek's disease virus IRES, Fibroblast growth factor (FGF-1 IRES and FGF-2 IRES), Platelet-derived growth factor B (PDGF/c-sis IRES), Vascular endothelial growth factor (VEGF IRES), and an Insulin-like growth factor 2 (IGF-II IRES).
Embodiment 104 is the fusion RNA of any one of embodiments 98-103, further comprising a linker sequence RNA located between the guide nucleotide sequence-programmable RNA and the IRES.
Embodiment 105 is the fusion RNA of embodiment 104, wherein the fusion RNA comprises the structure 5′-[guide nucleotide sequence-programmable RNA]-[linker sequence]-[IRES]-3′.
Embodiment 106 is the fusion RNA of embodiment 104, wherein the fusion RNA comprises the structure 5′-[IRES]-[linker sequence]-[guide nucleotide sequence-programmable RNA]-3′.
Embodiment 107 is the fusion RNA of any one of embodiments 98-106, wherein the guide nucleotide sequence-programmable RNA comprises a nucleotide sequence complementary to a target RNA.
Embodiment 108 is a polynucleotide encoding the fusion RNA of any one of embodiments 98-107.
Embodiment 109 is a vector comprising the polynucleotide of embodiment 108, optionally wherein the vector is an adenoviral vector, an adeno-associated viral vector, or a lentiviral vector.
Embodiment 110 is the vector of embodiment 109, further comprising an expression control element.
Embodiment 111 is the vector of embodiment 109 or 110, further comprising a selectable marker.
Embodiment 112 is the vector of any one of embodiments 109-111, further comprising a polynucleotide encoding a tracrRNA.
Embodiment 113 is a viral particle comprising the fusion RNA of any one of embodiments 98-107, the polynucleotide of embodiment 108, or the vector of any one of embodiments 109-112.
Embodiment 114 is a cell comprising the fusion RNA of any one of embodiments 98-107, the polynucleotide of embodiment 108, the vector of any one of embodiments 109-112, or the viral particle of embodiment 113.
Embodiment 115 is the cell of embodiment 114, wherein the cell is a eukaryotic cell.
Embodiment 116 is the cell of embodiment 114, wherein the cell is a prokaryotic cell.
Embodiment 117 is the cell of embodiment 115, wherein the cell is a mammalian cell, optionally a bovine, murine, feline, equine, porcine, canine, simian, or human cell.
Embodiment 118 is a system for post-transcriptional gene regulation, the system comprising:
   (i) a fusion RNA according to any one of embodiments 98-107; and
   (ii) guide nucleotide sequence-programmable RNA binding protein,
      wherein the fusion RNA comprises a sequence complementary to a target mRNA.
Embodiment 119 is a system for increasing translation of a target mRNA, the system comprising:
   (i) a fusion RNA according to any one of embodiments 98-107; and
   (ii) guide nucleotide sequence-programmable RNA binding protein,
      wherein the fusion RNA comprises a sequence complementary to a target mRNA.
Embodiment 120 is the system of embodiment 118 or 119, further comprising a PAMmer.
Embodiment 121 is the system of embodiment 118 or 119, wherein the target mRNA does not comprise a PAM sequence or its complement.
Embodiment 122 is the system of any one of embodiments 118-121, wherein the guide nucleotide-sequence programmable RNA binding protein is selected from: Cas9, modified Cas9, Cas13a, Cas13b, CasRX/Cas13d, and a biological equivalent of each thereof.
Embodiment 123 is the system of any one of embodiments 118-122, wherein the guide nucleotide sequence-programmable RNA binding protein is selected from: Steptococcus pyogenes Cas9 (spCas9), Staphilococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 124 is the system of any one of embodiments 118-123, wherein the guide nucleotide sequence-programmable RNA binding protein is nuclease inactive.
Embodiment 125 is a method for post-transcriptionally increasing gene expression, the method comprising contacting a target mRNA with a fusion RNA according to any one of embodiments 98-107 and a guide nucleotide sequence-programmable RNA binding protein.
Embodiment 126 is a method for post-transcriptionally decreasing gene expression, the method comprising contacting a target mRNA with a fusion RNA according to any one of embodiments 98-107 and a guide nucleotide sequence-programmable RNA binding protein.
Embodiment 127 is the method of embodiment 125 or 126, further comprising contacting the guide nucleotide sequence-programmable RNA binding protein with a PAMmer.
Embodiment 128 is the method of embodiment 125 or 126, wherein the target mRNA does not comprise a PAM sequence.
Embodiment 129 is the method of any one of embodiments 125-128, wherein the guide nucleotide-sequence programmable RNA binding protein is selected from: Cas9, modified Cas9, Cas13a, Cas13b, CasRX/Cas13d, and a biological equivalent of each thereof.
Embodiment 130 is the method of any one of embodiments 125-129, wherein the guide nucleotide sequence-programmable RNA binding protein is selected from: Steptococcus pyogenes Cas9 (spCas9), Staphilococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 131 is the method of any one of embodiments 125-130, wherein the guide nucleotide sequence-programmable RNA binding protein is nuclease inactive.
Embodiment 132 is the method of any one of embodiments 125-131, wherein the target mRNA is in a cell.
Embodiment 133 is the method of embodiment 132, wherein the cell is a eukaryotic cell.
Embodiment 134 is the method of embodiment 132, wherein the cell is a prokaryotic cell.
Embodiment 135 is the method of embodiment 133, wherein the eukaryotic cell is a mammalian cell, optionally a bovine, murine, feline, equine, porcine, canine, simian, or human cell.
Embodiment 136 is the method of any one of embodiments 125-135, wherein the cell is in a subject.
Embodiment 137 is a method for treating a disease or condition in a subject in need thereof, the method comprising administering to the subject:
   (i) a guide nucleotide sequence-programmable RNA binding protein; and
   (ii) the fusion RNA of any one of embodiments 98-107, the polynucleotide of embodiment 108, the vector of any one of embodiments 109-112, or the viral particle of embodiment 113, wherein the fusion RNA is complementary to a target mRNA in the subject,
   thereby increasing translation of a target mRNA in the subject.
Embodiment 138 is the method of embodiment 137, wherein the subject is a human.
Embodiment 139 is the method of embodiment 137 or 138, further comprising administering to the subject one or more of: (i) tracrRNA and (ii) a PAMmer.
Embodiment 140 is a kit comprising one or more of: fusion RNA of any one of embodiments 98-107, the polynucleotide of embodiment 108, the vector of any one of embodiments 109-112, or the viral particle of embodiment 113, and optionally instructions for use.
Embodiment 141 is the kit embodiment 140, further comprising one or more nucleic acids selected from:
   (i) PAMmer;
   (ii) a tracrRNA; and
   (iii) a vector for expressing the nucleic acid of (i) or (ii).
Embodiment 142 is the kit embodiment 140 or 141, further comprising a guide nucleotide sequence-programmable RNA binding protein.
Embodiment 143 is a fusion protein comprising:
   (iii) a guide nucleotide sequence-programmable RNA binding protein; and
   (iv) a ubiquitin-associated protein 2-like (UBAP2L) protein.
Embodiment 144 is the fusion protein of embodiment 143, wherein the guide nucleotide sequence-programmable RNA binding protein is selected from: Cas9, modified Cas9, Cas13a, Cas13b, CasRX/Cas13d, and a biological equivalent of each thereof.
Embodiment 145 is the fusion protein of embodiment 144, wherein the guide nucleotide sequence-programmable RNA binding protein is selected from: Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).
Embodiment 146 is the fusion protein of embodiment 144 or 145, wherein the guide nucleotide sequence-programmable RNA binding protein is nuclease inactive.
Embodiment 147 is the fusion protein of any one of embodiments 143-146, further comprising a linker.
Embodiment 148 is the fusion protein of embodiment 147, wherein the linker is a peptide linker.
Embodiment 149 is the fusion protein of embodiment 148, wherein the peptide linker comprises one or more repeats of the tri-peptide GGS.
Embodiment 150 is the fusion protein of embodiment 147, wherein the linker is a non-peptide linker.
Embodiment 151 is the fusion protein of embodiment 150, wherein the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.
Embodiment 152 is the fusion protein of any one of embodiments 147-151, wherein the fusion protein comprises the structure NH₂-[UBAP2L]-[linker]-[guide nucleotide sequence-programmable RNA binding protein]-COOH.
Embodiment 153 is the fusion protein of any one of embodiments 147-151, wherein the fusion protein comprises the structure NH₂-[guide nucleotide sequence-programmable RNA binding protein]-[linker]-[UBAP2L]-COOH.
Embodiment 154 is the fusion protein of any one of embodiments 143-153, wherein the guide nucleotide sequence-programmable RNA binding protein is bound to a guide RNA (gRNA), a crisprRNA (crRNA), or a trans-activating crRNA (tracrRNA).
Embodiment 155 is the fusion protein of any one of embodiments 143-154, wherein the UBAP2L protein is encoded by a polynucleotide having a sequence comprising all or part of a sequence selected from SEQ ID NO: 64, SEQ ID NO: 65, SEQ ID NO: 66, SEQ ID NO: 67, and a biological equivalent of each thereof.
Embodiment 156 is the fusion protein of any one of embodiments 34-46, wherein the UBAP2L protein has an amino acid sequence comprising all or part of a sequence selected from SEQ ID NO: 68, SEQ ID NO: 69, SEQ ID NO: 70, SEQ ID NO: 71, and a biological equivalent of each thereof.
Embodiment 157 is the fusion protein of any one of embodiments 143-156, wherein one or more kinase phosphorylation domains of the UBAP2L is mutated.
Embodiment 158 is the fusion protein of embodiment 157, wherein the mutated UBAP2L is constituitively active.

### EXAMPLES

The following examples are non-limiting and illustrative procedures which can be used in various instances in carrying the disclosure into effect.

Exemplary polynucleotide and polypeptide sequences used in the examples described herein are listed in Table 3.

TABLE 3

| V5 Tag | GKPIPNPLLGLDST (SEQ ID NO: 73) |
|---|---|
| dCas9 | MDKKYSIGLAIGTNSVGWAVITDEYKVPSKKFKVLGNTDRHSIKK |
| | NLIGALLFDSGETAEATRLKRTARRRYTRRKNRICYLQEIFSNEM |
| | AKVDDSFFHRLEESFLVEEDKKHERHPIFGNIVDEVAYHEKYPTI |
| | YHLRKKLVDSTDKADLRLIYLALAHMIKFRGHFLIEGDLNPDNSD |
| | VDKLFIQLVQTYNQLFEENPINASGVDAKAILSARLSKSRRLENL |
| | IAQLPGEKKNGLFGNLIALSLGLTPNFKSNFDLAEDAKLQLSKDT |
| | YDDDLDNLLAQIGDQYADLFLAAKNLSDAILLSDILRVNTEITKA |
| | PLSASMIKRYDEHHQDLTLLKALVRQQLPEKYKEIFFDQSKNGYA |
| | GYIDGGASQEEFYKFIKPILEKMDGTEELLVKLNREDLLRKQRTF |
| | DNGSIPHQIHLGELHAILRRQEDFYPFLKDNREKIEKILTFRIPY |
| | YVGPLARGNSRFAWMTRKSEETITPWNFEEVVDKGASAQSFIERM |
| | TNFDKNLPNEKVLPKHSLLYEYFTVYNELTKVKYVTEGMRKPAFL |
| | SGEQKKAIVDLLFKTNRKVTVKQLKEDYFKKIECFDSVEISGVED |
| | RFNASLGTYHDLLKIIKDKDFLDNEENEDILEDIVLTLTLFEDRE |
| | MIEERLKTYAHLFDDKVMKQLKRRRYTGWGRLSRKLINGIRDKQS |
| | GKTILDFLKSDGFANRNFMQLIHDDSLTFKEDIQKAQVSGQGDSL |
| | HEHIANLAGSPAIKKGILQTVKVVDELVKVMGRHKPENIVIEMAR |
| | ENQTTQKGQKNSRERMKRIEEGIKELGSQILKEHPVENTQLQNEK |
| | LYLYYLQNGRDMYVDQELDINRLSDYDVDAIVPQSFLKDDSIDNK |
| | VLTRSDKNRGKSDNVPSEEVVKKMKNYWRQLLNAKLITQRKFDNL |
| | TKAERGGLSELDKAGFIKRQLVETRQITKHVAQILDSRMNTKYDE |
| | NDKLIREVKVITLKSKLVSDFRKDFQFYKVREINNYHHAHDAYLN |
| | AVVGTALIKKYPKLESEFVYGDYKVYDVRKMIAKSEQEIGKATAK |
| | YFFYSNIMNFFKTEITLANGEIRKRPLIETNGETGEIVWDKGRDF |
| | ATVRKVLSMPQVNIVKKTEVQTGGFSKESILPKRNSDKLIARKKD |
| | WDPKKYGGFDSPTVAYSVLVVAKVEKGKSKKLKSVKELLGITIME |
| | RSSFEKNPIDFLEAKGYKEVKKDLIIKLPKYSLFELENGRKRMLA |
| | SAGELQKGNELALPSKYVNFLYLASHYEKLKGSPEDNEQKQLFVE |
| | QHKHYLDEIIEQISEFSKRVILADANLDKVLSAYNKHRDKPIREQ |
| | AENIIHLFTLTNLGAPAAFKYFDTTIDRKRYTSTKEVLDATLIHQ |
| | SITGLYETRIDLSQLGGD(SEQ ID NO: 74) |
| NLS | MVSKGGSSDDEATADSQHAAPPKKKRKVGDPRVPVAT (SEQ ID |
| | NO: 75) |
| PB-TRE-V5- | ACTCTTCCTTTTTCAATATTATTGAAGCATTTATCAGGGTTATTG |
| rCas9- | TCTCATGAGCGGATACATATTTGAATGTATTTAGAAAAATAAACA |
| mut (EIF4E) EF | AATAGGGGTTCCGCGCACATTTCCCCGAAAAGTGCCACCTAAATT |
| NLS-Turq | GTAAGCGTTAATATTTTGTTAAAATTCGCGTTAAATTTTTGTTAA |
| | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTTCTACCGGGTAGGGGAGGCGCTTTTCCCAAGGC |
| | AGTCTGGAGCATGCGCTTTAGCAGCCCCGCTGGGCACTTGGCGCT |
| | ACACAAGTGGCCTCTGGCCTCGCACACATTCCACATCCACCGGTA |
| | GGCGCCAACCGGCTCCGTTCTTTGGTGGCCCCTTCGCGCCACCTT |
| | CTACTCCTCCCCTAGTCAGGAAGTTCCCCCCCGCCCCGCAGCTCG |
| | CGTCGTGCAGGACGTGACAAATGGAAGTAGCACGTCTCATTAGTC |
| | TCGTGCAGATGGACAGCACCGCTGAGCAATGGAAGCGGGTAGGCC |
| | TTTGGGGCAGCGGCCAATAGCAGCTTTGCTCCTTCGCTTTCTGGG |
| | CTCAGAGGCTGGGAAGGGGTGGGTCCGGGGGCGGGCTCAGGGGCG |
| | GGCTCAGGGGCGGGGCGGGCGCCCGAAGGTCCTCCGGAGGCCCGG |
| | CATTCTGCACGCTTCAAAAGCGCACGTCTGCCGCGCTGTTCTCCT |
| | CTTCCTCATCTCCGGGCCTTTCGACCTGCAGGTTAATTAAATGGG |
| | TAAGCCTATCCCTAACCCTCTCCTCGGTCTCGATTCTACGGCTAG |
| | CATGGACAAGAAGTACAGCATCGGCCTGGCCATCGGCACCAACTC |
| | TGTGGGCTGGGCCGTGATCACCGACGAGTACAAGGTGCCCAGCAA |
| | GAAATTCAAGGTGCTGGGCAACACCGACCGGCACAGCATCAAGAA |
| | GAACCTGATCGGCGCCCTGCTGTTCGACAGCGGAGAAACAGCCGA |
| | GGCCACCCGGCTGAAGAGAACCGCCAGAAGAAGATACACCAGACG |
| | GAAGAACCGGATCTGCTATCTGCAAGAGATCTTCAGCAACGAGAT |
| | GGCCAAGGTGGACGACAGCTTCTTCCACAGACTGGAAGAGTCCTT |
| | CCTGGTGGAAGAGGATAAGAAGCACGAGCGGCACCCCATCTTCGG |
| | CAACATCGTGGACGAGGTGGCCTACCACGAGAAGTACCCCACCAT |
| | CTACCACCTGAGAAAGAAACTGGTGGACAGCACCGACAAGGCCGA |
| | CCTGCGGCTGATCTATCTGGCCCTGGCCCACATGATCAAGTTCCG |
| | GGGCCACTTCCTGATCGAGGGCGACCTGAACCCCGACAACAGCGA |
| | CGTGGACAAGCTGTTCATCCAGCTGGTGCAGACCTACAACCAGCT |
| | GTTCGAGGAAAACCCCATCAACGCCAGCGGCGTGGACGCCAAGGC |
| | CATCCTGTCTGCCAGACTGAGCAAGAGCAGACGGCTGGAAAATCT |
| | GATCGCCCAGCTGCCCGGCGAGAAGAAGAATGGCCTGTTCGGCAA |
| | CCTGATTGCCCTGAGCCTGGGCCTGACCCCCAACTTCAAGAGCAA |
| | CTTCGACCTGGCCGAGGATGCCAAACTGCAGCTGAGCAAGGACAC |
| | CTACGACGACGACCTGGACAACCTGCTGGCCCAGATCGGCGACCA |
| | GTACGCCGACCTGTTTCTGGCCGCCAAGAACCTGTCCGACGCCAT |
| | CCTGCTGAGCGACATCCTGAGAGTGAACACCGAGATCACCAAGGC |
| | CCCCCTGAGCGCCTCTATGATCAAGAGATACGACGAGCACCACCA |
| | GGACCTGACCCTGCTGAAAGCTCTCGTGCGGCAGCAGCTGCCTGA |
| | GAAGTACAAAGAGATTTTCTTCGACCAGAGCAAGAACGGCTACGC |
| | CGGCTACATCGATGGCGGAGCCAGCCAGGAAGAGTTCTACAAGTT |
| | CATCAAGCCCATCCTGGAAAAGATGGACGGCACCGAGGAACTGCT |
| | CGTGAAGCTGAACAGAGAGGACCTGCTGCGGAAGCAGCGGACCTT |
| | CGACAACGGCAGCATCCCCCACCAGATCCACCTGGGAGAGCTGCA |
| | CGCCATTCTGCGGCGGCAGGAAGATTTTTACCCATTCCTGAAGGA |
| | CAACCGGGAAAAGATCGAGAAGATCCTGACCTTCCGCATCCCCTA |
| | CTACGTGGGCCCTCTGGCCAGGGGAAACAGCAGATTCGCCTGGAT |
| | GACCAGAAAGAGCGAGGAAACCATCACCCCCTGGAACTTCGAGGA |
| | AGTGGTGGACAAGGGCGCCAGCGCCCAGAGCTTCATCGAGCGGAT |
| | GACCAACTTCGATAAGAACCTGCCCAACGAGAAGGTGCTGCCCAA |
| | GCACAGCCTGCTGTACGAGTACTTCACCGTGTACAACGAGCTGAC |
| | CAAAGTGAAATACGTGACCGAGGGAATGAGAAAGCCCGCCTTCCT |
| | GAGCGGCGAGCAGAAAAAAGCCATCGTGGACCTGCTGTTCAAGAC |
| | CAACCGGAAAGTGACCGTGAAGCAGCTGAAAGAGGACTACTTCAA |
| | GAAAATCGAGTGCTTCGACTCCGTGGAAATCTCCGGCGTGGAAGA |
| | TCGGTTCAACGCCTCCCTGGGCACATACCACGATCTGCTGAAAAT |
| | TATCAAGGACAAGGACTTCCTGGACAATGAGGAAAACGAGGACAT |
| | TCTGGAAGATATCGTGCTGACCCTGACACTGTTTGAGGACAGAGA |
| | GATGATCGAGGAACGGCTGAAAACCTATGCCCACCTGTTCGACGA |
| | CAAAGTGATGAAGCAGCTGAAGCGGCGGAGATACACCGGCTGGGG |
| | CAGGCTGAGCCGGAAGCTGATCAACGGCATCCGGGACAAGCAGTC |
| | CGGCAAGACAATCCTGGATTTCCTGAAGTCCGACGGCTTCGCCAA |
| | CAGAAACTTCATGCAGCTGATCCACGACGACAGCCTGACCTTTAA |
| | AGAGGACATCCAGAAAGCCCAGGTGTCCGGCCAGGGCGATAGCCT |
| | GCACGAGCACATTGCCAATCTGGCCGGCAGCCCCGCCATTAAGAA |
| | GGGCATCCTGCAGACAGTGAAGGTGGTGGACGAGCTCGTGAAAGT |
| | GATGGGCCGGCACAAGCCCGAGAACATCGTGATCGAAATGGCCAG |
| | AGAGAACCAGACCACCCAGAAGGGACAGAAGAACAGCCGCGAGAG |
| | AATGAAGCGGATCGAAGAGGGCATCAAAGAGCTGGGCAGCCAGAT |
| | CCTGAAAGAACACCCCGTGGAAAACACCCAGCTGCAGAACGAGAA |
| | GCTGTACCTGTACTACCTGCAGAATGGGCGGGATATGTACGTGGA |
| | CCAGGAACTGGACATCAACCGGCTGTCCGACTACGATGTGGACGC |
| | TATCGTGCCTCAGAGCTTTCTGAAGGACGACTCCATCGATAACAA |
| | AGTGCTGACTCGGAGCGACAAGAACCGGGGCAAGAGCGACAACGT |
| | GCCCTCCGAAGAGGTCGTGAAGAAGATGAAGAACTACTGGCGCCA |
| | GCTGCTGAATGCCAAGCTGATTACCCAGAGGAAGTTCGACAATCT |
| | GACCAAGGCCGAGAGAGGCGGCCTGAGCGAACTGGATAAGGCCGG |
| | CTTCATCAAGAGACAGCTGGTGGAAACCCGGCAGATCACAAAGCA |
| | CGTGGCACAGATCCTGGACTCCCGGATGAACACTAAGTACGACGA |
| | GAACGACAAACTGATCCGGGAAGTGAAAGTGATCACCCTGAAGTC |
| | CAAGCTGGTGTCCGATTTCCGGAAGGATTTCCAGTTTTACAAAGT |
| | GCGCGAGATCAACAACTACCACCACGCCCACGACGCCTACCTGAA |
| | CGCCGTCGTGGGAACCGCCCTGATCAAAAAGTACCCTAAGCTGGA |
| | AAGCGAGTTCGTGTACGGCGACTACAAGGTGTACGACGTGCGGAA |
| | GATGATCGCCAAGAGCGAGCAGGAAATCGGCAAGGCTACCGCCAA |
| | GTACTTCTTCTACAGCAACATCATGAACTTTTTCAAGACCGAGAT |
| | TACCCTGGCCAACGGCGAGATCCGGAAGCGGCCTCTGATCGAGAC |
| | AAACGGCGAAACAGGCGAGATCGTGTGGGATAAGGGCCGGGACTT |
| | TGCCACCGTGCGGAAAGTGCTGTCTATGCCCCAAGTGAATATCGT |
| | GAAAAAGACCGAGGTGCAGACAGGCGGCTTCAGCAAAGAGTCTAT |
| | CCTGCCCAAGAGGAACAGCGACAAGCTGATCGCCAGAAAGAAGGA |
| | CTGGGACCCTAAGAAGTACGGCGGCTTCGACAGCCCCACCGTGGC |
| | CTATTCTGTGCTGGTGGTGGCCAAAGTGGAAAAGGGCAAGTCCAA |
| | GAAACTGAAGAGTGTGAAAGAGCTGCTGGGGATCACCATCATGGA |
| | AAGAAGCAGCTTCGAGAAGAATCCCATCGACTTTCTGGAAGCCAA |
| | GGGCTACAAAGAAGTGAAAAAGGACCTGATCATCAAGCTGCCTAA |
| | GTACTCCCTGTTCGAGCTGGAAAACGGCCGGAAGAGAATGCTGGC |
| | CTCTGCCGGCGAACTGCAGAAGGGAAACGAACTGGCCCTGCCCTC |
| | CAAATATGTGAACTTCCTGTACCTGGCCAGCCACTATGAGAAGCT |
| | GAAGGGCTCCCCCGAGGATAATGAGCAGAAACAGCTGTTTGTGGA |
| | ACAGCACAAACACTACCTGGACGAGATCATCGAGCAGATCAGCGA |
| | GTTCTCCAAGAGAGTGATCCTGGCCGACGCTAATCTGGACAAGGT |
| | GCTGAGCGCCTACAACAAGCACAGAGACAAGCCTATCAGAGAGCA |
| | GGCCGAGAATATCATCCACCTGTTTACCCTGACCAATCTGGGAGC |
| | CCCTGCCGCCTTCAAGTACTTTGACACCACCATCGACCGGAAGAG |
| | GTACACCAGCACCAAAGAGGTGCTGGACGCCACCCTGATCCACCA |
| | GAGCATCACCGGCCTGTACGAGACACGGATCGACCTGTCTCAGCT |
| | GGGAGGCGACCTCGAGGGCGGATCCGGTGGTTCCGGAGGAGCTGT |
| | CGACATGGCGACTGTCGAACCGGAAACCACCCCTACTCCTAATCC |
| | CCCGACTACAGAAGAGGAGAAAACGGAATCTAATCAGGAGGTTGC |
| | TAACCCAGAACACTATATTAAACGGCCCCTACAGAACAGATGGGC |
| | ACTCTGGTTTTTTAAAAATGATAAAAGCAAAACTTGGCAAGCAAA |
| | CCTGCGGCTGATCTCCAAGTTTGATACTGCTGAAGACTTTTTTGC |
| | TCTGTACAACCATATCCAGTTGTCTAGTAATTTAATGCCTGGCTG |
| | TGACTACTCACTTTTTAAGGATGGTATTGAGCCTATGTGGGAAGA |
| | TGAGAAAAACAAACGGGGAGGACGATGGCTAATTACATTGAACAA |
| | ACAGCAGAGACGAAGTGACCTCGATCGCTTTTGGCTAGAGACACT |
| | TCTGTGCCTTATTGGAGAATCTTTTGATGACTACAGTGATGATGT |
| | ATGTGGCGCTGTTGTTAATGTTAGAGCTAAAGGTGATAAGATAGC |
| | AATATGGACTACTGAATGTGAAAACAGAGAAGCTGTTACACATAT |
| | AGGGAGGGTATACAAGGAAAGGTTAGGACTTCCTCCAAAGATAGT |
| | GATTGGTTATCAGTCCCACGCAGACACAGCTACTAAGAGCGGCGA |
| | CACCACTAAAAATAGGTTTGTTGTTTCTAGACTTAAGTAAGCCTC |
| | GACTGTGCCTTCTAGTTGCCAGCCATCTGTTGTTTGCCCCTCCCC |
| | CGTGCCTTCCTTGACCCTGGAAGGTGCCACTCCCACTGTCCTTTC |
| | CTAATAAAATGAGGAAATTGCATCGCATTGTCTGAGTAGGTGTCA |
| | TTCTATTCTGGGGGGTGGGGTGGGGCAGGACAGCAAGGGGGAGGA |
| | TTGGGAAGACAATAGCAGGCATGCTGGGGAGTGCCCGTCAGTGGG |
| | CAGAGCGCACATCGCCCACAGTCCCCGAGAAGTTGGGGGGAGGGG |
| | TCGGCAATTGAACGGGTGCCTAGAGAAGGTGGCGCGGGGTAAACT |
| | GGGAAAGTGATGTCGTGTACTGGCTCCGCCTTTTTCCCGAGGGTG |
| | GGGGAGAACCGTATATAAGTGCAGTAGTCGCCGTGAACGTTCTTT |
| | TTCGCAACGGGTTTGCCGCCAGAACACAGATGGTCTCTAAAGGAG |
| | GTTCGTCCGACGACGAAGCAACAGCGGACTCGCAGCACGCCGCAC |
| | CTCCTAAGAAGAAAAGGAAGGTAGGGGATCCCCGGGTACCGGTCG |
| | CCACCATGGTGAGCAAGGGCGAGGAGCTGTTCACCGGGGTGGTGC |
| | CCATCCTGGTCGAGCTGGACGGCGACGTAAACGGCCACAAGTTCA |
| | GCGTGTCCGGCGAGGGCGAGGGCGATGCCACCTACGGCAAGCTGA |
| | CCCTGAAGTTCATCTGCACCACCGGCAAGCTGCCCGTGCCCTGGC |
| | CCACCCTCGTGACCACCCTGTCCTGGGGCGTGCAGTGCTTCGCCC |
| | GCTACCCCGACCACATGAAGCAGCACGACTTCTTCAAGTCCGCCA |
| | TGCCCGAAGGCTACGTCCAGGAGCGCACCATCTTCTTCAAGGACG |
| | ACGGCAACTACAAGACCCGCGCCGAGGTGAAGTTCGAGGGCGACA |
| | CCCTGGTGAACCGCATCGAGCTGAAGGGCATCGACTTCAAGGAGG |
| | ACGGCAACATCCTGGGGCACAAGCTGGAGTACAACTACTTTAGCG |
| | ACAACGTCTATATCACCGCCGACAAGCAGAAGAACGGCATCAAGG |
| | CCAACTTCAAGATCCGCCACAACATCGAGGACGGCGGCGTGCAGC |
| | TCGCCGACCACTACCAGCAGAACACCCCCATCGGCGACGGCCCCG |
| | TGCTGCTGCCCGACAACCACTACCTGAGCACCCAGTCCAAGCTGA |
| | GCAAAGACCCCAACGAGAAGCGCGATCACATGGTCCTGCTGGAGT |
| | TCGTGACCGCCGCCGGGATCACTCTCGGCATGGACGAGCTGTACA |
| | AGTAAAATCAACCTCTGGATTACAAAATTTGTGAAAGATTGACTG |
| | GTATTCTTAACTATGTTGCTCCTTTTACGCTATGTGGATACGCTG |
| | CTTTAATGCCTTTGTATCATGTTAACTAAACTTGTTTATTGCAGC |
| | TTATAATGGTTACAAATAAAGCAATAGCATCACAAATTTCACAAA |
| | TAAAGCATTTTTTTCACTGCATTCTAGTTGTGGTTTGTCCAAACT |
| | CATCAATGTATCTTATCATGTCTGGAATTGACTCAAATGATGTCA |
| | ATTAGTCTATCAGAAGCTCATCTGGTCTCCCTTCCGGGGGACAAG |
| | ACATCCCTGTTTAATATTTAAACAGCAGTGTTCCCAAACTGGGTT |
| | CTTATATCCCTTGCTCTGGTCAACCAGGTTGCAGGGTTTCCTGTC |
| | CTCACAGGAACGAAGTCCCTAAAGAAACAGTGGCAGCCAGGTTTA |
| | GCCCCGGAATTGACTGGATTCCTTTTTTAGGGCCCATTGGTATGG |
| | CTTTTTCCCCGTATCCCCCCAGGTGTCTGCAGGCTCAAAGAGCAG |
| | CGAGAAGCGTTCAGAGGAAAGCGATCCCGTGCCACCTTCCCCGTG |
| | CCCGGGCTGTCCCCGCACGCTGCCGGCTCGGGGATGCGGGGGGAG |
| | CGCCGGACCGGAGCGGAGCCCCGGGCGGCTCGCTGCTGCCCCCTA |
| | GCGGGGGAGGGACGTAATTACATCCCTGGGGGCTTTGGGGGGGGG |
| | CTGTCCCTGATATCTATAACAAGAAAATATATATATAATAAGTTA |
| | TCACGTAAGTAGAACATGAAATAACAATATAATTATCGTATGAGT |
| | TAAATCTTAAAAGTCACGTAAAAGATAATCATGCGTCATTTTGAC |
| | TCACGCGGTCGTTATAGTTCAAAATCAGTGACACTTACCGCATTG |
| | ACAAGCACGCCTCACGGGAGCTCCAAGCGGCGACTGAGATGTCCT |
| | AAATGCACAGCGACGGATTCGCGCTATTTAGAAAGAGAGAGCAAT |
| | ATTTCAAGAATGCATGCGTCAATTTTACGCAGACTATCTTTCTAG |
| | GGTTAATCTAGCTGCATCAGGATCATATCGTCGGGTCTTTTTTCC |
| | GGCTCAGTCATCGCCCAAGCTGGCGCTATCTGGGCATCGGGGAGG |
| | AAGAAGCCCGTGCCTTTTCCCGCGAGGTTGAAGCGGCATGGAAAG |
| | AGTTTGCCGAGGATGACTGCTGCTGCATTGACGTTGAGCGAAAAC |
| | GCACGTTTACCATGATGATTCGGGAAGGTGTGGCCATGCACGCCT |
| | TTAACGGTGAACTGTTCGTTCAGGCCACCTGGGATACCAGTTCGT |
| | CGCGGCTTTTCCGGACACAGTTCCGGATGGTCAGCCCGAAGCGCA |
| | TCAGCAACCCGAACAATACCGGCGACAGCCGGAACTGCCGTGCCG |
| | GTGTGCAGATTAATGACAGCGGTGCGGCGCTGGGATATTACGTCA |
| | GCGAGGACGGGTATCCTGGCTGGATGCCGCAGAAATGGACATGGA |
| | TACCCCGTGAGTTACCCGGCGGGCGCGCTTGGCGTAATCATGGTC |
| | ATAGCTGTTTCCTGTGTGAAATTGTTATCCGCTCACAATTCCACA |
| | CAACATACGAGCCGGAAGCATAAAGTGTAAAGCCTGGGGTGCCTA |
| | ATGAGTGAGCTAACTCACATTAATTGCGTTGCGCTCACTGCCCGC |
| | TTTCCAGTCGGGAAACCTGTCGTGCCAGCTGCATTAATGAATCGG |
| | CCAACGCGCGGGGAGAGGCGGTTTGCGTATTGGGCGCTCTTCCGC |
| | TTCCTCGCTCACTGACTCGCTGCGCTCGGTCGTTCGGCTGCGGCG |
| | AGCGGTATCAGCTCACTCAAAGGCGGTAATACGGTTATCCACAGA |
| | ATCAGGGGATAACGCAGGAAAGAACATGTGAGCAAAAGGCCAGCA |
| | AAAGGCCAGGAACCGTAAAAAGGCCGCGTTGCTGGCGTTTTTCCA |
| | TAGGCTCCGCCCCCCTGACGAGCATCACAAAAATCGACGCTCAAG |
| | TCAGAGGTGGCGAAACCCGACAGGACTATAAAGATACCAGGCGTT |
| | TCCCCCTGGAAGCTCCCTCGTGCGCTCTCCTGTTCCGACCCTGCC |
| | GCTTACCGGATACCTGTCCGCCTTTCTCCCTTCGGGAAGCGTGGC |
| | GCTTTCTCATAGCTCACGCTGTAGGTATCTCAGTTCGGTGTAGGT |
| | CGTTCGCTCCAAGCTGGGCTGTGTGCACGAACCCCCCGTTCAGCC |
| | CGACCGCTGCGCCTTATCCGGTAACTATCGTCTTGAGTCCAACCC |
| | GGTAAGACACGACTTATCGCCACTGGCAGCAGCCACTGGTAACAG |
| | GATTAGCAGAGCGAGGTATGTAGGCGGTGCTACAGAGTTCTTGAA |
| | GTGGTGGCCTAACTACGGCTACACTAGAAGGACAGTATTTGGTAT |
| | CTGCGCTCTGCTGAAGCCAGTTACCTTCGGAAAAAGAGTTGGTAG |
| | CTCTTGATCCGGCAAACAAACCACCGCTGGTAGCGGTGGTTTTTT |
| | TGTTTGCAAGCAGCAGATTACGCGCAGAAAAAAAGGATCTCAAGA |
| | AGATCCTTTGATCTTTTCTACGGGGTCTGACGCTCAGTGGAACGA |
| | AAACTCACGTTAAGGGATTTTGGTCATGAGATTATCAAAAAGGAT |
| | CTTCACCTAGATCCTTTTAAATTAAAAATGAAGTTTTAAATCAAT |
| | CTAAAGTATATATGAGTAAACTTGGTCTGACAGTTACCAATGCTT |
| | AATCAGTGAGGCACCTATCTCAGCGATCTGTCTATTTCGTTCATC |
| | CATAGTTGCCTGACTCCCCGTCGTGTAGATAACTACGATACGGGA |
| | GGGCTTACCATCTGGCCCCAGTGCTGCAATGATACCGCGAGACCC |
| | ACGCTCACCGGCTCCAGATTTATCAGCAATAAACCAGCCAGCCGG |
| | AAGGGCCGAGCGCAGAAGTGGTCCTGCAACTTTATCCGCCTCCAT |
| | CCAGTCTATTAATTGTTGCCGGGAAGCTAGAGTAAGTAGTTCGCC |
| | AGTTAATAGTTTGCGCAACGTTGTTGCCATTGCTACAGGCATCGT |
| | GGTGTCACGCTCGTCGTTTGGTATGGCTTCATTCAGCTCCGGTTC |
| | CCAACGATCAAGGCGAGTTACATGATCCCCCATGTTGTGCAAAAA |
| | AGCGGTTAGCTCCTTCGGTCCTCCGATCGTTGTCAGAAGTAAGTT |
| | GGCCGCAGTGTTATCACTCATGGTTATGGCAGCACTGCATAATTC |
| | TCTTACTGTCATGCCATCCGTAAGATGCTTTTCTGTGACTGGTGA |
| | GTACTCAACCAAGTCATTCTGAGAATAGTGTATGCGGCGACCGAG |
| | TTGCTCTTGCCCGGCGTCAATACGGGATAATACCGCGCCACATAG |
| | CAGAACTTTAAAAGTGCTCATCATTGGAAAACGTTCTTCGGGGCG |
| | AAAACTCTCAAGGATCTTACCGCTGTTGAGATCCAGTTCGATGTA |
| | ACCCACTCGTGCACCCAACTGATCTTCAGCATCTTTTACTTTCAC |
| | CAGCGTTTCTGGGTGAGCAAAAACAGGAAGGCAAAATGCCGCAAA |
| | AAAGGGAATAAGGGCGACACGGAAATGTTGAATACTCAT (SEQ |
| | ID NO: 76) |
| EIF4E-BP1 | MSGGSSCSQTPSAAAAATRRVVLGAGVQLPPGDYSTAPGGTLFST |
| | APGGTRIIYDRKFLMECRNAPVTKAPPRDLPTIPGVTSPSSDEPP |
| | MEASQSHLRNSPEDKRAGGEESQAEMDI (SEQ ID NO: 77) |
| PB-TRE-V5- | ACTCTTCCTTTTTCAATATTATTGAAGCATTTATCAGGGTTATTGTCTCA |
| rCas9-mut (c- | TGAGCGGATACATATTTGAATGTATTTAGAAAAATAAACAAATAGGGGTT |
| 4EBP1-phosMUT) | CCGCGCACATTTCCCCGAAAAGTGCCACCTAAATTGTAAGCGTTAATATT |
| EF1a NLS-Turq | TTGTTAAAATTCGCGTTAAATTTTTGTTAAATCAGCTCATTTTTTAACCA |
| | ATAGGCCGAAATCGGCAAAATCCCTTATAAATCAAAAGAATAGACCGAGA |
| | TAGGGTTGAGTGTTGTTCCAGTTTGGAACAAGAGTCCACTATTAAAGAAC |
| | GTGGACTCCAACGTCAAAGGGCGAAAAACCGTCTATCAGGGCGATGGCCC |
| | ACTACGTGAACCATCACCCTAATCAAGTTTTTTGGGGTCGAGGTGCCGTA |
| | AAGCACTAAATCGGAACCCTAAAGGGAGCCCCCGATTTAGAGCTTGACGG |
| | GGAAAGCCGGCGAACGTGGCGAGAAAGGAAGGGAAGAAAGCGAAAGGAGC |
| | GGGCGCTAGGGCGCTGGCAAGTGTAGCGGTCACGCTGCGCGTAACCACCA |
| | CACCCGCCGCGCTTAATGCGCCGCTACAGGGCGCGTCCCATTCGCCATTC |
| | AGGCTGCGCAACTGTTGGGAAGGGCGATCGGTGCGGGCCTCTTCGCTATT |
| | ACGCCAGCTGGCGAAAGGGGGATGTGCTGCAAGGCGATTAAGTTGGGTAA |
| | CGCCAGGGTTTTCCCAGTCACGACGTTGTAAAACGACGGCCAGTGAGCGC |
| | GCCTCGTTCATTCACGTTTTTGAACCCGTGGAGGACGGGCAGACTCGCGG |
| | TGCAAATGTGTTTTACAGCGTGATGGAGCAGATGAAGATGCTCGACACGC |
| | TGCAGAACACGCAGCTAGATTAACCCTAGAAAGATAATCATATTGTGACG |
| | TACGTTAAAGATAATCATGTGTAAAATTGACGCATGTGTTTTATCGGTCT |
| | GTATATCGAGGTTTATTTATTAATTTGAATAGATATTAAGTTTTATTATA |
| | TTTACACTTACATACTAATAATAAATTCAACAAACAATTTATTTATGTTT |
| | ATTTATTTATTAAAAAAAACAAAAACTCAAAATTTCTTCTATAAAGTAAC |
| | AAAACTTTTATGAGGGACAGCCCCCCCCCAAAGCCCCCAGGGATGTAATT |
| | ACGTCCCTCCCCCGCTAGGGGGCAGCAGCGAGCCGCCCGGGGCTCCGCTC |
| | CGGTCCGGCGCTCCCCCCGCATCCCCGAGCCGGCAGCGTGCGGGGACAGC |
| | CCGGGCACGGGGAAGGTGGCACGGGATCGCTTTCCTCTGAACGCTTCTCG |
| | CTGCTCTTTGAGCCTGCAGACACCTGGGGGGATACGGGGAAAAGGCCTCC |
| | ACGGCCACTAGTTCTACCGGGTAGGGGAGGCGCTTTTCCCAAGGCAGTCT |
| | GGAGCATGCGCTTTAGCAGCCCCGCTGGGCACTTGGCGCTACACAAGTGG |
| | CCTCTGGCCTCGCACACATTCCACATCCACCGGTAGGCGCCAACCGGCTC |
| | CGTTCTTTGGTGGCCCCTTCGCGCCACCTTCTACTCCTCCCCTAGTCAGG |
| | AAGTTCCCCCCCGCCCCGCAGCTCGCGTCGTGCAGGACGTGACAAATGGA |
| | AGTAGCACGTCTCATTAGTCTCGTGCAGATGGACAGCACCGCTGAGCAAT |
| | GGAAGCGGGTAGGCCTTTGGGGCAGCGGCCAATAGCAGCTTTGCTCCTTC |
| | GCTTTCTGGGCTCAGAGGCTGGGAAGGGGTGGGTCCGGGGGCGGGCTCAG |
| | GGGCGGGCTCAGGGGCGGGGCGGGCGCCCGAAGGTCCTCCGGAGGCCCGG |
| | CATTCTGCACGCTTCAAAAGCGCACGTCTGCCGCGCTGTTCTCCTCTTCC |
| | TCATCTCCGGGCCTTTCGACCTGCAGGTTAATTAAATGGGTAAGCCTATC |
| | CCTAACCCTCTCCTCGGTCTCGATTCTACGGCTAGCATGGACAAGAAGTA |
| | CAGCATCGGCCTGGCCATCGGCACCAACTCTGTGGGCTGGGCCGTGATCA |
| | CCGACGAGTACAAGGTGCCCAGCAAGAAATTCAAGGTGCTGGGCAACACC |
| | GACCGGCACAGCATCAAGAAGAACCTGATCGGCGCCCTGCTGTTCGACAG |
| | CGGAGAAACAGCCGAGGCCACCCGGCTGAAGAGAACCGCCAGAAGAAGAT |
| | ACACCAGACGGAAGAACCGGATCTGCTATCTGCAAGAGATCTTCAGCAAC |
| | GAGATGGCCAAGGTGGACGACAGCTTCTTCCACAGACTGGAAGAGTCCTT |
| | CCTGGTGGAAGAGGATAAGAAGCACGAGCGGCACCCCATCTTCGGCAACA |
| | TCGTGGACGAGGTGGCCTACCACGAGAAGTACCCCACCATCTACCACCTG |
| | AGAAAGAAACTGGTGGACAGCACCGACAAGGCCGACCTGCGGCTGATCTA |
| | TCTGGCCCTGGCCCACATGATCAAGTTCCGGGGCCACTTCCTGATCGAGG |
| | GCGACCTGAACCCCGACAACAGCGACGTGGACAAGCTGTTCATCCAGCTG |
| | GTGCAGACCTACAACCAGCTGTTCGAGGAAAACCCCATCAACGCCAGCGG |
| | CGTGGACGCCAAGGCCATCCTGTCTGCCAGACTGAGCAAGAGCAGACGGC |
| | TGGAAAATCTGATCGCCCAGCTGCCCGGCGAGAAGAAGAATGGCCTGTTC |
| | GGCAACCTGATTGCCCTGAGCCTGGGCCTGACCCCCAACTTCAAGAGCAA |
| | CTTCGACCTGGCCGAGGATGCCAAACTGCAGCTGAGCAAGGACACCTACG |
| | ACGACGACCTGGACAACCTGCTGGCCCAGATCGGCGACCAGTACGCCGAC |
| | CTGTTTCTGGCCGCCAAGAACCTGTCCGACGCCATCCTGCTGAGCGACAT |
| | CCTGAGAGTGAACACCGAGATCACCAAGGCCCCCCTGAGCGCCTCTATGA |
| | TCAAGAGATACGACGAGCACCACCAGGACCTGACCCTGCTGAAAGCTCTC |
| | GTGCGGCAGCAGCTGCCTGAGAAGTACAAAGAGATTTTCTTCGACCAGAG |
| | CAAGAACGGCTACGCCGGCTACATCGATGGCGGAGCCAGCCAGGAAGAGT |
| | TCTACAAGTTCATCAAGCCCATCCTGGAAAAGATGGACGGCACCGAGGAA |
| | CTGCTCGTGAAGCTGAACAGAGAGGACCTGCTGCGGAAGCAGCGGACCTT |
| | CGACAACGGCAGCATCCCCCACCAGATCCACCTGGGAGAGCTGCACGCCA |
| | TTCTGCGGCGGCAGGAAGATTTTTACCCATTCCTGAAGGACAACCGGGAA |
| | AAGATCGAGAAGATCCTGACCTTCCGCATCCCCTACTACGTGGGCCCTCT |
| | GGCCAGGGGAAACAGCAGATTCGCCTGGATGACCAGAAAGAGCGAGGAAA |
| | CCATCACCCCCTGGAACTTCGAGGAAGTGGTGGACAAGGGCGCCAGCGCC |
| | CAGAGCTTCATCGAGCGGATGACCAACTTCGATAAGAACCTGCCCAACGA |
| | GAAGGTGCTGCCCAAGCACAGCCTGCTGTACGAGTACTTCACCGTGTACA |
| | ACGAGCTGACCAAAGTGAAATACGTGACCGAGGGAATGAGAAAGCCCGCC |
| | TTCCTGAGCGGCGAGCAGAAAAAAGCCATCGTGGACCTGCTGTTCAAGAC |
| | CAACCGGAAAGTGACCGTGAAGCAGCTGAAAGAGGACTACTTCAAGAAAA |
| | TCGAGTGCTTCGACTCCGTGGAAATCTCCGGCGTGGAAGATCGGTTCAAC |
| | GCCTCCCTGGGCACATACCACGATCTGCTGAAAATTATCAAGGACAAGGA |
| | CTTCCTGGACAATGAGGAAAACGAGGACATTCTGGAAGATATCGTGCTGA |
| | CCCTGACACTGTTTGAGGACAGAGAGATGATCGAGGAACGGCTGAAAACC |
| | TATGCCCACCTGTTCGACGACAAAGTGATGAAGCAGCTGAAGCGGCGGAG |
| | ATACACCGGCTGGGGCAGGCTGAGCCGGAAGCTGATCAACGGCATCCGGG |
| | ACAAGCAGTCCGGCAAGACAATCCTGGATTTCCTGAAGTCCGACGGCTTC |
| | GCCAACAGAAACTTCATGCAGCTGATCCACGACGACAGCCTGACCTTTAA |
| | AGAGGACATCCAGAAAGCCCAGGTGTCCGGCCAGGGCGATAGCCTGCACG |
| | AGCACATTGCCAATCTGGCCGGCAGCCCCGCCATTAAGAAGGGCATCCTG |
| | CAGACAGTGAAGGTGGTGGACGAGCTCGTGAAAGTGATGGGCCGGCACAA |
| | GCCCGAGAACATCGTGATCGAAATGGCCAGAGAGAACCAGACCACCCAGA |
| | AGGGACAGAAGAACAGCCGCGAGAGAATGAAGCGGATCGAAGAGGGCATC |
| | AAAGAGCTGGGCAGCCAGATCCTGAAAGAACACCCCGTGGAAAACACCCA |
| | GCTGCAGAACGAGAAGCTGTACCTGTACTACCTGCAGAATGGGCGGGATA |
| | TGTACGTGGACCAGGAACTGGACATCAACCGGCTGTCCGACTACGATGTG |
| | GACGCTATCGTGCCTCAGAGCTTTCTGAAGGACGACTCCATCGATAACAA |
| | AGTGCTGACTCGGAGCGACAAGAACCGGGGCAAGAGCGACAACGTGCCCT |
| | CCGAAGAGGTCGTGAAGAAGATGAAGAACTACTGGCGCCAGCTGCTGAAT |
| | GCCAAGCTGATTACCCAGAGGAAGTTCGACAATCTGACCAAGGCCGAGAG |
| | AGGCGGCCTGAGCGAACTGGATAAGGCCGGCTTCATCAAGAGACAGCTGG |
| | TGGAAACCCGGCAGATCACAAAGCACGTGGCACAGATCCTGGACTCCCGG |
| | ATGAACACTAAGTACGACGAGAACGACAAACTGATCCGGGAAGTGAAAGT |
| | GATCACCCTGAAGTCCAAGCTGGTGTCCGATTTCCGGAAGGATTTCCAGT |
| | TTTACAAAGTGCGCGAGATCAACAACTACCACCACGCCCACGACGCCTAC |
| | CTGAACGCCGTCGTGGGAACCGCCCTGATCAAAAAGTACCCTAAGCTGGA |
| | AAGCGAGTTCGTGTACGGCGACTACAAGGTGTACGACGTGCGGAAGATGA |
| | TCGCCAAGAGCGAGCAGGAAATCGGCAAGGCTACCGCCAAGTACTTCTTC |
| | TACAGCAACATCATGAACTTTTTCAAGACCGAGATTACCCTGGCCAACGG |
| | CGAGATCCGGAAGCGGCCTCTGATCGAGACAAACGGCGAAACAGGCGAGA |
| | TCGTGTGGGATAAGGGCCGGGACTTTGCCACCGTGCGGAAAGTGCTGTCT |
| | ATGCCCCAAGTGAATATCGTGAAAAAGACCGAGGTGCAGACAGGCGGCTT |
| | CAGCAAAGAGTCTATCCTGCCCAAGAGGAACAGCGACAAGCTGATCGCCA |
| | GAAAGAAGGACTGGGACCCTAAGAAGTACGGCGGCTTCGACAGCCCCACC |
| | GTGGCCTATTCTGTGCTGGTGGTGGCCAAAGTGGAAAAGGGCAAGTCCAA |
| | GAAACTGAAGAGTGTGAAAGAGCTGCTGGGGATCACCATCATGGAAAGAA |
| | GCAGCTTCGAGAAGAATCCCATCGACTTTCTGGAAGCCAAGGGCTACAAA |
| | GAAGTGAAAAAGGACCTGATCATCAAGCTGCCTAAGTACTCCCTGTTCGA |
| | GCTGGAAAACGGCCGGAAGAGAATGCTGGCCTCTGCCGGCGAACTGCAGA |
| | AGGGAAACGAACTGGCCCTGCCCTCCAAATATGTGAACTTCCTGTACCTG |
| | GCCAGCCACTATGAGAAGCTGAAGGGCTCCCCCGAGGATAATGAGCAGAA |
| | ACAGCTGTTTGTGGAACAGCACAAACACTACCTGGACGAGATCATCGAGC |
| | AGATCAGCGAGTTCTCCAAGAGAGTGATCCTGGCCGACGCTAATCTGGAC |
| | AAGGTGCTGAGCGCCTACAACAAGCACAGAGACAAGCCTATCAGAGAGCA |
| | GGCCGAGAATATCATCCACCTGTTTACCCTGACCAATCTGGGAGCCCCTG |
| | CCGCCTTCAAGTACTTTGACACCACCATCGACCGGAAGAGGTACACCAGC |
| | ACCAAAGAGGTGCTGGACGCCACCCTGATCCACCAGAGCATCACCGGCCT |
| | GTACGAGACACGGATCGACCTGTCTCAGCTGGGAGGCGACCTCGAGGGCG |
| | GATCCGGTGGTTCCGGAGGAGCTGTCGACATGTCCGGGGGCAGCAGCTGC |
| | AGCCAGACCCCAAGCGCTGCCGCAGCCGCCACTCGCCGGGTGGTGCTCGG |
| | CGCCGGCGTGCAGCTCCCGCCCGGGGACTACAGCACGGCCCCCGGCGGCA |
| | CGCTCTTCAGCACCGCCCCGGGAGGTACCAGGATCATCTATGACCGGAAA |
| | TTCCTGATGGAGTGTCGGAACGCACCTGTGACCAAAGCACCCCCAAGGGA |
| | TCTGCCCACCATTCCGGGGGTCACCAGCCCTTCCAGTGATGAGCCCCCCA |
| | TGGAAGCCAGCCAGAGCCACCTGCGCAATAGCCCAGAAGATAAGCGGGCG |
| | GGCGGTGAAGAGTCACAGGCTGAGATGGACATTTCTAGACTTAAGTAAGC |
| | CTCGACTGTGCCTTCTAGTTGCCAGCCATCTGTTGTTTGCCCCTCCCCCG |
| | TGCCTTCCTTGACCCTGGAAGGTGCCACTCCCACTGTCCTTTCCTAATAA |
| | AATGAGGAAATTGCATCGCATTGTCTGAGTAGGTGTCATTCTATTCTGGG |
| | GGGTGGGGTGGGGCAGGACAGCAAGGGGGAGGATTGGGAAGACAATAGCA |
| | GGCATGCTGGGGAGTGCCCGTCAGTGGGCAGAGCGCACATCGCCCACAGT |
| | CCCCGAGAAGTTGGGGGGAGGGGTCGGCAATTGAACGGGTGCCTAGAGAA |
| | GGTGGCGCGGGGTAAACTGGGAAAGTGATGTCGTGTACTGGCTCCGCCTT |
| | TTTCCCGAGGGTGGGGGAGAACCGTATATAAGTGCAGTAGTCGCCGTGAA |
| | CGTTCTTTTTCGCAACGGGTTTGCCGCCAGAACACAGATGGTCTCTAAAG |
| | GAGGTTCGTCCGACGACGAAGCAACAGCGGACTCGCAGCACGCCGCACCT |
| | CCTAAGAAGAAAAGGAAGGTAGGGGATCCCCGGGTACCGGTCGCCACCAT |
| | GGTGAGCAAGGGCGAGGAGCTGTTCACCGGGGTGGTGCCCATCCTGGTCG |
| | AGCTGGACGGCGACGTAAACGGCCACAAGTTCAGCGTGTCCGGCGAGGGC |
| | GAGGGCGATGCCACCTACGGCAAGCTGACCCTGAAGTTCATCTGCACCAC |
| | CGGCAAGCTGCCCGTGCCCTGGCCCACCCTCGTGACCACCCTGTCCTGGG |
| | GCGTGCAGTGCTTCGCCCGCTACCCCGACCACATGAAGCAGCACGACTTC |
| | TTCAAGTCCGCCATGCCCGAAGGCTACGTCCAGGAGCGCACCATCTTCTT |
| | CAAGGACGACGGCAACTACAAGACCCGCGCCGAGGTGAAGTTCGAGGGCG |
| | ACACCCTGGTGAACCGCATCGAGCTGAAGGGCATCGACTTCAAGGAGGAC |
| | GGCAACATCCTGGGGCACAAGCTGGAGTACAACTACTTTAGCGACAACGT |
| | CTATATCACCGCCGACAAGCAGAAGAACGGCATCAAGGCCAACTTCAAGA |
| | TCCGCCACAACATCGAGGACGGCGGCGTGCAGCTCGCCGACCACTACCAG |
| | CAGAACACCCCCATCGGCGACGGCCCCGTGCTGCTGCCCGACAACCACTA |
| | CCTGAGCACCCAGTCCAAGCTGAGCAAAGACCCCAACGAGAAGCGCGATC |
| | ACATGGTCCTGCTGGAGTTCGTGACCGCCGCCGGGATCACTCTCGGCATG |
| | GACGAGCTGTACAAGTAAAATCAACCTCTGGATTACAAAATTTGTGAAAG |
| | ATTGACTGGTATTCTTAACTATGTTGCTCCTTTTACGCTATGTGGATACG |
| | CTGCTTTAATGCCTTTGTATCATGTTAACTAAACTTGTTTATTGCAGCTT |
| | ATAATGGTTACAAATAAAGCAATAGCATCACAAATTTCACAAATAAAGCA |
| | TTTTTTTCACTGCATTCTAGTTGTGGTTTGTCCAAACTCATCAATGTATC |
| | TTATCATGTCTGGAATTGACTCAAATGATGTCAATTAGTCTATCAGAAGC |
| | TCATCTGGTCTCCCTTCCGGGGGACAAGACATCCCTGTTTAATATTTAAA |
| | CAGCAGTGTTCCCAAACTGGGTTCTTATATCCCTTGCTCTGGTCAACCAG |
| | GTTGCAGGGTTTCCTGTCCTCACAGGAACGAAGTCCCTAAAGAAACAGTG |
| | GCAGCCAGGTTTAGCCCCGGAATTGACTGGATTCCTTTTTTAGGGCCCAT |
| | TGGTATGGCTTTTTCCCCGTATCCCCCCAGGTGTCTGCAGGCTCAAAGAG |
| | CAGCGAGAAGCGTTCAGAGGAAAGCGATCCCGTGCCACCTTCCCCGTGCC |
| | CGGGCTGTCCCCGCACGCTGCCGGCTCGGGGATGCGGGGGGAGCGCCGGA |
| | CCGGAGCGGAGCCCCGGGCGGCTCGCTGCTGCCCCCTAGCGGGGGAGGGA |
| | CGTAATTACATCCCTGGGGGCTTTGGGGGGGGGCTGTCCCTGATATCTAT |
| | AACAAGAAAATATATATATAATAAGTTATCACGTAAGTAGAACATGAAAT |
| | AACAATATAATTATCGTATGAGTTAAATCTTAAAAGTCACGTAAAAGATA |
| | ATCATGCGTCATTTTGACTCACGCGGTCGTTATAGTTCAAAATCAGTGAC |
| | ACTTACCGCATTGACAAGCACGCCTCACGGGAGCTCCAAGCGGCGACTGA |
| | GATGTCCTAAATGCACAGCGACGGATTCGCGCTATTTAGAAAGAGAGAGC |
| | AATATTTCAAGAATGCATGCGTCAATTTTACGCAGACTATCTTTCTAGGG |
| | TTAATCTAGCTGCATCAGGATCATATCGTCGGGTCTTTTTTCCGGCTCAG |
| | TCATCGCCCAAGCTGGCGCTATCTGGGCATCGGGGAGGAAGAAGCCCGTG |
| | CCTTTTCCCGCGAGGTTGAAGCGGCATGGAAAGAGTTTGCCGAGGATGAC |
| | TGCTGCTGCATTGACGTTGAGCGAAAACGCACGTTTACCATGATGATTCG |
| | GGAAGGTGTGGCCATGCACGCCTTTAACGGTGAACTGTTCGTTCAGGCCA |
| | CCTGGGATACCAGTTCGTCGCGGCTTTTCCGGACACAGTTCCGGATGGTC |
| | AGCCCGAAGCGCATCAGCAACCCGAACAATACCGGCGACAGCCGGAACTG |
| | CCGTGCCGGTGTGCAGATTAATGACAGCGGTGCGGCGCTGGGATATTACG |
| | TCAGCGAGGACGGGTATCCTGGCTGGATGCCGCAGAAATGGACATGGATA |
| | CCCCGTGAGTTACCCGGCGGGCGCGCTTGGCGTAATCATGGTCATAGCTG |
| | TTTCCTGTGTGAAATTGTTATCCGCTCACAATTCCACACAACATACGAGC |
| | CGGAAGCATAAAGTGTAAAGCCTGGGGTGCCTAATGAGTGAGCTAACTCA |
| | CATTAATTGCGTTGCGCTCACTGCCCGCTTTCCAGTCGGGAAACCTGTCG |
| | TGCCAGCTGCATTAATGAATCGGCCAACGCGCGGGGAGAGGCGGTTTGCG |
| | TATTGGGCGCTCTTCCGCTTCCTCGCTCACTGACTCGCTGCGCTCGGTCG |
| | TTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCA |
| | GCAAAAGGCCAGGAACCGTAAAAAGGCCGCGTTGCTGGCGTTTTTCCATA |
| | GGCTCCGCCCCCCTGACGAGCATCACAAAAATCGACGCTCAAGTCAGAGG |
| | TGGCGAAACCCGACAGGACTATAAAGATACCAGGCGTTTCCCCCTGGAAG |
| | CTCCCTCGTGCGCTCTCCTGTTCCGACCCTGCCGCTTACCGGATACCTGT |
| | CCGCCTTTCTCCCTTCGGGAAGCGTGGCGCTTTCTCATAGCTCACGCTGT |
| | AGGTATCTCAGTTCGGTGTAGGTCGTTCGCTCCAAGCTGGGCTGTGTGCA |
| | CGAACCCCCCGTTCAGCCCGACCGCTGCGCCTTATCCGGTAACTATCGTC |
| | TTGAGTCCAACCCGGTAAGACACGACTTATCGCCACTGGCAGCAGCCACT |
| | GGTAACAGGATTAGCAGAGCGAGGTATGTAGGCGGTGCTACAGAGTTCTT |
| | GAAGTGGTGGCCTAACTACGGCTACACTAGAAGGACAGTATTTGGTATCT |
| | GCGCTCTGCTGAAGCCAGTTACCTTCGGAAAAAGAGTTGGTAGCTCTTGA |
| | TCCGGCAAACAAACCACCGCTGGTAGCGGTGGTTTTTTTGTTTGCAAGCA |
| | GCAGATTACGCGCAGAAAAAAAGGATCTCAAGAAGATCCTTTGATCTTTT |
| | CTACGGGGTCTGACGCTCAGTGGAACGAAAACTCACGTTAAGGGATTTTG |
| | GTCATGAGATTATCAAAAAGGATCTTCACCTAGATCCTTTTAAATTAAAA |
| | ATGAAGTTTTAAATCAATCTAAAGTATATATGAGTAAACTTGGTCTGACA |
| | GTTACCAATGCTTAATCAGTGAGGCACCTATCTCAGCGATCTGTCTATTT |
| | CGTTCATCCATAGTTGCCTGACTCCCCGTCGTGTAGATAACTACGATACG |
| | GGAGGGCTTACCATCTGGCCCCAGTGCTGCAATGATACCGCGAGACCCAC |
| | GCTCACCGGCTCCAGATTTATCAGCAATAAACCAGCCAGCCGGAAGGGCC |
| | GAGCGCAGAAGTGGTCCTGCAACTTTATCCGCCTCCATCCAGTCTATTAA |
| | TTGTTGCCGGGAAGCTAGAGTAAGTAGTTCGCCAGTTAATAGTTTGCGCA |
| | ACGTTGTTGCCATTGCTACAGGCATCGTGGTGTCACGCTCGTCGTTTGGT |
| | ATGGCTTCATTCAGCTCCGGTTCCCAACGATCAAGGCGAGTTACATGATC |
| | CCCCATGTTGTGCAAAAAAGCGGTTAGCTCCTTCGGTCCTCCGATCGTTG |
| | TCAGAAGTAAGTTGGCCGCAGTGTTATCACTCATGGTTATGGCAGCACTG |
| | CATAATTCTCTTACTGTCATGCCATCCGTAAGATGCTTTTCTGTGACTGG |
| | TGAGTACTCAACCAAGTCATTCTGAGAATAGTGTATGCGGCGACCGAGTT |
| | GCTCTTGCCCGGCGTCAATACGGGATAATACCGCGCCACATAGCAGAACT |
| | TTAAAAGTGCTCATCATTGGAAAACGTTCTTCGGGGCGAAAACTCTCAAG |
| | GATCTTACCGCTGTTGAGATCCAGTTCGATGTAACCCACTCGTGCACCCA |
| | ACTGATCTTCAGCATCTTTTACTTTCACCAGCGTTTCTGGGTGAGCAAAA |
| | ACAGGAAGGCAAAATGCCGCAAAAAAGGGAATAAGGGCGACACGGAAATG |
| | TTGAATACTCAT (SEQ ID NO: 78) |
| PspCas13b | MNIPALVENQKKYFGTYSVMAMLNAQTVLDHIQKVADIEGEQNEN |
| | NENLWFHPVMSHLYNAKNGYDKQPEKTMFIIERLQSYFPFLKIMA |
| | ENQREYSNGKYKQNRVEVNSNDIFEVLKRAFGVLKMYRDLTNAYK |
| | TYEEKLNDGCEFLTSTEQPLSGMINNYYTVALRNMNERYGYKTED |
| | LAFIQDKRFKFVKDAYGKKKSQVNTGFFLSLQDYNGDTQKKLHLS |
| | GVGIALLICLFLDKQYINIFLSRLPIFSSYNAQSEERRIIIRSFG |
| | INSIKLPKDRIHSEKSNKSVAMDMLNEVKRCPDELFTTLSAEKQS |
| | RFRIISDDHNEVLMKRSSDRFVPLLLQYIDYGKLFDHIRFHVNMG |
| | KLRYLLKADKTCIDGQTRVRVIEQPLNGFGRLEEAETMRKQENGT |
| | FGNSGIRIRDFENMKRDDANPANYPYIVDTYTHYILENNKVEMFI |
| | NDKEDSAPLLPVIEDDRYVVKTIPSCRMSTLEIPAMAFHMFLFGS |
| | KKTEKLIVDVHNRYKRLFQAMQKEEVTAENIASFGIAESDLPQKI |
| | LDLISGNAHGKDVDAFIRLTVDDMLTDTERRIKRFKDDRKSIRSA |
| | DNKMGKRGFKQISTGKLADFLAKDIVLFQPSVNDGENKITGLNYR |
| | IMQSAIAVYDSGDDYEAKQQFKLMFEKARLIGKGTTEPHPFLYKV |
| | FARSIPANAVEFYERYLIERKFYLTGLSNEIKKGNRVDVPFIRRD |
| | QNKWKTPAMKTLGRIYSEDLPVELPRQMFDNEIKSHLKSLPQMEG |
| | IDFNNANVTYLIAEYMKRVLDDDFQTFYQWNRNYRYMDMLKGEYD |
| | RKGSLQHCFTSVEEREGLWKERASRTERYRKQASNKIRSNRQMRN |
| | ASSEEIETILDKRLSNSRNEYQKSEKVIRRYRVQDALLFLLAKKT |
| | LTELADFDGERFKLKEIMPDAEKGILSEIMPMSFTFEKGGKKYTI |
| | TSEGMKLKNYGDFFVLASDKRIGNLLELVGSDIVSKEDIMEEFNK |
| | YDQCRPEISSIVFNLEKWAFDTYPELSARVDREEKVDFKSILKIL |
| | LNNKNINKEQSDILRKIRNAFDANNYPDKGVVEIKALPEIAMSIK |
| | KAFGEYAIMK (SEQ ID NO: 79) |
| PB-TRE-V5- | ACTCTTCCTTTTTCAATATTATTGAAGCATTTATCAGGGTTATTG |
| dCas13b- | TCTCATGAGCGGATACATATTTGAATGTATTTAGAAAAATAAACA |
| mut (EIF4E) EF | AATAGGGGTTCCGCGCACATTTCCCCGAAAAGTGCCACCTAAATT |
| NLS-Turq | GTAAGCGTTAATATTTTGTTAAAATTCGCGTTAAATTTTTGTTAA |
| | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTTCTACCGGGTAGGGGAGGCGCTTTTCCCAAGGC |
| | AGTCTGGAGCATGCGCTTTAGCAGCCCCGCTGGGCACTTGGCGCT |
| | ACACAAGTGGCCTCTGGCCTCGCACACATTCCACATCCACCGGTA |
| | GGCGCCAACCGGCTCCGTTCTTTGGTGGCCCCTTCGCGCCACCTT |
| | CTACTCCTCCCCTAGTCAGGAAGTTCCCCCCCGCCCCGCAGCTCG |
| | CGTCGTGCAGGACGTGACAAATGGAAGTAGCACGTCTCATTAGTC |
| | TCGTGCAGATGGACAGCACCGCTGAGCAATGGAAGCGGGTAGGCC |
| | TTTGGGGCAGCGGCCAATAGCAGCTTTGCTCCTTCGCTTTCTGGG |
| | CTCAGAGGCTGGGAAGGGGTGGGTCCGGGGGCGGGCTCAGGGGCG |
| | GGCTCAGGGGCGGGGCGGGCGCCCGAAGGTCCTCCGGAGGCCCGG |
| | CATTCTGCACGCTTCAAAAGCGCACGTCTGCCGCGCTGTTCTCCT |
| | CTTCCTCATCTCCGGGCCTTTCGACCTGCAGGTTAATTAAATGGG |
| | TAAGCCTATCCCTAACCCTCTCCTCGGTCTCGATTCTACGGCTAG |
| | CATGAACATCCCCGCTCTGGTGGAAAACCAGAAGAAGTACTTTGG |
| | CACCTACAGCGTGATGGCCATGCTGAACGCTCAGACCGTGCTGGA |
| | CCACATCCAGAAGGTGGCCGATATTGAGGGCGAGCAGAACGAGAA |
| | CAACGAGAATCTGTGGTTTCACCCCGTGATGAGCCACCTGTACAA |
| | CGCCAAGAACGGCTACGACAAGCAGCCCGAGAAAACCATGTTCAT |
| | CATCGAGCGGCTGCAGAGCTACTTCCCATTCCTGAAGATCATGGC |
| | CGAGAACCAGAGAGAGTACAGCAACGGCAAGTACAAGCAGAACCG |
| | CGTGGAAGTGAACAGCAACGACATCTTCGAGGTGCTGAAGCGCGC |
| | CTTCGGCGTGCTGAAGATGTACAGGGACCTGACCAACGCATACAA |
| | GACCTACGAGGAAAAGCTGAACGACGGCTGCGAGTTCCTGACCAG |
| | CACAGAGCAACCTCTGAGCGGCATGATCAACAACTACTACACAGT |
| | GGCCCTGCGGAACATGAACGAGAGATACGGCTACAAGACAGAGGA |
| | CCTGGCCTTCATCCAGGACAAGCGGTTCAAGTTCGTGAAGGACGC |
| | CTACGGCAAGAAAAAGTCCCAAGTGAATACCGGATTCTTCCTGAG |
| | CCTGCAGGACTACAACGGCGACACACAGAAGAAGCTGCACCTGAG |
| | CGGAGTGGGAATCGCCCTGCTGATCTGCCTGTTCCTGGACAAGCA |
| | GTACATCAACATCTTTCTGAGCAGGCTGCCCATCTTCTCCAGCTA |
| | CAATGCCCAGAGCGAGGAACGGCGGATCATCATCAGATCCTTCGG |
| | CATCAACAGCATCAAGCTGCCCAAGGACCGGATCCACAGCGAGAA |
| | GTCCAACAAGAGCGTGGCCATGGATATGCTCAACGAAGTGAAGCG |
| | GTGCCCCGACGAGCTGTTCACAACACTGTCTGCCGAGAAGCAGTC |
| | CCGGTTCAGAATCATCAGCGACGACCACAATGAAGTGCTGATGAA |
| | GCGGAGCAGCGACAGATTCGTGCCTCTGCTGCTGCAGTATATCGA |
| | TTACGGCAAGCTGTTCGACCACATCAGGTTCCACGTGAACATGGG |
| | CAAGCTGAGATACCTGCTGAAGGCCGACAAGACCTGCATCGACGG |
| | CCAGACCAGAGTCAGAGTGATCGAGCAGCCCCTGAACGGCTTCGG |
| | CAGACTGGAAGAGGCCGAGACAATGCGGAAGCAAGAGAACGGCAC |
| | CTTCGGCAACAGCGGCATCCGGATCAGAGACTTCGAGAACATGAA |
| | GCGGGACGACGCCAATCCTGCCAACTATCCCTACATCGTGGACAC |
| | CTACACACACTACATCCTGGAAAACAACAAGGTCGAGATGTTTAT |
| | CAACGACAAAGAGGACAGCGCCCCACTGCTGCCCGTGATCGAGGA |
| | TGATAGATACGTGGTCAAGACAATCCCCAGCTGCCGGATGAGCAC |
| | CCTGGAAATTCCAGCCATGGCCTTCCACATGTTTCTGTTCGGCAG |
| | CAAGAAAACCGAGAAGCTGATCGTGGACGTGCACAACCGGTACAA |
| | GAGACTGTTCCAGGCCATGCAGAAAGAAGAAGTGACCGCCGAGAA |
| | TATCGCCAGCTTCGGAATCGCCGAGAGCGACCTGCCTCAGAAGAT |
| | CCTGGATCTGATCAGCGGCAATGCCCACGGCAAGGATGTGGACGC |
| | CTTCATCAGACTGACCGTGGACGACATGCTGACCGACACCGAGCG |
| | GAGAATCAAGAGATTCAAGGACGACCGGAAGTCCATTCGGAGCGC |
| | CGACAACAAGATGGGAAAGAGAGGCTTCAAGCAGATCTCCACAGG |
| | CAAGCTGGCCGACTTCCTGGCCAAGGACATCGTGCTGTTTCAGCC |
| | CAGCGTGAACGATGGCGAGAACAAGATCACCGGCCTGAACTACCG |
| | GATCATGCAGAGCGCCATTGCCGTGTACGATAGCGGCGACGATTA |
| | CGAGGCCAAGCAGCAGTTCAAGCTGATGTTCGAGAAGGCCCGGCT |
| | GATCGGCAAGGGCACAACAGAGCCTCATCCATTTCTGTACAAGGT |
| | GTTCGCCCGCAGCATCCCCGCCAATGCCGTCGAGTTCTACGAGCG |
| | CTACCTGATCGAGCGGAAGTTCTACCTGACCGGCCTGTCCAACGA |
| | GATCAAGAAAGGCAACAGAGTGGATGTGCCCTTCATCCGGCGGGA |
| | CCAGAACAAGTGGAAAACACCCGCCATGAAGACCCTGGGCAGAAT |
| | CTACAGCGAGGATCTGCCCGTGGAACTGCCCAGACAGATGTTCGA |
| | CAATGAGATCAAGTCCCACCTGAAGTCCCTGCCACAGATGGAAGG |
| | CATCGACTTCAACAATGCCAACGTGACCTATCTGATCGCCGAGTA |
| | CATGAAGAGAGTGCTGGACGACGACTTCCAGACCTTCTACCAGTG |
| | GAACCGCAACTACCGGTACATGGACATGCTTAAGGGCGAGTACGA |
| | CAGAAAGGGCTCCCTGCAGCACTGCTTCACCAGCGTGGAAGAGAG |
| | AGAAGGCCTCTGGAAAGAGCGGGCCTCCAGAACAGAGCGGTACAG |
| | AAAGCAGGCCAGCAACAAGATCCGCAGCAACCGGCAGATGAGAAA |
| | CGCCAGCAGCGAAGAGATCGAGACAATCCTGGATAAGCGGCTGAG |
| | CAACAGCCGGAACGAGTACCAGAAAAGCGAGAAAGTGATCCGGCG |
| | CTACAGAGTGCAGGATGCCCTGCTGTTTCTGCTGGCCAAAAAGAC |
| | CCTGACCGAACTGGCCGATTTCGACGGCGAGAGGTTCAAACTGAA |
| | AGAAATCATGCCCGACGCCGAGAAGGGAATCCTGAGCGAGATCAT |
| | GCCCATGAGCTTCACCTTCGAGAAAGGCGGCAAGAAGTACACCAT |
| | CACCAGCGAGGGCATGAAGCTGAAGAACTACGGCGACTTCTTTGT |
| | GCTGGCTAGCGACAAGAGGATCGGCAACCTGCTGGAACTCGTGGG |
| | CAGCGACATCGTGTCCAAAGAGGATATCATGGAAGAGTTCAACAA |
| | ATACGACCAGTGCAGGCCCGAGATCAGCTCCATCGTGTTCAACCT |
| | GGAAAAGTGGGCCTTCGACACATACCCCGAGCTGTCTGCCAGAGT |
| | GGACCGGGAAGAGAAGGTGGACTTCAAGAGCATCCTGAAAATCCT |
| | GCTGAACAACAAGAACATCAACAAAGAGCAGAGCGACATCCTGCG |
| | GAAGATCCGGAACGCCTTCGATGCAAACAATTACCCCGACAAAGG |
| | CGTGGTGGAAATCAAGGCCCTGCCTGAGATCGCCATGAGCATCAA |
| | GAAGGCCTTTGGGGAGTACGCCATCATGAAGCTCGAGGGCGGATC |
| | CGGTGGTTCCGGAGGAGCTGTCGACATGGCGACTGTCGAACCGGA |
| | AACCACCCCTACTCCTAATCCCCCGACTACAGAAGAGGAGAAAAC |
| | GGAATCTAATCAGGAGGTTGCTAACCCAGAACACTATATTAAACG |
| | GCCCCTACAGAACAGATGGGCACTCTGGTTTTTTAAAAATGATAA |
| | AAGCAAAACTTGGCAAGCAAACCTGCGGCTGATCTCCAAGTTTGA |
| | TACTGCTGAAGACTTTTTTGCTCTGTACAACCATATCCAGTTGTC |
| | TAGTAATTTAATGCCTGGCTGTGACTACTCACTTTTTAAGGATGG |
| | TATTGAGCCTATGTGGGAAGATGAGAAAAACAAACGGGGAGGACG |
| | ATGGCTAATTACATTGAACAAACAGCAGAGACGAAGTGACCTCGA |
| | TCGCTTTTGGCTAGAGACACTTCTGTGCCTTATTGGAGAATCTTT |
| | TGATGACTACAGTGATGATGTATGTGGCGCTGTTGTTAATGTTAG |
| | AGCTAAAGGTGATAAGATAGCAATATGGACTACTGAATGTGAAAA |
| | CAGAGAAGCTGTTACACATATAGGGAGGGTATACAAGGAAAGGTT |
| | AGGACTTCCTCCAAAGATAGTGATTGGTTATCAGTCCCACGCAGA |
| | CACAGCTACTAAGAGCGGCGACACCACTAAAAATAGGTTTGTTGT |
| | TTCTAGACTTAAGTAAGCCTCGACTGTGCCTTCTAGTTGCCAGCC |
| | ATCTGTTGTTTGCCCCTCCCCCGTGCCTTCCTTGACCCTGGAAGG |
| | TGCCACTCCCACTGTCCTTTCCTAATAAAATGAGGAAATTGCATC |
| | GCATTGTCTGAGTAGGTGTCATTCTATTCTGGGGGGTGGGGTGGG |
| | GCAGGACAGCAAGGGGGAGGATTGGGAAGACAATAGCAGGCATGC |
| | TGGGGAGTGCCCGTCAGTGGGCAGAGCGCACATCGCCCACAGTCC |
| | CCGAGAAGTTGGGGGGAGGGGTCGGCAATTGAACGGGTGCCTAGA |
| | GAAGGTGGCGCGGGGTAAACTGGGAAAGTGATGTCGTGTACTGGC |
| | TCCGCCTTTTTCCCGAGGGTGGGGGAGAACCGTATATAAGTGCAG |
| | TAGTCGCCGTGAACGTTCTTTTTCGCAACGGGTTTGCCGCCAGAA |
| | CACAGATGGTCTCTAAAGGAGGTTCGTCCGACGACGAAGCAACAG |
| | CGGACTCGCAGCACGCCGCACCTCCTAAGAAGAAAAGGAAGGTAG |
| | GGGATCCCCGGGTACCGGTCGCCACCATGGTGAGCAAGGGCGAGG |
| | AGCTGTTCACCGGGGTGGTGCCCATCCTGGTCGAGCTGGACGGCG |
| | ACGTAAACGGCCACAAGTTCAGCGTGTCCGGCGAGGGCGAGGGCG |
| | ATGCCACCTACGGCAAGCTGACCCTGAAGTTCATCTGCACCACCG |
| | GCAAGCTGCCCGTGCCCTGGCCCACCCTCGTGACCACCCTGTCCT |
| | GGGGCGTGCAGTGCTTCGCCCGCTACCCCGACCACATGAAGCAGC |
| | ACGACTTCTTCAAGTCCGCCATGCCCGAAGGCTACGTCCAGGAGC |
| | GCACCATCTTCTTCAAGGACGACGGCAACTACAAGACCCGCGCCG |
| | AGGTGAAGTTCGAGGGCGACACCCTGGTGAACCGCATCGAGCTGA |
| | AGGGCATCGACTTCAAGGAGGACGGCAACATCCTGGGGCACAAGC |
| | TGGAGTACAACTACTTTAGCGACAACGTCTATATCACCGCCGACA |
| | AGCAGAAGAACGGCATCAAGGCCAACTTCAAGATCCGCCACAACA |
| | TCGAGGACGGCGGCGTGCAGCTCGCCGACCACTACCAGCAGAACA |
| | CCCCCATCGGCGACGGCCCCGTGCTGCTGCCCGACAACCACTACC |
| | TGAGCACCCAGTCCAAGCTGAGCAAAGACCCCAACGAGAAGCGCG |
| | ATCACATGGTCCTGCTGGAGTTCGTGACCGCCGCCGGGATCACTC |
| | TCGGCATGGACGAGCTGTACAAGTAAAATCAACCTCTGGATTACA |
| | AAATTTGTGAAAGATTGACTGGTATTCTTAACTATGTTGCTCCTT |
| | TTACGCTATGTGGATACGCTGCTTTAATGCCTTTGTATCATGTTA |
| | ACTAAACTTGTTTATTGCAGCTTATAATGGTTACAAATAAAGCAA |
| | TAGCATCACAAATTTCACAAATAAAGCATTTTTTTCACTGCATTC |
| | TAGTTGTGGTTTGTCCAAACTCATCAATGTATCTTATCATGTCTG |
| | GAATTGACTCAAATGATGTCAATTAGTCTATCAGAAGCTCATCTG |
| | GTCTCCCTTCCGGGGGACAAGACATCCCTGTTTAATATTTAAACA |
| | GCAGTGTTCCCAAACTGGGTTCTTATATCCCTTGCTCTGGTCAAC |
| | CAGGTTGCAGGGTTTCCTGTCCTCACAGGAACGAAGTCCCTAAAG |
| | AAACAGTGGCAGCCAGGTTTAGCCCCGGAATTGACTGGATTCCTT |
| | TTTTAGGGCCCATTGGTATGGCTTTTTCCCCGTATCCCCCCAGGT |
| | GTCTGCAGGCTCAAAGAGCAGCGAGAAGCGTTCAGAGGAAAGCGA |
| | TCCCGTGCCACCTTCCCCGTGCCCGGGCTGTCCCCGCACGCTGCC |
| | GGCTCGGGGATGCGGGGGGAGCGCCGGACCGGAGCGGAGCCCCGG |
| | GCGGCTCGCTGCTGCCCCCTAGCGGGGGAGGGACGTAATTACATC |
| | CCTGGGGGCTTTGGGGGGGGGCTGTCCCTGATATCTATAACAAGA |
| | AAATATATATATAATAAGTTATCACGTAAGTAGAACATGAAATAA |
| | CAATATAATTATCGTATGAGTTAAATCTTAAAAGTCACGTAAAAG |
| | ATAATCATGCGTCATTTTGACTCACGCGGTCGTTATAGTTCAAAA |
| | TCAGTGACACTTACCGCATTGACAAGCACGCCTCACGGGAGCTCC |
| | AAGCGGCGACTGAGATGTCCTAAATGCACAGCGACGGATTCGCGC |
| | TATTTAGAAAGAGAGAGCAATATTTCAAGAATGCATGCGTCAATT |
| | TTACGCAGACTATCTTTCTAGGGTTAATCTAGCTGCATCAGGATC |
| | ATATCGTCGGGTCTTTTTTCCGGCTCAGTCATCGCCCAAGCTGGC |
| | GCTATCTGGGCATCGGGGAGGAAGAAGCCCGTGCCTTTTCCCGCG |
| | AGGTTGAAGCGGCATGGAAAGAGTTTGCCGAGGATGACTGCTGCT |
| | GCATTGACGTTGAGCGAAAACGCACGTTTACCATGATGATTCGGG |
| | AAGGTGTGGCCATGCACGCCTTTAACGGTGAACTGTTCGTTCAGG |
| | CCACCTGGGATACCAGTTCGTCGCGGCTTTTCCGGACACAGTTCC |
| | GGATGGTCAGCCCGAAGCGCATCAGCAACCCGAACAATACCGGCG |
| | ACAGCCGGAACTGCCGTGCCGGTGTGCAGATTAATGACAGCGGTG |
| | CGGCGCTGGGATATTACGTCAGCGAGGACGGGTATCCTGGCTGGA |
| | TGCCGCAGAAATGGACATGGATACCCCGTGAGTTACCCGGCGGGC |
| | GCGCTTGGCGTAATCATGGTCATAGCTGTTTCCTGTGTGAAATTG |
| | TTATCCGCTCACAATTCCACACAACATACGAGCCGGAAGCATAAA |
| | GTGTAAAGCCTGGGGTGCCTAATGAGTGAGCTAACTCACATTAAT |
| | TGCGTTGCGCTCACTGCCCGCTTTCCAGTCGGGAAACCTGTCGTG |
| | CCAGCTGCATTAATGAATCGGCCAACGCGCGGGGAGAGGCGGTTT |
| | GCGTATTGGGCGCTCTTCCGCTTCCTCGCTCACTGACTCGCTGCG |
| | CTCGGTCGTTCGGCTGCGGCGAGCGGTATCAGCTCACTCAAAGGC |
| | GGTAATACGGTTATCCACAGAATCAGGGGATAACGCAGGAAAGAA |
| | CATGTGAGCAAAAGGCCAGCAAAAGGCCAGGAACCGTAAAAAGGC |
| | CGCGTTGCTGGCGTTTTTCCATAGGCTCCGCCCCCCTGACGAGCA |
| | TCACAAAAATCGACGCTCAAGTCAGAGGTGGCGAAACCCGACAGG |
| | ACTATAAAGATACCAGGCGTTTCCCCCTGGAAGCTCCCTCGTGCG |
| | CTCTCCTGTTCCGACCCTGCCGCTTACCGGATACCTGTCCGCCTT |
| | TCTCCCTTCGGGAAGCGTGGCGCTTTCTCATAGCTCACGCTGTAG |
| | GTATCTCAGTTCGGTGTAGGTCGTTCGCTCCAAGCTGGGCTGTGT |
| | GCACGAACCCCCCGTTCAGCCCGACCGCTGCGCCTTATCCGGTAA |
| | CTATCGTCTTGAGTCCAACCCGGTAAGACACGACTTATCGCCACT |
| | GGCAGCAGCCACTGGTAACAGGATTAGCAGAGCGAGGTATGTAGG |
| | CGGTGCTACAGAGTTCTTGAAGTGGTGGCCTAACTACGGCTACAC |
| | TAGAAGGACAGTATTTGGTATCTGCGCTCTGCTGAAGCCAGTTAC |
| | CTTCGGAAAAAGAGTTGGTAGCTCTTGATCCGGCAAACAAACCAC |
| | CGCTGGTAGCGGTGGTTTTTTTGTTTGCAAGCAGCAGATTACGCG |
| | CAGAAAAAAAGGATCTCAAGAAGATCCTTTGATCTTTTCTACGGG |
| | GTCTGACGCTCAGTGGAACGAAAACTCACGTTAAGGGATTTTGGT |
| | CATGAGATTATCAAAAAGGATCTTCACCTAGATCCTTTTAAATTA |
| | AAAATGAAGTTTTAAATCAATCTAAAGTATATATGAGTAAACTTG |
| | GTCTGACAGTTACCAATGCTTAATCAGTGAGGCACCTATCTCAGC |
| | GATCTGTCTATTTCGTTCATCCATAGTTGCCTGACTCCCCGTCGT |
| | GTAGATAACTACGATACGGGAGGGCTTACCATCTGGCCCCAGTGC |
| | TGCAATGATACCGCGAGACCCACGCTCACCGGCTCCAGATTTATC |
| | AGCAATAAACCAGCCAGCCGGAAGGGCCGAGCGCAGAAGTGGTCC |
| | TGCAACTTTATCCGCCTCCATCCAGTCTATTAATTGTTGCCGGGA |
| | AGCTAGAGTAAGTAGTTCGCCAGTTAATAGTTTGCGCAACGTTGT |
| | TGCCATTGCTACAGGCATCGTGGTGTCACGCTCGTCGTTTGGTAT |
| | GGCTTCATTCAGCTCCGGTTCCCAACGATCAAGGCGAGTTACATG |
| | ATCCCCCATGTTGTGCAAAAAAGCGGTTAGCTCCTTCGGTCCTCC |
| | GATCGTTGTCAGAAGTAAGTTGGCCGCAGTGTTATCACTCATGGT |
| | TATGGCAGCACTGCATAATTCTCTTACTGTCATGCCATCCGTAAG |
| | ATGCTTTTCTGTGACTGGTGAGTACTCAACCAAGTCATTCTGAGA |
| | ATAGTGTATGCGGCGACCGAGTTGCTCTTGCCCGGCGTCAATACG |
| | GGATAATACCGCGCCACATAGCAGAACTTTAAAAGTGCTCATCAT |
| | TGGAAAACGTTCTTCGGGGCGAAAACTCTCAAGGATCTTACCGCT |
| | GTTGAGATCCAGTTCGATGTAACCCACTCGTGCACCCAACTGATC |
| | TTCAGCATCTTTTACTTTCACCAGCGTTTCTGGGTGAGCAAAAAC |
| | AGGAAGGCAAAATGCCGCAAAAAAGGGAATAAGGGCGACACGGAA |
| | ATGTTGAATACTCAT(SEQ ID NO: 80) |
| PB-TRE-V5- | ACTCTTCCTTTTTCAATATTATTGAAGCATTTATCAGGGTTATTG |
| dCas13b-mut (c - | TCTCATGAGCGGATACATATTTGAATGTATTTAGAAAAATAAACA |
| 4EBP1-pho sMUT) | AATAGGGGTTCCGCGCACATTTCCCCGAAAAGTGCCACCTAAATT |
| EF1a NLS-Turq | GTAAGCGTTAATATTTTGTTAAAATTCGCGTTAAATTTTTGTTAA |
| | ATCAGCTCATTTTTTAACCAATAGGCCGAAATCGGCAAAATCCCT |
| | TATAAATCAAAAGAATAGACCGAGATAGGGTTGAGTGTGTTGCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTTCTACCGGGTAGGGGAGGCGCTTTTCCCAAGGC |
| | AGTCTGGAGCATGCGCTTTAGCAGCCCCGCTGGGCACTTGGCGCT |
| | ACACAAGTGGCCTCTGGCCTCGCACACATTCCACATCCACCGGTA |
| | GGCGCCAACCGGCTCCGTTCTTTGGTGGCCCCTTCGCGCCACCTT |
| | CTACTCCTCCCCTAGTCAGGAAGTTCCCCCCCGCCCCGCAGCTCG |
| | CGTCGTGCAGGACGTGACAAATGGAAGTAGCACGTCTCATTAGTC |
| | TCGTGCAGATGGACAGCACCGCTGAGCAATGGAAGCGGGTAGGCC |
| | TTTGGGGCAGCGGCCAATAGCAGCTTTGCTCCTTCGCTTTCTGGG |
| | CTCAGAGGCTGGGAAGGGGTGGGTCCGGGGGCGGGCTCAGGGGCG |
| | GGCTCAGGGGCGGGGCGGGCGCCCGAAGGTCCTCCGGAGGCCCGG |
| | CATTCTGCACGCTTCAAAAGCGCACGTCTGCCGCGCTGTTCTCCT |
| | CTTCCTCATCTCCGGGCCTTTCGACCTGCAGGTTAATTAAATGGG |
| | TAAGCCTATCCCTAACCCTCTCCTCGGTCTCGATTCTACGGCTAG |
| | CATGAACATCCCCGCTCTGGTGGAAAACCAGAAGAAGTACTTTGG |
| | CACCTACAGCGTGATGGCCATGCTGAACGCTCAGACCGTGCTGGA |
| | CCACATCCAGAAGGTGGCCGATATTGAGGGCGAGCAGAACGAGAA |
| | CAACGAGAATCTGTGGTTTCACCCCGTGATGAGCCACCTGTACAA |
| | CGCCAAGAACGGCTACGACAAGCAGCCCGAGAAAACCATGTTCAT |
| | CATCGAGCGGCTGCAGAGCTACTTCCCATTCCTGAAGATCATGGC |
| | CGAGAACCAGAGAGAGTACAGCAACGGCAAGTACAAGCAGAACCG |
| | CGTGGAAGTGAACAGCAACGACATCTTCGAGGTGCTGAAGCGCGC |
| | CTTCGGCGTGCTGAAGATGTACAGGGACCTGACCAACGCATACAA |
| | GACCTACGAGGAAAAGCTGAACGACGGCTGCGAGTTCCTGACCAG |
| | CACAGAGCAACCTCTGAGCGGCATGATCAACAACTACTACACAGT |
| | GGCCCTGCGGAACATGAACGAGAGATACGGCTACAAGACAGAGGA |
| | CCTGGCCTTCATCCAGGACAAGCGGTTCAAGTTCGTGAAGGACGC |
| | CTACGGCAAGAAAAAGTCCCAAGTGAATACCGGATTCTTCCTGAG |
| | CCTGCAGGACTACAACGGCGACACACAGAAGAAGCTGCACCTGAG |
| | CGGAGTGGGAATCGCCCTGCTGATCTGCCTGTTCCTGGACAAGCA |
| | GTACATCAACATCTTTCTGAGCAGGCTGCCCATCTTCTCCAGCTA |
| | CAATGCCCAGAGCGAGGAACGGCGGATCATCATCAGATCCTTCGG |
| | CATCAACAGCATCAAGCTGCCCAAGGACCGGATCCACAGCGAGAA |
| | GTCCAACAAGAGCGTGGCCATGGATATGCTCAACGAAGTGAAGCG |
| | GTGCCCCGACGAGCTGTTCACAACACTGTCTGCCGAGAAGCAGTC |
| | CCGGTTCAGAATCATCAGCGACGACCACAATGAAGTGCTGATGAA |
| | GCGGAGCAGCGACAGATTCGTGCCTCTGCTGCTGCAGTATATCGA |
| | TTACGGCAAGCTGTTCGACCACATCAGGTTCCACGTGAACATGGG |
| | CAAGCTGAGATACCTGCTGAAGGCCGACAAGACCTGCATCGACGG |
| | CCAGACCAGAGTCAGAGTGATCGAGCAGCCCCTGAACGGCTTCGG |
| | CAGACTGGAAGAGGCCGAGACAATGCGGAAGCAAGAGAACGGCAC |
| | CTTCGGCAACAGCGGCATCCGGATCAGAGACTTCGAGAACATGAA |
| | GCGGGACGACGCCAATCCTGCCAACTATCCCTACATCGTGGACAC |
| | CTACACACACTACATCCTGGAAAACAACAAGGTCGAGATGTTTAT |
| | CAACGACAAAGAGGACAGCGCCCCACTGCTGCCCGTGATCGAGGA |
| | TGATAGATACGTGGTCAAGACAATCCCCAGCTGCCGGATGAGCAC |
| | CCTGGAAATTCCAGCCATGGCCTTCCACATGTTTCTGTTCGGCAG |
| | CAAGAAAACCGAGAAGCTGATCGTGGACGTGCACAACCGGTACAA |
| | GAGACTGTTCCAGGCCATGCAGAAAGAAGAAGTGACCGCCGAGAA |
| | TATCGCCAGCTTCGGAATCGCCGAGAGCGACCTGCCTCAGAAGAT |
| | CCTGGATCTGATCAGCGGCAATGCCCACGGCAAGGATGTGGACGC |
| | CTTCATCAGACTGACCGTGGACGACATGCTGACCGACACCGAGCG |
| | GAGAATCAAGAGATTCAAGGACGACCGGAAGTCCATTCGGAGCGC |
| | CGACAACAAGATGGGAAAGAGAGGCTTCAAGCAGATCTCCACAGG |
| | CAAGCTGGCCGACTTCCTGGCCAAGGACATCGTGCTGTTTCAGCC |
| | CAGCGTGAACGATGGCGAGAACAAGATCACCGGCCTGAACTACCG |
| | GATCATGCAGAGCGCCATTGCCGTGTACGATAGCGGCGACGATTA |
| | CGAGGCCAAGCAGCAGTTCAAGCTGATGTTCGAGAAGGCCCGGCT |
| | GATCGGCAAGGGCACAACAGAGCCTCATCCATTTCTGTACAAGGT |
| | GTTCGCCCGCAGCATCCCCGCCAATGCCGTCGAGTTCTACGAGCG |
| | CTACCTGATCGAGCGGAAGTTCTACCTGACCGGCCTGTCCAACGA |
| | GATCAAGAAAGGCAACAGAGTGGATGTGCCCTTCATCCGGCGGGA |
| | CCAGAACAAGTGGAAAACACCCGCCATGAAGACCCTGGGCAGAAT |
| | CTACAGCGAGGATCTGCCCGTGGAACTGCCCAGACAGATGTTCGA |
| | CAATGAGATCAAGTCCCACCTGAAGTCCCTGCCACAGATGGAAGG |
| | CATCGACTTCAACAATGCCAACGTGACCTATCTGATCGCCGAGTA |
| | CATGAAGAGAGTGCTGGACGACGACTTCCAGACCTTCTACCAGTG |
| | GAACCGCAACTACCGGTACATGGACATGCTTAAGGGCGAGTACGA |
| | CAGAAAGGGCTCCCTGCAGCACTGCTTCACCAGCGTGGAAGAGAG |
| | AGAAGGCCTCTGGAAAGAGCGGGCCTCCAGAACAGAGCGGTACAG |
| | AAAGCAGGCCAGCAACAAGATCCGCAGCAACCGGCAGATGAGAAA |
| | CGCCAGCAGCGAAGAGATCGAGACAATCCTGGATAAGCGGCTGAG |
| | CAACAGCCGGAACGAGTACCAGAAAAGCGAGAAAGTGATCCGGCG |
| | CTACAGAGTGCAGGATGCCCTGCTGTTTCTGCTGGCCAAAAAGAC |
| | CCTGACCGAACTGGCCGATTTCGACGGCGAGAGGTTCAAACTGAA |
| | AGAAATCATGCCCGACGCCGAGAAGGGAATCCTGAGCGAGATCAT |
| | GCCCATGAGCTTCACCTTCGAGAAAGGCGGCAAGAAGTACACCAT |
| | CACCAGCGAGGGCATGAAGCTGAAGAACTACGGCGACTTCTTTGT |
| | GCTGGCTAGCGACAAGAGGATCGGCAACCTGCTGGAACTCGTGGG |
| | CAGCGACATCGTGTCCAAAGAGGATATCATGGAAGAGTTCAACAA |
| | ATACGACCAGTGCAGGCCCGAGATCAGCTCCATCGTGTTCAACCT |
| | GGAAAAGTGGGCCTTCGACACATACCCCGAGCTGTCTGCCAGAGT |
| | GGACCGGGAAGAGAAGGTGGACTTCAAGAGCATCCTGAAAATCCT |
| | GCTGAACAACAAGAACATCAACAAAGAGCAGAGCGACATCCTGCG |
| | GAAGATCCGGAACGCCTTCGATGCAAACAATTACCCCGACAAAGG |
| | CGTGGTGGAAATCAAGGCCCTGCCTGAGATCGCCATGAGCATCAA |
| | GAAGGCCTTTGGGGAGTACGCCATCATGAAGCTCGAGGGCGGATC |
| | CGGTGGTTCCGGAGGAGCTGTCGACATGTCCGGGGGCAGCAGCTG |
| | CAGCCAGACCCCAAGCGCTGCCGCAGCCGCCACTCGCCGGGTGGT |
| | GCTCGGCGCCGGCGTGCAGCTCCCGCCCGGGGACTACAGCACGGC |
| | CCCCGGCGGCACGCTCTTCAGCACCGCCCCGGGAGGTACCAGGAT |
| | CATCTATGACCGGAAATTCCTGATGGAGTGTCGGAACGCACCTGT |
| | GACCAAAGCACCCCCAAGGGATCTGCCCACCATTCCGGGGGTCAC |
| | CAGCCCTTCCAGTGATGAGCCCCCCATGGAAGCCAGCCAGAGCCA |
| | CCTGCGCAATAGCCCAGAAGATAAGCGGGCGGGCGGTGAAGAGTC |
| | ACAGGCTGAGATGGACATTTCTAGACTTAAGTAAGCCTCGACTGT |
| | GCCTTCTAGTTGCCAGCCATCTGTTGTTTGCCCCTCCCCCGTGCC |
| | TTCCTTGACCCTGGAAGGTGCCACTCCCACTGTCCTTTCCTAATA |
| | AAATGAGGAAATTGCATCGCATTGTCTGAGTAGGTGTCATTCTAT |
| | TCTGGGGGGTGGGGTGGGGCAGGACAGCAAGGGGGAGGATTGGGA |
| | AGACAATAGCAGGCATGCTGGGGAGTGCCCGTCAGTGGGCAGAGC |
| | GCACATCGCCCACAGTCCCCGAGAAGTTGGGGGGAGGGGTCGGCA |
| | ATTGAACGGGTGCCTAGAGAAGGTGGCGCGGGGTAAACTGGGAAA |
| | GTGATGTCGTGTACTGGCTCCGCCTTTTTCCCGAGGGTGGGGGAG |
| | AACCGTATATAAGTGCAGTAGTCGCCGTGAACGTTCTTTTTCGCA |
| | ACGGGTTTGCCGCCAGAACACAGATGGTCTCTAAAGGAGGTTCGT |
| | CCGACGACGAAGCAACAGCGGACTCGCAGCACGCCGCACCTCCTA |
| | AGAAGAAAAGGAAGGTAGGGGATCCCCGGGTACCGGTCGCCACCA |
| | TGGTGAGCAAGGGCGAGGAGCTGTTCACCGGGGTGGTGCCCATCC |
| | TGGTCGAGCTGGACGGCGACGTAAACGGCCACAAGTTCAGCGTGT |
| | CCGGCGAGGGCGAGGGCGATGCCACCTACGGCAAGCTGACCCTGA |
| | AGTTCATCTGCACCACCGGCAAGCTGCCCGTGCCCTGGCCCACCC |
| | TCGTGACCACCCTGTCCTGGGGCGTGCAGTGCTTCGCCCGCTACC |
| | CCGACCACATGAAGCAGCACGACTTCTTCAAGTCCGCCATGCCCG |
| | AAGGCTACGTCCAGGAGCGCACCATCTTCTTCAAGGACGACGGCA |
| | ACTACAAGACCCGCGCCGAGGTGAAGTTCGAGGGCGACACCCTGG |
| | TGAACCGCATCGAGCTGAAGGGCATCGACTTCAAGGAGGACGGCA |
| | ACATCCTGGGGCACAAGCTGGAGTACAACTACTTTAGCGACAACG |
| | TCTATATCACCGCCGACAAGCAGAAGAACGGCATCAAGGCCAACT |
| | TCAAGATCCGCCACAACATCGAGGACGGCGGCGTGCAGCTCGCCG |
| | ACCACTACCAGCAGAACACCCCCATCGGCGACGGCCCCGTGCTGC |
| | TGCCCGACAACCACTACCTGAGCACCCAGTCCAAGCTGAGCAAAG |
| | ACCCCAACGAGAAGCGCGATCACATGGTCCTGCTGGAGTTCGTGA |
| | CCGCCGCCGGGATCACTCTCGGCATGGACGAGCTGTACAAGTAAA |
| | ATCAACCTCTGGATTACAAAATTTGTGAAAGATTGACTGGTATTC |
| | TTAACTATGTTGCTCCTTTTACGCTATGTGGATACGCTGCTTTAA |
| | TCGGTTTGTATCATGTTAACTAAACTTGTTTATTGCAGCTTATAA |
| | TGGTTACAAATAAAGCAATAGCATCACAAATTTCACAAATAAAGC |
| | ATTTTTTTCACTGCATTCTAGTTGTGGTTTGTCCAAACTCATCAA |
| | TGTATCTTATCATGTCTGGAATTGACTCAAATGATGTCAATTAGT |
| | CTATCAGAAGCTCATCTGGTCTCCCTTCCGGGGGACAAGACATCC |
| | CTGTTTAATATTTAAACAGCAGTGTTCCCAAACTGGGTTCTTATA |
| | TCCCTTGCTCTGGTCAACCAGGTTGCAGGGTTTCCTGTCCTCACA |
| | GGAACGAAGTCCCTAAAGAAACAGTGGCAGCCAGGTTTAGCCCCG |
| | GAATTGACTGGATTCCTTTTTTAGGGCCCATTGGTATGGCTTTTT |
| | CCCCGTATCCCCCCAGGTGTCTGCAGGCTCAAAGAGCAGCGAGAA |
| | GCGTTCAGAGGAAAGCGATCCCGTGCCACCTTCCCCGTGCCCGGG |
| | CTGTCCCCGCACGCTGCCGGCTCGGGGATGCGGGGGGAGCGCCGG |
| | ACCGGAGCGGAGCCCCGGGCGGCTCGCTGCTGCCCCCTAGCGGGG |
| | GAGGGACGTAATTACATCCCTGGGGGCTTTGGGGGGGGGCTGTCC |
| | CTGATATCTATAACAAGAAAATATATATATAATAAGTTATCACGT |
| | AAGTAGAACATGAAATAACAATATAATTATCGTATGAGTTAAATC |
| | TTAAAAGTCACGTAAAAGATAATCATGCGTCATTTTGACTCACGC |
| | GGTCGTTATAGTTCAAAATCAGTGACACTTACCGCATTGACAAGC |
| | ACGCCTCACGGGAGCTCCAAGCGGCGACTGAGATGTCCTAAATGC |
| | ACAGCGACGGATTCGCGCTATTTAGAAAGAGAGAGCAATATTTCA |
| | AGAATGCATGCGTCAATTTTACGCAGACTATCTTTCTAGGGTTAA |
| | TCTAGCTGCATCAGGATCATATCGTCGGGTCTTTTTTCCGGCTCA |
| | GTCATCGCCCAAGCTGGCGCTATCTGGGCATCGGGGAGGAAGAAG |
| | CCCGTGCCTTTTCCCGCGAGGTTGAAGCGGCATGGAAAGAGTTTG |
| | CCGAGGATGACTGCTGCTGCATTGACGTTGAGCGAAAACGCACGT |
| | TTACCATGATGATTCGGGAAGGTGTGGCCATGCACGCCTTTAACG |
| | GTGAACTGTTCGTTCAGGCCACCTGGGATACCAGTTCGTCGCGGC |
| | TTTTCCGGACACAGTTCCGGATGGTCAGCCCGAAGCGCATCAGCA |
| | ACCCGAACAATACCGGCGACAGCCGGAACTGCCGTGCCGGTGTGC |
| | AGATTAATGACAGCGGTGCGGCGCTGGGATATTACGTCAGCGAGG |
| | ACGGGTATCCTGGCTGGATGCCGCAGAAATGGACATGGATACCCC |
| | GTGAGTTACCCGGCGGGCGCGCTTGGCGTAATCATGGTCATAGCT |
| | GTTTCCTGTGTGAAATTGTTATCCGCTCACAATTCCACACAACAT |
| | ACGAGCCGGAAGCATAAAGTGTAAAGCCTGGGGTGCCTAATGAGT |
| | GAGCTAACTCACATTAATTGCGTTGCGCTCACTGCCCGCTTTCCA |
| | GTCGGGAAACCTGTCGTGCCAGCTGCATTAATGAATCGGCCAACG |
| | CGCGGGGAGAGGCGGTTTGCGTATTGGGCGCTCTTCCGCTTCCTC |
| | GCTCACTGACTCGCTGCGCTCGGTCGTTCGGCTGCGGCGAGCGGT |
| | ATCAGCTCACTCAAAGGCGGTAATACGGTTATCCACAGAATCAGG |
| | GGATAACGCAGGAAAGAACATGTGAGCAAAAGGCCAGCAAAAGGC |
| | CAGGAACCGTAAAAAGGCCGCGTTGCTGGCGTTTTTCCATAGGCT |
| | CCGCCCCCCTGACGAGCATCACAAAAATCGACGCTCAAGTCAGAG |
| | GTGGCGAAACCCGACAGGACTATAAAGATACCAGGCGTTTCCCCC |
| | TGGAAGCTCCCTCGTGCGCTCTCCTGTTCCGACCCTGCCGCTTAC |
| | CGGATACCTGTCCGCCTTTCTCCCTTCGGGAAGCGTGGCGCTTTC |
| | TCATAGCTCACGCTGTAGGTATCTCAGTTCGGTGTAGGTCGTTCG |
| | CTCCAAGCTGGGCTGTGTGCACGAACCCCCCGTTCAGCCCGACCG |
| | CTGCGCCTTATCCGGTAACTATCGTCTTGAGTCCAACCCGGTAAG |
| | ACACGACTTATCGCCACTGGCAGCAGCCACTGGTAACAGGATTAG |
| | CAGAGCGAGGTATGTAGGCGGTGCTACAGAGTTCTTGAAGTGGTG |
| | GCCTAACTACGGCTACACTAGAAGGACAGTATTTGGTATCTGCGC |
| | TCTGCTGAAGCCAGTTACCTTCGGAAAAAGAGTTGGTAGCTCTTG |
| | ATCCGGCAAACAAACCACCGCTGGTAGCGGTGGTTTTTTTGTTTG |
| | CAAGCAGCAGATTACGCGCAGAAAAAAAGGATCTCAAGAAGATCC |
| | TTTGATCTTTTCTACGGGGTCTGACGCTCAGTGGAACGAAAACTC |
| | ACGTTAAGGGATTTTGGTCATGAGATTATCAAAAAGGATCTTCAC |
| | CTAGATCCTTTTAAATTAAAAATGAAGTTTTAAATCAATCTAAAG |
| | TATATATGAGTAAACTTGGTCTGACAGTTACCAATGCTTAATCAG |
| | TGAGGCACCTATCTCAGCGATCTGTCTATTTCGTTCATCCATAGT |
| | TGCCTGACTCCCCGTCGTGTAGATAACTACGATACGGGAGGGCTT |
| | ACCATCTGGCCCCAGTGCTGCAATGATACCGCGAGACCCACGCTC |
| | ACCGGCTCCAGATTTATCAGCAATAAACCAGCCAGCCGGAAGGGC |
| | CGAGCGCAGAAGTGGTCCTGCAACTTTATCCGCCTCCATCCAGTC |
| | TATTAATTGTTGCCGGGAAGCTAGAGTAAGTAGTTCGCCAGTTAA |
| | TAGTTTGCGCAACGTTGTTGCCATTGCTACAGGCATCGTGGTGTC |
| | ACGCTCGTCGTTTGGTATGGCTTCATTCAGCTCCGGTTCCCAACG |
| | ATCAAGGCGAGTTACATGATCCCCCATGTTGTGCAAAAAAGCGGT |
| | TAGCTCCTTCGGTCCTCCGATCGTTGTCAGAAGTAAGTTGGCCGC |
| | AGTGTTATCACTCATGGTTATGGCAGCACTGCATAATTCTCTTAC |
| | TGTCATGCCATCCGTAAGATGCTTTTCTGTGACTGGTGAGTACTC |
| | AACCAAGTCATTCTGAGAATAGTGTATGCGGCGACCGAGTTGCTC |
| | TTGCCCGGCGTCAATACGGGATAATACCGCGCCACATAGCAGAAC |
| | TTTAAAAGTGCTCATCATTGGAAAACGTTCTTCGGGGCGAAAACT |
| | CTCAAGGATCTTACCGCTGTTGAGATCCAGTTCGATGTAACCCAC |
| | TCGTGCACCCAACTGATCTTCAGCATCTTTTACTTTCACCAGCGT |
| | TTCTGGGTGAGCAAAAACAGGAAGGCAAAATGCCGCAAAAAAGGG |
| | AATAAGGGCGACACGGAAATGTTGAATACTCAT (SEQ ID NO: |
| | 81) |
| PB-TRE-H2B- | ACTCTTCCTTTTTCAATATTATTGAAGCATTTATCAGGGTTATTG |
| Citrine- | TCTCATGAGCGGATACATATTTGAATGTATTTAGAAAAATAAACA |
| (Mod.SDHA_Lambda2)- | AATAGGGGTTCCGCGCACATTTCCCCGAAAAGTGCCACCTAAATT |
| EF1a-Puro- | GTAAGCGTTAATATTTTGTTAAAATTCGCGTTAAATTTTTGTTAA |
| rtTA-Cherry + U6- | ATCAGCTCATTTTTTAACCAATAGGCCGAAATCGGCAAAATCCCT |
| sgLUC | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATATAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCAAAAAAAGCACCGACTCGGTGCCACTTTTTCAAGTTGAT |
| | AACGGACTAGCCTTATTTAAACTTGCTATGCTGTTTCCAGCATAG |
| | CTCTTAAACATTCTTACGCTGAGTACTTCGGTGTTTCGTCCTTTC |
| | CACAAGATATATAAAGCCAAGAAATCGAAATACTTTCAAGTTACG |
| | GTAAGCATATGATAGTCCATTTTAAAACATAATTTTAAAACTGCA |
| | AACTACCCAAGAAATTATTACTTTCTACGTCACGTATTTTGTACT |
| | AATATCTTTGTGTTTACAGTCAAATTAATTCTAATTATCTCTCTA |
| | ACAGCCTTGTATCGTATATGCAAATATGAAGGAATCATGGGAAAT |
| | AGGCCCTCTTCCTGCCCGACCTGACTAGTACTTTCACTTTTCTCT |
| | ATCACTGATAGGGAGTGGTAAACTCGACTTTCACTTTTCTCTATC |
| | ACTGATAGGGAGTGGTAAACTCGACTTTCACTTTTCTCTATCACT |
| | GATAGGGAGTGGTAAACTCGACTTTCACTTTTCTCTATCACTGAT |
| | AGGGAGTGGTAAACTCGACTTTCACTTTTCTCTATCACTGATAGG |
| | GAGTGGTAAACTCGACTTTCACTTTTCTCTATCACTGATAGGGAG |
| | TGGTAAACTCGACTTTCACTTTTCTCTATCACTGATAGGGAGTGG |
| | TAAACTCGACCTATATAAGCAGAGCTCGTTTAGTGAACCGTCAGA |
| | TCGCCTGGAGACGCCATCCACGCTGTTTTGACCTCCATAGAAGAC |
| | ACCGGGACCGATCCAGCCTCCGCGGCCCCGAATTCATGCCAGAGC |
| | CAGCGAAGTCTGCTCCCGCCCCGAAAAAGGGCTCCAAGAAGGCGG |
| | TGACTAAGGCGCAGAAGAAAGGCGGCAAGAAGCGCAAGCGCAGCC |
| | GCAAGGAGAGCTATTCCATCTATGTGTACAAGGTTCTGAAGCAGG |
| | TCCACCCTGACACCGGCATTTCGTCCAAGGCCATGGGCATCATGA |
| | ATTCGTTTGTGAACGACATTTTCGAGCGCATCGCAGGTGAGGCTT |
| | CCCGCCTGGCGCATTACAACAAGCGCTCGACCATCACCTCCAGGG |
| | AGATCCAGACGGCCGTGCGCCTGCTGCTGCCTGGGGAGTTGGCCA |
| | AGCACGCCGTGTCCGAGGGTACTAAGGCCATCACCAAGTACACCA |
| | GCGCTAAGGATCCCCGGGTACCGGTCGCCACCATGGTGAGCAAGG |
| | GCGAGGAGCTGTTCACCGGGGTGGTGCCCATCCTGGTCGAGCTGG |
| | ACGGCGACGTAAACGGCCACAAGTTCAGCGTGTCCGGCGAGGGCG |
| | AGGGCGATGCCACCTACGGCAAGCTGACCCTGAAGTTCATCTGCA |
| | CCACCGGCAAGCTGCCCGTGCCCTGGCCCACCCTCGTGACCACCT |
| | TCGGCTACGGCCTGATGTGCTTCGCCCGCTACCCCGACCACATGA |
| | AGCAGCACGACTTCTTCAAGTCCGCCATGCCCGAAGGCTACGTCC |
| | AGGAGCGCACCATCTTCTTCAAGGACGACGGCAACTACAAGACCC |
| | GCGCCGAGGTGAAGTTCGAGGGCGACACCCTGGTGAACCGCATCG |
| | AGCTGAAGGGCATCGACTTCAAGGAGGACGGCAACATCCTGGGGC |
| | ACAAGCTGGAGTACAACTACAACAGCCACAACGTCTATATCATGG |
| | CCGACAAGCAGAAGAACGGCATCAAGGTGAACTTCAAGATCCGCC |
| | ACAACATCGAGGACGGCAGCGTGCAGCTCGCCGACCACTACCAGC |
| | AGAACACCCCCATCGGCGACGGCCCCGTGCTGCTGCCCGACAACC |
| | ACTACCTGAGCTACCAGTCCGCCCTGAGCAAAGACCCCAACGAGA |
| | AGCGCGATCACATGGTCCTGCTGGAGTTCGTGACCGCCGCCGGGA |
| | TCACTCTCGGCATGGACGAGCTGTACAAGTAATCTAGAGCTAGCG |
| | CATATGTCCACTTAAGGGCGCTGACATGCGCATGTGAGGATCAAT |
| | TCTTACGCTGAGTACTTCGATTCCTCAAATAGCAAGACAGCCCAC |
| | ATGGCATTCCACTTATCACTGGCATCCTAGATCTGATAGCTTTGT |
| | TCTCAAAGTCTCGAGAAATTCGAATTTAAATCGGCCTCGACTGTG |
| | CCTTCTAGTTGCCAGCCATCTGTTGTTTGCCCCTCCCCCGTGCCT |
| | TCCTTGACCCTGGAAGGTGCCACTCCCACTGTCCTTTCCTAATAA |
| | AATGAGGAAATTGCATCGCATTGTCTGAGTAGGTGTCATTCTATT |
| | CTGGGGGGTGGGGTGGGGCAGGACAGCAAGGGGGAGGATTGGGAA |
| | GACAATAGCAGGCATGCTGGGGACGCGGCCGCGAAGGATCTGCGA |
| | TCGCTCCGGTGCCCGTCAGTGGGCAGAGCGCACATCGCCCACAGT |
| | CCCCGAGAAGTTGGGGGGAGGGGTCGGCAATTGAACGGGTGCCTA |
| | GAGAAGGTGGCGCGGGGTAAACTGGGAAAGTGATGTCGTGTACTG |
| | GCTCCGCCTTTTTCCCGAGGGTGGGGGAGAACCGTATATAAGTGC |
| | AGTAGTCGCCGTGAACGTTCTTTTTCGCAACGGGTTTGCCGCCAG |
| | AACACAGCTGAAGCTTCGAGGGGCTCGCATCTCTCCTTCACGCGC |
| | CCGCCGCCCTACCTGAGGCCGCCATCCACGCCGGTTGAGTCGCGT |
| | TCTGCCGCCTCCCGCCTGTGGTGCCTCCTGAACTGCGTCCGCCGT |
| | CTAGGTAAGTTTAAAGCTCAGGTCGAGACCGGGCCTTTGTCCGGC |
| | GCTCCCTTGGAGCCTACCTAGACTCAGCCGGCTCTCCACGCTTTG |
| | CCTGACCCTGCTTGCTCAACTCTACGTCTTTGTTTCGTTTTCTGT |
| | TCTGCGCCGTTACAGATCCAAGCTGTGACCGGCGCCTACGCTAGA |
| | TGACCGAGTACAAGCCCACGGTGCGCCTCGCCACCCGCGACGACG |
| | TCCCCAGGGCCGTACGCACCCTCGCCGCCGCGTTCGCCGACTACC |
| | CCGCCACGCGCCACACCGTCGATCCGGACCGCCACATCGAGCGGG |
| | TCACCGAGCTGCAAGAACTCTTCCTCACGCGCGTCGGGCTCGACA |
| | TCGGCAAGGTGTGGGTCGCGGACGACGGCGCCGCGGTGGCGGTCT |
| | GGACCACGCCGGAGAGCGTCGAAGCGGGGGCGGTGTTCGCCGAGA |
| | TCGGCCCGCGCATGGCCGAGTTGAGCGGTTCCCGGCTGGCCGCGC |
| | AGCAACAGATGGAAGGCCTCCTGGCGCCGCACCGGCCCAAGGAGC |
| | CCGCGTGGTTCCTGGCCACCGTCGGCGTCTCGCCCGACCACCAGG |
| | GCAAGGGTCTGGGCAGCGCCGTCGTGCTCCCCGGAGTGGAGGCGG |
| | CCGAGCGCGCCGGGGTGCCCGCCTTCCTGGAGACCTCCGCGCCCC |
| | GCAACCTCCCCTTCTACGAGCGGCTCGGCTTCACCGTCACCGCCG |
| | ACGTCGAGGTGCCCGAAGGACCGCGCACCTGGTGCATGACCCGCA |
| | AGCCCGGTGCCGGAAGCGGAGCTACTAACTTCAGCCTGCTGAAGC |
| | AGGCTGGAGACGTGGAGGAGAACCCTGGACCTATGTCTAGACTGG |
| | ACAAGAGCAAAGTCATAAACGGCGCTCTGGAATTACTCAATGGAG |
| | TCGGTATCGAAGGCCTGACGACAAGGAAACTCGCTCAAAAGCTGG |
| | GAGTTGAGCAGCCTACCCTGTACTGGCACGTGAAGAACAAGCGGG |
| | CCCTGCTCGATGCCCTGCCAATCGAGATGCTGGACAGGCATCATA |
| | CCCACTTCTGCCCCCTGGAAGGCGAGTCATGGCAAGACTTTCTGC |
| | GGAACAACGCCAAGTCATTCCGCTGTGCTCTCCTCTCACATCGCG |
| | ACGGGGCTAAAGTGCATCTCGGCACCCGCCCAACAGAGAAACAGT |
| | ACGAAACCCTGGAAAATCAGCTCGCGTTCCTGTGTCAGCAAGGCT |
| | TCTCCCTGGAGAACGCACTGTACGCTCTGTCCGCCGTGGGCCACT |
| | TTACACTGGGCTGCGTATTGGAGGAACAGGAGCATCAAGTAGCAA |
| | AAGAGGAAAGAGAGACACCTACCACCGATTCTATGCCCCCACTTC |
| | TGAGACAAGCAATTGAGCTGTTCGACCGGCAGGGAGCCGAACCTG |
| | CCTTCCTTTTCGGCCTGGAACTAATCATATGTGGCCTGGAGAAAC |
| | AGCTAAAGTGCGAAAGCGGCGGGCCGGCCGACGCCCTTGACGATT |
| | TTGACTTAGACATGCTCCCAGCCGATGCCCTTGACGACTTTGACC |
| | TTGATATGCTGCCTGCTGACGCTCTTGACGATTTTGACCTTGACA |
| | TGCTCCCCGGGTAACTAAGTAACCCTCTCCCTCCCCCCCCCCTAA |
| | CGTTACTGGCCGAAGCCGCTTGGAATAAGGCCGGTGTGCGTTTGT |
| | CTATATGTTATTTTCCACCATATTGCCGTCTTTTGGCAATGTGAG |
| | GGCCCGGAAACCTGGCCCTGTCTTCTTGACGAGCATTCCTAGGGG |
| | TCTTTCCCCTCTCGCCAAAGGAATGCAAGGTCTGTTGAATGTCGT |
| | GAAGGAAGCAGTTCCTCTGGAAGCTTCTTGAAGACAAACAACGTC |
| | TGTAGCGACCCTTTGCAGGCAGCGGAACCCCCCACCTGGCGACAG |
| | GTGCCTCTGCGGCCAAAAGCCACGTGTATAAGATACACCTGCAAA |
| | GGCGGCACAACCCCAGTGCCACGTTGTGAGTTGGATAGTTGTGGA |
| | AAGAGTCAAATGGCTCTCCTCAAGCGTATTCAACAAGGGGCTGAA |
| | GGATGCCCAGAAGGTACCCCATTGTATGGGATCTGATCTGGGGCC |
| | TCGGTGCACATGCTTTACATGTGTTTAGTCGAGGTTAAAAAAACG |
| | TCTAGGCCCCCCGAACCACGGGGACGTGGTTTTCCTTTGAAAAAC |
| | ACGATGATAATATGGCCACAACCATGGTCTCTAAAGGAGGTTCGT |
| | CCGACGACGAAGCAACAGCGGACTCGCAGCACGCCGCACCTCCTA |
| | AGAAGAAAAGGAAGGTAGGGGATCCCCGGGTACCGGTCGCCACCA |
| | TGGCCATCATCAAGGAGTTCATGCGCTTCAAGGTGCACATGGAGG |
| | GCTCCGTGAACGGCCACGAGTTCGAGATCGAGGGCGAGGGCGAGG |
| | GCCGCCCCTACGAGGGCACCCAGACCGCCAAGCTGAAGGTGACCA |
| | AGGGTGGCCCCCTGCCCTTCGCCTGGGACATCCTGTCCCCTCAGT |
| | TCATGTACGGCTCCAAGGCCTACGTGAAGCACCCCGCCGACATCC |
| | CCGACTACTTGAAGCTGTCCTTCCCCGAGGGCTTCAAGTGGGAGC |
| | GCGTGATGAACTTCGAGGACGGCGGCGTGGTGACCGTGACCCAGG |
| | ACTCCTCCCTGCAGGACGGCGAGTTCATCTACAAGGTGAAGCTGC |
| | GCGGCACCAACTTCCCCTCCGACGGCCCCGTAATGCAGAAGAAGA |
| | CCATGGGCTGGGAGGCCTCCTCCGAGCGGATGTACCCCGAGGACG |
| | GCGCCCTGAAGGGCGAGATCAAGCAGAGGCTGAAGCTGAAGGACG |
| | GCGGCCACTACGACGCTGAGGTCAAGACCACCTACAAGGCCAAGA |
| | AGCCCGTGCAGCTGCCCGGCGCCTACAACGTCAACATCAAGTTGG |
| | ACATCACCTCCCACAACGAGGACTACACCATCGTGGAACAGTACG |
| | AACGCGCCGAGGGCCGCCACTCCACCGGCGGCATGGACGAGCTGT |
| | ACAAGTAAAATCAACCTCTGGATTACAAAATTTGTGAAAGATTGA |
| | CTGGTATTCTTAACTATGTTGCTCCTTTTACGCTATGTGGATACG |
| | CTGCTTTAATGCCTTTGTATCATGCGTTAACTAAACTTGTTTATT |
| | GCAGCTTATAATGGTTACAAATAAAGCAATAGCATCACAAATTTC |
| | ACAAATAAAGCATTTTTTTCACTGCATTCTAGTTGTGGTTTGTCC |
| | AAACTCATCAATGTATCTTATCATGTCTGGAATTGACTCAAATGA |
| | TGTCAATTAGTCTATCAGAAGCTCATCTGGTCTCCCTTCCGGGGG |
| | ACAAGACATCCCTGTTTAATATTTAAACAGCAGTGTTCCCAAACT |
| | GGGTTCTTATATCCCTTGCTCTGGTCAACCAGGTTGCAGGGTTTC |
| | CTGTCCTCACAGGAACGAAGTCCCTAAAGAAACAGTGGCAGCCAG |
| | GTTTAGCCCCGGAATTGACTGGATTCCTTTTTTAGGGCCCATTGG |
| | TATGGCTTTTTCCCCGTATCCCCCCAGGTGTCTGCAGGCTCAAAG |
| | AGCAGCGAGAAGCGTTCAGAGGAAAGCGATCCCGTGCCACCTTCC |
| | CCGTGCCCGGGCTGTCCCCGCACGCTGCCGGCTCGGGGATGCGGG |
| | GGGAGCGCCGGACCGGAGCGGAGCCCCGGGCGGCTCGCTGCTGCC |
| | CCCTAGCGGGGGAGGGACGTAATTACATCCCTGGGGGCTTTGGGG |
| | GGGGGCTGTCCCTGATATCTATAACAAGAAAATATATATATAATA |
| | AGTTATCACGTAAGTAGAACATGAAATAACAATATAATTATCGTA |
| | TGAGTTAAATCTTAAAAGTCACGTAAAAGATAATCATGCGTCATT |
| | TTGACTCACGCGGTCGTTATAGTTCAAAATCAGTGACACTTACCG |
| | CATTGACAAGCACGCCTCACGGGAGCTCCAAGCGGCGACTGAGAT |
| | GTCCTAAATGCACAGCGACGGATTCGCGCTATTTAGAAAGAGAGA |
| | GCAATATTTCAAGAATGCATGCGTCAATTTTACGCAGACTATCTT |
| | TCTAGGGTTAATCTAGCTGCATCAGGATCATATCGTCGGGTCTTT |
| | TTTCCGGCTCAGTCATCGCCCAAGCTGGCGCTATCTGGGCATCGG |
| | GGAGGAAGAAGCCCGTGCCTTTTCCCGCGAGGTTGAAGCGGCATG |
| | GAAAGAGTTTGCCGAGGATGACTGCTGCTGCATTGACGTTGAGCG |
| | AAAACGCACGTTTACCATGATGATTCGGGAAGGTGTGGCCATGCA |
| | CGCCTTTAACGGTGAACTGTTCGTTCAGGCCACCTGGGATACCAG |
| | TTCGTCGCGGCTTTTCCGGACACAGTTCCGGATGGTCAGCCCGAA |
| | GCGCATCAGCAACCCGAACAATACCGGCGACAGCCGGAACTGCCG |
| | TGCCGGTGTGCAGATTAATGACAGCGGTGCGGCGCTGGGATATTA |
| | CGTCAGCGAGGACGGGTATCCTGGCTGGATGCCGCAGAAATGGAC |
| | ATGGATACCCCGTGAGTTACCCGGCGGGCGCGCTTGGCGTAATCA |
| | TGGTCATAGCTGTTTCCTGTGTGAAATTGTTATCCGCTCACAATT |
| | CCACACAACATACGAGCCGGAAGCATAAAGTGTAAAGCCTGGGGT |
| | GCCTAATGAGTGAGCTAACTCACATTAATTGCGTTGCGCTCACTG |
| | CCCGCTTTCCAGTCGGGAAACCTGTCGTGCCAGCTGCATTAATGA |
| | ATCGGCCAACGCGCGGGGAGAGGCGGTTTGCGTATTGGGCGCTCT |
| | TCCGCTTCCTCGCTCACTGACTCGCTGCGCTCGGTCGTTCGGCTG |
| | CGGCGAGCGGTATCAGCTCACTCAAAGGCGGTAATACGGTTATCC |
| | ACAGAATCAGGGGATAACGCAGGAAAGAACATGTGAGCAAAAGGC |
| | CAGCAAAAGGCCAGGAACCGTAAAAAGGCCGCGTTGCTGGCGTTT |
| | TTCCATAGGCTCCGCCCCCCTGACGAGCATCACAAAAATCGACGC |
| | TCAAGTCAGAGGTGGCGAAACCCGACAGGACTATAAAGATACCAG |
| | GCGTTTCCCCCTGGAAGCTCCCTCGTGCGCTCTCCTGTTCCGACC |
| | CTGCCGCTTACCGGATACCTGTCCGCCTTTCTCCCTTCGGGAAGC |
| | GTGGCGCTTTCTCATAGCTCACGCTGTAGGTATCTCAGTTCGGTG |
| | TAGGTCGTTCGCTCCAAGCTGGGCTGTGTGCACGAACCCCCCGTT |
| | CAGCCCGACCGCTGCGCCTTATCCGGTAACTATCGTCTTGAGTCC |
| | AACCCGGTAAGACACGACTTATCGCCACTGGCAGCAGCCACTGGT |
| | AACAGGATTAGCAGAGCGAGGTATGTAGGCGGTGCTACAGAGTTC |
| | TTGAAGTGGTGGCCTAACTACGGCTACACTAGAAGGACAGTATTT |
| | GGTATCTGCGCTCTGCTGAAGCCAGTTACCTTCGGAAAAAGAGTT |
| | GGTAGCTCTTGATCCGGCAAACAAACCACCGCTGGTAGCGGTGGT |
| | TTTTTTGTTTGCAAGCAGCAGATTACGCGCAGAAAAAAAGGATCT |
| | CAAGAAGATCCTTTGATCTTTTCTACGGGGTCTGACGCTCAGTGG |
| | AACGAAAACTCACGTTAAGGGATTTTGGTCATGAGATTATCAAAA |
| | AGGATCTTCACCTAGATCCTTTTAAATTAAAAATGAAGTTTTAAA |
| | TCAATCTAAAGTATATATGAGTAAACTTGGTCTGACAGTTACCAA |
| | TGCTTAATCAGTGAGGCACCTATCTCAGCGATCTGTCTATTTCGT |
| | TCATCCATAGTTGCCTGACTCCCCGTCGTGTAGATAACTACGATA |
| | CGGGAGGGCTTACCATCTGGCCCCAGTGCTGCAATGATACCGCGA |
| | GACCCACGCTCACCGGCTCCAGATTTATCAGCAATAAACCAGCCA |
| | GCCGGAAGGGCCGAGCGCAGAAGTGGTCCTGCAACTTTATCCGCC |
| | TCCATCCAGTCTATTAATTGTTGCCGGGAAGCTAGAGTAAGTAGT |
| | TCGCCAGTTAATAGTTTGCGCAACGTTGTTGCCATTGCTACAGGC |
| | ATCGTGGTGTCACGCTCGTCGTTTGGTATGGCTTCATTCAGCTCC |
| | GGTTCCCAACGATCAAGGCGAGTTACATGATCCCCCATGTTGTGC |
| | AAAAAAGCGGTTAGCTCCTTCGGTCCTCCGATCGTTGTCAGAAGT |
| | AAGTTGGCCGCAGTGTTATCACTCATGGTTATGGCAGCACTGCAT |
| | AATTCTCTTACTGTCATGCCATCCGTAAGATGCTTTTCTGTGACT |
| | GGTGAGTACTCAACCAAGTCATTCTGAGAATAGTGTATGCGGCGA |
| | CCGAGTTGCTCTTGCCCGGCGTCAATACGGGATAATACCGCGCCA |
| | CATAGCAGAACTTTAAAAGTGCTCATCATTGGAAAACGTTCTTCG |
| | GGGCGAAAACTCTCAAGGATCTTACCGCTGTTGAGATCCAGTTCG |
| | ATGTAACCCACTCGTGCACCCAACTGATCTTCAGCATCTTTTACT |
| | TTCACCAGCGTTTCTGGGTGAGCAAAAACAGGAAGGCAAAATGCC |
| | GCAAAAAAGGGAATAAGGGCGACACGGAAATGTTGAATACTCAT |
| | (SEQ ID NO: 82) |
| PB-TRE-H2B- | ACTCTTCCTTTTTCAATATTATTGAAGCATTTATCAGGGTTATTG |
| Citrine- | TCTCATGAGCGGATACATATTTGAATGTATTTAGAAAAATAAACA |
| (Mod.SDHA_Lambda2)- | AATAGGGGTTCCGCGCACATTTCCCCGAAAAGTGCCACCTAAATT |
| EF1a-Puro- | GTAAGCGTTAATATTTTGTTAAAATTCGCGTTAAATTTTTGTTAA |
| rtTA-Cherry + U6- | ATCAGCTCATTTTTTAACCAATAGGCCGAAATCGGCAAAATCCCT |
| crRNA | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTTAATTTGAATAGATATTAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCAAAAAAATCGACCTGTTCTCGAACATGGCATTGGGAACA |
| | CGTTTTAGTCCCCTTCGTTTTTGGGGTAGTCTAAATCGGTGTTTC |
| | GTCCTTTCCACAAGATATATAAAGCCAAGAAATCGAAATACTTTC |
| | AAGTTACGGTAAGCATATGATAGTCCATTTTAAAACATAATTTTA |
| | AAACTGCAAACTACCCAAGAAATTATTACTTTCTACGTCACGTAT |
| | TTTGTACTAATATCTTTGTGTTTACAGTCAAATTAATTCTAATTA |
| | TCTCTCTAACAGCCTTGTATCGTATATGCAAATATGAAGGAATCA |
| | TGGGAAATAGGCCCTCTTCCTGCCCGACCTGACTAGTACTTTCAC |
| | TTTTCTCTATCACTGATAGGGAGTGGTAAACTCGACTTTCACTTT |
| | TCTCTATCACTGATAGGGAGTGGTAAACTCGACTTTCACTTTTCT |
| | CTATCACTGATAGGGAGTGGTAAACTCGACTTTCACTTTTCTCTA |
| | TCACTGATAGGGAGTGGTAAACTCGACTTTCACTTTTCTCTATCA |
| | CTGATAGGGAGTGGTAAACTCGACTTTCACTTTTCTCTATCACTG |
| | ATAGGGAGTGGTAAACTCGACTTTCACTTTTCTCTATCACTGATA |
| | GGGAGTGGTAAACTCGACCTATATAAGCAGAGCTCGTTTAGTGAA |
| | CCGTCAGATCGCCTGGAGACGCCATCCACGCTGTTTTGACCTCCA |
| | TAGAAGACACCGGGACCGATCCAGCCTCCGCGGCCCCGAATTCAT |
| | GCCAGAGCCAGCGAAGTCTGCTCCCGCCCCGAAAAAGGGCTCCAA |
| | GAAGGCGGTGACTAAGGCGCAGAAGAAAGGCGGCAAGAAGCGCAA |
| | GCGCAGCCGCAAGGAGAGCTATTCCATCTATGTGTACAAGGTTCT |
| | GAAGCAGGTCCACCCTGACACCGGCATTTCGTCCAAGGCCATGGG |
| | CATCATGAATTCGTTTGTGAACGACATTTTCGAGCGCATCGCAGG |
| | TGAGGCTTCCCGCCTGGCGCATTACAACAAGCGCTCGACCATCAC |
| | CTCCAGGGAGATCCAGACGGCCGTGCGCCTGCTGCTGCCTGGGGA |
| | GTTGGCCAAGCACGCCGTGTCCGAGGGTACTAAGGCCATCACCAA |
| | GTACACCAGCGCTAAGGATCCCCGGGTACCGGTCGCCACCATGGT |
| | GAGCAAGGGCGAGGAGCTGTTCACCGGGGTGGTGCCCATCCTGGT |
| | CGAGCTGGACGGCGACGTAAACGGCCACAAGTTCAGCGTGTCCGG |
| | CGAGGGCGAGGGCGATGCCACCTACGGCAAGCTGACCCTGAAGTT |
| | CATCTGCACCACCGGCAAGCTGCCCGTGCCCTGGCCCACCCTCGT |
| | GACCACCTTCGGCTACGGCCTGATGTGCTTCGCCCGCTACCCCGA |
| | CCACATGAAGCAGCACGACTTCTTCAAGTCCGCCATGCCCGAAGG |
| | CTACGTCCAGGAGCGCACCATCTTCTTCAAGGACGACGGCAACTA |
| | CAAGACCCGCGCCGAGGTGAAGTTCGAGGGCGACACCCTGGTGAA |
| | CCGCATCGAGCTGAAGGGCATCGACTTCAAGGAGGACGGCAACAT |
| | CCTGGGGCACAAGCTGGAGTACAACTACAACAGCCACAACGTCTA |
| | TATCATGGCCGACAAGCAGAAGAACGGCATCAAGGTGAACTTCAA |
| | GATCCGCCACAACATCGAGGACGGCAGCGTGCAGCTCGCCGACCA |
| | CTACCAGCAGAACACCCCCATCGGCGACGGCCCCGTGCTGCTGCC |
| | CGACAACCACTACCTGAGCTACCAGTCCGCCCTGAGCAAAGACCC |
| | CAACGAGAAGCGCGATCACATGGTCCTGCTGGAGTTCGTGACCGC |
| | CGCCGGGATCACTCTCGGCATGGACGAGCTGTACAAGTAATCTAG |
| | AGCTAGCGCATATGTCCACTTAAGGGCGCTGACATGCGCATGTGA |
| | GGATCAATTCTTACGCTGAGTACTTCGATTCCTCAAATAGCAAGA |
| | CAGCCCACATGGCATTCCACTTATCACTGGCATCCTAGATCTGAT |
| | AGCTTTGTTCTCAAAGTCTCGAGAAATTCGAATTTAAATCGGCCT |
| | CGACTGTGCCTTCTAGTTGCCAGCCATCTGTTGTTTGCCCCTCCC |
| | CCGTGCCTTCCTTGACCCTGGAAGGTGCCACTCCCACTGTCCTTT |
| | CCTAATAAAATGAGGAAATTGCATCGCATTGTCTGAGTAGGTGTC |
| | ATTCTATTCTGGGGGGTGGGGTGGGGCAGGACAGCAAGGGGGAGG |
| | ATTGGGAAGACAATAGCAGGCATGCTGGGGACGCGGCCGCGAAGG |
| | ATCTGCGATCGCTCCGGTGCCCGTCAGTGGGCAGAGCGCACATCG |
| | CCCACAGTCCCCGAGAAGTTGGGGGGAGGGGTCGGCAATTGAACG |
| | GGTGCCTAGAGAAGGTGGCGCGGGGTAAACTGGGAAAGTGATGTC |
| | GTGTACTGGCTCCGCCTTTTTCCCGAGGGTGGGGGAGAACCGTAT |
| | ATAAGTGCAGTAGTCGCCGTGAACGTTCTTTTTCGCAACGGGTTT |
| | GCCGCCAGAACACAGCTGAAGCTTCGAGGGGCTCGCATCTCTCCT |
| | TCACGCGCCCGCCGCCCTACCTGAGGCCGCCATCCACGCCGGTTG |
| | AGTCGCGTTCTGCCGCCTCCCGCCTGTGGTGCCTCCTGAACTGCG |
| | TCCGCCGTCTAGGTAAGTTTAAAGCTCAGGTCGAGACCGGGCCTT |
| | TGTCCGGCGCTCCCTTGGAGCCTACCTAGACTCAGCCGGCTCTCC |
| | ACGCTTTGCCTGACCCTGCTTGCTCAACTCTACGTCTTTGTTTCG |
| | TTTTCTGTTCTGCGCCGTTACAGATCCAAGCTGTGACCGGCGCCT |
| | ACGCTAGATGACCGAGTACAAGCCCACGGTGCGCCTCGCCACCCG |
| | CGACGACGTCCCCAGGGCCGTACGCACCCTCGCCGCCGCGTTCGC |
| | CGACTACCCCGCCACGCGCCACACCGTCGATCCGGACCGCCACAT |
| | CGAGCGGGTCACCGAGCTGCAAGAACTCTTCCTCACGCGCGTCGG |
| | GCTCGACATCGGCAAGGTGTGGGTCGCGGACGACGGCGCCGCGGT |
| | GGCGGTCTGGACCACGCCGGAGAGCGTCGAAGCGGGGGCGGTGTT |
| | CGCCGAGATCGGCCCGCGCATGGCCGAGTTGAGCGGTTCCCGGCT |
| | GGCCGCGCAGCAACAGATGGAAGGCCTCCTGGCGCCGCACCGGCC |
| | CAAGGAGCCCGCGTGGTTCCTGGCCACCGTCGGCGTCTCGCCCGA |
| | CCACCAGGGCAAGGGTCTGGGCAGCGCCGTCGTGCTCCCCGGAGT |
| | GGAGGCGGCCGAGCGCGCCGGGGTGCCCGCCTTCCTGGAGACCTC |
| | CGCGCCCCGCAACCTCCCCTTCTACGAGCGGCTCGGCTTCACCGT |
| | CACCGCCGACGTCGAGGTGCCCGAAGGACCGCGCACCTGGTGCAT |
| | GACCCGCAAGCCCGGTGCCGGAAGCGGAGCTACTAACTTCAGCCT |
| | GCTGAAGCAGGCTGGAGACGTGGAGGAGAACCCTGGACCTATGTC |
| | TAGACTGGACAAGAGCAAAGTCATAAACGGCGCTCTGGAATTACT |
| | CAATGGAGTCGGTATCGAAGGCCTGACGACAAGGAAACTCGCTCA |
| | AAAGCTGGGAGTTGAGCAGCCTACCCTGTACTGGCACGTGAAGAA |
| | CAAGCGGGCCCTGCTCGATGCCCTGCCAATCGAGATGCTGGACAG |
| | GCATCATACCCACTTCTGCCCCCTGGAAGGCGAGTCATGGCAAGA |
| | CTTTCTGCGGAACAACGCCAAGTCATTCCGCTGTGCTCTCCTCTC |
| | ACATCGCGACGGGGCTAAAGTGCATCTCGGCACCCGCCCAACAGA |
| | GAAACAGTACGAAACCCTGGAAAATCAGCTCGCGTTCCTGTGTCA |
| | GCAAGGCTTCTCCCTGGAGAACGCACTGTACGCTCTGTCCGCCGT |
| | GGGCCACTTTACACTGGGCTGCGTATTGGAGGAACAGGAGCATCA |
| | AGTAGCAAAAGAGGAAAGAGAGACACCTACCACCGATTCTATGCC |
| | CCCACTTCTGAGACAAGCAATTGAGCTGTTCGACCGGCAGGGAGC |
| | CGAACCTGCCTTCCTTTTCGGCCTGGAACTAATCATATGTGGCCT |
| | GGAGAAACAGCTAAAGTGCGAAAGCGGCGGGCCGGCCGACGCCCT |
| | TGACGATTTTGACTTAGACATGCTCCCAGCCGATGCCCTTGACGA |
| | CTTTGACCTTGATATGCTGCCTGCTGACGCTCTTGACGATTTTGA |
| | CCTTGACATGCTCCCCGGGTAACTAAGTAACCCTCTCCCTCCCCC |
| | CCCCCTAACGTTACTGGCCGAAGCCGCTTGGAATAAGGCCGGTGT |
| | GCGTTTGTCTATATGTTATTTTCCACCATATTGCCGTCTTTTGGC |
| | AATGTGAGGGCCCGGAAACCTGGCCCTGTCTTCTTGACGAGCATT |
| | CCTAGGGGTCTTTCCCCTCTCGCCAAAGGAATGCAAGGTCTGTTG |
| | AATGTCGTGAAGGAAGCAGTTCCTCTGGAAGCTTCTTGAAGACAA |
| | ACAACGTCTGTAGCGACCCTTTGCAGGCAGCGGAACCCCCCACCT |
| | GGCGACAGGTGCCTCTGCGGCCAAAAGCCACGTGTATAAGATACA |
| | CCTGCAAAGGCGGCACAACCCCAGTGCCACGTTGTGAGTTGGATA |
| | GTTGTGGAAAGAGTCAAATGGCTCTCCTCAAGCGTATTCAACAAG |
| | GGGCTGAAGGATGCCCAGAAGGTACCCCATTGTATGGGATCTGAT |
| | CTGGGGCCTCGGTGCACATGCTTTACATGTGTTTAGTCGAGGTTA |
| | AAAAAACGTCTAGGCCCCCCGAACCACGGGGACGTGGTTTTCCTT |
| | TGAAAAACACGATGATAATATGGCCACAACCATGGTCTCTAAAGG |
| | AGGTTCGTCCGACGACGAAGCAACAGCGGACTCGCAGCACGCCGC |
| | ACCTCCTAAGAAGAAAAGGAAGGTAGGGGATCCCCGGGTACCGGT |
| | CGCCACCATGGCCATCATCAAGGAGTTCATGCGCTTCAAGGTGCA |
| | CATGGAGGGCTCCGTGAACGGCCACGAGTTCGAGATCGAGGGCGA |
| | GGGCGAGGGCCGCCCCTACGAGGGCACCCAGACCGCCAAGCTGAA |
| | GGTGACCAAGGGTGGCCCCCTGCCCTTCGCCTGGGACATCCTGTC |
| | CCCTCAGTTCATGTACGGCTCCAAGGCCTACGTGAAGCACCCCGC |
| | CGACATCCCCGACTACTTGAAGCTGTCCTTCCCCGAGGGCTTCAA |
| | GTGGGAGCGCGTGATGAACTTCGAGGACGGCGGCGTGGTGACCGT |
| | GACCCAGGACTCCTCCCTGCAGGACGGCGAGTTCATCTACAAGGT |
| | GAAGCTGCGCGGCACCAACTTCCCCTCCGACGGCCCCGTAATGCA |
| | GAAGAAGACCATGGGCTGGGAGGCCTCCTCCGAGCGGATGTACCC |
| | CGAGGACGGCGCCCTGAAGGGCGAGATCAAGCAGAGGCTGAAGCT |
| | GAAGGACGGCGGCCACTACGACGCTGAGGTCAAGACCACCTACAA |
| | GGCCAAGAAGCCCGTGCAGCTGCCCGGCGCCTACAACGTCAACAT |
| | CAAGTTGGACATCACCTCCCACAACGAGGACTACACCATCGTGGA |
| | ACAGTACGAACGCGCCGAGGGCCGCCACTCCACCGGCGGCATGGA |
| | CGAGCTGTACAAGTAAAATCAACCTCTGGATTACAAAATTTGTGA |
| | AAGATTGACTGGTATTCTTAACTATGTTGCTCCTTTTACGCTATG |
| | TGGATACGCTGCTTTAATGCCTTTGTATCATGCGTTAACTAAACT |
| | TGTTTATTGCAGCTTATAATGGTTACAAATAAAGCAATAGCATCA |
| | CAAATTTCACAAATAAAGCATTTTTTTCACTGCATTCTAGTTGTG |
| | GTTTGTCCAAACTCATCAATGTATCTTATCATGTCTGGAATTGAC |
| | TCAAATGATGTCAATTAGTCTATCAGAAGCTCATCTGGTCTCCCT |
| | TCCGGGGGACAAGACATCCCTGTTTAATATTTAAACAGCAGTGTT |
| | CCCAAACTGGGTTCTTATATCCCTTGCTCTGGTCAACCAGGTTGC |
| | AGGGTTTCCTGTCCTCACAGGAACGAAGTCCCTAAAGAAACAGTG |
| | GCAGCCAGGTTTAGCCCCGGAATTGACTGGATTCCTTTTTTAGGG |
| | CCCATTGGTATGGCTTTTTCCCCGTATCCCCCCAGGTGTCTGCAG |
| | GCTCAAAGAGCAGCGAGAAGCGTTCAGAGGAAAGCGATCCCGTGC |
| | CACCTTCCCCGTGCCCGGGCTGTCCCCGCACGCTGCCGGCTCGGG |
| | GATGCGGGGGGAGCGCCGGACCGGAGCGGAGCCCCGGGCGGCTCG |
| | CTGCTGCCCCCTAGCGGGGGAGGGACGTAATTACATCCCTGGGGG |
| | CTTTGGGGGGGGGCTGTCCCTGATATCTATAACAAGAAAATATAT |
| | ATATAATAAGTTATCACGTAAGTAGAACATGAAATAACAATATAA |
| | TTATCGTATGAGTTAAATCTTAAAAGTCACGTAAAAGATAATCAT |
| | GCGTCATTTTGACTCACGCGGTCGTTATAGTTCAAAATCAGTGAC |
| | ACTTACCGCATTGACAAGCACGCCTCACGGGAGCTCCAAGCGGCG |
| | ACTGAGATGTCCTAAATGCACAGCGACGGATTCGCGCTATTTAGA |
| | AAGAGAGAGCAATATTTCAAGAATGCATGCGTCAATTTTACGCAG |
| | ACTATCTTTCTAGGGTTAATCTAGCTGCATCAGGATCATATCGTC |
| | GGGTCTTTTTTCCGGCTCAGTCATCGCCCAAGCTGGCGCTATCTG |
| | GGCATCGGGGAGGAAGAAGCCCGTGCCTTTTCCCGCGAGGTTGAA |
| | GCGGCATGGAAAGAGTTTGCCGAGGATGACTGCTGCTGCATTGAC |
| | GTTGAGCGAAAACGCACGTTTACCATGATGATTCGGGAAGGTGTG |
| | GCCATGCACGCCTTTAACGGTGAACTGTTCGTTCAGGCCACCTGG |
| | GATACCAGTTCGTCGCGGCTTTTCCGGACACAGTTCCGGATGGTC |
| | AGCCCGAAGCGCATCAGCAACCCGAACAATACCGGCGACAGCCGG |
| | AACTGCCGTGCCGGTGTGCAGATTAATGACAGCGGTGCGGCGCTG |
| | GGATATTACGTCAGCGAGGACGGGTATCCTGGCTGGATGCCGCAG |
| | AAATGGACATGGATACCCCGTGAGTTACCCGGCGGGCGCGCTTGG |
| | CGTAATCATGGTCATAGCTGTTTCCTGTGTGAAATTGTTATCCGC |
| | TCACAATTCCACACAACATACGAGCCGGAAGCATAAAGTGTAAAG |
| | CCTGGGGTGCCTAATGAGTGAGCTAACTCACATTAATTGCGTTGC |
| | GCTCACTGCCCGCTTTCCAGTCGGGAAACCTGTCGTGCCAGCTGC |
| | ATTAATGAATCGGCCAACGCGCGGGGAGAGGCGGTTTGCGTATTG |
| | GGCGCTCTTCCGCTTCCTCGCTCACTGACTCGCTGCGCTCGGTCG |
| | TTCGGCTGCGGCGAGCGGTATCAGCTCACTCAAAGGCGGTAATAC |
| | GGTTATCCACAGAATCAGGGGATAACGCAGGAAAGAACATGTGAG |
| | CAAAAGGCCAGCAAAAGGCCAGGAACCGTAAAAAGGCCGCGTTGC |
| | TGGCGTTTTTCCATAGGCTCCGCCCCCCTGACGAGCATCACAAAA |
| | ATCGACGCTCAAGTCAGAGGTGGCGAAACCCGACAGGACTATAAA |
| | GATACCAGGCGTTTCCCCCTGGAAGCTCCCTCGTGCGCTCTCCTG |
| | TTCCGACCCTGCCGCTTACCGGATACCTGTCCGCCTTTCTCCCTT |
| | CGGGAAGCGTGGCGCTTTCTCATAGCTCACGCTGTAGGTATCTCA |
| | GTTCGGTGTAGGTCGTTCGCTCCAAGCTGGGCTGTGTGCACGAAC |
| | CCCCCGTTCAGCCCGACCGCTGCGCCTTATCCGGTAACTATCGTC |
| | TTGAGTCCAACCCGGTAAGACACGACTTATCGCCACTGGCAGCAG |
| | CCACTGGTAACAGGATTAGCAGAGCGAGGTATGTAGGCGGTGCTA |
| | CAGAGTTCTTGAAGTGGTGGCCTAACTACGGCTACACTAGAAGGA |
| | CAGTATTTGGTATCTGCGCTCTGCTGAAGCCAGTTACCTTCGGAA |
| | AAAGAGTTGGTAGCTCTTGATCCGGCAAACAAACCACCGCTGGTA |
| | GCGGTGGTTTTTTTGTTTGCAAGCAGCAGATTACGCGCAGAAAAA |
| | AAGGATCTCAAGAAGATCCTTTGATCTTTTCTACGGGGTCTGACG |
| | CTCAGTGGAACGAAAACTCACGTTAAGGGATTTTGGTCATGAGAT |
| | TATCAAAAAGGATCTTCACCTAGATCCTTTTAAATTAAAAATGAA |
| | GTTTTAAATCAATCTAAAGTATATATGAGTAAACTTGGTCTGACA |
| | GTTACCAATGCTTAATCAGTGAGGCACCTATCTCAGCGATCTGTC |
| | TATTTCGTTCATCCATAGTTGCCTGACTCCCCGTCGTGTAGATAA |
| | CTACGATACGGGAGGGCTTACCATCTGGCCCCAGTGCTGCAATGA |
| | TACCGCGAGACCCACGCTCACCGGCTCCAGATTTATCAGCAATAA |
| | ACCAGCCAGCCGGAAGGGCCGAGCGCAGAAGTGGTCCTGCAACTT |
| | TATCCGCCTCCATCCAGTCTATTAATTGTTGCCGGGAAGCTAGAG |
| | TAAGTAGTTCGCCAGTTAATAGTTTGCGCAACGTTGTTGCCATTG |
| | CTACAGGCATCGTGGTGTCACGCTCGTCGTTTGGTATGGCTTCAT |
| | TCAGCTCCGGTTCCCAACGATCAAGGCGAGTTACATGATCCCCCA |
| | TGTTGTGCAAAAAAGCGGTTAGCTCCTTCGGTCCTCCGATCGTTG |
| | TCAGAAGTAAGTTGGCCGCAGTGTTATCACTCATGGTTATGGCAG |
| | CACTGCATAATTCTCTTACTGTCATGCCATCCGTAAGATGCTTTT |
| | CTGTGACTGGTGAGTACTCAACCAAGTCATTCTGAGAATAGTGTA |
| | TGCGGCGACCGAGTTGCTCTTGCCCGGCGTCAATACGGGATAATA |
| | CCGCGCCACATAGCAGAACTTTAAAAGTGCTCATCATTGGAAAAC |
| | GTTCTTCGGGGCGAAAACTCTCAAGGATCTTACCGCTGTTGAGAT |
| | CCAGTTCGATGTAACCCACTCGTGCACCCAACTGATCTTCAGCAT |
| | CTTTTACTTTCACCAGCGTTTCTGGGTGAGCAAAAACAGGAAGGC |
| | AAAATGCCGCAAAAAAGGGAATAAGGGCGACACGGAAATGTTGAA |
| | TACTCAT (SEQ ID NO: 83) |
| PB-TRE-V5- | ACTCTTCCTTTTTCAATATTATTGAAGCATTTATCAGGGTTATTG |
| dCas13b-NES EF | TCTCATGAGCGGATACATATTTGAATGTATTTAGAAAAATAAACA |
| NLS-Turq | AATAGGGGTTCCGCGCACATTTCCCCGAAAAGTGCCACCTAAATT |
| | GTAAGCGTTAATATTTTGTTAAAATTCGCGTTAAATTTTTGTTAA |
| | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTTCTACCGGGTAGGGGAGGCGCTTTTCCCAAGGC |
| | AGTCTGGAGCATGCGCTTTAGCAGCCCCGCTGGGCACTTGGCGCT |
| | ACACAAGTGGCCTCTGGCCTCGCACACATTCCACATCCACCGGTA |
| | GGCGCCAACCGGCTCCGTTCTTTGGTGGCCCCTTCGCGCCACCTT |
| | CTACTCCTCCCCTAGTCAGGAAGTTCCCCCCCGCCCCGCAGCTCG |
| | CGTCGTGCAGGACGTGACAAATGGAAGTAGCACGTCTCATTAGTC |
| | TCGTGCAGATGGACAGCACCGCTGAGCAATGGAAGCGGGTAGGCC |
| | TTTGGGGCAGCGGCCAATAGCAGCTTTGCTCCTTCGCTTTCTGGG |
| | CTCAGAGGCTGGGAAGGGGTGGGTCCGGGGGCGGGCTCAGGGGCG |
| | GGCTCAGGGGCGGGGCGGGCGCCCGAAGGTCCTCCGGAGGCCCGG |
| | CATTCTGCACGCTTCAAAAGCGCACGTCTGCCGCGCTGTTCTCCT |
| | CTTCCTCATCTCCGGGCCTTTCGACCTGCAGGTTAATTAAATGGG |
| | TAAGCCTATCCCTAACCCTCTCCTCGGTCTCGATTCTACGGCTAG |
| | CATGAACATCCCCGCTCTGGTGGAAAACCAGAAGAAGTACTTTGG |
| | CACCTACAGCGTGATGGCCATGCTGAACGCTCAGACCGTGCTGGA |
| | CCACATCCAGAAGGTGGCCGATATTGAGGGCGAGCAGAACGAGAA |
| | CAACGAGAATCTGTGGTTTCACCCCGTGATGAGCCACCTGTACAA |
| | CGCCAAGAACGGCTACGACAAGCAGCCCGAGAAAACCATGTTCAT |
| | CATCGAGCGGCTGCAGAGCTACTTCCCATTCCTGAAGATCATGGC |
| | CGAGAACCAGAGAGAGTACAGCAACGGCAAGTACAAGCAGAACCG |
| | CGTGGAAGTGAACAGCAACGACATCTTCGAGGTGCTGAAGCGCGC |
| | CTTCGGCGTGCTGAAGATGTACAGGGACCTGACCAACGCATACAA |
| | GACCTACGAGGAAAAGCTGAACGACGGCTGCGAGTTCCTGACCAG |
| | CACAGAGCAACCTCTGAGCGGCATGATCAACAACTACTACACAGT |
| | GGCCCTGCGGAACATGAACGAGAGATACGGCTACAAGACAGAGGA |
| | CCTGGCCTTCATCCAGGACAAGCGGTTCAAGTTCGTGAAGGACGC |
| | CTACGGCAAGAAAAAGTCCCAAGTGAATACCGGATTCTTCCTGAG |
| | CCTGCAGGACTACAACGGCGACACACAGAAGAAGCTGCACCTGAG |
| | CGGAGTGGGAATCGCCCTGCTGATCTGCCTGTTCCTGGACAAGCA |
| | GTACATCAACATCTTTCTGAGCAGGCTGCCCATCTTCTCCAGCTA |
| | CAATGCCCAGAGCGAGGAACGGCGGATCATCATCAGATCCTTCGG |
| | CATCAACAGCATCAAGCTGCCCAAGGACCGGATCCACAGCGAGAA |
| | GTCCAACAAGAGCGTGGCCATGGATATGCTCAACGAAGTGAAGCG |
| | GTGCCCCGACGAGCTGTTCACAACACTGTCTGCCGAGAAGCAGTC |
| | CCGGTTCAGAATCATCAGCGACGACCACAATGAAGTGCTGATGAA |
| | GCGGAGCAGCGACAGATTCGTGCCTCTGCTGCTGCAGTATATCGA |
| | TTACGGCAAGCTGTTCGACCACATCAGGTTCCACGTGAACATGGG |
| | CAAGCTGAGATACCTGCTGAAGGCCGACAAGACCTGCATCGACGG |
| | CCAGACCAGAGTCAGAGTGATCGAGCAGCCCCTGAACGGCTTCGG |
| | CAGACTGGAAGAGGCCGAGACAATGCGGAAGCAAGAGAACGGCAC |
| | CTTCGGCAACAGCGGCATCCGGATCAGAGACTTCGAGAACATGAA |
| | GCGGGACGACGCCAATCCTGCCAACTATCCCTACATCGTGGACAC |
| | CTACACACACTACATCCTGGAAAACAACAAGGTCGAGATGTTTAT |
| | CAACGACAAAGAGGACAGCGCCCCACTGCTGCCCGTGATCGAGGA |
| | TGATAGATACGTGGTCAAGACAATCCCCAGCTGCCGGATGAGCAC |
| | CCTGGAAATTCCAGCCATGGCCTTCCACATGTTTCTGTTCGGCAG |
| | CAAGAAAACCGAGAAGCTGATCGTGGACGTGCACAACCGGTACAA |
| | GAGACTGTTCCAGGCCATGCAGAAAGAAGAAGTGACCGCCGAGAA |
| | TATCGCCAGCTTCGGAATCGCCGAGAGCGACCTGCCTCAGAAGAT |
| | CCTGGATCTGATCAGCGGCAATGCCCACGGCAAGGATGTGGACGC |
| | CTTCATCAGACTGACCGTGGACGACATGCTGACCGACACCGAGCG |
| | GAGAATCAAGAGATTCAAGGACGACCGGAAGTCCATTCGGAGCGC |
| | CGACAACAAGATGGGAAAGAGAGGCTTCAAGCAGATCTCCACAGG |
| | CAAGCTGGCCGACTTCCTGGCCAAGGACATCGTGCTGTTTCAGCC |
| | CAGCGTGAACGATGGCGAGAACAAGATCACCGGCCTGAACTACCG |
| | GATCATGCAGAGCGCCATTGCCGTGTACGATAGCGGCGACGATTA |
| | CGAGGCCAAGCAGCAGTTCAAGCTGATGTTCGAGAAGGCCCGGCT |
| | GATCGGCAAGGGCACAACAGAGCCTCATCCATTTCTGTACAAGGT |
| | GTTCGCCCGCAGCATCCCCGCCAATGCCGTCGAGTTCTACGAGCG |
| | CTACCTGATCGAGCGGAAGTTCTACCTGACCGGCCTGTCCAACGA |
| | GATCAAGAAAGGCAACAGAGTGGATGTGCCCTTCATCCGGCGGGA |
| | CCAGAACAAGTGGAAAACACCCGCCATGAAGACCCTGGGCAGAAT |
| | CTACAGCGAGGATCTGCCCGTGGAACTGCCCAGACAGATGTTCGA |
| | CAATGAGATCAAGTCCCACCTGAAGTCCCTGCCACAGATGGAAGG |
| | CATCGACTTCAACAATGCCAACGTGACCTATCTGATCGCCGAGTA |
| | CATGAAGAGAGTGCTGGACGACGACTTCCAGACCTTCTACCAGTG |
| | GAACCGCAACTACCGGTACATGGACATGCTTAAGGGCGAGTACGA |
| | CAGAAAGGGCTCCCTGCAGCACTGCTTCACCAGCGTGGAAGAGAG |
| | AGAAGGCCTCTGGAAAGAGCGGGCCTCCAGAACAGAGCGGTACAG |
| | AAAGCAGGCCAGCAACAAGATCCGCAGCAACCGGCAGATGAGAAA |
| | CGCCAGCAGCGAAGAGATCGAGACAATCCTGGATAAGCGGCTGAG |
| | CAACAGCCGGAACGAGTACCAGAAAAGCGAGAAAGTGATCCGGCG |
| | CTACAGAGTGCAGGATGCCCTGCTGTTTCTGCTGGCCAAAAAGAC |
| | CCTGACCGAACTGGCCGATTTCGACGGCGAGAGGTTCAAACTGAA |
| | AGAAATCATGCCCGACGCCGAGAAGGGAATCCTGAGCGAGATCAT |
| | GCCCATGAGCTTCACCTTCGAGAAAGGCGGCAAGAAGTACACCAT |
| | CACCAGCGAGGGCATGAAGCTGAAGAACTACGGCGACTTCTTTGT |
| | GCTGGCTAGCGACAAGAGGATCGGCAACCTGCTGGAACTCGTGGG |
| | CAGCGACATCGTGTCCAAAGAGGATATCATGGAAGAGTTCAACAA |
| | ATACGACCAGTGCAGGCCCGAGATCAGCTCCATCGTGTTCAACCT |
| | GGAAAAGTGGGCCTTCGACACATACCCCGAGCTGTCTGCCAGAGT |
| | GGACCGGGAAGAGAAGGTGGACTTCAAGAGCATCCTGAAAATCCT |
| | GCTGAACAACAAGAACATCAACAAAGAGCAGAGCGACATCCTGCG |
| | GAAGATCCGGAACGCCTTCGATGCAAACAATTACCCCGACAAAGG |
| | CGTGGTGGAAATCAAGGCCCTGCCTGAGATCGCCATGAGCATCAA |
| | GAAGGCCTTTGGGGAGTACGCCATCATGAAGGGATCCCTTCAACT |
| | GCCTCCACTTGAAAGACTGACACTGCTCGAGAGAGATTAGATCTG |
| | TTGGACGATGATCGGAGAGTGACTGGGTTTAGTATAACCGGTGGT |
| | GAACATAGGCTGAGGAATTATAAATCGGTTACGACGGTTCATAGA |
| | TTTGAGAAAGAAGAAGAAGAAGAAAGGATCTGGACCGTTGTTTTG |
| | GAATCTTATGTTGTTGATGTACCGGAAGGTAATTCGGAGGAAGAT |
| | ACGAGATTGTTTGCTGATACGGTTATTAGATTGAATCTTCAGAAA |
| | CTTGCTTCGATCACTGAAGCTATGAACTCTAGACTTAAGCAACAT |
| | GCTGCTCCGCCGAAAAAGAAGAGAAAAGGTTAAGCCTCGACTGTG |
| | CCTTCTAGTTGCCAGCCATCTGTTGTTTGCCCCTCCCCCGTGCCT |
| | TCCTTGACCCTGGAAGGTGCCACTCCCACTGTCCTTTCCTAATAA |
| | AATGAGGAAATTGCATCGCATTGTCTGAGTAGGTGTCATTCTATT |
| | CTGGGGGGTGGGGTGGGGCAGGACAGCAAGGGGGAGGATTGGGAA |
| | GACAATAGCAGGCATGCTGGGGAGTGCCCGTCAGTGGGCAGAGCG |
| | CACATCGCCCACAGTCCCCGAGAAGTTGGGGGGAGGGGTCGGCAA |
| | TTGAACGGGTGCCTAGAGAAGGTGGCGCGGGGTAAACTGGGAAAG |
| | TGATGTCGTGTACTGGCTCCGCCTTTTTCCCGAGGGTGGGGGAGA |
| | ACCGTATATAAGTGCAGTAGTCGCCGTGAACGTTCTTTTTCGCAA |
| | CGGGTTTGCCGCCAGAACACAGATGGTCTCTAAAGGAGGTTCGTC |
| | CGACGACGAAGCAACAGCGGACTCGCAGCACGCCGCACCTCCTAA |
| | GAAGAAAAGGAAGGTAGGGGATCCCCGGGTACCGGTCGCCACCAT |
| | GGTGAGCAAGGGCGAGGAGCTGTTCACCGGGGTGGTGCCCATCCT |
| | GGTCGAGCTGGACGGCGACGTAAACGGCCACAAGTTCAGCGTGTC |
| | CGGCGAGGGCGAGGGCGATGCCACCTACGGCAAGCTGACCCTGAA |
| | GTTCATCTGCACCACCGGCAAGCTGCCCGTGCCCTGGCCCACCCT |
| | CGTGACCACCCTGTCCTGGGGCGTGCAGTGCTTCGCCCGCTACCC |
| | CGACCACATGAAGCAGCACGACTTCTTCAAGTCCGCCATGCCCGA |
| | AGGCTACGTCCAGGAGCGCACCATCTTCTTCAAGGACGACGGCAA |
| | CTACAAGACCCGCGCCGAGGTGAAGTTCGAGGGCGACACCCTGGT |
| | GAACCGCATCGAGCTGAAGGGCATCGACTTCAAGGAGGACGGCAA |
| | CATCCTGGGGCACAAGCTGGAGTACAACTACTTTAGCGACAACGT |
| | CTATATCACCGCCGACAAGCAGAAGAACGGCATCAAGGCCAACTT |
| | CAAGATCCGCCACAACATCGAGGACGGCGGCGTGCAGCTCGCCGA |
| | CCACTACCAGCAGAACACCCCCATCGGCGACGGCCCCGTGCTGCT |
| | GCCCGACAACCACTACCTGAGCACCCAGTCCAAGCTGAGCAAAGA |
| | CCCCAACGAGAAGCGCGATCACATGGTCCTGCTGGAGTTCGTGAC |
| | CGCCGCCGGGATCACTCTCGGCATGGACGAGCTGTACAAGTAAAA |
| | TCAACCTCTGGATTACAAAATTTGTGAAAGATTGACTGGTATTCT |
| | TAACTATGTTGCTCCTTTTACGCTATGTGGATACGCTGCTTTAAT |
| | GCCTTTGTATCATGTTAACTAAACTTGTTTATTGCAGCTTATAAT |
| | GGTTACAAATAAAGCAATAGCATCACAAATTTCACAAATAAAGCA |
| | TTTTTTTCACTGCATTCTAGTTGTGGTTTGTCCAAACTCATCAAT |
| | GTATCTTATCATGTCTGGAATTGACTCAAATGATGTCAATTAGTC |
| | TATCAGAAGCTCATCTGGTCTCCCTTCCGGGGGACAAGACATCCC |
| | TGTTTAATATTTAAACAGCAGTGTTCCCAAACTGGGTTCTTATAT |
| | CCCTTGCTCTGGTCAACCAGGTTGCAGGGTTTCCTGTCCTCACAG |
| | GAACGAAGTCCCTAAAGAAACAGTGGCAGCCAGGTTTAGCCCCGG |
| | AATTGACTGGATTCCTTTTTTAGGGCCCATTGGTATGGCTTTTTC |
| | CCCGTATCCCCCCAGGTGTCTGCAGGCTCAAAGAGCAGCGAGAAG |
| | CGTTCAGAGGAAAGCGATCCCGTGCCACCTTCCCCGTGCCCGGGC |
| | TGTCCCCGCACGCTGCCGGCTCGGGGATGCGGGGGGAGCGCCGGA |
| | CCGGAGCGGAGCCCCGGGCGGCTCGCTGCTGCCCCCTAGCGGGGG |
| | AGGGACGTAATTACATCCCTGGGGGCTTTGGGGGGGGGCTGTCCC |
| | TGATATCTATAACAAGAAAATATATATATAATAAGTTATCACGTA |
| | AGTAGAACATGAAATAACAATATAATTATCGTATGAGTTAAATCT |
| | TAAAAGTCACGTAAAAGATAATCATGCGTCATTTTGACTCACGCG |
| | GTCGTTATAGTTCAAAATCAGTGACACTTACCGCATTGACAAGCA |
| | CGCCTCACGGGAGCTCCAAGCGGCGACTGAGATGTCCTAAATGCA |
| | CAGCGACGGATTCGCGCTATTTAGAAAGAGAGAGCAATATTTCAA |
| | GAATGCATGCGTCAATTTTACGCAGACTATCTTTCTAGGGTTAAT |
| | CTAGCTGCATCAGGATCATATCGTCGGGTCTTTTTTCCGGCTCAG |
| | TCATCGCCCAAGCTGGCGCTATCTGGGCATCGGGGAGGAAGAAGC |
| | CCGTGCCTTTTCCCGCGAGGTTGAAGCGGCATGGAAAGAGTTTGC |
| | CGAGGATGACTGCTGCTGCATTGACGTTGAGCGAAAACGCACGTT |
| | TACCATGATGATTCGGGAAGGTGTGGCCATGCACGCCTTTAACGG |
| | TGAACTGTTCGTTCAGGCCACCTGGGATACCAGTTCGTCGCGGCT |
| | TTTCCGGACACAGTTCCGGATGGTCAGCCCGAAGCGCATCAGCAA |
| | CCCGAACAATACCGGCGACAGCCGGAACTGCCGTGCCGGTGTGCA |
| | GATTAATGACAGCGGTGCGGCGCTGGGATATTACGTCAGCGAGGA |
| | CGGGTATCCTGGCTGGATGCCGCAGAAATGGACATGGATACCCCG |
| | TGAGTTACCCGGCGGGCGCGCTTGGCGTAATCATGGTCATAGCTG |
| | TTTCCTGTGTGAAATTGTTATCCGCTCACAATTCCACACAACATA |
| | CGAGCCGGAAGCATAAAGTGTAAAGCCTGGGGTGCCTAATGAGTG |
| | AGCTAACTCACATTAATTGCGTTGCGCTCACTGCCCGCTTTCCAG |
| | TCGGGAAACCTGTCGTGCCAGCTGCATTAATGAATCGGCCAACGC |
| | GCGGGGAGAGGCGGTTTGCGTATTGGGCGCTCTTCCGCTTCCTCG |
| | CTCACTGACTCGCTGCGCTCGGTCGTTCGGCTGCGGCGAGCGGTA |
| | TCAGCTCACTCAAAGGCGGTAATACGGTTATCCACAGAATCAGGG |
| | GATAACGCAGGAAAGAACATGTGAGCAAAAGGCCAGCAAAAGGCC |
| | AGGAACCGTAAAAAGGCCGCGTTGCTGGCGTTTTTCCATAGGCTC |
| | CGCCCCCCTGACGAGCATCACAAAAATCGACGCTCAAGTCAGAGG |
| | TGGCGAAACCCGACAGGACTATAAAGATACCAGGCGTTTCCCCCT |
| | GGAAGCTCCCTCGTGCGCTCTCCTGTTCCGACCCTGCCGCTTACC |
| | GGATACCTGTCCGCCTTTCTCCCTTCGGGAAGCGTGGCGCTTTCT |
| | CATAGCTCACGCTGTAGGTATCTCAGTTCGGTGTAGGTCGTTCGC |
| | TCCAAGCTGGGCTGTGTGCACGAACCCCCCGTTCAGCCCGACCGC |
| | TGCGCCTTATCCGGTAACTATCGTCTTGAGTCCAACCCGGTAAGA |
| | CACGACTTATCGCCACTGGCAGCAGCCACTGGTAACAGGATTAGC |
| | AGAGCGAGGTATGTAGGCGGTGCTACAGAGTTCTTGAAGTGGTGG |
| | CCTAACTACGGCTACACTAGAAGGACAGTATTTGGTATCTGCGCT |
| | CTGCTGAAGCCAGTTACCTTCGGAAAAAGAGTTGGTAGCTCTTGA |
| | TCCGGCAAACAAACCACCGCTGGTAGCGGTGGTTTTTTTGTTTGC |
| | AAGCAGCAGATTACGCGCAGAAAAAAAGGATCTCAAGAAGATCCT |
| | TTGATCTTTTCTACGGGGTCTGACGCTCAGTGGAACGAAAACTCA |
| | CGTTAAGGGATTTTGGTCATGAGATTATCAAAAAGGATCTTCACC |
| | TAGATCCTTTTAAATTAAAAATGAAGTTTTAAATCAATCTAAAGT |
| | ATATATGAGTAAACTTGGTCTGACAGTTACCAATGCTTAATCAGT |
| | GAGGCACCTATCTCAGCGATCTGTCTATTTCGTTCATCCATAGTT |
| | GCCTGACTCCCCGTCGTGTAGATAACTACGATACGGGAGGGCTTA |
| | CCATCTGGCCCCAGTGCTGCAATGATACCGCGAGACCCACGCTCA |
| | CCGGCTCCAGATTTATCAGCAATAAACCAGCCAGCCGGAAGGGCC |
| | GAGCGCAGAAGTGGTCCTGCAACTTTATCCGCCTCCATCCAGTCT |
| | ATTAATTGTTGCCGGGAAGCTAGAGTAAGTAGTTCGCCAGTTAAT |
| | AGTTTGCGCAACGTTGTTGCCATTGCTACAGGCATCGTGGTGTCA |
| | CGCTCGTCGTTTGGTATGGCTTCATTCAGCTCCGGTTCCCAACGA |
| | TCAAGGCGAGTTACATGATCCCCCATGTTGTGCAAAAAAGCGGTT |
| | AGCTCCTTCGGTCCTCCGATCGTTGTCAGAAGTAAGTTGGCCGCA |
| | GTGTTATCACTCATGGTTATGGCAGCACTGCATAATTCTCTTACT |
| | GTCATGCCATCCGTAAGATGCTTTTCTGTGACTGGTGAGTACTCA |
| | ACCAAGTCATTCTGAGAATAGTGTATGCGGCGACCGAGTTGCTCT |
| | TGCCCGGCGTCAATACGGGATAATACCGCGCCACATAGCAGAACT |
| | TTAAAAGTGCTCATCATTGGAAAACGTTCTTCGGGGCGAAAACTC |
| | TCAAGGATCTTACCGCTGTTGAGATCCAGTTCGATGTAACCCACT |
| | CGTGCACCCAACTGATCTTCAGCATCTTTTACTTTCACCAGCGTT |
| | TCTGGGTGAGCAAAAACAGGAAGGCAAAATGCCGCAAAAAAGGGA |
| | ATAAGGGCGACACGGAAATGTTGAATACTCAT (SEQ ID NO: |
| | 84) |
| 5′UTR | MDYKDDDDK*AAGPTDGR (SEQ ID NO: 85 and 351) |
| FLAG ORF | MDYKDDDDK (SEQ ID NO: 86) |
| Feature 42 | MMMINKQLDQRTDASAKDPRVPVAT (SEQ ID NO: 349) |
| Citrine | MVSKGEELFTGVVPILVELDGDVNGHKFSVSGEGEGDATYGKLTL |
| | KFICTTGKLPVPWPTLVTTFGYGLMCFARYPDHMKQHDFFKSAMP |
| | EGYVQERTIFFKDDGNYKTRAEVKFEGDTLVNRIELKGIDFKEDG |
| | NILGHKLEYNYNSHNVYIMADKQKNGIKVNFKIRHNIEDGSVQLA |
| | DHYQQNTPIGDGPVLLPDNHYLSYQSALSKDPNEKRDHMVLLEFV |
| | TAAGITLGMDELYPYDVPDYA (SEQ ID NO: 87) |
| HA (human | YPYDVPDYA (SEQ ID NO: 88) |
| influenza | |
| hemagglutinin) | |
| epitope tag | |
| Mod SDHA 3′UTR | AYVHLRALTCACEDQFLR*VLRFLK*QDSPHGIPLITGILDLIAL |
| | FSK (SEQ ID NOs: 89 and 352-353) |
| rtTA | MSRLDKSKVINGALELLNGVGIEGLTTRKLAQKLGVEQPTLYWHV |
| | KNKRALLDALPIEMLDRHHTHFCPLEGESWQDFLRNNAKSFRCAL |
| | LSHRDGAKVHLGTRPTEKQYETLENQLAFLCQQGFSLENALYALS |
| | AVGHFTLGCVLEEQEHQVAKEERETPTTDSMPPLLRQAIELFDRQ |
| | GAEPAFLFGLELIICGLEKQLKCESGGPADALDDFDLDMLPADAL |
| | DDFDLDMLPADALDDFDLDMLPG (SEQ ID NO: 90) |
| PB-PGK-(5UTR- | ACTCTTCCTTTTTCAATATTATTGAAGCATTTATCAGGGTTATTG |
| uORF)-Citrine- | TCTCATGAGCGGATACATATTTGAATGTATTTAGAAAAATAAACA |
| U6-crRNA13b- | AATAGGGGTTCCGCGCACATTTCCCCGAAAAGTGCCACCTAAATT |
| IRES-EF-Puro- | GTAAGCGTTAATATTTTGTTAAAATTCGCGTTAAATTTTTGTTAA |
| NLS-Cherry | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTACTTTCACTTTTCTCTATCACTGATAGGGAGTG |
| | GTAAACTCGACTTTCACTTTTCTCTATCACTGATAGGGAGTGGTA |
| | AACTCGACTTTCACTTTTCTCTATCACTGATAGGGAGTGGTAAAC |
| | TCGACTTTCACTTTTCTCTATCACTGATAGGGAGTGGTAAACTCG |
| | ACTTTCACTTTTCTCTATCACTGATAGGGAGTGGTAAACTCGACT |
| | TTCACTTTTCTCTATCACTGATAGGGAGTGGTAAACTCGACTTTC |
| | ACTTTTCTCTATCACTGATAGGGAGTGGTAAACTCGACCTATATA |
| | AGCAGAGCTCGTTTAGTGAACCGTCAGATCGCCTGGAGACGCCAT |
| | CCACGCTGTTTTGACCTCCATAGAAGACACCGGGACCGATCCAGC |
| | CTCCGCGGCCCCGAATTCATGGATTATAAAGATGATGATGATAAA |
| | TAAGCAGCTGGACCAACGGACGGACGCCAGCGCTAAGGATCCCCG |
| | GGTACCGGTCGCCACCATGGTGAGCAAGGGCGAGGAGCTGTTCAC |
| | CGGGGTGGTGCCCATCCTGGTCGAGCTGGACGGCGACGTAAACGG |
| | CCACAAGTTCAGCGTGTCCGGCGAGGGCGAGGGCGATGCCACCTA |
| | CGGCAAGCTGACCCTGAAGTTCATCTGCACCACCGGCAAGCTGCC |
| | CGTGCCCTGGCCCACCCTCGTGACCACCTTCGGCTACGGCCTGAT |
| | GTGCTTCGCCCGCTACCCCGACCACATGAAGCAGCACGACTTCTT |
| | CAAGTCCGCCATGCCCGAAGGCTACGTCCAGGAGCGCACCATCTT |
| | CTTCAAGGACGACGGCAACTACAAGACCCGCGCCGAGGTGAAGTT |
| | CGAGGGCGACACCCTGGTGAACCGCATCGAGCTGAAGGGCATCGA |
| | CTTCAAGGAGGACGGCAACATCCTGGGGCACAAGCTGGAGTACAA |
| | CTACAACAGCCACAACGTCTATATCATGGCCGACAAGCAGAAGAA |
| | CGGCATCAAGGTGAACTTCAAGATCCGCCACAACATCGAGGACGG |
| | CAGCGTGCAGCTCGCCGACCACTACCAGCAGAACACCCCCATCGG |
| | CGACGGCCCCGTGCTGCTGCCCGACAACCACTACCTGAGCTACCA |
| | GTCCGCCCTGAGCAAAGACCCCAACGAGAAGCGCGATCACATGGT |
| | CCTGCTGGAGTTCGTGACCGCCGCCGGGATCACTCTCGGCATGGA |
| | CGAGCTGTACCCATACGATGTTCCAGATTACGCTTAAGCTAGCGC |
| | ATATGTCCACTTAAGGGCGCTGACATGCGCATGTGAGGATCAATT |
| | CTTACGCTGAGTACTTCGATTCCTCAAATAGCAAGACAGCCCACA |
| | TGGCATTCCACTTATCACTGGCATCCTAGATCTGATAGCTTTGTT |
| | CTCAAAGTCTCGAGAAATTCGAATTTAAATCGACGCGTGCCTCGA |
| | CTGTGCCTTCTAGTTGCCAGCCATCTGTTGTTTGCCCCTCCCCCG |
| | TGCCTTCCTTGACCCTGGAAGGTGCCACTCCCACTGTCCTTTCCT |
| | AATAAAATGAGGAAATTGCATCGCATTGTCTGAGTAGGTGTCATT |
| | CTATTCTGGGGGGTGGGGTGGGGCAGGACAGCAAGGGGGAGGATT |
| | GGGAAGACAATAGCAGGCATGCTGGGGAAATAAAGCAAAAAAAAA |
| | AATATCATCGTGTTCTTCAAAGGAAAACCACGTCCCCGTGGTTCG |
| | GGGGGCCTAGACGTTTTTTTAACCTCGACTAAACACATGTAAAGC |
| | ATGTGCACCGAGGCCCCAGATCAGATCCCATACAATGGGGTACCT |
| | TCTGGGCATCCTTCAGCCCCTTGTTGAATACGCTTGAGGAGAGCC |
| | ATTTGACTCTTTCCACAACTATCCAACTCACAACGTGGCACTGGG |
| | GTTGTGCCGCCTTTGCAGGTGTATCTTATACACGTGGCTTTTGGC |
| | CGCAGAGGCACCTGTCGCCAGGTGGGGGGTTCCGCTGCCTGCAAA |
| | GGGTCGCTACAGACGTTGTTTGTCTTCAAGAAGCTTCCAGAGGAA |
| | CTGCTTCCTTCACGACATTCAACAGACCTTGCATTCCTTTGGCGA |
| | GAGGGGAAAGACCCCTAGGAATGCTCGTCAAGAAGACAGGGCCAG |
| | GTTTCCGGGCCCTCACATTGCCAAAAGACGGCAATATGGTGGAAA |
| | ATAACATATAGACAAACGCACACCGGCCTTATTCCAAGCGGCTTC |
| | GGCCAGTAACGTTAGGGGGGGGGGAGGGAGAGGGGTCGACGTTGT |
| | AATAGCCCCTCAAAACTGGACCTTCCACAACTAGTGTTCTCGAAC |
| | ATGGCATTGGGAACACGGTGTTTCGTCCTTTCCACAAGATATATA |
| | AAGCCAAGAAATCGAAATACTTTCAAGTTACGGTAAGCATATGAT |
| | AGTCCATTTTAAAACATAATTTTAAAACTGCAAACTACCCAAGAA |
| | ATTATTACTTTCTACGTCACGTATTTTGTACTAATATCTTTGTGT |
| | TTACAGTCAAATTAATTCTAATTATCTCTCTAACAGCCTTGTATC |
| | GTATATGCAAATATGAAGGAATCATGGGAAATAGGCCCTCTTCCT |
| | GCCCGACCTCGCGGCCGCGAAGGATCTGCGATCGCTCCGGTGCCC |
| | GTCAGTGGGCAGAGCGCACATCGCCCACAGTCCCCGAGAAGTTGG |
| | GGGGAGGGGTCGGCAATTGAACGGGTGCCTAGAGAAGGTGGCGCG |
| | GGGTAAACTGGGAAAGTGATGTCGTGTACTGGCTCCGCCTTTTTC |
| | CCGAGGGTGGGGGAGAACCGTATATAAGTGCAGTAGTCGCCGTGA |
| | ACGTTCTTTTTCGCAACGGGTTTGCCGCCAGAACACAGCTGAAGC |
| | TTCGAGGGGCTCGCATCTCTCCTTCACGCGCCCGCCGCCCTACCT |
| | GAGGCCGCCATCCACGCCGGTTGAGTCGCGTTCTGCCGCCTCCCG |
| | CCTGTGGTGCCTCCTGAACTGCGTCCGCCGTCTAGGTAAGTTTAA |
| | AGCTCAGGTCGAGACCGGGCCTTTGTCCGGCGCTCCCTTGGAGCC |
| | TACCTAGACTCAGCCGGCTCTCCACGCTTTGCCTGACCCTGCTTG |
| | CTCAACTCTACGTCTTTGTTTCGTTTTCTGTTCTGCGCCGTTACA |
| | GATCCAAGCTGTGACCGGCGCCTACGCTAGATGACCGAGTACAAG |
| | CCCACGGTGCGCCTCGCCACCCGCGACGACGTCCCCAGGGCCGTA |
| | CGCACCCTCGCCGCCGCGTTCGCCGACTACCCCGCCACGCGCCAC |
| | ACCGTCGATCCGGACCGCCACATCGAGCGGGTCACCGAGCTGCAA |
| | GAACTCTTCCTCACGCGCGTCGGGCTCGACATCGGCAAGGTGTGG |
| | GTCGCGGACGACGGCGCCGCGGTGGCGGTCTGGACCACGCCGGAG |
| | AGCGTCGAAGCGGGGGCGGTGTTCGCCGAGATCGGCCCGCGCATG |
| | GCCGAGTTGAGCGGTTCCCGGCTGGCCGCGCAGCAACAGATGGAA |
| | GGCCTCCTGGCGCCGCACCGGCCCAAGGAGCCCGCGTGGTTCCTG |
| | GCCACCGTCGGCGTCTCGCCCGACCACCAGGGCAAGGGTCTGGGC |
| | AGCGCCGTCGTGCTCCCCGGAGTGGAGGCGGCCGAGCGCGCCGGG |
| | GTGCCCGCCTTCCTGGAGACCTCCGCGCCCCGCAACCTCCCCTTC |
| | TACGAGCGGCTCGGCTTCACCGTCACCGCCGACGTCGAGGTGCCC |
| | GAAGGACCGCGCACCTGGTGCATGACCCGCAAGCCCGGTGCCGGA |
| | AGCGGAGCTACTAACTTCAGCCTGCTGAAGCAGGCTGGAGACGTG |
| | GAGGAGAACCCTGGACCTATGTCTAGACTGGACAAGAGCAAAGTC |
| | ATAAACGGCGCTCTGGAATTACTCAATGGAGTCGGTATCGAAGGC |
| | CTGACGACAAGGAAACTCGCTCAAAAGCTGGGAGTTGAGCAGCCT |
| | ACCCTGTACTGGCACGTGAAGAACAAGCGGGCCCTGCTCGATGCC |
| | CTGCCAATCGAGATGCTGGACAGGCATCATACCCACTTCTGCCCC |
| | CTGGAAGGCGAGTCATGGCAAGACTTTCTGCGGAACAACGCCAAG |
| | TCATTCCGCTGTGCTCTCCTCTCACATCGCGACGGGGCTAAAGTG |
| | CATCTCGGCACCCGCCCAACAGAGAAACAGTACGAAACCCTGGAA |
| | AATCAGCTCGCGTTCCTGTGTCAGCAAGGCTTCTCCCTGGAGAAC |
| | GCACTGTACGCTCTGTCCGCCGTGGGCCACTTTACACTGGGCTGC |
| | GTATTGGAGGAACAGGAGCATCAAGTAGCAAAAGAGGAAAGAGAG |
| | ACACCTACCACCGATTCTATGCCCCCACTTCTGAGACAAGCAATT |
| | GAGCTGTTCGACCGGCAGGGAGCCGAACCTGCCTTCCTTTTCGGC |
| | CTGGAACTAATCATATGTGGCCTGGAGAAACAGCTAAAGTGCGAA |
| | AGCGGCGGGCCGGCCGACGCCCTTGACGATTTTGACTTAGACATG |
| | CTCCCAGCCGATGCCCTTGACGACTTTGACCTTGATATGCTGCCT |
| | GCTGACGCTCTTGACGATTTTGACCTTGACATGCTCCCCGGGTAA |
| | CTAAGTAACCCTCTCCCTCCCCCCCCCCTAACGTTACTGGCCGAA |
| | GCCGCTTGGAATAAGGCCGGTGTGCGTTTGTCTATATGTTATTTT |
| | CCACCATATTGCCGTCTTTTGGCAATGTGAGGGCCCGGAAACCTG |
| | GCCCTGTCTTCTTGACGAGCATTCCTAGGGGTCTTTCCCCTCTCG |
| | CCAAAGGAATGCAAGGTCTGTTGAATGTCGTGAAGGAAGCAGTTC |
| | CTCTGGAAGCTTCTTGAAGACAAACAACGTCTGTAGCGACCCTTT |
| | GCAGGCAGCGGAACCCCCCACCTGGCGACAGGTGCCTCTGCGGCC |
| | AAAAGCCACGTGTATAAGATACACCTGCAAAGGCGGCACAACCCC |
| | AGTGCCACGTTGTGAGTTGGATAGTTGTGGAAAGAGTCAAATGGC |
| | TCTCCTCAAGCGTATTCAACAAGGGGCTGAAGGATGCCCAGAAGG |
| | TACCCCATTGTATGGGATCTGATCTGGGGCCTCGGTGCACATGCT |
| | TTACATGTGTTTAGTCGAGGTTAAAAAAACGTCTAGGCCCCCCGA |
| | ACCACGGGGACGTGGTTTTCCTTTGAAAAACACGATGATAATATG |
| | GCCACAACCATGGTCTCTAAAGGAGGTTCGTCCGACGACGAAGCA |
| | ACAGCGGACTCGCAGCACGCCGCACCTCCTAAGAAGAAAAGGAAG |
| | GTAGGGGATCCCCGGGTACCGGTCGCCACCATGGCCATCATCAAG |
| | GAGTTCATGCGCTTCAAGGTGCACATGGAGGGCTCCGTGAACGGC |
| | CACGAGTTCGAGATCGAGGGCGAGGGCGAGGGCCGCCCCTACGAG |
| | GGCACCCAGACCGCCAAGCTGAAGGTGACCAAGGGTGGCCCCCTG |
| | CCCTTCGCCTGGGACATCCTGTCCCCTCAGTTCATGTACGGCTCC |
| | AAGGCCTACGTGAAGCACCCCGCCGACATCCCCGACTACTTGAAG |
| | CTGTCCTTCCCCGAGGGCTTCAAGTGGGAGCGCGTGATGAACTTC |
| | GAGGACGGCGGCGTGGTGACCGTGACCCAGGACTCCTCCCTGCAG |
| | GACGGCGAGTTCATCTACAAGGTGAAGCTGCGCGGCACCAACTTC |
| | CCCTCCGACGGCCCCGTAATGCAGAAGAAGACCATGGGCTGGGAG |
| | GCCTCCTCCGAGCGGATGTACCCCGAGGACGGCGCCCTGAAGGGC |
| | GAGATCAAGCAGAGGCTGAAGCTGAAGGACGGCGGCCACTACGAC |
| | GCTGAGGTCAAGACCACCTACAAGGCCAAGAAGCCCGTGCAGCTG |
| | CCCGGCGCCTACAACGTCAACATCAAGTTGGACATCACCTCCCAC |
| | AACGAGGACTACACCATCGTGGAACAGTACGAACGCGCCGAGGGC |
| | CGCCACTCCACCGGCGGCATGGACGAGCTGTACAAGTAAAATCAA |
| | CCTCTGGATTACAAAATTTGTGAAAGATTGACTGGTATTCTTAAC |
| | TATGTTGCTCCTTTTACGCTATGTGGATACGCTGCTTTAATGCCT |
| | TTGTATCATGCGTTAACTAAACTTGTTTATTGCAGCTTATAATGG |
| | TTACAAATAAAGCAATAGCATCACAAATTTCACAAATAAAGCATT |
| | TTTTTCACTGCATTCTAGTTGTGGTTTGTCCAAACTCATCAATGT |
| | ATCTTATCATGTCTGGAATTGACTCAAATGATGTCAATTAGTCTA |
| | TCAGAAGCTCATCTGGTCTCCCTTCCGGGGGACAAGACATCCCTG |
| | TTTAATATTTAAACAGCAGTGTTCCCAAACTGGGTTCTTATATCC |
| | CTTGCTCTGGTCAACCAGGTTGCAGGGTTTCCTGTCCTCACAGGA |
| | ACGAAGTCCCTAAAGAAACAGTGGCAGCCAGGTTTAGCCCCGGAA |
| | TTGACTGGATTCCTTTTTTAGGGCCCATTGGTATGGCTTTTTCCC |
| | CGTATCCCCCCAGGTGTCTGCAGGCTCAAAGAGCAGCGAGAAGCG |
| | TTCAGAGGAAAGCGATCCCGTGCCACCTTCCCCGTGCCCGGGCTG |
| | TCCCCGCACGCTGCCGGCTCGGGGATGCGGGGGGAGCGCCGGACC |
| | GGAGCGGAGCCCCGGGCGGCTCGCTGCTGCCCCCTAGCGGGGGAG |
| | GGACGTAATTACATCCCTGGGGGCTTTGGGGGGGGGCTGTCCCTG |
| | ATATCTATAACAAGAAAATATATATATAATAAGTTATCACGTAAG |
| | TAGAACATGAAATAACAATATAATTATCGTATGAGTTAAATCTTA |
| | AAAGTCACGTAAAAGATAATCATGCGTCATTTTGACTCACGCGGT |
| | CGTTATAGTTCAAAATCAGTGACACTTACCGCATTGACAAGCACG |
| | CCTCACGGGAGCTCCAAGCGGCGACTGAGATGTCCTAAATGCACA |
| | GCGACGGATTCGCGCTATTTAGAAAGAGAGAGCAATATTTCAAGA |
| | ATGCATGCGTCAATTTTACGCAGACTATCTTTCTAGGGTTAATCT |
| | AGCTGCATCAGGATCATATCGTCGGGTCTTTTTTCCGGCTCAGTC |
| | ATCGCCCAAGCTGGCGCTATCTGGGCATCGGGGAGGAAGAAGCCC |
| | GTGCCTTTTCCCGCGAGGTTGAAGCGGCATGGAAAGAGTTTGCCG |
| | AGGATGACTGCTGCTGCATTGACGTTGAGCGAAAACGCACGTTTA |
| | CCATGATGATTCGGGAAGGTGTGGCCATGCACGCCTTTAACGGTG |
| | AACTGTTCGTTCAGGCCACCTGGGATACCAGTTCGTCGCGGCTTT |
| | TCCGGACACAGTTCCGGATGGTCAGCCCGAAGCGCATCAGCAACC |
| | CGAACAATACCGGCGACAGCCGGAACTGCCGTGCCGGTGTGCAGA |
| | TTAATGACAGCGGTGCGGCGCTGGGATATTACGTCAGCGAGGACG |
| | GGTATCCTGGCTGGATGCCGCAGAAATGGACATGGATACCCCGTG |
| | AGTTACCCGGCGGGCGCGCTTGGCGTAATCATGGTCATAGCTGTT |
| | TCCTGTGTGAAATTGTTATCCGCTCACAATTCCACACAACATACG |
| | AGCCGGAAGCATAAAGTGTAAAGCCTGGGGTGCCTAATGAGTGAG |
| | CTAACTCACATTAATTGCGTTGCGCTCACTGCCCGCTTTCCAGTC |
| | GGGAAACCTGTCGTGCCAGCTGCATTAATGAATCGGCCAACGCGC |
| | GGGGAGAGGCGGTTTGCGTATTGGGCGCTCTTCCGCTTCCTCGCT |
| | CACTGACTCGCTGCGCTCGGTCGTTCGGCTGCGGCGAGCGGTATC |
| | AGCTCACTCAAAGGCGGTAATACGGTTATCCACAGAATCAGGGGA |
| | TAACGCAGGAAAGAACATGTGAGCAAAAGGCCAGCAAAAGGCCAG |
| | GAACCGTAAAAAGGCCGCGTTGCTGGCGTTTTTCCATAGGCTCCG |
| | CCCCCCTGACGAGCATCACAAAAATCGACGCTCAAGTCAGAGGTG |
| | GCGAAACCCGACAGGACTATAAAGATACCAGGCGTTTCCCCCTGG |
| | AAGCTCCCTCGTGCGCTCTCCTGTTCCGACCCTGCCGCTTACCGG |
| | ATACCTGTCCGCCTTTCTCCCTTCGGGAAGCGTGGCGCTTTCTCA |
| | TAGCTCACGCTGTAGGTATCTCAGTTCGGTGTAGGTCGTTCGCTC |
| | CAAGCTGGGCTGTGTGCACGAACCCCCCGTTCAGCCCGACCGCTG |
| | CGCCTTATCCGGTAACTATCGTCTTGAGTCCAACCCGGTAAGACA |
| | CGACTTATCGCCACTGGCAGCAGCCACTGGTAACAGGATTAGCAG |
| | AGCGAGGTATGTAGGCGGTGCTACAGAGTTCTTGAAGTGGTGGCC |
| | TAACTACGGCTACACTAGAAGGACAGTATTTGGTATCTGCGCTCT |
| | GCTGAAGCCAGTTACCTTCGGAAAAAGAGTTGGTAGCTCTTGATC |
| | CGGCAAACAAACCACCGCTGGTAGCGGTGGTTTTTTTGTTTGCAA |
| | GCAGCAGATTACGCGCAGAAAAAAAGGATCTCAAGAAGATCCTTT |
| | GATCTTTTCTACGGGGTCTGACGCTCAGTGGAACGAAAACTCACG |
| | TTAAGGGATTTTGGTCATGAGATTATCAAAAAGGATCTTCACCTA |
| | GATCCTTTTAAATTAAAAATGAAGTTTTAAATCAATCTAAAGTAT |
| | ATATGAGTAAACTTGGTCTGACAGTTACCAATGCTTAATCAGTGA |
| | GGCACCTATCTCAGCGATCTGTCTATTTCGTTCATCCATAGTTGC |
| | CTGACTCCCCGTCGTGTAGATAACTACGATACGGGAGGGCTTACC |
| | ATCTGGCCCCAGTGCTGCAATGATACCGCGAGACCCACGCTCACC |
| | GGCTCCAGATTTATCAGCAATAAACCAGCCAGCCGGAAGGGCCGA |
| | GCGCAGAAGTGGTCCTGCAACTTTATCCGCCTCCATCCAGTCTAT |
| | TAATTGTTGCCGGGAAGCTAGAGTAAGTAGTTCGCCAGTTAATAG |
| | TTTGCGCAACGTTGTTGCCATTGCTACAGGCATCGTGGTGTCACG |
| | CTCGTCGTTTGGTATGGCTTCATTCAGCTCCGGTTCCCAACGATC |
| | AAGGCGAGTTACATGATCCCCCATGTTGTGCAAAAAAGCGGTTAG |
| | CTCCTTCGGTCCTCCGATCGTTGTCAGAAGTAAGTTGGCCGCAGT |
| | GTTATCACTCATGGTTATGGCAGCACTGCATAATTCTCTTACTGT |
| | CATGCCATCCGTAAGATGCTTTTCTGTGACTGGTGAGTACTCAAC |
| | CAAGTCATTCTGAGAATAGTGTATGCGGCGACCGAGTTGCTCTTG |
| | CCCGGCGTCAATACGGGATAATACCGCGCCACATAGCAGAACTTT |
| | AAAAGTGCTCATCATTGGAAAACGTTCTTCGGGGCGAAAACTCTC |
| | AAGGATCTTACCGCTGTTGAGATCCAGTTCGATGTAACCCACTCG |
| | TGCACCCAACTGATCTTCAGCATCTTTTACTTTCACCAGCGTTTC |
| | TGGGTGAGCAAAAACAGGAAGGCAAAATGCCGCAAAAAAGGGAAT |
| | AAGGGCGACACGGAAATGTTGAATACTCAT (SEQ ID NO: |
| | 91) |
| PB-TRE-V5- | ACTCTTCCTTTTTCAATATTATTGAAGCATTTATCAGGGTTATTG |
| rCas9-NLS EF | TCTCATGAGCGGATACATATTTGAATGTATTTAGAAAAATAAACA |
| NLS-Turq | AATAGGGGTTCCGCGCACATTTCCCCGAAAAGTGCCACCTAAATT |
| | GTAAGCGTTAATATTTTGTTAAAATTCGCGTTAAATTTTTGTTAA |
| | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTAATTTGAATAGATATTAAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTTCTACCGGGTAGGGGAGGCGCTTTTCCCAAGGC |
| | AGTCTGGAGCATGCGCTTTAGCAGCCCCGCTGGGCACTTGGCGCT |
| | ACACAAGTGGCCTCTGGCCTCGCACACATTCCACATCCACCGGTA |
| | GGCGCCAACCGGCTCCGTTCTTTGGTGGCCCCTTCGCGCCACCTT |
| | CTACTCCTCCCCTAGTCAGGAAGTTCCCCCCCGCCCCGCAGCTCG |
| | CGTCGTGCAGGACGTGACAAATGGAAGTAGCACGTCTCATTAGTC |
| | TCGTGCAGATGGACAGCACCGCTGAGCAATGGAAGCGGGTAGGCC |
| | TTTGGGGCAGCGGCCAATAGCAGCTTTGCTCCTTCGCTTTCTGGG |
| | CTCAGAGGCTGGGAAGGGGTGGGTCCGGGGGCGGGCTCAGGGGCG |
| | GGCTCAGGGGCGGGGCGGGCGCCCGAAGGTCCTCCGGAGGCCCGG |
| | CATTCTGCACGCTTCAAAAGCGCACGTCTGCCGCGCTGTTCTCCT |
| | CTTCCTCATCTCCGGGCCTTTCGACCTGCAGGTTAATTAAATGGG |
| | TAAGCCTATCCCTAACCCTCTCCTCGGTCTCGATTCTACGGCTAG |
| | CATGGACAAGAAGTACAGCATCGGCCTGGCCATCGGCACCAACTC |
| | TGTGGGCTGGGCCGTGATCACCGACGAGTACAAGGTGCCCAGCAA |
| | GAAATTCAAGGTGCTGGGCAACACCGACCGGCACAGCATCAAGAA |
| | GAACCTGATCGGCGCCCTGCTGTTCGACAGCGGAGAAACAGCCGA |
| | GGCCACCCGGCTGAAGAGAACCGCCAGAAGAAGATACACCAGACG |
| | GAAGAACCGGATCTGCTATCTGCAAGAGATCTTCAGCAACGAGAT |
| | GGCCAAGGTGGACGACAGCTTCTTCCACAGACTGGAAGAGTCCTT |
| | CCTGGTGGAAGAGGATAAGAAGCACGAGCGGCACCCCATCTTCGG |
| | CAACATCGTGGACGAGGTGGCCTACCACGAGAAGTACCCCACCAT |
| | CTACCACCTGAGAAAGAAACTGGTGGACAGCACCGACAAGGCCGA |
| | CCTGCGGCTGATCTATCTGGCCCTGGCCCACATGATCAAGTTCCG |
| | GGGCCACTTCCTGATCGAGGGCGACCTGAACCCCGACAACAGCGA |
| | CGTGGACAAGCTGTTCATCCAGCTGGTGCAGACCTACAACCAGCT |
| | GTTCGAGGAAAACCCCATCAACGCCAGCGGCGTGGACGCCAAGGC |
| | CATCCTGTCTGCCAGACTGAGCAAGAGCAGACGGCTGGAAAATCT |
| | GATCGCCCAGCTGCCCGGCGAGAAGAAGAATGGCCTGTTCGGCAA |
| | CCTGATTGCCCTGAGCCTGGGCCTGACCCCCAACTTCAAGAGCAA |
| | CTTCGACCTGGCCGAGGATGCCAAACTGCAGCTGAGCAAGGACAC |
| | CTACGACGACGACCTGGACAACCTGCTGGCCCAGATCGGCGACCA |
| | GTACGCCGACCTGTTTCTGGCCGCCAAGAACCTGTCCGACGCCAT |
| | CCTGCTGAGCGACATCCTGAGAGTGAACACCGAGATCACCAAGGC |
| | CCCCCTGAGCGCCTCTATGATCAAGAGATACGACGAGCACCACCA |
| | GGACCTGACCCTGCTGAAAGCTCTCGTGCGGCAGCAGCTGCCTGA |
| | GAAGTACAAAGAGATTTTCTTCGACCAGAGCAAGAACGGCTACGC |
| | CGGCTACATCGATGGCGGAGCCAGCCAGGAAGAGTTCTACAAGTT |
| | CATCAAGCCCATCCTGGAAAAGATGGACGGCACCGAGGAACTGCT |
| | CGTGAAGCTGAACAGAGAGGACCTGCTGCGGAAGCAGCGGACCTT |
| | CGACAACGGCAGCATCCCCCACCAGATCCACCTGGGAGAGCTGCA |
| | CGCCATTCTGCGGCGGCAGGAAGATTTTTACCCATTCCTGAAGGA |
| | CAACCGGGAAAAGATCGAGAAGATCCTGACCTTCCGCATCCCCTA |
| | CTACGTGGGCCCTCTGGCCAGGGGAAACAGCAGATTCGCCTGGAT |
| | GACCAGAAAGAGCGAGGAAACCATCACCCCCTGGAACTTCGAGGA |
| | AGTGGTGGACAAGGGCGCCAGCGCCCAGAGCTTCATCGAGCGGAT |
| | GACCAACTTCGATAAGAACCTGCCCAACGAGAAGGTGCTGCCCAA |
| | GCACAGCCTGCTGTACGAGTACTTCACCGTGTACAACGAGCTGAC |
| | CAAAGTGAAATACGTGACCGAGGGAATGAGAAAGCCCGCCTTCCT |
| | GAGCGGCGAGCAGAAAAAAGCCATCGTGGACCTGCTGTTCAAGAC |
| | CAACCGGAAAGTGACCGTGAAGCAGCTGAAAGAGGACTACTTCAA |
| | GAAAATCGAGTGCTTCGACTCCGTGGAAATCTCCGGCGTGGAAGA |
| | TCGGTTCAACGCCTCCCTGGGCACATACCACGATCTGCTGAAAAT |
| | TATCAAGGACAAGGACTTCCTGGACAATGAGGAAAACGAGGACAT |
| | TCTGGAAGATATCGTGCTGACCCTGACACTGTTTGAGGACAGAGA |
| | GATGATCGAGGAACGGCTGAAAACCTATGCCCACCTGTTCGACGA |
| | CAAAGTGATGAAGCAGCTGAAGCGGCGGAGATACACCGGCTGGGG |
| | CAGGCTGAGCCGGAAGCTGATCAACGGCATCCGGGACAAGCAGTC |
| | CGGCAAGACAATCCTGGATTTCCTGAAGTCCGACGGCTTCGCCAA |
| | CAGAAACTTCATGCAGCTGATCCACGACGACAGCCTGACCTTTAA |
| | AGAGGACATCCAGAAAGCCCAGGTGTCCGGCCAGGGCGATAGCCT |
| | GCACGAGCACATTGCCAATCTGGCCGGCAGCCCCGCCATTAAGAA |
| | GGGCATCCTGCAGACAGTGAAGGTGGTGGACGAGCTCGTGAAAGT |
| | GATGGGCCGGCACAAGCCCGAGAACATCGTGATCGAAATGGCCAG |
| | AGAGAACCAGACCACCCAGAAGGGACAGAAGAACAGCCGCGAGAG |
| | AATGAAGCGGATCGAAGAGGGCATCAAAGAGCTGGGCAGCCAGAT |
| | CCTGAAAGAACACCCCGTGGAAAACACCCAGCTGCAGAACGAGAA |
| | GCTGTACCTGTACTACCTGCAGAATGGGCGGGATATGTACGTGGA |
| | CCAGGAACTGGACATCAACCGGCTGTCCGACTACGATGTGGACGC |
| | TATCGTGCCTCAGAGCTTTCTGAAGGACGACTCCATCGATAACAA |
| | AGTGCTGACTCGGAGCGACAAGAACCGGGGCAAGAGCGACAACGT |
| | GCCCTCCGAAGAGGTCGTGAAGAAGATGAAGAACTACTGGCGCCA |
| | GCTGCTGAATGCCAAGCTGATTACCCAGAGGAAGTTCGACAATCT |
| | GACCAAGGCCGAGAGAGGCGGCCTGAGCGAACTGGATAAGGCCGG |
| | CTTCATCAAGAGACAGCTGGTGGAAACCCGGCAGATCACAAAGCA |
| | CGTGGCACAGATCCTGGACTCCCGGATGAACACTAAGTACGACGA |
| | GAACGACAAACTGATCCGGGAAGTGAAAGTGATCACCCTGAAGTC |
| | CAAGCTGGTGTCCGATTTCCGGAAGGATTTCCAGTTTTACAAAGT |
| | GCGCGAGATCAACAACTACCACCACGCCCACGACGCCTACCTGAA |
| | CGCCGTCGTGGGAACCGCCCTGATCAAAAAGTACCCTAAGCTGGA |
| | AAGCGAGTTCGTGTACGGCGACTACAAGGTGTACGACGTGCGGAA |
| | GATGATCGCCAAGAGCGAGCAGGAAATCGGCAAGGCTACCGCCAA |
| | GTACTTCTTCTACAGCAACATCATGAACTTTTTCAAGACCGAGAT |
| | TACCCTGGCCAACGGCGAGATCCGGAAGCGGCCTCTGATCGAGAC |
| | AAACGGCGAAACAGGCGAGATCGTGTGGGATAAGGGCCGGGACTT |
| | TGCCACCGTGCGGAAAGTGCTGTCTATGCCCCAAGTGAATATCGT |
| | GAAAAAGACCGAGGTGCAGACAGGCGGCTTCAGCAAAGAGTCTAT |
| | CCTGCCCAAGAGGAACAGCGACAAGCTGATCGCCAGAAAGAAGGA |
| | CTGGGACCCTAAGAAGTACGGCGGCTTCGACAGCCCCACCGTGGC |
| | CTATTCTGTGCTGGTGGTGGCCAAAGTGGAAAAGGGCAAGTCCAA |
| | GAAACTGAAGAGTGTGAAAGAGCTGCTGGGGATCACCATCATGGA |
| | AAGAAGCAGCTTCGAGAAGAATCCCATCGACTTTCTGGAAGCCAA |
| | GGGCTACAAAGAAGTGAAAAAGGACCTGATCATCAAGCTGCCTAA |
| | GTACTCCCTGTTCGAGCTGGAAAACGGCCGGAAGAGAATGCTGGC |
| | CTCTGCCGGCGAACTGCAGAAGGGAAACGAACTGGCCCTGCCCTC |
| | CAAATATGTGAACTTCCTGTACCTGGCCAGCCACTATGAGAAGCT |
| | GAAGGGCTCCCCCGAGGATAATGAGCAGAAACAGCTGTTTGTGGA |
| | ACAGCACAAACACTACCTGGACGAGATCATCGAGCAGATCAGCGA |
| | GTTCTCCAAGAGAGTGATCCTGGCCGACGCTAATCTGGACAAGGT |
| | GCTGAGCGCCTACAACAAGCACAGAGACAAGCCTATCAGAGAGCA |
| | GGCCGAGAATATCATCCACCTGTTTACCCTGACCAATCTGGGAGC |
| | CCCTGCCGCCTTCAAGTACTTTGACACCACCATCGACCGGAAGAG |
| | GTACACCAGCACCAAAGAGGTGCTGGACGCCACCCTGATCCACCA |
| | GAGCATCACCGGCCTGTACGAGACACGGATCGACCTGTCTCAGCT |
| | GGGAGGCGACCTCGAGTAAGCCTCGACTGTGCCTTCTAGTTGCCA |
| | GCCATCTGTTGTTTGCCCCTCCCCCGTGCCTTCCTTGACCCTGGA |
| | AGGTGCCACTCCCACTGTCCTTTCCTAATAAAATGAGGAAATTGC |
| | ATCGCATTGTCTGAGTAGGTGTCATTCTATTCTGGGGGGTGGGGT |
| | GGGGCAGGACAGCAAGGGGGAGGATTGGGAAGACAATAGCAGGCA |
| | TGCTGGGGAACGCGTTCTAGACTTAAGCAACATGCTGCTCCGCCG |
| | AAAAAGAAGAGAAAAGGTTAAGCCTCGACTGTGCCTTCTAGTTGC |
| | CAGCCATCTGTTGTTTGCCCCTCCCCCGTGCCTTCCTTGACCCTG |
| | GAAGGTGCCACTCCCACTGTCCTTTCCTAATAAAATGAGGAAATT |
| | GCATCGCATTGTCTGAGTAGGTGTCATTCTATTCTGGGGGGTGGG |
| | GTGGGGCAGGACAGCAAGGGGGAGGATTGGGAAGACAATAGCAGG |
| | CATGCTGGGGAGTGCCCGTCAGTGGGCAGAGCGCACATCGCCCAC |
| | AGTCCCCGAGAAGTTGGGGGGAGGGGTCGGCAATTGAACGGGTGC |
| | CTAGAGAAGGTGGCGCGGGGTAAACTGGGAAAGTGATGTCGTGTA |
| | CTGGCTCCGCCTTTTTCCCGAGGGTGGGGGAGAACCGTATATAAG |
| | TGCAGTAGTCGCCGTGAACGTTCTTTTTCGCAACGGGTTTGCCGC |
| | CAGAACACAGATGGTCTCTAAAGGAGGTTCGTCCGACGACGAAGC |
| | AACAGCGGACTCGCAGCACGCCGCACCTCCTAAGAAGAAAAGGAA |
| | GGTAGGGGATCCCCGGGTACCGGTCGCCACCATGGTGAGCAAGGG |
| | CGAGGAGCTGTTCACCGGGGTGGTGCCCATCCTGGTCGAGCTGGA |
| | CGGCGACGTAAACGGCCACAAGTTCAGCGTGTCCGGCGAGGGCGA |
| | GGGCGATGCCACCTACGGCAAGCTGACCCTGAAGTTCATCTGCAC |
| | CACCGGCAAGCTGCCCGTGCCCTGGCCCACCCTCGTGACCACCCT |
| | GTCCTGGGGCGTGCAGTGCTTCGCCCGCTACCCCGACCACATGAA |
| | GCAGCACGACTTCTTCAAGTCCGCCATGCCCGAAGGCTACGTCCA |
| | GGAGCGCACCATCTTCTTCAAGGACGACGGCAACTACAAGACCCG |
| | CGCCGAGGTGAAGTTCGAGGGCGACACCCTGGTGAACCGCATCGA |
| | GCTGAAGGGCATCGACTTCAAGGAGGACGGCAACATCCTGGGGCA |
| | CAAGCTGGAGTACAACTACTTTAGCGACAACGTCTATATCACCGC |
| | CGACAAGCAGAAGAACGGCATCAAGGCCAACTTCAAGATCCGCCA |
| | CAACATCGAGGACGGCGGCGTGCAGCTCGCCGACCACTACCAGCA |
| | GAACACCCCCATCGGCGACGGCCCCGTGCTGCTGCCCGACAACCA |
| | CTACCTGAGCACCCAGTCCAAGCTGAGCAAAGACCCCAACGAGAA |
| | GCGCGATCACATGGTCCTGCTGGAGTTCGTGACCGCCGCCGGGAT |
| | CACTCTCGGCATGGACGAGCTGTACAAGTAAAATCAACCTCTGGA |
| | TTACAAAATTTGTGAAAGATTGACTGGTATTCTTAACTATGTTGC |
| | TCCTTTTACGCTATGTGGATACGCTGCTTTAATGCCTTTGTATCA |
| | TGTTAACTAAACTTGTTTATTGCAGCTTATAATGGTTACAAATAA |
| | AGCAATAGCATCACAAATTTCACAAATAAAGCATTTTTTTCACTG |
| | CATTCTAGTTGTGGTTTGTCCAAACTCATCAATGTATCTTATCAT |
| | GTCTGGAATTGACTCAAATGATGTCAATTAGTCTATCAGAAGCTC |
| | ATCTGGTCTCCCTTCCGGGGGACAAGACATCCCTGTTTAATATTT |
| | AAACAGCAGTGTTCCCAAACTGGGTTCTTATATCCCTTGCTCTGG |
| | TCAACCAGGTTGCAGGGTTTCCTGTCCTCACAGGAACGAAGTCCC |
| | TAAAGAAACAGTGGCAGCCAGGTTTAGCCCCGGAATTGACTGGAT |
| | TCCTTTTTTAGGGCCCATTGGTATGGCTTTTTCCCCGTATCCCCC |
| | CAGGTGTCTGCAGGCTCAAAGAGCAGCGAGAAGCGTTCAGAGGAA |
| | AGCGATCCCGTGCCACCTTCCCCGTGCCCGGGCTGTCCCCGCACG |
| | CTGCCGGCTCGGGGATGCGGGGGGAGCGCCGGACCGGAGCGGAGC |
| | CCCGGGCGGCTCGCTGCTGCCCCCTAGCGGGGGAGGGACGTAATT |
| | ACATCCCTGGGGGCTTTGGGGGGGGGCTGTCCCTGATATCTATAA |
| | CAAGAAAATATATATATAATAAGTTATCACGTAAGTAGAACATGA |
| | AATAACAATATAATTATCGTATGAGTTAAATCTTAAAAGTCACGT |
| | AAAAGATAATCATGCGTCATTTTGACTCACGCGGTCGTTATAGTT |
| | CAAAATCAGTGACACTTACCGCATTGACAAGCACGCCTCACGGGA |
| | GCTCCAAGCGGCGACTGAGATGTCCTAAATGCACAGCGACGGATT |
| | CGCGCTATTTAGAAAGAGAGAGCAATATTTCAAGAATGCATGCGT |
| | CAATTTTACGCAGACTATCTTTCTAGGGTTAATCTAGCTGCATCA |
| | GGATCATATCGTCGGGTCTTTTTTCCGGCTCAGTCATCGCCCAAG |
| | CTGGCGCTATCTGGGCATCGGGGAGGAAGAAGCCCGTGCCTTTTC |
| | CCGCGAGGTTGAAGCGGCATGGAAAGAGTTTGCCGAGGATGACTG |
| | CTGCTGCATTGACGTTGAGCGAAAACGCACGTTTACCATGATGAT |
| | TCGGGAAGGTGTGGCCATGCACGCCTTTAACGGTGAACTGTTCGT |
| | TCAGGCCACCTGGGATACCAGTTCGTCGCGGCTTTTCCGGACACA |
| | GTTCCGGATGGTCAGCCCGAAGCGCATCAGCAACCCGAACAATAC |
| | CGGCGACAGCCGGAACTGCCGTGCCGGTGTGCAGATTAATGACAG |
| | CGGTGCGGCGCTGGGATATTACGTCAGCGAGGACGGGTATCCTGG |
| | CTGGATGCCGCAGAAATGGACATGGATACCCCGTGAGTTACCCGG |
| | CGGGCGCGCTTGGCGTAATCATGGTCATAGCTGTTTCCTGTGTGA |
| | AATTGTTATCCGCTCACAATTCCACACAACATACGAGCCGGAAGC |
| | ATAAAGTGTAAAGCCTGGGGTGCCTAATGAGTGAGCTAACTCACA |
| | TTAATTGCGTTGCGCTCACTGCCCGCTTTCCAGTCGGGAAACCTG |
| | TCGTGCCAGCTGCATTAATGAATCGGCCAACGCGCGGGGAGAGGC |
| | GGTTTGCGTATTGGGCGCTCTTCCGCTTCCTCGCTCACTGACTCG |
| | CTGCGCTCGGTCGTTCGGCTGCGGCGAGCGGTATCAGCTCACTCA |
| | AAGGCGGTAATACGGTTATCCACAGAATCAGGGGATAACGCAGGA |
| | AAGAACATGTGAGCAAAAGGCCAGCAAAAGGCCAGGAACCGTAAA |
| | AAGGCCGCGTTGCTGGCGTTTTTCCATAGGCTCCGCCCCCCTGAC |
| | GAGCATCACAAAAATCGACGCTCAAGTCAGAGGTGGCGAAACCCG |
| | ACAGGACTATAAAGATACCAGGCGTTTCCCCCTGGAAGCTCCCTC |
| | GTGCGCTCTCCTGTTCCGACCCTGCCGCTTACCGGATACCTGTCC |
| | GCCTTTCTCCCTTCGGGAAGCGTGGCGCTTTCTCATAGCTCACGC |
| | TGTAGGTATCTCAGTTCGGTGTAGGTCGTTCGCTCCAAGCTGGGC |
| | TGTGTGCACGAACCCCCCGTTCAGCCCGACCGCTGCGCCTTATCC |
| | GGTAACTATCGTCTTGAGTCCAACCCGGTAAGACACGACTTATCG |
| | CCACTGGCAGCAGCCACTGGTAACAGGATTAGCAGAGCGAGGTAT |
| | GTAGGCGGTGCTACAGAGTTCTTGAAGTGGTGGCCTAACTACGGC |
| | TACACTAGAAGGACAGTATTTGGTATCTGCGCTCTGCTGAAGCCA |
| | GTTACCTTCGGAAAAAGAGTTGGTAGCTCTTGATCCGGCAAACAA |
| | ACCACCGCTGGTAGCGGTGGTTTTTTTGTTTGCAAGCAGCAGATT |
| | ACGCGCAGAAAAAAAGGATCTCAAGAAGATCCTTTGATCTTTTCT |
| | ACGGGGTCTGACGCTCAGTGGAACGAAAACTCACGTTAAGGGATT |
| | TTGGTCATGAGATTATCAAAAAGGATCTTCACCTAGATCCTTTTA |
| | AATTAAAAATGAAGTTTTAAATCAATCTAAAGTATATATGAGTAA |
| | ACTTGGTCTGACAGTTACCAATGCTTAATCAGTGAGGCACCTATC |
| | TCAGCGATCTGTCTATTTCGTTCATCCATAGTTGCCTGACTCCCC |
| | GTCGTGTAGATAACTACGATACGGGAGGGCTTACCATCTGGCCCC |
| | AGTGCTGCAATGATACCGCGAGACCCACGCTCACCGGCTCCAGAT |
| | TTATCAGCAATAAACCAGCCAGCCGGAAGGGCCGAGCGCAGAAGT |
| | GGTCCTGCAACTTTATCCGCCTCCATCCAGTCTATTAATTGTTGC |
| | CGGGAAGCTAGAGTAAGTAGTTCGCCAGTTAATAGTTTGCGCAAC |
| | GTTGTTGCCATTGCTACAGGCATCGTGGTGTCACGCTCGTCGTTT |
| | GGTATGGCTTCATTCAGCTCCGGTTCCCAACGATCAAGGCGAGTT |
| | ACATGATCCCCCATGTTGTGCAAAAAAGCGGTTAGCTCCTTCGGT |
| | CCTCCGATCGTTGTCAGAAGTAAGTTGGCCGCAGTGTTATCACTC |
| | ATGGTTATGGCAGCACTGCATAATTCTCTTACTGTCATGCCATCC |
| | GTAAGATGCTTTTCTGTGACTGGTGAGTACTCAACCAAGTCATTC |
| | TGAGAATAGTGTATGCGGCGACCGAGTTGCTCTTGCCCGGCGTCA |
| | ATACGGGATAATACCGCGCCACATAGCAGAACTTTAAAAGTGCTC |
| | ATCATTGGAAAACGTTCTTCGGGGCGAAAACTCTCAAGGATCTTA |
| | CCGCTGTTGAGATCCAGTTCGATGTAACCCACTCGTGCACCCAAC |
| | TGATCTTCAGCATCTTTTACTTTCACCAGCGTTTCTGGGTGAGCA |
| | AAAACAGGAAGGCAAAATGCCGCAAAAAAGGGAATAAGGGCGACA |
| | CGGAAATGTTGAATACTCAT (SEQ ID NO: 92) |
| PB-PGK-(5UTR- | ACTCTTCCTTTTTCAATATTATTGAAGCATTTATCAGGGTTATTG |
| uORF)-Citrine- | TCTCATGAGCGGATACATATTTGAATGTATTTAGAAAAATAAACA |
| U6-sgRNA-IRES- | AATAGGGGTTCCGCGCACATTTCCCCGAAAAGTGCCACCTAAATT |
| EF-Puro-NLS- | GTAAGCGTTAATATTTTGTTAAAATTCGCGTTAAATTTTTGTTAA |
| Cherry | ATCAGCTCATTTTTTAACCAATAGGCCGAAATCGGCAAAATCCCT |
| | TATAAATCAAAAGAATAGACCGAGATAGGGTTGAGTGTTGTTCCA |
| | GTTTGGAACAAGAGTCCACTATTAAAGAACGTGGACTCCAACGTC |
| | AAAGGGCGAAAAACCGTCTATCAGGGCGATGGCCCACTACGTGAA |
| | CCATCACCCTAATCAAGTTTTTTGGGGTCGAGGTGCCGTAAAGCA |
| | CTAAATCGGAACCCTAAAGGGAGCCCCCGATTTAGAGCTTGACGG |
| | GGAAAGCCGGCGAACGTGGCGAGAAAGGAAGGGAAGAAAGCGAAA |
| | GGAGCGGGCGCTAGGGCGCTGGCAAGTGTAGCGGTCACGCTGCGC |
| | GTAACCACCACACCCGCCGCGCTTAATGCGCCGCTACAGGGCGCG |
| | TCCCATTCGCCATTCAGGCTGCGCAACTGTTGGGAAGGGCGATCG |
| | GTGCGGGCCTCTTCGCTATTACGCCAGCTGGCGAAAGGGGGATGT |
| | GCTGCAAGGCGATTAAGTTGGGTAACGCCAGGGTTTTCCCAGTCA |
| | CGACGTTGTAAAACGACGGCCAGTGAGCGCGCCTCGTTCATTCAC |
| | GTTTTTGAACCCGTGGAGGACGGGCAGACTCGCGGTGCAAATGTG |
| | TTTTACAGCGTGATGGAGCAGATGAAGATGCTCGACACGCTGCAG |
| | AACACGCAGCTAGATTAACCCTAGAAAGATAATCATATTGTGACG |
| | TACGTTAAAGATAATCATGTGTAAAATTGACGCATGTGTTTTATC |
| | GGTCTGTATATCGAGGTTTATTTATTTAATTTGAATAGATATTAG |
| | TTTTATTATATTTACACTTACATACTAATAATAAATTCAACAAAC |
| | AATTTATTTATGTTTATTTATTTATTAAAAAAAACAAAAACTCAA |
| | AATTTCTTCTATAAAGTAACAAAACTTTTATGAGGGACAGCCCCC |
| | CCCCAAAGCCCCCAGGGATGTAATTACGTCCCTCCCCCGCTAGGG |
| | GGCAGCAGCGAGCCGCCCGGGGCTCCGCTCCGGTCCGGCGCTCCC |
| | CCCGCATCCCCGAGCCGGCAGCGTGCGGGGACAGCCCGGGCACGG |
| | GGAAGGTGGCACGGGATCGCTTTCCTCTGAACGCTTCTCGCTGCT |
| | CTTTGAGCCTGCAGACACCTGGGGGGATACGGGGAAAAGGCCTCC |
| | ACGGCCACTAGTACTTTCACTTTTCTCTATCACTGATAGGGAGTG |
| | GTAAACTCGACTTTCACTTTTCTCTATCACTGATAGGGAGTGGTA |
| | AACTCGACTTTCACTTTTCTCTATCACTGATAGGGAGTGGTAAAC |
| | TCGACTTTCACTTTTCTCTATCACTGATAGGGAGTGGTAAACTCG |
| | ACTTTCACTTTTCTCTATCACTGATAGGGAGTGGTAAACTCGACT |
| | TTCACTTTTCTCTATCACTGATAGGGAGTGGTAAACTCGACTTTC |
| | ACTTTTCTCTATCACTGATAGGGAGTGGTAAACTCGACCTATATA |
| | AGCAGAGCTCGTTTAGTGAACCGTCAGATCGCCTGGAGACGCCAT |
| | CCACGCTGTTTTGACCTCCATAGAAGACACCGGGACCGATCCAGC |
| | CTCCGCGGCCCCGAATTCATGGATTATAAAGATGATGATGATAAA |
| | TAAGCAGCTGGACCAACGGACGGACGCCAGCGCTAAGGATCCCCG |
| | GGTACCGGTCGCCACCATGGTGAGCAAGGGCGAGGAGCTGTTCAC |
| | CGGGGTGGTGCCCATCCTGGTCGAGCTGGACGGCGACGTAAACGG |
| | CCACAAGTTCAGCGTGTCCGGCGAGGGCGAGGGCGATGCCACCTA |
| | CGGCAAGCTGACCCTGAAGTTCATCTGCACCACCGGCAAGCTGCC |
| | CGTGCCCTGGCCCACCCTCGTGACCACCTTCGGCTACGGCCTGAT |
| | GTGCTTCGCCCGCTACCCCGACCACATGAAGCAGCACGACTTCTT |
| | CAAGTCCGCCATGCCCGAAGGCTACGTCCAGGAGCGCACCATCTT |
| | CTTCAAGGACGACGGCAACTACAAGACCCGCGCCGAGGTGAAGTT |
| | CGAGGGCGACACCCTGGTGAACCGCATCGAGCTGAAGGGCATCGA |
| | CTTCAAGGAGGACGGCAACATCCTGGGGCACAAGCTGGAGTACAA |
| | CTACAACAGCCACAACGTCTATATCATGGCCGACAAGCAGAAGAA |
| | CGGCATCAAGGTGAACTTCAAGATCCGCCACAACATCGAGGACGG |
| | CAGCGTGCAGCTCGCCGACCACTACCAGCAGAACACCCCCATCGG |
| | CGACGGCCCCGTGCTGCTGCCCGACAACCACTACCTGAGCTACCA |
| | GTCCGCCCTGAGCAAAGACCCCAACGAGAAGCGCGATCACATGGT |
| | CCTGCTGGAGTTCGTGACCGCCGCCGGGATCACTCTCGGCATGGA |
| | CGAGCTGTACCCATACGATGTTCCAGATTACGCTTAAGCTAGCGC |
| | ATATGTCCACTTAAGGGCGCTGACATGCGCATGTGAGGATCAATT |
| | CTTACGCTGAGTACTTCGATTCCTCAAATAGCAAGACAGCCCACA |
| | TGGCATTCCACTTATCACTGGCATCCTAGATCTGATAGCTTTGTT |
| | CTCAAAGTCTCGAGAAATTCGAATTTAAATCGACGCGTGCCTCGA |
| | CTGTGCCTTCTAGTTGCCAGCCATCTGTTGTTTGCCCCTCCCCCG |
| | TGCCTTCCTTGACCCTGGAAGGTGCCACTCCCACTGTCCTTTCCT |
| | AATAAAATGAGGAAATTGCATCGCATTGTCTGAGTAGGTGTCATT |
| | CTATTCTGGGGGGTGGGGTGGGGCAGGACAGCAAGGGGGAGGATT |
| | GGGAAGACAATAGCAGGCATGCTGGGGAAATAAAGCAAAAAAAAA |
| | AATATCATCGTGTTCTTCAAAGGAAAACCACGTCCCCGTGGTTCG |
| | GGGGGCCTAGACGTTTTTTTAACCTCGACTAAACACATGTAAAGC |
| | ATGTGCACCGAGGCCCCAGATCAGATCCCATACAATGGGGTACCT |
| | TCTGGGCATCCTTCAGCCCCTTGTTGAATACGCTTGAGGAGAGCC |
| | ATTTGACTCTTTCCACAACTATCCAACTCACAACGTGGCACTGGG |
| | GTTGTGCCGCCTTTGCAGGTGTATCTTATACACGTGGCTTTTGGC |
| | CGCAGAGGCACCTGTCGCCAGGTGGGGGGTTCCGCTGCCTGCAAA |
| | GGGTCGCTACAGACGTTGTTTGTCTTCAAGAAGCTTCCAGAGGAA |
| | CTGCTTCCTTCACGACATTCAACAGACCTTGCATTCCTTTGGCGA |
| | GAGGGGAAAGACCCCTAGGAATGCTCGTCAAGAAGACAGGGCCAG |
| | GTTTCCGGGCCCTCACATTGCCAAAAGACGGCAATATGGTGGAAA |
| | ATAACATATAGACAAACGCACACCGGCCTTATTCCAAGCGGCTTC |
| | GGCCAGTAACGTTAGGGGGGGGGGAGGGAGAGGGGCACCGACTCG |
| | GTGCCACTTTTTCAAGTTGATAACGGACTAGCCTTATTTAAACTT |
| | GCTATGCTGTTTCCAGCATAGCTCTTAAATGTTCTCGAACATGGC |
| | ATTGGGAACACGGTGTTTCGTCCTTTCCACAAGATATATAAAGCC |
| | AAGAAATCGAAATACTTTCAAGTTACGGTAAGCATATGATAGTCC |
| | ATTTTAAAACATAATTTTAAAACTGCAAACTACCCAAGAAATTAT |
| | TACTTTCTACGTCACGTATTTTGTACTAATATCTTTGTGTTTACA |
| | GTCAAATTAATTCTAATTATCTCTCTAACAGCCTTGTATCGTATA |
| | TGCAAATATGAAGGAATCATGGGAAATAGGCCCTCTTCCTGCCCG |
| | ACCTCGCGGCCGCGAAGGATCTGCGATCGCTCCGGTGCCCGTCAG |
| | TGGGCAGAGCGCACATCGCCCACAGTCCCCGAGAAGTTGGGGGGA |
| | GGGGTCGGCAATTGAACGGGTGCCTAGAGAAGGTGGCGCGGGGTA |
| | AACTGGGAAAGTGATGTCGTGTACTGGCTCCGCCTTTTTCCCGAG |
| | GGTGGGGGAGAACCGTATATAAGTGCAGTAGTCGCCGTGAACGTT |
| | CTTTTTCGCAACGGGTTTGCCGCCAGAACACAGCTGAAGCTTCGA |
| | GGGGCTCGCATCTCTCCTTCACGCGCCCGCCGCCCTACCTGAGGC |
| | CGCCATCCACGCCGGTTGAGTCGCGTTCTGCCGCCTCCCGCCTGT |
| | GGTGCCTCCTGAACTGCGTCCGCCGTCTAGGTAAGTTTAAAGCTC |
| | AGGTCGAGACCGGGCCTTTGTCCGGCGCTCCCTTGGAGCCTACCT |
| | AGACTCAGCCGGCTCTCCACGCTTTGCCTGACCCTGCTTGCTCAA |
| | CTCTACGTCTTTGTTTCGTTTTCTGTTCTGCGCCGTTACAGATCC |
| | AAGCTGTGACCGGCGCCTACGCTAGATGACCGAGTACAAGCCCAC |
| | GGTGCGCCTCGCCACCCGCGACGACGTCCCCAGGGCCGTACGCAC |
| | CCTCGCCGCCGCGTTCGCCGACTACCCCGCCACGCGCCACACCGT |
| | CGATCCGGACCGCCACATCGAGCGGGTCACCGAGCTGCAAGAACT |
| | CTTCCTCACGCGCGTCGGGCTCGACATCGGCAAGGTGTGGGTCGC |
| | GGACGACGGCGCCGCGGTGGCGGTCTGGACCACGCCGGAGAGCGT |
| | CGAAGCGGGGGCGGTGTTCGCCGAGATCGGCCCGCGCATGGCCGA |
| | GTTGAGCGGTTCCCGGCTGGCCGCGCAGCAACAGATGGAAGGCCT |
| | CCTGGCGCCGCACCGGCCCAAGGAGCCCGCGTGGTTCCTGGCCAC |
| | CGTCGGCGTCTCGCCCGACCACCAGGGCAAGGGTCTGGGCAGCGC |
| | CGTCGTGCTCCCCGGAGTGGAGGCGGCCGAGCGCGCCGGGGTGCC |
| | CGCCTTCCTGGAGACCTCCGCGCCCCGCAACCTCCCCTTCTACGA |
| | GCGGCTCGGCTTCACCGTCACCGCCGACGTCGAGGTGCCCGAAGG |
| | ACCGCGCACCTGGTGCATGACCCGCAAGCCCGGTGCCGGAAGCGG |
| | AGCTACTAACTTCAGCCTGCTGAAGCAGGCTGGAGACGTGGAGGA |
| | GAACCCTGGACCTATGTCTAGACTGGACAAGAGCAAAGTCATAAA |
| | CGGCGCTCTGGAATTACTCAATGGAGTCGGTATCGAAGGCCTGAC |
| | GACAAGGAAACTCGCTCAAAAGCTGGGAGTTGAGCAGCCTACCCT |
| | GTACTGGCACGTGAAGAACAAGCGGGCCCTGCTCGATGCCCTGCC |
| | AATCGAGATGCTGGACAGGCATCATACCCACTTCTGCCCCCTGGA |
| | AGGCGAGTCATGGCAAGACTTTCTGCGGAACAACGCCAAGTCATT |
| | CCGCTGTGCTCTCCTCTCACATCGCGACGGGGCTAAAGTGCATCT |
| | CGGCACCCGCCCAACAGAGAAACAGTACGAAACCCTGGAAAATCA |
| | GCTCGCGTTCCTGTGTCAGCAAGGCTTCTCCCTGGAGAACGCACT |
| | GTACGCTCTGTCCGCCGTGGGCCACTTTACACTGGGCTGCGTATT |
| | GGAGGAACAGGAGCATCAAGTAGCAAAAGAGGAAAGAGAGACACC |
| | TACCACCGATTCTATGCCCCCACTTCTGAGACAAGCAATTGAGCT |
| | GTTCGACCGGCAGGGAGCCGAACCTGCCTTCCTTTTCGGCCTGGA |
| | ACTAATCATATGTGGCCTGGAGAAACAGCTAAAGTGCGAAAGCGG |
| | CGGGCCGGCCGACGCCCTTGACGATTTTGACTTAGACATGCTCCC |
| | AGCCGATGCCCTTGACGACTTTGACCTTGATATGCTGCCTGCTGA |
| | CGCTCTTGACGATTTTGACCTTGACATGCTCCCCGGGTAACTAAG |
| | TAACCCTCTCCCTCCCCCCCCCCTAACGTTACTGGCCGAAGCCGC |
| | TTGGAATAAGGCCGGTGTGCGTTTGTCTATATGTTATTTTCCACC |
| | ATATTGCCGTCTTTTGGCAATGTGAGGGCCCGGAAACCTGGCCCT |
| | GTCTTCTTGACGAGCATTCCTAGGGGTCTTTCCCCTCTCGCCAAA |
| | GGAATGCAAGGTCTGTTGAATGTCGTGAAGGAAGCAGTTCCTCTG |
| | GAAGCTTCTTGAAGACAAACAACGTCTGTAGCGACCCTTTGCAGG |
| | CAGCGGAACCCCCCACCTGGCGACAGGTGCCTCTGCGGCCAAAAG |
| | CCACGTGTATAAGATACACCTGCAAAGGCGGCACAACCCCAGTGC |
| | CACGTTGTGAGTTGGATAGTTGTGGAAAGAGTCAAATGGCTCTCC |
| | TCAAGCGTATTCAACAAGGGGCTGAAGGATGCCCAGAAGGTACCC |
| | CATTGTATGGGATCTGATCTGGGGCCTCGGTGCACATGCTTTACA |
| | TGTGTTTAGTCGAGGTTAAAAAAACGTCTAGGCCCCCCGAACCAC |
| | GGGGACGTGGTTTTCCTTTGAAAAACACGATGATAATATGGCCAC |
| | AACCATGGTCTCTAAAGGAGGTTCGTCCGACGACGAAGCAACAGC |
| | GGACTCGCAGCACGCCGCACCTCCTAAGAAGAAAAGGAAGGTAGG |
| | GGATCCCCGGGTACCGGTCGCCACCATGGCCATCATCAAGGAGTT |
| | CATGCGCTTCAAGGTGCACATGGAGGGCTCCGTGAACGGCCACGA |
| | GTTCGAGATCGAGGGCGAGGGCGAGGGCCGCCCCTACGAGGGCAC |
| | CCAGACCGCCAAGCTGAAGGTGACCAAGGGTGGCCCCCTGCCCTT |
| | CGCCTGGGACATCCTGTCCCCTCAGTTCATGTACGGCTCCAAGGC |
| | CTACGTGAAGCACCCCGCCGACATCCCCGACTACTTGAAGCTGTC |
| | CTTCCCCGAGGGCTTCAAGTGGGAGCGCGTGATGAACTTCGAGGA |
| | CGGCGGCGTGGTGACCGTGACCCAGGACTCCTCCCTGCAGGACGG |
| | CGAGTTCATCTACAAGGTGAAGCTGCGCGGCACCAACTTCCCCTC |
| | CGACGGCCCCGTAATGCAGAAGAAGACCATGGGCTGGGAGGCCTC |
| | CTCCGAGCGGATGTACCCCGAGGACGGCGCCCTGAAGGGCGAGAT |
| | CAAGCAGAGGCTGAAGCTGAAGGACGGCGGCCACTACGACGCTGA |
| | GGTCAAGACCACCTACAAGGCCAAGAAGCCCGTGCAGCTGCCCGG |
| | CGCCTACAACGTCAACATCAAGTTGGACATCACCTCCCACAACGA |
| | GGACTACACCATCGTGGAACAGTACGAACGCGCCGAGGGCCGCCA |
| | CTCCACCGGCGGCATGGACGAGCTGTACAAGTAAGTCGACAATCA |
| | ACCTCTGGATTACAAAATTTGTGAAAGATTGACTGGTATTCTTAA |
| | CTATGTTGCTCCTTTTACGCTATGTGGATACGCTGCTTTAATGCC |
| | TTTGTATCATGCGTTAACTAAACTTGTTTATTGCAGCTTATAATG |
| | GTTACAAATAAAGCAATAGCATCACAAATTTCACAAATAAAGCAT |
| | TTTTTTCACTGCATTCTAGTTGTGGTTTGTCCAAACTCATCAATG |
| | TATCTTATCATGTCTGGAATTGACTCAAATGATGTCAATTAGTCT |
| | ATCAGAAGCTCATCTGGTCTCCCTTCCGGGGGACAAGACATCCCT |
| | GTTTAATATTTAAACAGCAGTGTTCCCAAACTGGGTTCTTATATC |
| | CCTTGCTCTGGTCAACCAGGTTGCAGGGTTTCCTGTCCTCACAGG |
| | AACGAAGTCCCTAAAGAAACAGTGGCAGCCAGGTTTAGCCCCGGA |
| | ATTGACTGGATTCCTTTTTTAGGGCCCATTGGTATGGCTTTTTCC |
| | CCGTATCCCCCCAGGTGTCTGCAGGCTCAAAGAGCAGCGAGAAGC |
| | GTTCAGAGGAAAGCGATCCCGTGCCACCTTCCCCGTGCCCGGGCT |
| | GTCCCCGCACGCTGCCGGCTCGGGGATGCGGGGGGAGCGCCGGAC |
| | CGGAGCGGAGCCCCGGGCGGCTCGCTGCTGCCCCCTAGCGGGGGA |
| | GGGACGTAATTACATCCCTGGGGGCTTTGGGGGGGGGCTGTCCCT |
| | GATATCTATAACAAGAAAATATATATATAATAAGTTATCACGTAA |
| | GTAGAACATGAAATAACAATATAATTATCGTATGAGTTAAATCTT |
| | AAAAGTCACGTAAAAGATAATCATGCGTCATTTTGACTCACGCGG |
| | TCGTTATAGTTCAAAATCAGTGACACTTACCGCATTGACAAGCAC |
| | GCCTCACGGGAGCTCCAAGCGGCGACTGAGATGTCCTAAATGCAC |
| | AGCGACGGATTCGCGCTATTTAGAAAGAGAGAGCAATATTTCAAG |
| | AATGCATGCGTCAATTTTACGCAGACTATCTTTCTAGGGTTAATC |
| | TAGCTGCATCAGGATCATATCGTCGGGTCTTTTTTCCGGCTCAGT |
| | CATCGCCCAAGCTGGCGCTATCTGGGCATCGGGGAGGAAGAAGCC |
| | CGTGCCTTTTCCCGCGAGGTTGAAGCGGCATGGAAAGAGTTTGCC |
| | GAGGATGACTGCTGCTGCATTGACGTTGAGCGAAAACGCACGTTT |
| | ACCATGATGATTCGGGAAGGTGTGGCCATGCACGCCTTTAACGGT |
| | GAACTGTTCGTTCAGGCCACCTGGGATACCAGTTCGTCGCGGCTT |
| | TTCCGGACACAGTTCCGGATGGTCAGCCCGAAGCGCATCAGCAAC |
| | CCGAACAATACCGGCGACAGCCGGAACTGCCGTGCCGGTGTGCAG |
| | ATTAATGACAGCGGTGCGGCGCTGGGATATTACGTCAGCGAGGAC |
| | GGGTATCCTGGCTGGATGCCGCAGAAATGGACATGGATACCCCGT |
| | GAGTTACCCGGCGGGCGCGCTTGGCGTAATCATGGTCATAGCTGT |
| | TTCCTGTGTGAAATTGTTATCCGCTCACAATTCCACACAACATAC |
| | GAGCCGGAAGCATAAAGTGTAAAGCCTGGGGTGCCTAATGAGTGA |
| | GCTAACTCACATTAATTGCGTTGCGCTCACTGCCCGCTTTCCAGT |
| | CGGGAAACCTGTCGTGCCAGCTGCATTAATGAATCGGCCAACGCG |
| | CGGGGAGAGGCGGTTTGCGTATTGGGCGCTCTTCCGCTTCCTCGC |
| | TCACTGACTCGCTGCGCTCGGTCGTTCGGCTGCGGCGAGCGGTAT |
| | CAGCTCACTCAAAGGCGGTAATACGGTTATCCACAGAATCAGGGG |
| | ATAACGCAGGAAAGAACATGTGAGCAAAAGGCCAGCAAAAGGCCA |
| | GGAACCGTAAAAAGGCCGCGTTGCTGGCGTTTTTCCATAGGCTCC |
| | GCCCCCCTGACGAGCATCACAAAAATCGACGCTCAAGTCAGAGGT |
| | GGCGAAACCCGACAGGACTATAAAGATACCAGGCGTTTCCCCCTG |
| | GAAGCTCCCTCGTGCGCTCTCCTGTTCCGACCCTGCCGCTTACCG |
| | GATACCTGTCCGCCTTTCTCCCTTCGGGAAGCGTGGCGCTTTCTC |
| | ATAGCTCACGCTGTAGGTATCTCAGTTCGGTGTAGGTCGTTCGCT |
| | CCAAGCTGGGCTGTGTGCACGAACCCCCCGTTCAGCCCGACCGCT |
| | GCGCCTTATCCGGTAACTATCGTCTTGAGTCCAACCCGGTAAGAC |
| | ACGACTTATCGCCACTGGCAGCAGCCACTGGTAACAGGATTAGCA |
| | GAGCGAGGTATGTAGGCGGTGCTACAGAGTTCTTGAAGTGGTGGC |
| | CTAACTACGGCTACACTAGAAGGACAGTATTTGGTATCTGCGCTC |
| | TGCTGAAGCCAGTTACCTTCGGAAAAAGAGTTGGTAGCTCTTGAT |
| | CCGGCAAACAAACCACCGCTGGTAGCGGTGGTTTTTTTGTTTGCA |
| | AGCAGCAGATTACGCGCAGAAAAAAAGGATCTCAAGAAGATCCTT |
| | TGATCTTTTCTACGGGGTCTGACGCTCAGTGGAACGAAAACTCAC |
| | GTTAAGGGATTTTGGTCATGAGATTATCAAAAAGGATCTTCACCT |
| | AGATCCTTTTAAATTAAAAATGAAGTTTTAAATCAATCTAAAGTA |
| | TATATGAGTAAACTTGGTCTGACAGTTACCAATGCTTAATCAGTG |
| | AGGCACCTATCTCAGCGATCTGTCTATTTCGTTCATCCATAGTTG |
| | CCTGACTCCCCGTCGTGTAGATAACTACGATACGGGAGGGCTTAC |
| | CATCTGGCCCCAGTGCTGCAATGATACCGCGAGACCCACGCTCAC |
| | CGGCTCCAGATTTATCAGCAATAAACCAGCCAGCCGGAAGGGCCG |
| | AGCGCAGAAGTGGTCCTGCAACTTTATCCGCCTCCATCCAGTCTA |
| | TTAATTGTTGCCGGGAAGCTAGAGTAAGTAGTTCGCCAGTTAATA |
| | GTTTGCGCAACGTTGTTGCCATTGCTACAGGCATCGTGGTGTCAC |
| | GCTCGTCGTTTGGTATGGCTTCATTCAGCTCCGGTTCCCAACGAT |
| | CAAGGCGAGTTACATGATCCCCCATGTTGTGCAAAAAAGCGGTTA |
| | GCTCCTTCGGTCCTCCGATCGTTGTCAGAAGTAAGTTGGCCGCAG |
| | TGTTATCACTCATGGTTATGGCAGCACTGCATAATTCTCTTACTG |
| | TCATGCCATCCGTAAGATGCTTTTCTGTGACTGGTGAGTACTCAA |
| | CCAAGTCATTCTGAGAATAGTGTATGCGGCGACCGAGTTGCTCTT |
| | GCCCGGCGTCAATACGGGATAATACCGCGCCACATAGCAGAACTT |
| | TAAAAGTGCTCATCATTGGAAAACGTTCTTCGGGGCGAAAACTCT |
| | CAAGGATCTTACCGCTGTTGAGATCCAGTTCGATGTAACCCACTC |
| | GTGCACCCAACTGATCTTCAGCATCTTTTACTTTCACCAGCGTTT |
| | CTGGGTGAGCAAAAACAGGAAGGCAAAATGCCGCAAAAAAGGGAA |
| | TAAGGGCGACACGGAAATGTTGAATACTCAT (SEQ ID NO: |
| | 93) |

### Example 1

### EIF4E Fusion Protein

This example is based on the 5′ Cap binding biology of EIF4E protein, which enhances translation of a target mRNA. The EIF4E protein in this example comprises mutated amino acid residues known to be regulated by cellular kinases, to make its regulation constitutive. Experiments were performed with nuclease dead Cas9 (dCas9), with protein effectors fused to the C-terminus. Any messenger RNA of interest can be targeted with this system, given the selection of an appropriate mRNA targeting spacer sequence, which is specific to each CRISPR-Cas system.

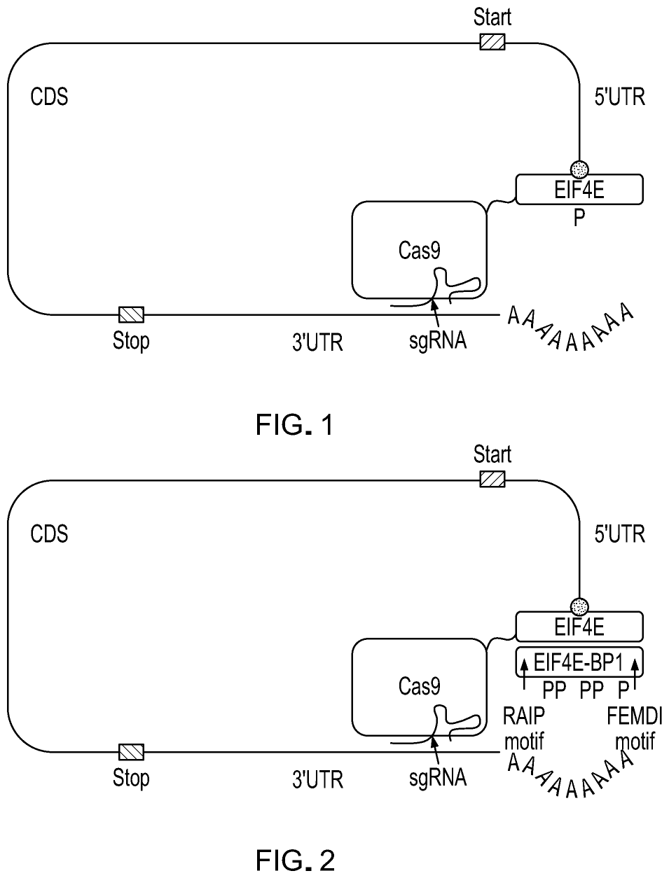

An exemplary system is composed of a nuclease-dead Cas9 (dCas9) protein fused to a modified EIF4E (FIG. 1), which can enhance translation. These dCas9 fusion proteins bind a single guide RNA (sgRNA) driven by a U6 polymerase III promoter, and may co-bind an antisense synthetic oligonucleotide composed alternating 2′OMe RNA and DNA bases (PAMmer). Together, these components form an RCas9-RNA recognition complex that binds messenger RNA.

Without being bound by theory, a PAMmer likely increases binding affinity of dCas9 to RNA in vivo as well as in vitro, but likely it is not absolutely required for RNA targeting. Preliminary experiments were performed in the absence of a PAMmer.

A schematic of the anticipated mechanism is shown in FIG. 1. Without being bound by theory, dCas9-EIF4E targets the 3′UTR of a representative target transcript mRNA. Modified EIF4E facilitates transcript circularization and the recruitment of EIF4G and ribosomal pre-initiation complexes.

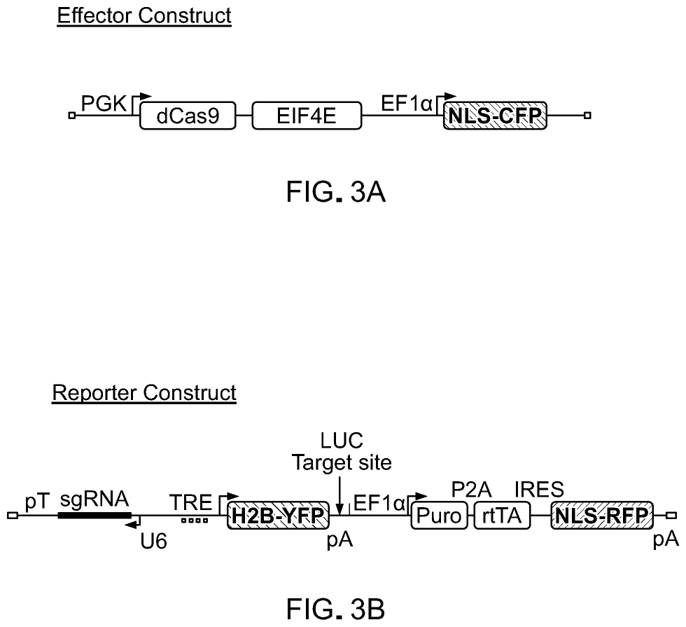

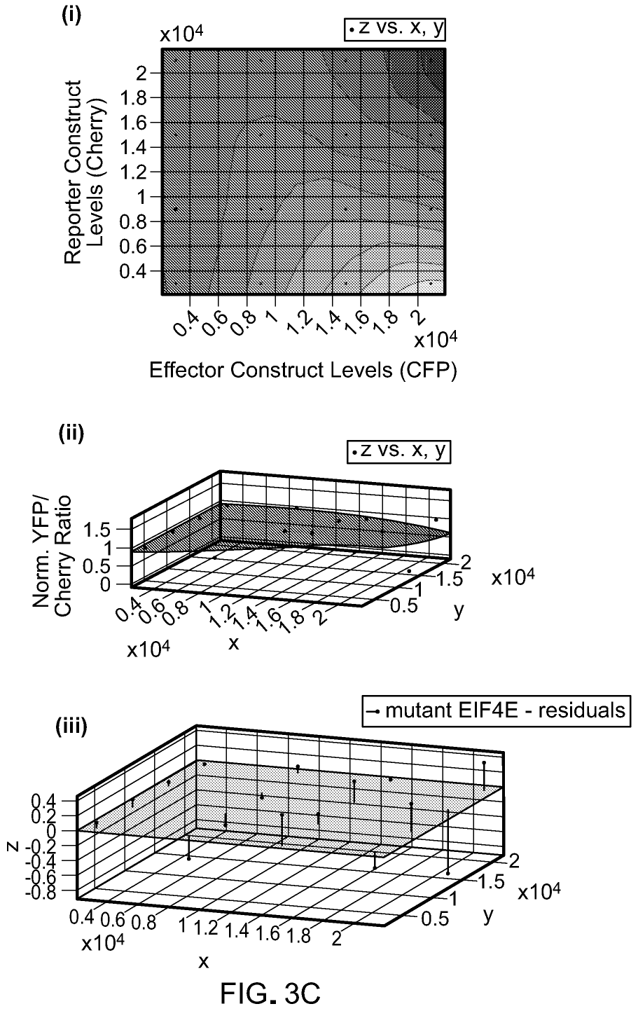

DNA constructs were prepared as shown in FIG. 3A and FIG. 3B. Cas9-EIF4E expression level was correlated to a co-expressed CFP fluorophore on the Effector plasmid. YFP and RFP are co-expressed from different promoters on the Reporter. However, only YFP messenger RNA carries a target site (LUC target site) that is complementary to the spacer of the single guide RNA (sgRNA).

Results of the experiments are shown in FIG. 3C: (i) Heatmap showing how the fold change in YFP/RFP ratio relate to Reporter (x-axis) and Effector (y-axis) DNA construct levels. Datapoints used for the heatmap represent the average fluorescence of single cells that fall within defined bins. (ii) Same data as presented in (i), but with YFP/RFP ratio plotted as third variable (z-axis). (iii) Residuals for datapoints used to generate heatmap.

### Example 2

### EIF4E-BP1 Fusion Protein

This technology is based on the 5′ Cap binding biology of EIF4E-BP1, which represses translation. To adapt this protein to the specific application described herein, amino acid residues known to be regulated by cellular kinases were mutated, to make its regulation constitutive. Experiments were performed with nuclease dead Cas9 (dCas9), with protein effectors fused to the C-terminus. Any messenger RNA of interest can be targeted, given the selection of an appropriate gRNA spacer sequence, which is specific to each CRISPR-Cas system.

An exemplary system is composed of a nuclease-dead Cas9 (dCas9) protein fused to a modified EIF4E-BP1 (FIG. 2), which can enhance or repress translation, respectively. These dCas9 fusion proteins bind a single guide RNA (sgRNA) driven by a U6 polymerase III promoter, and may co-bind an antisense synthetic oligonucleotide composed alternating 2′OMe RNA and DNA bases (PAMmer). Together, these components form an RCas9-RNA recognition complex that binds messenger RNA.

FIG. 2 depicts the anticipated mechanism of this system. Without being bound by theory, dCas9 fused to a modified EIF4E-BP1. The schematic shows dCas9-EIF4E-BP1 targeting the 3′ UTR of a representative target transcript. Modified EIF4E-BP1 facilitates transcript mRNA circularization, and prevents the disengagement of EIF4E-BP1 from EIF4E. Constitutive binding prevents the recruitment of EIF4G and ribosomal pre-initiation complexes.

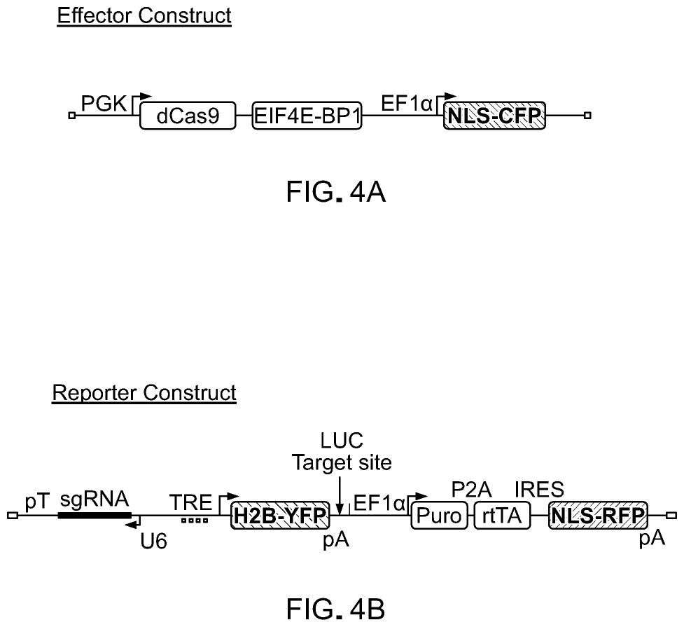

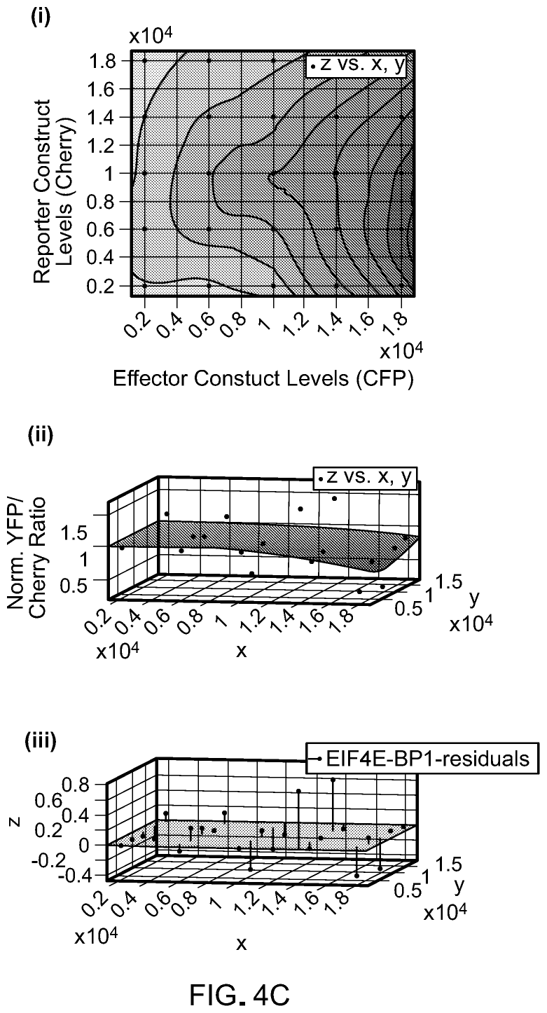

DNA constructs for Effector and Reporter constructs used for characterization studies were prepared as shown in FIG. 4A and 4B. Cas9-EIF4E-BP1 expression level was correlated to a co-expressed CFP fluorophore on the Effector. YFP and RFP were coexpressed from different promoters on the Reporter. However, only YFP messenger RNA carries a target site (LUC target site) that is complementary to the spacer of the single guide RNA (sgRNA).

Results of these experiments are shown in FIG. 4C: (i) Heatmap showing how the fold change in YFP/RFP ratio relate to Reporter (x-axis) and Effector (y-axis) DNA construct levels. Datapoints used for the heatmap represent the average fluorescence of single cells that fall within defined bins. (ii) Same data as presented in (i), but with YFP/RFP ratio plotted as third variable (z-axis). (iii) Residuals for datapoints used to generate heatmap.

### Example 3

### UBAP2L Fusion Protein

This example is based on a screen that implicated the ubiquitin-associated protein 2-like (UBAP2L) as a previously unknown RNA binding protein (RBP) that enhances translation. Experiments were performed with a RNA-targeting Cas9 (rCas9) with UBAP2L fused to the C-terminus. Any messenger RNA of interest can be targeted with this system, given the selection of an appropriate mRNA targeting spacer sequence, which s specific to each CRISPR-Cas system.

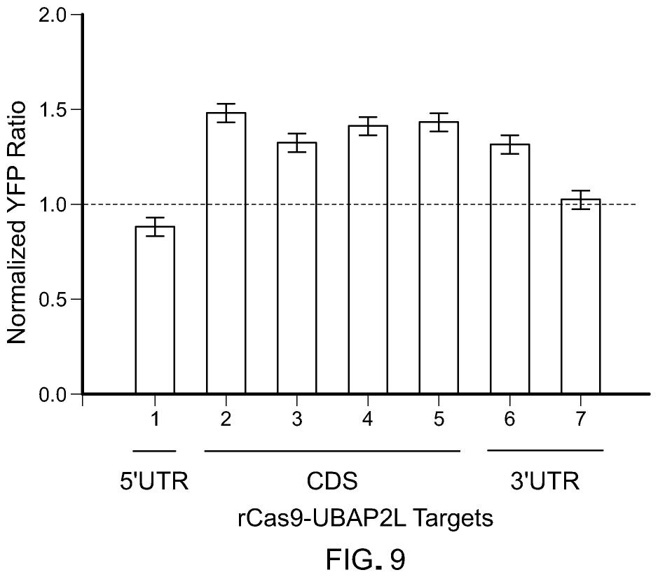

An exemplary system is composed of a RNA-targeting Cas9 (rCas9) fused to UBAP2L, which can enhance translation (FIG. 8). HEK293T cells lines expressing a Cas9-UBAP2L fusion or Cas9 only were derived via transposase-mediated piggyback genomic integration of a plasmid construct with an rCas9-UBAP2L or rCas9 expression cassette. A second construct was then transfected containing a reporter that stably expresses RFP transcripts not regulated by Cas9, a guide RNA, and tetracycline-inducible YFP transcripts with the guide RNA target sequences. Seven different guide RNAs were designed, targeting different locations within the YFP transcripts, and a non-targeting guide RNA. Post-transcriptional regulation was measured as changes in the normalized YFP/RFP fluorescence ratio using analytical flow cytometry. Due to the random nature of piggyback-mediated integration in terms of construct integration sites and numbers, regulation for various rCas9 construct levels (CFP) and reporter construct levels (RFP) were quantified across thousands of data points (cells). The extent of the effect of UBAP2L on YFP reporter expression was observed to be dependent on UBAP2L directed targeting to sites within the coding region (FIG. 9).

### Example 4

### Fusion RNAs

This example relates to a fusion RNA platform that is capable of enhancing the translation of a specific messenger RNA in cells. This technology depends on the ability of CRISPR-Cas systems to bind target messenger RNA via a single stranded guide, to which a ribonucleic acid sequence is fused that recruits translational pre-initiation complexes to the bound messenger RNA. This technology can thus initiate translation in trans.

This technology is built on the RNA targeting abilities of CRISPR-Cas systems, which uses a single stranded guide RNA to provide a simple and rapidly programmable system for regulating messenger RNA molecules in cells. CRISPR-Cas systems also have neutral effects on messenger RNA stability, which makes any measured change to gene expression a function of the nucleic acid effector fused to the guide RNA. Due to its highly encodable nature, as well as its adaptability to multiple CRISPR/Cas systems, the exemplary fusion RNA platform promises high utility and versatility when compared to other methods.

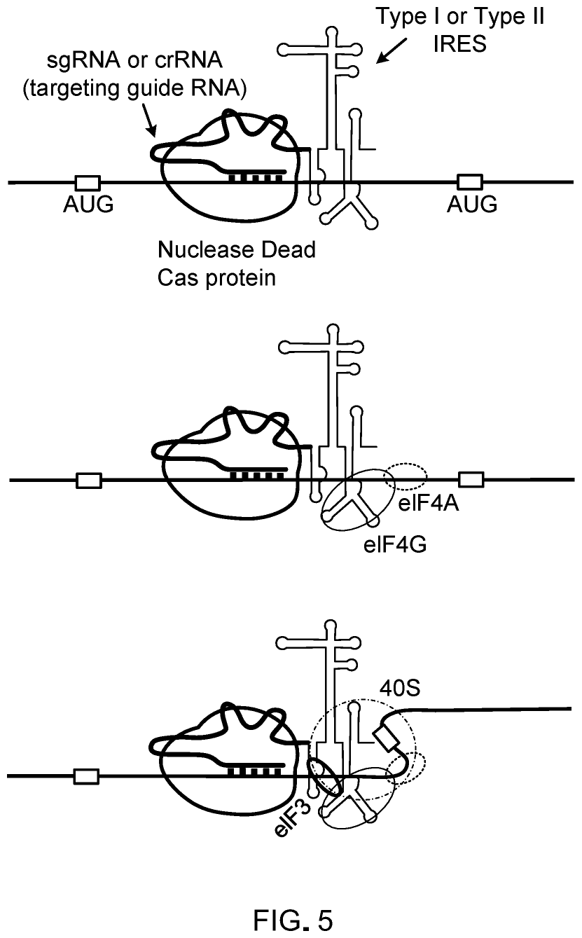

A fusion RNA was designed comprising a single stranded RNA guide (sgRNA) or a single stranded CRISPR RNA (crRNA) fused to a ribonucleic acid sequence based on Type I or Type II viral internal ribosome entry sequences (IRES). These modified sgRNA and crRNA are bound by nuclease-dead Cas9 (dCas9) protein and nuclease-dead Cas13b (dCas13b), respectively. Messenger RNA target specificity is conferred by a suitable spacer sequence, which is present at the 5′ end of sgRNA and crRNA. When the fusion RNA is expressed in cells, it binds to a target messenger RNA specifically. Fused ribonucleic acid sequence effectors then recruit pre-initiation complexes to the bound messenger RNA to promote protein translation as shown in FIG. 5.

Exemplary characterization was carried out using ribonucleic acid sequences derived from Type II Encephalomyocarditis Virus (EMCV-IRES). However, this technology is not limited to a particular type of IRES and may comprise any ribonucleic acid sequence that comprises the functional abilities and/or structural properties of an IRES.

For fusion RNA systems based on dCas9, an antisense synthetic oligonucleotide composed of alternating 2′OMe RNA and DNA bases (PAMmer) may also be provided. To simplify the delivery strategy, however, preliminary experiments involving dCas9 were performed without PAMmer. Without being bound by theory, it is thought that a PAMmer likely increases binding affinity of dCas9 to RNA in vivo as well as in vitro, but is has been found that it is not absolutely required for RNA targeting. Preliminary experiments were performed in the absence of a PAMmer. PAMmer is not required for systems based on dCas13b.

Fusion RNA systems were prepared with sgRNA or crRNA fused to PV-IRES, FMDV-IRES or EMCV-IRES. In this example, no specific modification was made to dCas9 or dCas13b except for the inclusion of a nuclear export sequence.

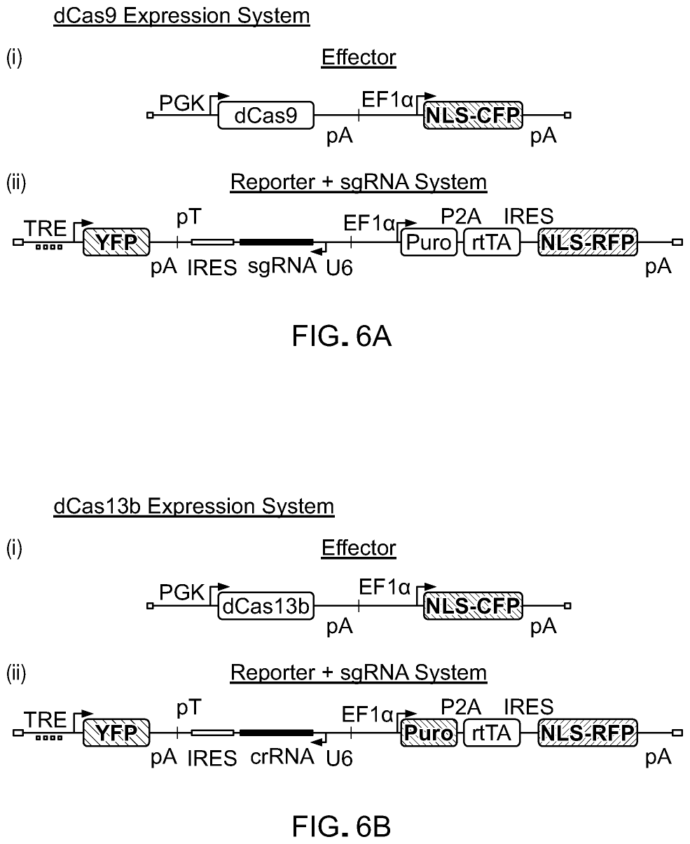

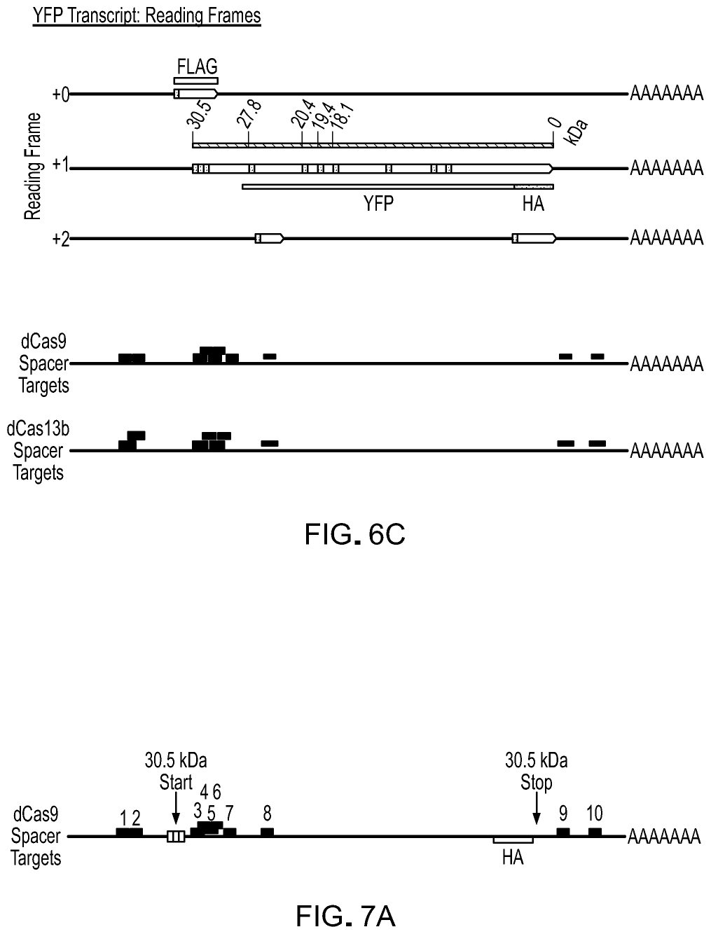

To quantify regulation by the fusion RNAs, a dual-fluorescence assay based on yellow fluorescent protein (YFP) and red fluorescent protein (RFP) expression was developed (FIG. 6A and FIG. 6B). Spacer sequences were designed to target the fusion RNA to YFP mRNA and regulate YFP expression (FIG. 6C). In contrast, RFP mRNA remains unbound, thus allowing RFP fluorescence and protein levels to serve as a transfection control. An HA-tag was appended to the C-terminus of YFP, which can be used to assay regulation of different YFP translation reading frames as a result of initiation at alternative start codons. Different YFP isoforms can be distinguished via Western blot. Changes in overall post-transcriptional regulation can also be represented as changes in the YFP to RFP fluorescence ratio.

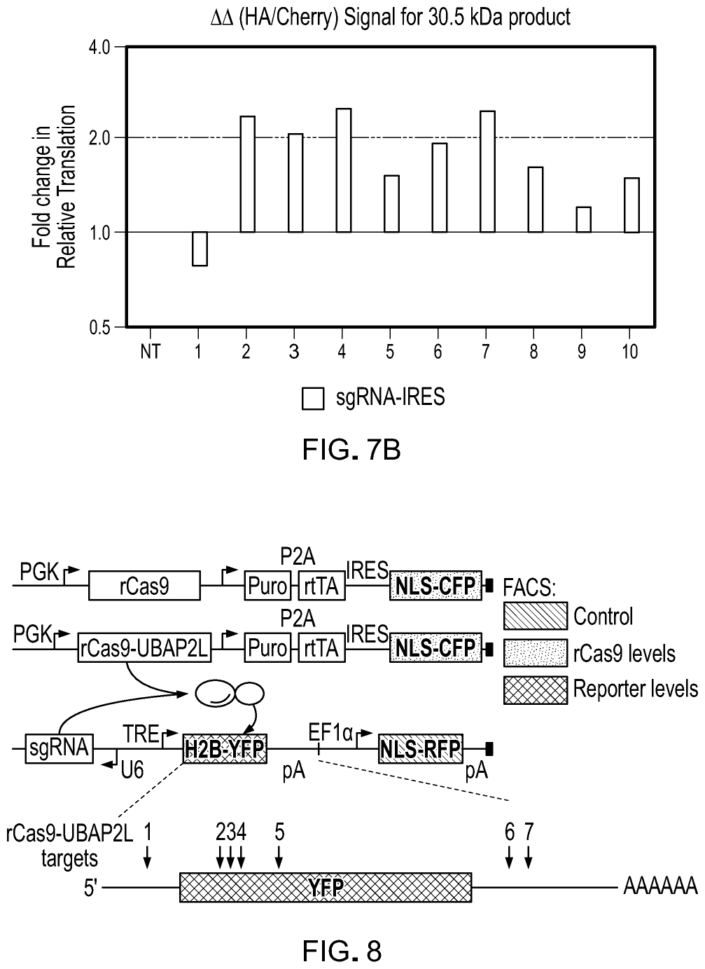

As shown in FIGS. 7A-7B, regulation by dCas9 and dCas13b fusion RNAs that use EMCV-IRES successfully mediate an enhancement in protein translation (FIG. 7A and FIG. 7B).

### REFERENCES

All references disclosed herein and throughout the disclosure are incorporated by reference in their entirety.
1. Cooke et al. 2011. “Targeted translational regulation using the PUF protein family scaffold” PNAS 108(38): 15870-15875.
2. Cao et al. 2015. “A universal strategy for regulating mRNA translation in prokaryotic and eukaryotic cells” NARS 43(8): 4353-4362.
3. WO/2015/089277
4. WO/2016/183402

1. A composition comprising one or more polynucleotides encoding:
(i) a guide nucleotide sequence-programmable RNA binding protein; and
(ii) a translation modifier protein.

2. The composition of claim 1, wherein the guide nucleotide sequence-programmable RNA binding protein comprises at least one of Cas9, modified Cas9, Cas13a, Cas13b, CasRX/Cas13d, CasM and a biological equivalent of each thereof.

3. The composition of claim 2, wherein the guide nucleotide sequence-programmable RNA binding protein comprises at least one of Steptococcus pyogenes Cas9 (spCas9), Staphylococcus aureus Cas9 (saCas9), Francisella novicida Cas9 (FnCas9), Neisseria meningitidis Cas9 (nmCas9), Streptococcus thermophilus 1 Cas9 (St1Cas9), Streptococcus thermophilus 3 Cas9 (St3Cas9), and Brevibacillus laterosporus Cas9 (BlatCas9).

4. The composition of claim 2, wherein the guide nucleotide sequence-programmable RNA binding protein is nuclease inactive.

5. The composition of claim 1, wherein the translation modifier protein is at least one of eukaryotic translation initiation factor 4E (EIF4E) (SEQ ID NO: 52-59), eukaryotic translation initiation factor 4E-binding protein (EIF4E-BP1) (SEQ ID NO: 61-62), ubiquitin-associated protein 2-like (UBAP2L) (SEQ ID NO: 64-71), and a biological equivalent of each thereof.

6. The composition of claim 5, wherein the translation modifier protein is encoded by a polynucleotide having a sequence comprising all or part of at least one of SEQ ID NO: 52-55, SEQ ID NO: 61, SEQ ID NO: 64-67, SEQ ID NO: 94-193, SEQ ID NO: 285, SEQ ID NO: 320-348, and a biological equivalent of each thereof.

7. The composition of claim 5, wherein the translation modifier protein has an amino acid sequence comprising all or part of at least one of SEQ ID NO: 56-59, SEQ ID NO: 62, SEQ ID NO: 68-71 and a biological equivalent of each thereof.

8. The composition of claim 1, further comprising a linker.

9. The composition of claim 8, wherein the linker is a peptide linker.

10. (canceled)

11. The composition of claim 8, wherein the linker is a non-peptide linker.

12. (canceled)

13. The composition of claim 1, wherein the guide nucleotide sequence-programmable RNA binding protein is bound to a guide RNA (gRNA), a crisprRNA (crRNA), or a trans-activating crRNA (tracrRNA).

14. The composition of claim 1, wherein one or more kinase phosphorylation domains of the translation modifier protein is mutated.

15. The composition of claim 1, further comprising a vector.

16. The vector of claim 15, wherein the vector is an adenoviral vector, an adeno-associated viral vector, or a lentiviral vector.

17. (canceled)

18. (canceled)

19. (canceled)

20. (canceled)

21. A fusion protein comprising:
(i) a guide nucleotide sequence-programmable RNA binding protein; and
(ii) a translation modifier protein.

22. A system for post-transcriptional gene regulation, the system comprising:
(i) a fusion protein according to claim 21; and
(ii) a gRNA; or
(iii) a crRNA and a tracrRNA;
wherein the gRNA or the crRNA comprises a sequence complementary to a target mRNA.

23. (canceled)

24. A fusion RNA comprising:
(i) a guide nucleotide sequence-programmable RNA; and
(ii) one or more internal ribosome entry sites (IRES).

25. The fusion RNA of claim 24, wherein the guide nucleotide sequence-programmable RNA is a guide RNA (gRNA) or a crisprRNA (crRNA).

26. The fusion RNA of claim 24, wherein the guide nucleotide sequence-programmable RNA is derived from a guide RNA scaffold from Steptococcus pyogenes, Staphylococcus aureus, Francisella novicida, Neisseria meningitidis, Streptococcus thermophilus, or Brevibacillus laterosporus.

27. The fusion RNA of claim 24, wherein the IRES is at least one of a Poliovirus IRES, Rhinovirus IRES, Encephalomyocarditis virus IRES (EMCV-IRES), Picornavirus IRES, Foot-and-mouth disease virus IRES (FMDV-IRES), Aphthovirus IRES, Kaposi's sarcoma-associated herpesvirus IRES (KSHV-IRES), Hepatitis A IRES, Hepatitis C IRES, Classical swine fever virus IRES, Pestivirus IRES, Bovine viral diarrhea virus IRES, Friend murine leukemia IRES, Moloney murine leukemia IRES (MMLV-IRES), Rous sarcoma virus IRES, Human immunodeficiency virus IRES (HIV-IRES), Plautia stali intestine virus IRES, Cripavirus IRES, Cricket paralysis virus IRES, Triatoma virus IRES, Rhopalosiphum padi virus IRES, Marek's disease virus IRES, Fibroblast growth factor (FGF-1 IRES and FGF-2 IRES), Platelet-derived growth factor B (PDGF/c-sis IRES), Vascular endothelial growth factor (VEGF IRES), and an Insulin-like growth factor 2 (IGF-II IRES).

28. (canceled)
